(19) United States
(12) Patent Application Publication
(10) Pub. No.: US 2024/0325443 A1
(43) Pub. Date: Oct. 3, 2024

(54) CD3-EXPRESSING NATURAL KILLER CELLS WITH ENHANCED FUNCTION FOR ADOPTIVE IMMUNOTHERAPY

(71) Applicant: BOARD OF REGENTS, THE UNIVERSITY OF TEXAS SYSTEM, Austin, TX (US)

(72) Inventors: Enli LIU, Houston, TX (US); Katy REZVANI, Houston, TX (US); Rafet BASAR, Houston, TX (US); Bin LIU, Houston, TX (US); David MARIN COSTA, Houston, TX (US)

(73) Assignee: BOARD OF REGENTS, THE UNIVERSITY OF TEXAS SYSTEM, Austin, TX (US)

(21) Appl. No.: 18/290,898

(22) PCT Filed: Jul. 22, 2022
(86) PCT No.: PCT/US2022/074062

§ 371 Date: Jan. 22, 2024

Related U.S. Application Data
(60) Provisional application No. 63/344,931, filed on May 23, 2022.
(60) Provisional application No. 63/310,526, filed on Feb. 15, 2022.
(60) Provisional application No. 63/225,281, filed on Jul. 23, 2021.

(51) Int. Cl. A61K 35/17; A61K 39/00; A61K 39/395; A61P 35/00; C07K 14/54; C07K 14/705; C07K 14/725; C07K 16/28; C07K 16/30; C12N 5/0783

(52) U.S. Cl. CPC A61K 35/17; A61K 39/39558; A61K 39/4613; A61K 39/4632; A61K 39/4633; A61K 39/464412; A61K 39/464488; A61K 39/464489; A61P 35/00; C07K 14/5443; C07K 14/7051; C07K 14/70521; C07K 16/2809; C07K 16/30; C12N 5/0646; C07K 2317/31; C07K 2317/73; C07K 2319/02; C07K 2319/03; C12N 2510/00

(57) ABSTRACT

Embodiments of the disclosure include methods and compositions in which NK cells are modified by the hand of man to express the T-cell receptor and CD3 co-receptor on NK cells that do not naturally express them. Such modified NK cells work effectively with bispecific or multi-specific antibodies that are tailored to comprise anti-CD3 antibodies that bind the modified NK cells, thereby triggering signaling, activation, and cytotoxicity of target cells to which the antibodies also bind. Thus, the NK cells are specifically configured to be able to work effectively with Bispecific NK cell engagers (BiKEs) as well as Bispecific T cell Engagers (BiTEs).

This application claims priority to U.S. Provisional Patent Application Ser. No. 63/225,281, filed Jul. 23, 2021, and also claims priority to U.S. Provisional Patent Application Ser. No. 63/310,526, filed Feb. 15, 2022, and also claims priority to U.S. Provisional Patent Application Ser. No. 63/344,931, filed May 23, 2022, each of which are incorporated by reference herein in their entirety.

### I. TECHNICAL FIELD

This disclosure relates at least to the fields of immunology, cell biology, molecular biology, and medicine, including at least cancer medicine.

### II. BACKGROUND

Natural killer (NK) cells have been studied as potential anti-tumor effectors, yet a number of barriers limit their therapeutic exploitation, mainly related to their lack of antigen specificity. One approach to overcome this is to transduce NK cells with a chimeric antigen receptor (CAR) or an engineered T-cell receptor (TCR) to target a desired antigen. In T cells, one can utilize a bispecific or multi-specific antibody, such as a bispecific T cell engager (BiTE) that binds CD3 on the surface of T cells and that also binds an antigen on the surface of cancer cells. CD3 is composed of four distinct chains, and in mammals, the complex contains a CD3γ chain, a CD3δ chain, and two CD3ε chains. These chains associate with the T-cell receptor (TCR) and the ζ-chain (zeta-chain) to generate an activation signal in T lymphocytes. However, NK cells do not naturally express the CD3 receptor complex or TCRs.

The present disclosure satisfies a long-felt need in the art to improve upon immunotherapies including those that utilize NK cells.

### BRIEF SUMMARY

Embodiments of the disclosure include methods and compositions for treatment of an individual with cancer using adoptive cell therapy. In specific embodiments, the individual is provided a therapeutically effective amount of a bipartite therapy that includes both modified NK cells and antibodies that are capable of being able to bind the NK cells to initiate signaling, activation, and killing of target cells. The disclosure concerns NK cells that have been modified to express multiple proteins that are not naturally expressed in NK cells and that work in conjunction together, including heterologous proteins on the surface of the NK cells that are naturally not present in NK cells.

In specific embodiments, NK cells are engineered to express one or more proteins from a CD3 co-receptor complex and optionally a TCR receptor complex, each normally present on the surface of T cells. Such engineering provides greater versatility for the NK cells to be utilized in conjunction with a variety of bispecific or multi-specific antibodies, including those that comprise an anti-CD3 antibody (e.g., an anti-CD3 scFv). In particular embodiments, the modified NK cells are administered to an individual in need thereof in conjunction with one or more bispecific or multi-specific antibodies each having one antibody that targets CD3 and one antibody that binds a desired antigen, such as a cancer antigen. As a result, in specific cases the NK cells expressing CD3 are able to bind the anti-CD3 antibody part of the bispecific or multi-specific antibody, and the antibody that binds a cancer antigen binds the cancer antigen on the surface of a cancer cell. Such a coordinated binding between the NK cells and the antibody results in activation of cytotoxicity against the target cancer antigen.

In particular embodiments, the present disclosure concerns modified NK cells that express the full or partial CD3 complex with or without TCRs, and in some cases individual CD3 chain(s) are heterologously linked to an NK-relevant signaling domain, all of which allows the modified NK cells to be utilized with a variety of bispecific antibodies.

Embodiments of the disclosure include compositions comprising NK cells modified to express part or all of a single chain or any combination of CD3δ, CD3ε, CD3γ, or CD3ζ. In some cases, the NK cells are modified to express the T-cell receptor (TCR) αβ chains or the TCR γδ chains. The NK cells may be modified to express part or all of CD3ζ, two of CD3ε, CD3δ, and CD3γ. In some cases, the NK cells are modified to express full length of CD3ζ, CD3ε, CD3δ, and/or CD3γ. In particular cases, any one or more of the CD3ζ, CD3ε, CD3δ, and CD3γ are heterologously linked to one or more intracellular signaling domains. The intracellular signaling domain may be selected from the group consisting of CD16, NKG2D, DAP10, DAP12, 2B4, 4-1BB, CD2, CD28 and a combination thereof. In some embodiments, an intracellular signaling domain is fused to CD3ζ. In some embodiments, an intracellular signaling domain is derived from DAP10. In some embodiments, an intracellular signaling domain is derived from CD28. In some embodiments, an intracellular signaling domain comprises a sequence derived from DAP10 and a sequence derived from CD28. In some embodiments, the intracellular signaling domain could also include other costimulatory signals relevant to NK cell function such as but not limited to, 2B4, DNA, 4-1BB, DAP12, NKG2D, etc. In specific embodiments, the composition further comprises one or more bispecific or multi-specific antibodies, wherein the bispecific or multi-specific antibody comprises an anti-CD3 antibody. The NK cells may express the antibody and/or are complexed with the antibody. In some embodiments, the TCR is directed to a cancer antigen or a viral antigen. In specific embodiments, the NK cells are derived from cord blood (CB), peripheral blood (PB), bone marrow, stem cells, or a mixture thereof. In some embodiments, the TCR is directed to an NY-ESO antigen. In some embodiments, the TCR is directed to a PRAME antigen. The NK cells may be pre-activated, such as with one or more cytokines, including IL-2, IL-7, IL-12, IL-15, IL-18, IL-21, or a combination thereof, for example. In some embodiments, the NK cells are expanded, such as in the presence of IL-2. In specific embodiments, the NK cells are modified to express one or more heterologous proteins, such as one or more engineered antigen receptors, one or more cytokines, one or more homing receptors, and/or one or more chemokine receptors. In specific cases, the engineered antigen receptor is a chimeric antigen receptor and/or engineered T cell receptor. In some cases, the heterologous protein is a cytokine, such as one selected from the group consisting of IL-15, IL-12, IL-2, IL-18, IL-21, IL-23, GMCSF, or a combination thereof. The cytokine may be membrane-bound, and the membrane-bound cytokine may comprise a transmembrane domain from CD8, CD28, CD27, B7H3, IgG1, IgG4, CD4, DAP10, or DAP12. In specific cases, the NK cell expresses a chimeric antigen receptor and a cytokine. In some cases, the bispecific antibody comprises an antibody that targets a cancer antigen.

Embodiments of the disclosure include compositions comprising a complex, comprising: (1) NK cells modified to express part or all of the CD3 receptor complex and optionally modified to express the T-cell receptor (TCR) αβ chains or the TCR γδ chains; and (2) a bispecific or multi-specific antibody, wherein the bispecific or multi-specific antibody comprises an anti-CD3 antibody that is bound to CD3 on the NK cells. In specific embodiments, the complex is housed in a pharmaceutically acceptable excipient. The complex may be housed in a delivery device.

In particular embodiments, there is a method of treating cancer in an individual, comprising the step of administering to the individual a therapeutically effective amount of any one of the compositions encompassed herein. In some embodiments, the NK cells and the antibody are administered to the individual at the same time. The NK cells and the antibody may or may not be administered in the same formulation. The NK cells and the antibody may be pre-complexed prior to administration to the individual. In specific embodiments, the NK cells and the antibody are administered to the individual at different times. The NK cells and the antibody may be administered by infusion. In specific embodiments, the NK cells are autologous or allogeneic with respect to the individual.

Embodiments of the disclosure include methods of redirecting the specificity of NK cells against a cancer antigen for treatment of an individual with a bispecific or multi-specific anti-CD3 antibody, comprising the steps of administering to the individual the antibody and NK cells that express part or all of the CD3 receptor complex and that optionally express part or all of TCR αβ chains or the TCR γδ chains. In specific embodiments, the method further comprising the step of modifying NK cells to express part or all of the CD3 receptor complex. In specific embodiments, the method further comprises the step of modifying NK cells to express the TCR αβ chains or the TCR γδ chains. In some cases, the method further comprises the step of modifying the NK cells to express one or more heterologous proteins.

Other objects, features and advantages of the present invention will become apparent from the following detailed description. It should be understood, however, that the detailed description and the specific examples, while indicating specific embodiments of the invention, are given by way of illustration only, since various changes and modifications within the spirit and scope of the invention will become apparent to those skilled in the art from this detailed description.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIG. 1A illustrates various embodiments of NK cells engineered to express CD3, including for use with a variety of heterologous proteins, such as cytokines, bi-specific NK cell engagers, and engineered antigen receptors (CAR and/or TCR). FIG. 1B illustrates NK cells accommodated for CD3 and TCR for optimal cancer immunotherapy. FIG. 1C illustrates examples of single chimeric CD3 constructions.

FIG. 2A illustrates one example of an expression construct for CD3 receptor complex components for transduction or transfection of NK cells. FIG. 2B shows an example of a plasmid map for the representative expression construct.

FIG. 3 provides a table of various TCR/CD3 expression construct designs for NK-TCR engineering.

FIG. 4 shows CD3 expression at day 4 on engineered NK cells after transduction with one example of a CMV-directed TCR complex.

FIG. 5 demonstrates TCR expression at day 4 on engineered NK cells following CMV-directed TCR complex transduction.

FIG. 6 shows TCR/CD3 expression at day 6 on engineered NK cells after transduction of a CMV-directed TCR complex into the cells.

FIG. 7 demonstrates binding at different concentrations of one example of a CD3-CD19 BiTE on NK cells through the CD3/TCR complex on the NK cells.

FIG. 8 shows NK-TCR cytokine production of TNFα and CD107a after stimulation with plate-bound CD3 antibody.

FIG. 9 demonstrates phosphorylation of CD3z in NK TCR/CD3 cells after crosslinking CD3.

FIGS. 10A-10B show that pre-culturing CD3-CD19 BiTEs with TCR/CD3-expressing NK cells increased its killing activity against Raji cells. FIG. 10A represents a 1:1 Effector:Target ratio, and FIG. 10B represents a 1:5 Effector:Target ratio.

FIG. 11 provides a schematic overview of multiple retroviral transductions to generate NK cells expressing CD3, IL-15, and a TCR complex.

FIG. 12 shows expression of NY-ESO TCR on NK cells transduced with uTNK15. WT refers to wild type CD3 molecules with IL-15; A refers to CD3-CD28 with IL-15; B refers to CD3-DAP10 with IL-15; and C refers to CD3-CD28-Dap10 with IL-15.

FIG. 13 shows the number of TCR molecules per cell expressed on NK cells. WT refers to wild type CD3 molecules with IL-15; A refers to CD3-CD28 with IL-15; B refers to CD3-DAP10 with IL-15; and C refers to CD3-CD28-Dap10 with IL-15. Phycoerythrin Fluorescence Quantitation Kit (BD Biosciences) was used to determine the number of molecules of NY-ESO TCR on NK cells.

FIG. 14 shows expression of NY-ESO TCR on T cells.

FIG. 15 shows that NK cells transduced with NY-ESO TCR kill NY-ESO peptide-pulsed target cells in a dose-dependent manner. WT refers to wild type CD3 molecules with IL-15; A refers to CD3-CD28 with IL-15; B refers to CD3-DAP10 with IL-15; and C refers to CD3-CD28-Dap10 with IL-15.

FIG. 16 demonstrates endogenous NY-ESO expression on human tumor cell lines.

FIG. 17 demonstrates that NY-ESO TCR transduced T cells kill NY-ESO expressing tumor targets.

FIG. 18 provides results that NY-ESO TCR transduced NK cells kill NY-ESO expressing tumor targets even at low E:T ratios. WT refers to wild type CD3 molecules with IL-15; A refers to CD3-CD28 with IL-15; B refers to CD3-DAP10 with IL-15; and C refers to CD3-CD28-Dap10 with IL-15.

FIGS. 19A and 19B show that NY-ESO transduced NK cells have a similar phenotype (19A) and expression pattern (19B) to NT NK cells. WT refers to wild type CD3 molecules with IL-15; A refers to CD3-CD28 with IL-15; B refers to CD3-DAP10 with IL-15; and C refers to CD3-CD28-Dap10 with IL-15.

FIG. 20 provides a table representing the cellular composition of the expanded uTNK15 product. WT refers to wild type CD3 molecules with IL-15; A refers to CD3-CD28 with IL-15; B refers to CD3-DAP10 with IL-15; and C refers to CD3-CD28-Dap10 with IL-15.

FIG. 21A shows that NK cells can be successfully transduced with CD3 and TCR constant alpha-beta (TCRCab) (called TCR6 construct) and that the engineered NK cell can bind Blinatumumab (FIG. 21B) and selectively kill CD19+ lymphoma targets (FIG. 21C).

FIGS. 22A-22C shows the in vivo activity of effector cells (e.g., NK cells, or T cells) comprising NY-ESO targeted TCRs. FIG. 22A is a schematic outlining the experimental procedure performed. FIG. 22B displays bioluminescent imaging over time (day 1, day 7, day 14, and day 21) for the mice engrafted with U266B.1 cells transduced with FireFlyluciferase (FFluc) and treated with control, NY-ESO TCR NK cells, or NY-ESO TCR T cells (NK cells comprising WT, #A, or #B UT-NK15-NY ESO TCR constructs respectively; WT refers to wild type CD3 molecules with IL-15; #A refers to CD3-CD28 with IL-15; and #B refers to CD3-DAP10 with IL-15). FIG. 22C is a graphical quantification of the bioluminescence average radiance displayed in FIG. 22B. These results showed that effector cells comprising NY-ESO TCR constructs described herein robustly inhibited tumor growth in vivo.

FIGS. 23A-B shows the in vitro activity of effector cells (e.g., NK cells or T cells) comprising NY-ESO targeted TCRs and UT-NK15 constructs. FIG. 23A are images of spheroids formed by osteosarcoma tumor cell line Saos-2 stably transduced to express GFP that were used to test the activity of NY-ESO1-specific TCR expressing NK and T cells cytotoxicity. FIG. 23B is a graph showing percentage of cytotoxicity (Y axis) for representative images after 3 days of co-culture. NK cells were co-transduced with NY-ESO-TCR, and the UT-NK15 signaling complex co-expressing different co-stimulatory molecules fused to the CD3ζ signaling chain or the TCR complex without IL-15. T cells were only transduced with NY-ESO TCR. Abbreviation in the graph: 28=CD3ζ fused to a CD28 co-stimulatory domain; 10=CD3ζ fused to a Dap10 co-stimulatory domain; 8=CD8 alpha/beta co-receptor as part of the NY ESO TCR construct; wo IL-15=the construct only contains CD3 zeta, epsilon, gamma and delta TCR complex without co-stimulation or IL-15.

FIGS. 24A-D shows the in vivo activity of effector cells (e.g., NK cells or T cells) comprising NY-ESO targeted TCRs and UT-NK15 constructs. FIG. 24A depicts a plan for an in vivo study to test the activity of different NY ESO TCR transduced NK and T cells. FIG. 24B depicts BLI imaging results of the test outlined and performed according to FIG. 24A, Mice were injected with U266 tumor cells, and three days later received T cells transduced with NY-ESO-specific TCR, or NK cells co-transduced with NY-ESO TCR and UT-NK15 with CD3 fused to CD28 (labelled as NY-ESO NK UT-NK15 CD28 or NY-ESO TCR UTNK-15 CD28 NK cells). The tumor alone group was used as control. FIG. 24C depicts region of interest average radiance intensity for the animals tested according to FIG. 24A and imaged in FIG. 24B. FIG. 24D is a graph depicting the cohort survival curves for the aforementioned animals.

FIG. 25 shows the in vivo activity of effector cells (e.g., NK cells) engineered to express NY ESO TCR and CD3 complex with or without IL-15 transgene comprised in the construct. NSG mice were irradiated (300 cGy) and the next day were injected with 500,000 U266 cells (HLA-A2 positive, NY-ESO-expressing myeloma cell line) via the tail vein. Three days later, mice received 5 million TCR transduced T or NK cells. Mice were monitored for tumor control by BLI imaging. NK cells were transduced with NY-ESO-specific TCR with or without expression of CD8 alpha/beta co-receptors, co-transduced with CD3 complex without IL-15 transgene or with UT-NK15 expressing CD3 fused to CD28 (UT-NK15 CD28) or CD3ζ fused to DAP10 (UT-NK15 DAP10) co-stimulatory molecules.

FIGS. 26A-C shows in vitro expression of Preferentially Expressed Antigen in Melanoma (PRAME) TCRs on effector cells (e.g., NK cells or T cells) and the in vitro activity of said cells. FIG. 26A shows the expression of both UT-NK15 (x-axis, CD3) and PRAME-specific TCRs (y-axis, TCR) in NK cells (TCR clones 46, 54, or DSK3 respectively), or the expression of PRAME-specific TCRs in T cells transduced with the same (TCR clones 46 or 54). FIG. 26B shows the in vitro cytotoxicity of NK cells expressing a PRAME-specific TCR against the U266 myeloma cell line. Incucyte live cell imaging was used to measure the cytotoxicity of T cells transduced with PRAME-specific TCR and NK cells transduced with UT-NK15 and PRAME-specific TCR against U266 myeloma cells. GFP-expressing U266 cells were co-cultured with PRAME-specific TCR expressing T cell or NK cells at 1:1 effector target ratio. A reduction in GFP expression indicated cell death. After 26 hours, a second round of 50,000 tumor cells was added (noted as “rechallenging”) to each well for the tumor rechallenge assay. Open symbols represent T cells, while closed symbols represent NK cells. NT=non-transduced. FIG. 26C shows the in vitro cytotoxicity of NK cells expressing a PRAME-specific TCR against the UA375 melanoma cell line. Incucyte live cell imaging was used to measure the cytotoxicity of T cells transduced with PRAME-specific TCR and NK cells transduced with UT-NK15 and PRAME-specific TCR (PRAME-specific TCR clone 46 (TCR-46), PRAME-specific TCR clone 54 (TCR-54), or PRAME-specific TCR clone DSK3 (DSK)) against UA375 melanoma cells. GFP-expressing UA375 cells were co-cultured with PRAME-expressing T cell or NK cells at 1:1 effector:target ratio. A reduction in GFP expression indicated cell death. After 26 hours, a second round of 50,000 tumor cells was added to each well for the tumor rechallenge assay. Open symbols represent T cells, while closed symbols represent NK cells. NT=non-transduced.

### DETAILED DESCRIPTION

In keeping with long-standing patent law convention, the words “a” and “an” when used in the present specification in concert with the word comprising, including the claims, denote “one or more.” Some embodiments of the disclosure may consist of or consist essentially of one or more elements, method steps, and/or methods of the disclosure. It is contemplated that any method or composition described herein can be implemented with respect to any other method or composition described herein and that different embodiments may be combined.

Throughout this specification, unless the context requires otherwise, the words “comprise”, “comprises” and “comprising” will be understood to imply the inclusion of a stated step or element or group of steps or elements but not the exclusion of any other step or element or group of steps or elements. By “consisting of” is meant including, and limited to, whatever follows the phrase “consisting of” Thus, the phrase “consisting of” indicates that the listed elements are required or mandatory, and that no other elements may be present. By “consisting essentially of” is meant including any elements listed after the phrase, and limited to other elements that do not interfere with or contribute to the activity or action specified in the disclosure for the listed elements. Thus, the phrase “consisting essentially of” indicates that the listed elements are required or mandatory, but that no other elements are optional and may or may not be present depending upon whether or not they affect the activity or action of the listed elements.

Reference throughout this specification to “one embodiment,” “an embodiment,” “a particular embodiment,” “a related embodiment,” “a certain embodiment,” “an additional embodiment,” or “a further embodiment” or combinations thereof means that a particular feature, structure or characteristic described in connection with the embodiment is included in at least one embodiment of the present invention. Thus, the appearances of the foregoing phrases in various places throughout this specification are not necessarily all referring to the same embodiment. Furthermore, the particular features, structures, or characteristics may be combined in any suitable manner in one or more embodiments.

As used herein, the terms “or” and “and/or” are utilized to describe multiple components in combination or exclusive of one another. For example, “x, y, and/or z” can refer to “x” alone, “y” alone, “z” alone, “x, y, and z,” “(x and y) or z,” “x or (y and z),” or “x or y or z.” It is specifically contemplated that x, y, or z may be specifically excluded from an embodiment.

Throughout this application, the term “about” is used according to its plain and ordinary meaning in the area of cell and molecular biology to indicate that a value includes the standard deviation of error for the device or method being employed to determine the value.

As used herein, the term “CD3 receptor complex” or “CD3 co-receptor complex” refers to the protein complex that in nature acts as a T cell co-receptor and is comprised of CD3ζ chain, CD3γ chain, a CD3δ chain, and two CD3ε chains (although in alternatives only one CD3ε chain is used).

The term “engineered” as used herein refers to an entity that is generated by the hand of man, including a cell, nucleic acid, polypeptide, vector, and so forth. In at least some cases, an engineered entity is synthetic and comprises elements that are not naturally present or configured in the manner in which it is utilized in the disclosure. In specific embodiments, a vector is engineered through recombinant nucleic acid technologies, and a cell is engineered through transfection or transduction of an engineered vector. Cells may be engineered to express heterologous proteins that are not naturally expressed by the cells, either because the heterologous proteins are recombinant or synthetic or because the cells do not naturally express the proteins.

The phrases “pharmaceutical or pharmacologically acceptable” refers to molecular entities and compositions that do not produce an adverse, allergic, or other untoward reaction when administered to an animal, such as a human, as appropriate. The preparation of a pharmaceutical composition comprising an antibody or additional active ingredient will be known to those of skill in the art in light of the present disclosure. Moreover, for animal (e.g., human) administration, it will be understood that preparations should meet sterility, pyrogenicity, general safety, and purity standards as required by FDA Office of Biological Standards.

As used herein, “pharmaceutically acceptable carrier” includes any and all aqueous solvents (e.g., water, alcoholic/aqueous solutions, saline solutions, parenteral vehicles, such as sodium chloride, Ringer's dextrose, etc.), non-aqueous solvents (e.g., propylene glycol, polyethylene glycol, vegetable oil, and injectable organic esters, such as ethyloleate), dispersion media, coatings, surfactants, antioxidants, preservatives (e.g., antibacterial or antifungal agents, anti-oxidants, chelating agents, and inert gases), isotonic agents, absorption delaying agents, salts, drugs, drug stabilizers, gels, binders, excipients, disintegration agents, lubricants, sweetening agents, flavoring agents, dyes, fluid and nutrient replenishers, such like materials and combinations thereof, as would be known to one of ordinary skill in the art. The pH and exact concentration of the various components in a pharmaceutical composition are adjusted according to well-known parameters.

The term “subject,” as used herein, generally refers to an individual having a that has or is suspected of having cancer. The subject can be any organism or animal subject that is an object of a method or material, including mammals, e.g., humans, laboratory animals (e.g., primates, rats, mice, rabbits), livestock (e.g., cows, sheep, goats, pigs, turkeys, and chickens), household pets (e.g., dogs, cats, and rodents), horses, and transgenic non-human animals. The subject can be a patient, e.g., have or be suspected of having a disease (that may be referred to as a medical condition), such as benign or malignant neoplasias, or cancer. The subject may being undergoing or having undergone treatment. The subject may be asymptomatic. The subject may be healthy individuals but that are desirous of prevention of cancer. The term “individual” may be used interchangeably, in at least some cases. The “subject” or “individual”, as used herein, may or may not be housed in a medical facility and may be treated as an outpatient of a medical facility. The individual may be receiving one or more medical compositions via the internet. An individual may comprise any age of a human or non-human animal and therefore includes both adult and juveniles (i.e., children) and infants and includes in utero individuals. It is not intended that the term connote a need for medical treatment, therefore, an individual may voluntarily or involuntarily be part of experimentation whether clinical or in support of basic science studies.

As used herein “treatment” or “treating,” includes any beneficial or desirable effect on the symptoms or pathology of a disease or pathological condition, and may include even minimal reductions in one or more measurable markers of the disease or condition being treated, e.g., cancer. Treatment can involve optionally either the reduction or amelioration of one or more symptoms of the disease or condition, or the delaying of the progression of the disease or condition. “Treatment” does not necessarily indicate complete eradication or cure of the disease or condition, or associated symptoms thereof. Treating may mean alleviation of at least one symptom of the disease or condition.

As used herein “TCR/CD3 complex” refers to a protein complex naturally found on the surface of T cells and that comprises T-cell receptor α and β chains and/or a T-cell receptor γ and δ chains, in addition to CD3ζ, CD3γ, CD3δ, and CD3ε chains.

### I. EMBODIMENTS OF THE DISCLOSURE

Natural killer (NK) cells are an emerging cellular immunotherapy for patients with malignant hematologic disease, as well as solid tumors. The present disclosure specifically relates to NK cells that have been modified to render the NK cells to have enhanced function as an immunotherapy compared to NK cells not so modified. The modifications allow for the NK cells to have greater versatility when used with other therapeutic agents and at least in some embodiments to have T cell-like activity by utilizing the CD3/TCR receptor complex. In specific embodiments, the NK cells are modified to express (i) either a single CD3 chain (CD3zeta, CD3 epsilon, CD3 delta, or CD3 gamma) or part or all of the human CD3 receptor complex (including any combination of CD3 delta, epsilon (one or two copies of epsilon), gamma, and zeta); or (ii) either a single CD3 chain or the human CD3 receptor complex (including any combination of CD3 delta, epsilon (one or two molecules), gamma, and zeta) as a full length protein or as a partial protein heterologously linked to one or more intracellular signaling domains); and (iii) the CD3 complex may or may not include the T-cell receptor (αβ or γδ). The disclosure concerns the use of CD3-expressing NK cells in the diagnosis and treatment of disease, including use of the cells in combination with bispecific or multi-specific antibodies in which one epitope of the antibody binds CD3 on the CD3-expressing NK cells). The CD3-expressing NK cells can either be pre-complexed ex vivo with the bi/multi-specific antibody to redirect their specificity toward the target antigen and/or combined in vivo. In diagnostic embodiments, labeled NK cells may be loaded with bispecific or multi-specific antibodies of any kind, including that comprise at least an anti-CD3 antibody, and the loaded, labeled NK cells may be monitored for trafficking to the site of the target antigen for which another antibody on the bispecific or multi-specific antibody binds.

### II. COMPOSITIONS OF THE DISCLOSURE

The disclosure concerns compositions that at least include modified NK cells that express at least parts of the TCR/CD3 complex. In some cases, the compositions also include bispecific or multi-specific antibodies, including in the same formulation, although in alternative embodiments the NK cells and antibodies are utilized as physically separate compositions.

### A. NK Cell TCR/CD3 Modifications

In particular embodiments, provided herein are compositions that comprise NK cells that have been modified by the hand of man to express part or all of the TCR receptor complex and part or all of the CD3 co-receptor complex. In specific embodiments, the NK cells are modified to include all components of the CD3 complex, including CD3ζ, CD3ε, CD3γ and CD3δ. Although in particular cases the full lengths of CD3ζ, CD3ε, CD3γ and CD3δ are utilized, including their extracellular domain, transmembrane domain, and intracellular domain, in alternative embodiments only part of one or more of CD3ζ, CD3ε, CD3γ and CD3δ are utilized each of which that may or may not be combined with one or more intracellular signaling domains such as CD16, NKG2D, DAP10, DAP12, CD28, 41BB, 2B4, CD27, OX40, or any combination thereof. The NK cells may also be modified to express the TCR receptor complex, although in alternative embodiments none of the TCR receptor complex components are utilized.

In certain embodiments, an amino acid sequence (e.g., a polypeptide) may comprise an amino acid represented by a single letter “X” or a three letter code “Xaa”. In some embodiments, the amino acid represented by “X” or “Xaa” is any naturally occurring amino acid, such as but not limited to, Arginine (Arg, R), Histidine (His, H), Lysine (Lys, K), Aspartic Acid (Asp, D), Glutamic Acid (Glu, E), Serine (Ser, S), Threonine (Thr, T), Asparagine (Asn, N), Glutamine (Gln, Q), Glycine (Gly, G), Proline (Pro, P), Cysteine (Cys, C), Alanine (Ala, A), Valine (Val, V), Isoleucine (Ile, I), Leucine (Leu, L), Methionine (Met, M), Phenylalanine (Phe, F), Tyrosine (Tyr, Y), or Tryptophan (Trp, W).

In some embodiments, the amino acid represented by “X” or “Xaa” in SEQ ID NO: 25 or SEQ ID NO: 88 is Arginine (Arg, R). In some embodiments, the amino acid represented by “X” or “Xaa” in SEQ ID NO: 25 or SEQ ID NO: 88 is Histidine (His, H). In some embodiments, the amino acid represented by “X” or “Xaa” in SEQ ID NO: 25 or SEQ ID NO: 88 is Lysine (Lys, K). In some embodiments, the amino acid represented by “X” or “Xaa” in SEQ ID NO: 25 or SEQ ID NO: 88 is Aspartic Acid (Asp, D). In some embodiments, the amino acid represented by “X” or “Xaa” in SEQ ID NO: 25 or SEQ ID NO: 88 is Glutamic Acid (Glu, E). In some embodiments, the amino acid represented by “X” or “Xaa” in SEQ ID NO: 25 or SEQ ID NO: 88 is Serine (Ser, S). In some embodiments, the amino acid represented by “X” or “Xaa” in SEQ ID NO: 25 or SEQ ID NO: 88 is Threonine (Thr, T). In some embodiments, the amino acid represented by “X” or “Xaa” in SEQ ID NO: 25 or SEQ ID NO: 88 is Asparagine (Asn, N). In some embodiments, the amino acid represented by “X” or “Xaa” in SEQ ID NO: 25 or SEQ ID NO: 88 is Glutamine (Gln, Q). In some embodiments, the amino acid represented by “X” or “Xaa” in SEQ ID NO: 25 or SEQ ID NO: 88 is Glycine (Gly, G). In some embodiments, the amino acid represented by “X” or “Xaa” in SEQ ID NO: 25 or SEQ ID NO: 88 is Proline (Pro, P). In some embodiments, the amino acid represented by “X” or “Xaa” in SEQ ID NO: 25 or SEQ ID NO: 88 is Cysteine (Cys, C). In some embodiments, the amino acid represented by “X” or “Xaa” in SEQ ID NO: 25 or SEQ ID NO: 88 is Alanine (Ala, A). In some embodiments, the amino acid represented by “X” or “Xaa” in SEQ ID NO: 25 or SEQ ID NO: 88 is Valine (Val, V). In some embodiments, the amino acid represented by “X” or “Xaa” in SEQ ID NO: 25 or SEQ ID NO: 88 is Isoleucine (Ile, I). In some embodiments, the amino acid represented by “X” or “Xaa” in SEQ ID NO: 25 or SEQ ID NO: 88 is Leucine (Leu, L). In some embodiments, the amino acid represented by “X” or “Xaa” in SEQ ID NO: 25 or SEQ ID NO: 88 in SEQ ID NO: 25 or SEQ ID NO: 88 is Methionine (Met, M). In some embodiments, the amino acid represented by “X” or “Xaa” in SEQ ID NO: 25 or SEQ ID NO: 88 is Phenylalanine (Phe, F). In some embodiments, the amino acid represented by “X” or “Xaa” in SEQ ID NO: 25 or SEQ ID NO: 88 is Tyrosine (Tyr, Y). In some embodiments, the amino acid represented by “X” or “Xaa” in SEQ ID NO: 25 or SEQ ID NO: 88 is Tryptophan (Trp, W).

In certain embodiments, particular sequences for any of the CD3 receptor components are utilized, including wildtype or mutants of the components so long as the CD3 receptor having the mutant is able to allow signaling through the CD3 complex leading to activation and killing of targets. In some cases, the following examples of sequences for CD3δ, CD3δ, CD3γ, and CD3ζ and are utilized for modification of the NK cells.

CD3 Epsilon (UniProtKB-P07766 (CD3E_HUMAN))
Signal Peptide
(SEQ ID NO: 1)
MQSGTHWRVLGLCLLSVGVW
Extracellular Domain
sp|P07766|23-126
(SEQ ID NO: 2)
DGNEEMGGITQTPYKVSISGTTVILTCPQYPGSEILWQHNDKNIGGDEDDKNIGSDEDHLSL
KEFSELEQSGYYVCYPRGSKPEDANFYLYLRARVCENCMEMD
Transmembrane Domain
sp|P07766|127-152
(SEQ ID NO: 3)
VMSVATIVIVDICITGGLLLLVYYWS
Intracellular Domain
sp|P07766|153-207
(SEQ ID NO: 4)
KNRKAKAKPVTRGAGAGGRQRGQNKERPPPVPNPDYEPIRKGQRDLYSGLNQRRI
An example of a Homo sapiens CD3e molecule (CD3E), mRNA is at NCBI
Reference Sequence: GENBANK ® Accession No. NM_000733.4
(SEQ ID NO: 5)
ATGCAGTCGGGCACTCACTGGAGAGTTCTGGGCCTCTGCCTCTTATCAGTTGGCGTTTGGGG
GCAAGATGGTAATGAAGAAATGGGTGGTATTACACAGACACCATATAAAGTCTCCATCTCTG
GAACCACAGTAATATTGACATGCCCTCAGTATCCTGGATCTGAAATACTATGGCAACACAAT
GATAAAAACATAGGCGGTGATGAGGATGATAAAAACATAGGCAGTGATGAGGATCACCTGTC
ACTGAAGGAATTTTCAGAATTGGAGCAAAGTGGTTATTATGTCTGCTACCCCAGAGGAAGCA
AACCAGAAGATGCGAACTTTTATCTCTACCTGAGGGCAAGAGTGTGTGAGAACTGCATGGAG
ATGGATGTGATGTCGGTGGCCACAATTGTCATAGTGGACATCTGCATCACTGGGGGCTTGCT
GCTGCTGGTTTACTACTGGAGCAAGAATAGAAAGGCCAAGGCCAAGCCTGTGACACGAGGAG
CGGGTGCTGGCGGCAGGCAAAGGGGACAAAACAAGGAGAGGCCACCACCTGTTCCCAACCCA
GACTATGAGCCCATCCGGAAAGGCCAGCGGGACCTGTATTCTGGCCTGAATCAGAGACGCAT
CTGA
Examples of respective nucleic acid and amino acid CD3 epsilon sequences in their
entirety are as follows (underlining refers to signal peptide sequence):
(SEQ ID NO: 37)
ATGCAGAGCGGCACCCACTGGAGAGTGCTGGGCCTGTGCCTGCTGAGCGTGGGCGTGTGGGG
CCAGGACGGCAACGAGGAGATGGGCGGCATCACCCAGACCCCCTACAAGGTGAGCATCAGCG
GCACCACCGTGATCCTGACCTGCCCCCAGTACCCCGGCAGCGAGATCCTGTGGCAGCACAAC
GACAAGAACATCGGCGGCGACGAGGACGACAAGAACATCGGCAGCGACGAGGACCACCTGAG
CCTGAAGGAGTTCAGCGAGCTGGAGCAGAGCGGCTACTACGTGTGCTACCCCAGAGGCAGCA
AGCCCGAGGACGCCAACTTCTACCTGTACCTGAGAGCCAGAGTGTGCGAGAACTGCATGGAG
ATGGACGTGATGAGCGTGGCCACCATCGTGATCGTGGACATCTGCATCACCGGCGGCCTGCT
GCTGCTGGTGTACTACTGGAGCAAGAACAGAAAGGCCAAGGCCAAGCCCGTGACCAGAGGCG
CCGGCGCCGGCGGCAGACAGAGAGGCCAGAACAAGGAGAGACCCCCCCCCGTGCCCAACCCC
GACTACGAGCCCATCAGAAAGGGCCAGAGAGACCTGTACAGCGGCCTGAACCAGAGAAGAAT
C
(SEQ ID NO: 38)
MQSGTHWRVLGLCLLSVGVWGQDGNEEMGGITQTPYKVSISGTTVILTCPQYPGSEILWQHN
DKNIGGDEDDKNIGSDEDHLSLKEFSELEQSGYYVCYPRGSKPEDANFYLYLRARVCENCME
MDVMSVATIVIVDICITGGLLLLVYYWSKNRKAKAKPVTRGAGAGGRQRGQNKERPPPVPNP
DYEPIRKGQRDLYSGLNQRRI
CD3 Delta (UniProtKB-P04234 (CD3D_HUMAN))
Signal Peptide
(SEQ ID NO: 6)
MEHSTFLSGLVLATLLSQVS
Extracellular Domain
sp|P04234|22-105
(SEQ ID NO: 7)
FKIPIEELEDRVFVNCNTSITWVEGTVGTLLSDITRLDLGKRILDPRGIYRCNGTDIYKDKE
STVQVHYRMCQSCVELDPATVA
Transmembrane Domain
sp|P04234|106-126
(SEQ ID NO: 8)
GIIVTDVIATLLLALGVFCFA
Intracellular Domain
sp|P04234|127-171
(SEQ ID NO: 9)
GHETGRLSGAADTQALLRNDQVYQPLRDRDDAQYSHLGGNWARNK
Homo sapiens CD3d molecule, delta (CD3-TCR complex), mRNA (cDNA clone
MGC:88324 IMAGE:30412345), complete cds GENBANK ®: BC070321.1
(SEQ ID NO: 10)
ATGGAACATAGCACGTTTCTCTCTGGCCTGGTACTGGCTACCCTTCTCTCGCAAGTGAGCCC
CTTCAAGATACCTATAGAGGAACTTGAGGACAGAGTGTTTGTGAATTGCAATACCAGCATCA
CATGGGTAGAGGGAACGGTGGGAACACTGCTCTCAGACATTACAAGACTGGACCTGGGAAAA
CGCATCCTGGACCCACGAGGAATATATAGGTGTAATGGGACAGATATATACAAGGACAAAGA
ATCTACCGTGCAAGTTCATTATCGAATGTGCCAGAGCTGTGTGGAGCTGGATCCAGCCACCG
TGGCTGGCATCATTGTCACTGATGTCATTGCCACTCTGCTCCTTGCTTTGGGAGTCTTCTGC
TTTGCTGGACATGAGACTGGAAGGCTGTCTGGGGCTGCCGACACACAAGCTCTGTTGAGGAA
TGACCAGGTCTATCAGCCCCTCCGAGATCGAGATGATGCTCAGTACAGCCACCTTGGAGGAA
ACTGGGCTCGGAACAAGTGA
Examples of respective nucleic acid and amino acid CD3 delta sequences in their
entirety are as follows (underlining refers to signal peptide sequence):
(SEQ ID NO: 35)
ATGGAGCACAGCACCTTCCTGAGCGGCCTGGTGCTGGCCACCCTGCTGAGCCAGGTGAGCCC
CTTCAAGATCCCCATCGAGGAGCTGGAGGACAGAGTGTTCGTGAACTGCAACACCAGCATCA
CCTGGGTGGAGGGCACCGTGGGCACCCTGCTGAGCGACATCACCAGACTGGACCTGGGCAAG
AGAATCCTGGACCCCAGAGGCATCTACAGATGCAACGGCACCGACATCTACAAGGACAAGGA
GAGCACCGTGCAGGTGCACTACAGAATGTGCCAGAGCTGCGTGGAGCTGGACCCCGCCACCG
TGGCCGGCATCATCGTGACCGACGTGATCGCCACCCTGCTGCTGGCCCTGGGCGTGTTCTGC
TTCGCCGGCCACGAGACCGGCAGACTGAGCGGCGCCGCCGACACCCAGGCCCTGCTGAGAAA
CGACCAGGTGTACCAGCCCCTGAGAGACAGAGACGACGCCCAGTACAGCCACCTGGGCGGCA
ACTGGGCCAGAAACAAG
(SEQ ID NO: 36)
MEHSTFLSGLVLATLLSQVSPFKIPIEELEDRVFVNCNTSITWVEGTVGTLLSDITRLDLGK
RILDPRGIYRCNGTDIYKDKESTVQVHYRMCQSCVELDPATVAGIIVTDVIATLLLALGVFC
FAGHETGRLSGAADTQALLRNDQVYQPLRDRDDAQYSHLGGNWARNK
CD3 Gamma (T-cell surface glycoprotein CD3 gamma chain Gene CD3G P09693)
Signal Peptide
(SEQ ID NO: 11)
MEQGKGLAVLILAIILLQGTLA
Extracellular Domain
sp|P09693|23-116
(SEQ ID NO: 12)
QSIKGNHLVKVYDYQEDGSVLLTCDAEAKNITWFKDGKMIGFLTEDKKKWNLGSNAKDPRGM
YQCKGSQNKSKPLQVYYRMCQNCIELNAATIS
Transmembrane Domain
sp|P09693|117-137
(SEQ ID NO: 13)
GFLFAEIVSIFVLAVGVYFIA
Intracellular Domain
sp|P09693|138-182
(SEQ ID NO: 14)
GQDGVRQSRASDKQTLLPNDQLYQPLKDREDDQYSHLQGNQLRRN
Homo sapiens CD3g molecule (CD3G), mRNA; NM_000073.3:81-629 Homo
sapiens CD3g molecule (CD3G), mRNA
(SEQ ID NO: 15)
ATGGAACAGGGGAAGGGCCTGGCTGTCCTCATCCTGGCTATCATTCTTCTTCAAGGTACTTT
GGCCCAGTCAATCAAAGGAAACCACTTGGTTAAGGTGTATGACTATCAAGAAGATGGTTCGG
TACTTCTGACTTGTGATGCAGAAGCCAAAAATATCACATGGTTTAAAGATGGGAAGATGATC
GGCTTCCTAACTGAAGATAAAAAAAAATGGAATCTGGGAAGTAATGCCAAGGACCCTCGAGG
GATGTATCAGTGTAAAGGATCACAGAACAAGTCAAAACCACTCCAAGTGTATTACAGAATGT
GTCAGAACTGCATTGAACTAAATGCAGCCACCATATCTGGCTTTCTCTTTGCTGAAATCGTC
AGCATTTTCGTCCTTGCTGTTGGGGTCTACTTCATTGCTGGACAGGATGGAGTTCGCCAGTC
GAGAGCTTCAGACAAGCAGACTCTGTTGCCCAATGACCAGCTCTACCAGCCCCTCAAGGATC
GAGAAGATGACCAGTACAGCCACCTTCAAGGAAACCAGTTGAGGAGGAATTGA
Examples of respective nucleic acid and amino acid CD3 gamma sequences in their
entirety are as follows (underlining refers to signal peptide sequence):
(SEQ ID NO: 33)
ATGGAACAGGGGAAGGGCCTGGCTGTCCTCATCCTGGCTATCATTCTTCTTCAAGGTACTTT
GGCCCAGTCAATCAAAGGAAACCACTTGGTTAAGGTGTATGACTATCAAGAAGATGGTTCGG
TACTTCTGACTTGTGATGCAGAAGCCAAAAATATCACATGGTTTAAAGATGGGAAGATGATC
GGCTTCCTAACTGAAGATAAAAAAAAATGGAATCTGGGAAGTAATGCCAAGGACCCTCGTGG
GATGTATCAGTGTAAAGGATCACAGAACAAGTCAAAACCACTCCAAGTGTATTACAGAATGT
GTCAGAACTGCATTGAACTAAATGCAGCCACCATATCTGGCTTTCTCTTTGCTGAAATCGTC
AGCATTTTCGTCCTTGCTGTTGGGGTCTACTTCATTGCTGGACAGGATGGAGTTCGCCAGTC
GAGAGCTTCAGACAAGCAGACTCTGTTGCCCAATGACCAGCTCTACCAGCCCCTCAAGGATC
GAGAAGATGACCAGTACAGCCACCTTCAAGGAAACCAGTTGAGGAGGAAT
(SEQ ID NO: 34)
MEQGKGLAVLILAIILLQGTLAQSIKGNHLVKVYDYQEDGSVLLTCDAEAKNITWFKDGKMI
GFLTEDKKKWNLGSNAKDPRGMYQCKGSQNKSKPLQVYYRMCQNCIELNAATISGFLFAEIV
SIFVLAVGVYFIAGQDGVRQSRASDKQTLLPNDQLYQPLKDREDDQYSHLQGNQLRRN
CD3 Zeta
Signal Peptide
sp|P20963|SP
(SEQ ID NO: 16)
MKWKALFTAAILQAQLPITEA
Extracellular Domain
sp|P20963|22-30 ECD
(SEQ ID NO: 17)
QSFGLLDPK
Transmembrane Domain
sp|P20963|31-51 tmd
(SEQ ID NO: 18)
LCYLLDGILFIYGVILTALFL
Intracellular Domain
sp|P20963|52-164 ICD
(SEQ ID NO: 19)
RVKFSRSADAPAYQQGQNQLYNELNLGRREEYDVLDKRRGRDPEMGGKPQRRKNPQEGLYNE
LQKDKMAEAYSEIGMKGERRRGKGHDGLYQGLSTATKDTYDALHMQALPPR
Examples of respective nucleic acid and amino acid CD3 zeta sequences in their
entirety are as follows (underlining refers to signal peptide sequence):
(SEQ ID NO: 31)
ATGAAGTGGAAGGCGCTTTTCACCGCGGCCATCCTGCAGGCACAGTTGCCGATTACAGAGGC
ACAGAGCTTTGGCCTGCTGGATCCCAAACTCTGCTACCTGCTGGATGGAATCCTCTTCATCT
ATGGTGTCATTCTCACTGCCTTGTTCCTGAGAGTGAAGTTCAGCAGGAGCGCAGACGCCCCC
GCGTACCAGCAGGGCCAGAACCAGCTCTATAACGAGCTCAATCTAGGACGAAGAGAGGAGTA
CGATGTTTTGGACAAGAGACGTGGCCGGGACCCTGAGATGGGGGGAAAGCCGCAGAGAAGGA
AGAACCCTCAGGAAGGCCTGTACAATGAACTGCAGAAAGATAAGATGGCGGAGGCCTACAGT
GAGATTGGGATGAAAGGCGAGCGCCGGAGGGGCAAGGGGCACGATGGCCTTTACCAGGGTCT
CAGTACAGCCACCAAGGACACCTACGACGCCCTTCACATGCAGGCCCTGCCCCCTCGC
(SEQ ID NO: 32)
MKWKALFTAAILQAQLPITEAQSFGLLDPKLCYLLDGILFIYGVILTALFLRVKESRSADAP
AYQQGQNQLYNELNLGRREEYDVLDKRRGRDPEMGGKPQRRKNPQEGLYNELQKDKMAEAYS
EIGMKGERRRGKGHDGLYQGLSTATKDTYDALHMQALPPR
Homo sapiens CD247 molecule (CD247; also referred to as CD3 Zeta), transcript
variant 1, mRNA
NCBI Reference Sequence: NM_198053.3
NM_198053.3:65-559 Homo sapiens CD247 molecule (CD247), transcript variant 1, mRNA
(SEQ ID NO: 20)
ATGAAGTGGAAGGCGCTTTTCACCGCGGCCATCCTGCAGGCACAGTTGCCGATTACAGAGGC
ACAGAGCTTTGGCCTGCTGGATCCCAAACTCTGCTACCTGCTGGATGGAATCCTCTTCATCT
ATGGTGTCATTCTCACTGCCTTGTTCCTGAGAGTGAAGTTCAGCAGGAGCGCAGACGCCCCC
GCGTACCAGCAGGGCCAGAACCAGCTCTATAACGAGCTCAATCTAGGACGAAGAGAGGAGTA
CGATGTTTTGGACAAGAGACGTGGCCGGGACCCTGAGATGGGGGGAAAGCCGCAGAGAAGGA
AGAACCCTCAGGAAGGCCTGTACAATGAACTGCAGAAAGATAAGATGGCGGAGGCCTACAGT
GAGATTGGGATGAAAGGCGAGCGCCGGAGGGGCAAGGGGCACGATGGCCTTTACCAGGGTCT
CAGTACAGCCACCAAGGACACCTACGACGCCCTTCACATGCAGGCCCTGCCCCCTCGCTAA

In specific embodiments, the NK cells are modified to express one of more of the TCRα chain, the TCRβ chain, the TCRγ chain, and the TCR6 chain, and any combination thereof may be utilized. In a specific case, the NK cells are modified to express the T-cell receptor (TCR) αβ chains or the TCR γδ chains. In certain cases, the NK cells are modified to express part or all of only the constant region of one of more of the TCRα chain, the TCRβ chain, the TCRγ chain, and the TCR6 chain. The NK cells may be modified to express part or all of only the constant region of the T-cell receptor (TCR) αβ chains or the TCR γδ chains. In cases wherein part of the constant region is utilized, the part of the constant region may be at least 50, 75, 100, 125, 150, 175, 200, 225, 250, 275, 300, 325, 350, 375, or 400 amino acids, including contiguous amino acids of any constant region. The part of the constant region may comprise at least 50, 55, 60, 65, 70, 75, 80, 85, 90, 91, 92, 93, 94, 95, 96, 97, 98, or 99% of the amino acids of a constant region, including contiguous amino acids of a constant region.

In specific cases, any sequences encompassed herein are utilized to modify the NK cells, although in other cases sequences that are related to these in identity are utilized. For example, related sequences that are at least 80, 85, 90, 95, 96, 97, 98, 99% identical to any sequence encompassed herein may be utilized in the disclosure.

Particular constructs for the expression of various TCR/CD3 proteins in the NK cells may be utilized, and in a variety of configurations. In specific cases, the NK cells may be transduced or transfected with one or more vectors to express any of the various proteins encompassed herein, including at least any one or more components of the TCR/CD3 complex. In specific cases, the one or more vectors themselves may or may not be multicistronic by being able ultimately to produce more than one separate polypeptide. In cases wherein one or more multicistronic vectors are employed, they may utilize one or more internal ribosome entry sites (IRES) and/or one or more 2A self-cleaving peptide sites. In cases wherein one or more 2A sequences are utilized, the following may be used, where GSG is an optional linker:

T2A
(SEQ ID NO: 21)
(GSG) EGRGSLLTCGDVEENPGP
P2A
(SEQ ID NO: 22)
(GSG) ATNFSLLKQAGDVEENPGP
E2A
(SEQ ID NO: 23)
(GSG) QCTNYALLKLAGDVESNPGP
F2A
(SEQ ID NO: 24)
(GSG) VKQTLNFDLLKLAGDVESNPGP

In situations wherein multiple protein components are expressed from a multicistronic vector, the order in a 5′ to 3′ direction on the polynucleotide vector may be of any order, although in alternative cases they are present on the vector in a particular order. A multicistronic vector may express multiple components of the CD3 receptor complex and no other heterologous protein, or the multicistronic vector may express multiple components of the CD3 receptor complex and one or more other heterologous proteins. A multicistronic vector may express multiple components of the TCR receptor complex and no other heterologous protein, or the multicistronic vector may express multiple components of the TCR receptor complex and one or more other heterologous proteins. A multicistronic vector may or may not express one or more multiple components of the TCR receptor complex and one or more multiple components of the CD3 complex. In a specific embodiment, a multicistronic vector includes one or multiple components of the CD3 receptor complex and one or more heterologous proteins, such as a cytokine and an engineered antigen receptor, such as a CAR.

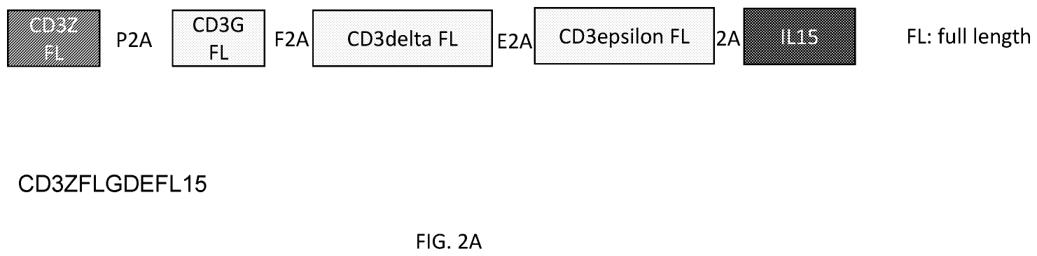

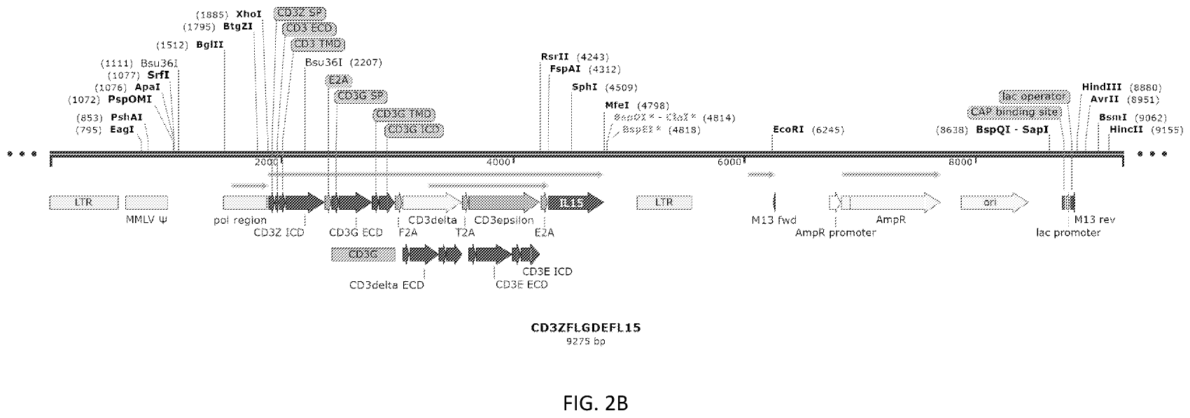

There is an example in FIG. 2A of a multicistronic vector in which full lengths of CD3ε, CD3δ, CD3γ, and CD3ζ are present and separated by the same or different 2A self-cleaving peptide sites. As further noted in the plasmid map of FIG. 2B, a multicistronic vector may include the signal peptide, extracellular domain, transmembrane domain, and intracellular domain of each of CD3ε, CD3δ, CD3γ, and CD3ζ.

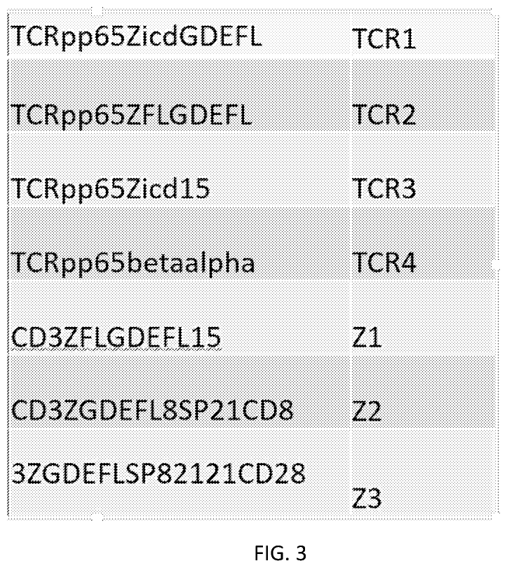

FIG. 3 provides a table showing examples of various TCR expression constructs for engineering of TCR-expressing NK cells. In particular embodiments of the disclosure, CD3 receptor components and TCR receptor components are expressed from different vectors in the NK cells. In any case, the vector(s) may express a TCR directed against a particular antigen, such as a cancer antigen or a viral antigen. The TCR may or may not comprise at least part of CD3ζ, including the intracellular domain of CD3ζ, in addition to the NK cells also expressing CD3ζ as a separate molecule from the TCR and as part of the CD3 receptor complex. Likewise, a CAR may or may not comprise at least part of CD3ζ, including the intracellular domain of CD3ζ, in addition to the NK cells also expressing CD3ζ as a separate molecule from the TCR and as part of the CD3 receptor complex.

In specific embodiments, a TCR of the modified NK cells is utilized not necessarily as a therapeutic aspect for the cells but as a structural support or scaffold to facilitate function or enhanced function of the CD3 receptor complex. That is, the TCR may be any TCR and may not be utilized for its ability to target a particularly desired antigen. In such cases, and as an example, a TCR that targets a viral antigen may be employed for NK cells that will be used for cancers that are not necessarily related to that particular virus. In other cases, the TCR is selected for the ability to target a particular cancer antigen. Examples of antigens to which the TCR may be directed are provided elsewhere herein.

In FIG. 3, the following examples of constructs are noted:

TCR1: refers to TCRpp65 (the TCR against the HLA-A2 restricted CMVpp65) linked to the intracellular CD3zeta domain and full length CD3 gamma, full length CD3 delta, and full length CD3 epsilon, and the construct may also be referred to as TCRpp65ZicdGDEFL that may comprise the following sequence:

(SEQ ID NO: 39)
MLEGVTQTPKFQVLKTGQSMTLQCAQDMNHEYMSWYRQDPGMGLR
LIHYSVGAGITDQGEVPNGYNVSRSTTEDFPLRLLSAAPSQTSVY
FCASSPVTGGIYGYTFGSGTRLTVVEDLNKVFPPEVAVFEPSEAE
ISHTQKATLVCLATGFFPDHVELSWWVNGKEVHSGVSTDPQPLKE
QPALNDSRYCLSSRLRVSATFWQNPRNHFRCQVQFYGLSENDEWT
QDRAKPVTQIVSAEAWGRADRVKFSRSADAPAYQQGQNQLYNELN
LGRREEYDVLDKRRGRDPEMGGKPQRRKNPQEGLYNELQKDKMAE
AYSEIGMKGERRRGKGHDGLYQGLSTATKDTYDALHMQALPPRAT
NFSLLKQAGDVEENPGPMILNVEQSPQSLHVQEGDSTNFTCSFPS
SNFYALHWYRWETAKSPEALFVMTLNGDEKKKGRISATLNTKEGY
SYLYIKGSQPEDSATYLCARNTGNQFYFGTGTSLTVIPNIQNPDP
AVYQLRDSKSSDKSVCLFTDFDSQTNVSQSKDSDAYITDKTVLDM
RSMDFKSNSAVAWSNKSDFACANAFNNSIIPEDTFFPSPESSRVK
FSRSADAPAYQQGQNQLYNELNLGRREEYDVLDKRRGRDPEMGGK
PQRRKNPQEGLYNELQKDKMAEAYSEIGMKGERRRGKGHDGLYQG
LSTATKDTYDALHMQALPPRQCTNYALLKLAGDVESNPGPMEQGK
GLAVLILAIILLQGTLAQSIKGNHLVKVYDYQEDGSVLLTCDAEA
KNITWFKDGKMIGFLTEDKKKWNLGSNAKDPRGMYQCKGSQNKSK
PLQVYYRMCQNCIELNAATISGFLFAEIVSIFVLAVGVYFIAGQD
GVRQSRASDKQTLLPNDQLYQPLKDREDDQYSHLQGNQLRRNVKQ
TLNFDLLKLAGDVESNPGPMEHSTFLSGLVLATLLSQVSPFKIPI
EELEDRVFVNCNTSITWVEGTVGTLLSDITRLDLGKRILDPRGIY
RCNGTDIYKDKESTVQVHYRMCQSCVELDPATVAGIIVTDVIATL
LLALGVFCFAGHETGRLSGAADTQALLRNDQVYQPLRDRDDAQYS
HLGGNWARNKEGRGSLLTCGDVEENPGPMQSGTHWRVLGLCLLSV
GVWGQDGNEEMGGITQTPYKVSISGTTVILTCPQYPGSEILWQHN
DKNIGGDEDDKNIGSDEDHLSLKEFSELEQSGYYVCYPRGSKPED
ANFYLYLRARVCENCMEMDVMSVATIVIVDICITGGLLLLVYYWS
KNRKAKAKPVTRGAGAGGRQRGQNKERPPPVPNPDYEPIRKGQRD
LYSGLNQRRIGPQCTNYALLKLAGDVESNPGPMRISKPHLRSISI
QCYLCLLLNSHELTEAGIHVFILGCFSAGLPKTEANWVNVISDLK
KIEDLIQSMHIDATLYTESDVHPSCKVTAMKCFLLELQVISLESG
DASIHDTVENLIILANNSLSSNGNVTESGCKECEELEEKNIKEFL
QSFVHIVQMFINTS*

In TCRpp65ZicdGDEFL, the corresponding component sequences are as follows, although these particular sequences or others may be utilized in this and/or other constructs:

TCRb-extracellular domain:
(SEQ ID NO: 40)
MLEGVTQTPKFQVLKTGQSMTLQCAQDMNHEYMSWYRQDPGMGLR
LIHYSVGAGITDQGEVPNGYNVSRSTTEDFPLRLLSAAPSQTSVY
FCASSPVTGGIYGYTFGSGTRLTVVEDLNKVEPPEVAVFEPSEAF
ISHTQKATLVCLATGFFPDHVELSWWVNGKEVHSGVSTDPQPLKE
QPALNDSRYCLSSRLRVSATFWQNPRNHFRCQVQFYGLSENDEWT
QDRAKPVTQIVSAEAWGRAD
(SEQ ID NO: 41)
ATGCTCGAGGGAGTGACCCAGACCCCCAAGTTCCAGGTGCTGAAG
ACCGGACAGAGCATGACCCTGCAGTGCGCCCAGGACATGAACCAC
GAGTACATGAGCTGGTACCGGCAGGACCCCGGAATGGGACTGCGG
CTGATCCACTACAGCGTGGGAGCCGGAATCACCGACCAGGGAGAG
GTGCCCAACGGATACAACGTGAGCCGGAGCACCACCGAGGACTTC
CCCCTGCGGCTGCTGAGCGCCGCCCCCAGCCAGACCAGCGTGTAC
TTCTGCGCCAGCAGCCCCGTGACCGGAGGAATCTACGGATACACC
TTCGGAAGCGGAACCCGGCTGACCGTGGTGGAGGACCTGAACAAG
GTGTTCCCCCCCGAGGTGGCCGTGTTCGAGCCCAGCGAGGCCGAG
ATCAGCCACACCCAGAAGGCCACCCTGGTGTGCCTGGCCACCGGA
TTCTTCCCCGACCACGTGGAGCTGAGCTGGTGGGTGAACGGAAAG
GAGGTGCACAGCGGAGTGAGCACCGACCCCCAGCCCCTGAAGGAG
CAGCCCGCCCTGAACGACAGCCGGTACTGCCTGAGCAGCCGGCTG
CGGGTGAGCGCCACCTTCTGGCAGAACCCCCGGAACCACTTCCGG
TGCCAGGTGCAGTTCTACGGACTGAGCGAGAACGACGAGTGGACC
CAGGACCGGGCCAAGCCCGTGACCCAGATCGTGAGCGCCGAGGCC
TGGGGACGGGCCGAC
CD3 zeta intracellular domain (Z-ICD):
(SEQ ID NO: 42)
RVKFSRSADAPAYQQGQNQLYNELNLGRREEYDVLDKRRGRDPEM
GGKPQRRKNPQEGLYNELQKDKMAEAYSEIGMKGERRRGKGHDGL
YQGLSTATKDTYDALHMQALPPRATNFSLLKQAGDVEENPGP
(where the P2A sequence is at the C-terminus)
(SEQ ID NO: 43)
AGAGTGAAGTTCAGCAGGAGCGCAGACGCCCCCGCGTACCAGCAG
GGCCAGAACCAGCTCTATAACGAGCTCAATCTAGGACGAAGAGAG
GAGTACGATGTTTTGGACAAGAGACGTGGCCGGGACCCTGAGATG
GGGGGAAAGCCGCAGAGAAGGAAGAACCCTCAGGAAGGCCTGTAC
AATGAACTGCAGAAAGATAAGATGGCGGAGGCCTACAGTGAGATT
GGGATGAAAGGCGAGCGCCGGAGGGGCAAGGGGCACGATGGCCTT
TACCAGGGTCTCAGTACAGCCACCAAGGACACCTACGACGCCCTT
CACATGCAGGCCCTGCCCCCTCGCgccaccaacttctccctgctg
aagcaggccggcgacgtggaggagaaccccggcccc
(where the lower case
sequence is the P2A sequence)
TCRa-extracellular domain:
(SEQ ID NO: 44)
MILNVEQSPQSLHVQEGDSTNFTCSFPSSNFYALHWYRWETAKSP
EALFVMTLNGDEKKKGRISATLNTKEGYSYLYIKGSQPEDSATYL
CARNTGNQFYFGTGTSLTVIPNIQNPDPAVYQLRDSKSSDKSVCL
FTDFDSQTNVSQSKDSDAYITDKTVLDMRSMDFKSNSAVAWSNKS
DFACANAFNNSIIPEDTFFPSPESS
(SEQ ID NO: 45)
ATGATCCTGAACGTGGAGCAGAGCCCCCAGAGCCTGCACGTGCAG
GAGGGAGACAGCACCAACTTCACCTGCAGCTTCCCCAGCAGCAAC
TTCTACGCCCTGCACTGGTACCGGTGGGAGACCGCCAAGAGCCCC
GAGGCCCTGTTCGTGATGACCCTGAACGGAGACGAGAAGAAGAAG
GGACGGATCAGCGCCACCCTGAACACCAAGGAGGGATACAGCTAC
CTGTACATCAAGGGAAGCCAGCCCGAGGACAGCGCCACCTACCTG
TGCGCCCGGAACACCGGAAACCAGTTCTACTTCGGAACCGGAACC
AGCCTGACCGTGATCCCCAACATCCAGAACCCCGACCCCGCCGTG
TACCAGCTGCGGGACAGCAAGAGCAGCGACAAGAGCGTGTGCCTG
TTCACCGACTTCGACAGCCAGACCAACGTGAGCCAGAGCAAGGAC
AGCGACGCCTACATCACCGACAAGACCGTGCTGGACATGCGGAGC
ATGGACTTCAAGAGCAACAGCGCCGTGGCCTGGAGCAACAAGAGC
GACTTCGCCTGCGCCAACGCCTTCAACAACAGCATCATCCCCGAG
GACACCTTCTTCCCCAGCCCCGAGAGCAGC
CD3 gamma delta epsilon (CD3GDE):
(SEQ ID NO: 46)
MEQGKGLAVLILAIILLQGTLAQSIKGNHLVKV
YDYQEDGSVLLTCDAEAKNITWEKDGKMIGFLTEDKKKWNLGSNA
KDPRGMYQCKGSQNKSKPLQVYYRMCQNCIELNAATISGELFAEI
VSIFVLAVGVYFIAGQDGVRQSRASDKQTLLPNDQLYQPLKDRED
DQYSHLQGNQLRRNVKQTLNFDLLKLAGDVESNPGPMEHSTFLSG
LVLATLLSQVSPFKIPIEELEDRVFVNCNTSITWVEGTVGTLLSD
ITRLDLGKRILDPRGIYRCNGTDIYKDKESTVQVHYRMCQSCVEL
DPATVAGIIVTDVIATLLLALGVFCFAGHETGRLSGAADTQALLR
NDQVYQPLRDRDDAQYSHLGGNWARNKEGRGSLLTCGDVEENPGP
MQSGTHWRVLGLCLLSVGVWGQDGNEEMGGITQTPYKVSISGTTV
ILTCPQYPGSEILWQHNDKNIGGDEDDKNIGSDEDHLSLKEFSEL
EQSGYYVCYPRGSKPEDANFYLYLRARVCENCMEMDVMSVATIVI
VDICITGGLLLLVYYWSKNRKAKAKPVTRGAGAGGRQRGQNKERP
PPVPNPDYEPIRKGQRDLYSGLNQRRIGPQCTNYALLKLAGDVES
NPGP
(where the E2A sequence is at the C-terminus)
(SEQ ID NO: 47)
ATGGAACAGGGGAAGGGCCTGGCTGTCCTCATCCTGGCTATCATT
CTTCTTCAAGGTACTTTGGCCCAGTCAATCAAAGGAAACCACTTG
GTTAAGGTGTATGACTATCAAGAAGATGGTTCGGTACTTCTGACT
TGTGATGCAGAAGCCAAAAATATCACATGGTTTAAAGATGGGAAG
ATGATCGGCTTCCTAACTGAAGATAAAAAAAAATGGAATCTGGGA
AGTAATGCCAAGGACCCTCGTGGGATGTATCAGTGTAAAGGATCA
CAGAACAAGTCAAAACCACTCCAAGTGTATTACAGAATGTGTCAG
AACTGCATTGAACTAAATGCAGCCACCATATCTGGCTTTCTCTTT
GCTGAAATCGTCAGCATTTTCGTCCTTGCTGTTGGGGTCTACTTC
ATTGCTGGACAGGATGGAGTTCGCCAGTCGAGAGCTTCAGACAAG
CAGACTCTGTTGCCCAATGACCAGCTCTACCAGCCCCTCAAGGAT
CGAGAAGATGACCAGTACAGCCACCTTCAAGGAAACCAGTTGAGG
AGGAATGTGAAGCAGACCCTGAACTTCGACCTGCTGAAGCTGGCC
GGCGACGTGGAGAGCAACCCCGGCCCCATGGAGCACAGCACCTTC
CTGAGCGGCCTGGTGCTGGCCACCCTGCTGAGCCAGGTGAGCCCC
TTCAAGATCCCCATCGAGGAGCTGGAGGACAGAGTGTTCGTGAAC
TGCAACACCAGCATCACCTGGGTGGAGGGCACCGTGGGCACCCTG
CTGAGCGACATCACCAGACTGGACCTGGGCAAGAGAATCCTGGAC
CCCAGAGGCATCTACAGATGCAACGGCACCGACATCTACAAGGAC
AAGGAGAGCACCGTGCAGGTGCACTACAGAATGTGCCAGAGCTGC
GTGGAGCTGGACCCCGCCACCGTGGCCGGCATCATCGTGACCGAC
GTGATCGCCACCCTGCTGCTGGCCCTGGGCGTGTTCTGCTTCGCC
GGCCACGAGACCGGCAGACTGAGCGGCGCCGCCGACACCCAGGCC
CTGCTGAGAAACGACCAGGTGTACCAGCCCCTGAGAGACAGAGAC
GACGCCCAGTACAGCCACCTGGGCGGCAACTGGGCCAGAAACAAG
GAGGGCAGAGGCAGCCTGCTGACCTGCGGCGACGTGGAGGAGAAC
CCCGGCCCCATGCAGAGCGGCACCCACTGGAGAGTGCTGGGCCTG
TGCCTGCTGAGCGTGGGCGTGTGGGGCCAGGACGGCAACGAGGAG
ATGGGCGGCATCACCCAGACCCCCTACAAGGTGAGCATCAGCGGC
ACCACCGTGATCCTGACCTGCCCCCAGTACCCCGGCAGCGAGATC
CTGTGGCAGCACAACGACAAGAACATCGGCGGCGACGAGGACGAC
AAGAACATCGGCAGCGACGAGGACCACCTGAGCCTGAAGGAGTTC
AGCGAGCTGGAGCAGAGCGGCTACTACGTGTGCTACCCCAGAGGC
AGCAAGCCCGAGGACGCCAACTTCTACCTGTACCTGAGAGCCAGA
GTGTGCGAGAACTGCATGGAGATGGACGTGATGAGCGTGGCCACC
ATCGTGATCGTGGACATCTGCATCACCGGCGGCCTGCTGCTGCTG
GTGTACTACTGGAGCAAGAACAGAAAGGCCAAGGCCAAGCCCGTG
ACCAGAGGCGCCGGCGCCGGCGGCAGACAGAGAGGCCAGAACAAG
GAGAGACCCCCCCCCGTGCCCAACCCCGACTACGAGCCCATCAGA
AAGGGCCAGAGAGACCTGTACAGCGGCCTGAACCAGAGAAGAATC
GGACCGcagtgtactaattatgctctcttgaaattggctggagat
gttgagagcaatcccgggccc
(where the lower case is the E2A sequence)
IL-15:
(SEQ ID NO: 48)
MRISKPHLRSISIQCYLCLLLNSHELTEAGIHVFILGCFSAGLPK
TEANWVNVISDLKKIEDLIQSMHIDATLYTESDVHPSCKVTAMKC
FLLELQVISLESGDASIHDTVENLIILANNSLSSNGNVTESGCKE
CEELEEKNIKEFLQSFVHIVQMFINTS*
(SEQ ID NO: 49)
ATGCGCATTAGCAAGCCCCACCTGCGGAGCATCAGCATCCAGTGC
TACCTGTGCCTGCTGCTGAACAGCCACTTCCTGACCGAGGCCGGC
ATCCACGTGTTCATCCTGGGCTGCTTCAGCGCCGGACTGCCCAAG
ACCGAGGCCAACTGGGTGAACGTGATCAGCGACCTGAAGAAGATC
GAGGACCTGATCCAGAGCATGCACATCGACGCCACCCTGTACACC
GAGAGCGACGTGCACCCCAGCTGCAAGGTGACCGCCATGAAGTGC
TTTCTGCTGGAACTGCAGGTGATCAGCCTGGAAAGCGGCGACGCC
AGCATCCACGACACCGTGGAGAACCTGATCATCCTGGCCAACAAC
AGCCTGAGCAGCAACGGCAACGTGACCGAGAGCGGCTGCAAAGAG
TGCGAGGAACTGGAAGAGAAGAACATCAAAGAGTTTCTGCAGAGC
TTCGTGCACATCGTGCAGATGTTCATCAACACCAGC

TCR2: refers to TCRpp65 linked to full length CD3zeta, full length CD3 gamma, full length CD3 delta, and full length CD3 epsilon; it lacks IL-15. Representative sequences are as follows:

(SEQ ID NO: 50)
CTCGAGGGAGTGACCCAGACCCCCAAGTTCCAGGTGCTGAAGACC
GGACAGAGCATGACCCTGCAGTGCGCCCAGGACATGAACCACGAG
TACATGAGCTGGTACCGGCAGGACCCCGGAATGGGACTGCGGCTG
ATCCACTACAGCGTGGGAGCCGGAATCACCGACCAGGGAGAGGTG
CCCAACGGATACAACGTGAGCCGGAGCACCACCGAGGACTTCCCC
CTGCGGCTGCTGAGCGCCGCCCCCAGCCAGACCAGCGTGTACTTC
TGCGCCAGCAGCCCCGTGACCGGAGGAATCTACGGATACACCTTC
GGAAGCGGAACCCGGCTGACCGTGGTGGAGGACCTGAACAAGGTG
TTCCCCCCCGAGGTGGCCGTGTTCGAGCCCAGCGAGGCCGAGATC
AGCCACACCCAGAAGGCCACCCTGGTGTGCCTGGCCACCGGATTC
TTCCCCGACCACGTGGAGCTGAGCTGGTGGGTGAACGGAAAGGAG
GTGCACAGCGGAGTGAGCACCGACCCCCAGCCCCTGAAGGAGCAG
CCCGCCCTGAACGACAGCCGGTACTGCCTGAGCAGCCGGCTGCGG
GTGAGCGCCACCTTCTGGCAGAACCCCCGGAACCACTTCCGGTGC
CAGGTGCAGTTCTACGGACTGAGCGAGAACGACGAGTGGACCCAG
GACCGGGCCAAGCCCGTGACCCAGATCGTGAGCGCCGAGGCCTGG
GGACGGGCCGACGCCACCAACTTCAGCCTGCTGAAGCAGGCCGGC
GACGTGGAGGAGAACCCCGGCCCCATGATCCTGAACGTGGAGCAG
AGCCCCCAGAGCCTGCACGTGCAGGAGGGAGACAGCACCAACTTC
ACCTGCAGCTTCCCCAGCAGCAACTTCTACGCCCTGCACTGGTAC
CGGTGGGAGACCGCCAAGAGCCCCGAGGCCCTGTTCGTGATGACC
CTGAACGGAGACGAGAAGAAGAAGGGACGGATCAGCGCCACCCTG
AACACCAAGGAGGGATACAGCTACCTGTACATCAAGGGAAGCCAG
CCCGAGGACAGCGCCACCTACCTGTGCGCCCGGAACACCGGAAAC
CAGTTCTACTTCGGAACCGGAACCAGCCTGACCGTGATCCCCAAC
ATCCAGAACCCCGACCCCGCCGTGTACCAGCTGCGGGACAGCAAG
AGCAGCGACAAGAGCGTGTGCCTGTTCACCGACTTCGACAGCCAG
ACCAACGTGAGCCAGAGCAAGGACAGCGACGCCTACATCACCGAC
AAGACCGTGCTGGACATGCGGAGCATGGACTTCAAGAGCAACAGC
GCCGTGGCCTGGAGCAACAAGAGCGACTTCGCCTGCGCCAACGCC
TTCAACAACAGCATCATCCCCGAGGACACCTTCTTCCCCAGCCCC
GAGAGCAGCGAGGGCAGAGGCAGCCTGCTGACCTGCGGCGACGTG
GAGGAGAACCCCGGCCCCATGAAGTGGAAGGCGCTTTTCACCGCG
GCCATCCTGCAGGCACAGTTGCCGATTACAGAGGCACAGAGCTTT
GGCCTGCTGGATCCCAAACTCTGCTACCTGCTGGATGGAATCCTC
TTCATCTATGGTGTCATTCTCACTGCCTTGTTCCTGAGAGTGAAG
TTCAGCAGGAGCGCAGACGCCCCCGCGTACCAGCAGGGCCAGAAC
CAGCTCTATAACGAGCTCAATCTAGGACGAAGAGAGGAGTACGAT
GTTTTGGACAAGAGACGTGGCCGGGACCCTGAGATGGGGGGAAAG
CCGCAGAGAAGGAAGAACCCTCAGGAAGGCCTGTACAATGAACTG
CAGAAAGATAAGATGGCGGAGGCCTACAGTGAGATTGGGATGAAA
GGCGAGCGCCGGAGGGGCAAGGGGCACGATGGCCTTTACCAGGGT
CTCAGTACAGCCACCAAGGACACCTACGACGCCCTTCACATGCAG
GCCCTGCCCCCTCGCCAGTGCACCAACTACGCCCTGCTGAAGCTG
GCCGGCGACGTGGAGAGCAACCCCGGCCCCATGGAACAGGGGAAG
GGCCTGGCTGTCCTCATCCTGGCTATCATTCTTCTTCAAGGTACT
TTGGCCCAGTCAATCAAAGGAAACCACTTGGTTAAGGTGTATGAC
TATCAAGAAGATGGTTCGGTACTTCTGACTTGTGATGCAGAAGCC
AAAAATATCACATGGTTTAAAGATGGGAAGATGATCGGCTTCCTA
ACTGAAGATAAAAAAAAATGGAATCTGGGAAGTAATGCCAAGGAC
CCTCGTGGGATGTATCAGTGTAAAGGATCACAGAACAAGTCAAAA
CCACTCCAAGTGTATTACAGAATGTGTCAGAACTGCATTGAACTA
AATGCAGCCACCATATCTGGCTTTCTCTTTGCTGAAATCGTCAGC
ATTTTCGTCCTTGCTGTTGGGGTCTACTTCATTGCTGGACAGGAT
GGAGTTCGCCAGTCGAGAGCTTCAGACAAGCAGACTCTGTTGCCC
AATGACCAGCTCTACCAGCCCCTCAAGGATCGAGAAGATGACCAG
TACAGCCACCTTCAAGGAAACCAGTTGAGGAGGAATGTGAAGCAG
ACCCTGAACTTCGACCTGCTGAAGCTGGCCGGCGACGTGGAGAGC
AACCCCGGCCCCATGGAGCACAGCACCTTCCTGAGCGGCCTGGTG
CTGGCCACCCTGCTGAGCCAGGTGAGCCCCTTCAAGATCCCCATC
GAGGAGCTGGAGGACAGAGTGTTCGTGAACTGCAACACCAGCATC
ACCTGGGTGGAGGGCACCGTGGGCACCCTGCTGAGCGACATCACC
AGACTGGACCTGGGCAAGAGAATCCTGGACCCCAGAGGCATCTAC
AGATGCAACGGCACCGACATCTACAAGGACAAGGAGAGCACCGTG
CAGGTGCACTACAGAATGTGCCAGAGCTGCGTGGAGCTGGACCCC
GCCACCGTGGCCGGCATCATCGTGACCGACGTGATCGCCACCCTG
CTGCTGGCCCTGGGCGTGTTCTGCTTCGCCGGCCACGAGACCGGC
AGACTGAGCGGCGCCGCCGACACCCAGGCCCTGCTGAGAAACGAC
CAGGTGTACCAGCCCCTGAGAGACAGAGACGACGCCCAGTACAGC
CACCTGGGCGGCAACTGGGCCAGAAACAAGGAGGGCAGAGGCAGC
CTGCTGACCTGCGGCGACGTGGAGGAGAACCCCGGCCCCATGCAG
AGCGGCACCCACTGGAGAGTGCTGGGCCTGTGCCTGCTGAGCGTG
GGCGTGTGGGGCCAGGACGGCAACGAGGAGATGGGCGGCATCACC
CAGACCCCCTACAAGGTGAGCATCAGCGGCACCACCGTGATCCTG
ACCTGCCCCCAGTACCCCGGCAGCGAGATCCTGTGGCAGCACAAC
GACAAGAACATCGGCGGCGACGAGGACGACAAGAACATCGGCAGC
GACGAGGACCACCTGAGCCTGAAGGAGTTCAGCGAGCTGGAGCAG
AGCGGCTACTACGTGTGCTACCCCAGAGGCAGCAAGCCCGAGGAC
GCCAACTTCTACCTGTACCTGAGAGCCAGAGTGTGCGAGAACTGC
ATGGAGATGGACGTGATGAGCGTGGCCACCATCGTGATCGTGGAC
ATCTGCATCACCGGCGGCCTGCTGCTGCTGGTGTACTACTGGAGC
AAGAACAGAAAGGCCAAGGCCAAGCCCGTGACCAGAGGCGCCGGC
GCCGGCGGCAGACAGAGAGGCCAGAACAAGGAGAGACCCCCCCCC
GTGCCCAACCCCGACTACGAGCCCATCAGAAAGGGCCAGAGAGAC
CTGTACAGCGGCCTGAACCAGAGAAGAATCGGACCG
(SEQ ID NO: 51)
LEGVTQTPKFQVLKTGQSMTLQCAQDMNHEYMSWYRQDPGMGLRL
IHYSVGAGITDQGEVPNGYNVSRSTTEDFPLRLLSAAPSQTSVYF
CASSPVTGGIYGYTFGSGTRLTVVEDLNKVEPPEVAVFEPSEAEI
SHTQKATLVCLATGFFPDHVELSWWVNGKEVHSGVSTDPQPLKEQ
PALNDSRYCLSSRLRVSATFWQNPRNHFRCQVQFYGLSENDEWTQ
DRAKPVTQIVSAEAWGRADATNFSLLKQAGDVEENPGPMILNVEQ
SPQSLHVQEGDSTNFTCSFPSSNFYALHWYRWETAKSPEALFVMT
LNGDEKKKGRISATLNTKEGYSYLYIKGSQPEDSATYLCARNTGN
QFYFGTGTSLTVIPNIQNPDPAVYQLRDSKSSDKSVCLFTDFDSQ
TNVSQSKDSDAYITDKTVLDMRSMDFKSNSAVAWSNKSDFACANA
FNNSIIPEDTFFPSPESSEGRGSLLTCGDVEENPGPMKWKALFTA
AILQAQLPITEAQSFGLLDPKLCYLLDGILFIYGVILTALFLRVK
ESRSADAPAYQQGQNQLYNELNLGRREEYDVLDKRRGRDPEMGGK
PQRRKNPQEGLYNELQKDKMAEAYSEIGMKGERRRGKGHDGLYQG
LSTATKDTYDALHMQALPPRQCTNYALLKLAGDVESNPGPMEQGK
GLAVLILAIILLQGTLAQSIKGNHLVKVYDYQEDGSVLLTCDAEA
KNITWFKDGKMIGFLTEDKKKWNLGSNAKDPRGMYQCKGSQNKSK
PLQVYYRMCQNCIELNAATISGFLFAEIVSIFVLAVGVYFIAGQD
GVRQSRASDKQTLLPNDQLYQPLKDREDDQYSHLQGNQLRRNVKQ
TLNFDLLKLAGDVESNPGPMEHSTFLSGLVLATLLSQVSPEKIPI
EELEDRVFVNCNTSITWVEGTVGTLLSDITRLDLGKRILDPRGIY
RCNGTDIYKDKESTVQVHYRMCQSCVELDPATVAGIIVTDVIATL
LLALGVFCFAGHETGRLSGAADTQALLRNDQVYQPLRDRDDAQYS
HLGGNWARNKEGRGSLLTCGDVEENPGPMQSGTHWRVLGLCLLSV
GVWGQDGNEEMGGITQTPYKVSISGTTVILTCPQYPGSEILWQHN
DKNIGGDEDDKNIGSDEDHLSLKEFSELEQSGYYVCYPRGSKPED
ANFYLYLRARVCENCMEMDVMSVATIVIVDICITGGLLLLVYYWS
KNRKAKAKPVTRGAGAGGRQRGQNKERPPPVPNPDYEPIRKGQRD
LYSGLNQRRIGP

TCR3: refers to TCRpp65 linked to the intracellular CD3z domain and IL-15, and it may also be referred to as TCRpp65Zicd15, with a representative sequence as follows:

(SEQ ID NO: 52)
MLEGVTQTPKFQVLKTGQSMTLQCAQDMNHEYMSWYRQDPGMGLR
LIHYSVGAGITDQGEVPNGYNVSRSTTEDFPLRLLSAAPSQTSVY
FCASSPVTGGIYGYTFGSGTRLTVVEDLNKVFPPEVAVFEPSEAE
ISHTQKATLVCLATGFFPDHVELSWWVNGKEVHSGVSTDPQPLKE
QPALNDSRYCLSSRLRVSATFWQNPRNHFRCQVQFYGLSENDEWT
QDRAKPVTQIVSAEAWGRADRVKFSRSADAPAYQQGQNQLYNELN
LGRREEYDVLDKRRGRDPEMGGKPQRRKNPQEGLYNELQKDKMAE
AYSEIGMKGERRRGKGHDGLYQGLSTATKDTYDALHMQALPPRAT
NFSLLKQAGDVEENPGPMILNVEQSPQSLHVQEGDSTNFTCSFPS
SNFYALHWYRWETAKSPEALFVMTLNGDEKKKGRISATLNTKEGY
SYLYIKGSQPEDSATYLCARNTGNQFYFGTGTSLTVIPNIQNPDP
AVYQLRDSKSSDKSVCLFTDFDSQTNVSQSKDSDAYITDKTVLDM
RSMDFKSNSAVAWSNKSDFACANAFNNSIIPEDTFFPSPESSRVK
ESRSADAPAYQQGQNQLYNELNLGRREEYDVLDKRRGRDPEMGGK
PQRRKNPQEGLYNELQKDKMAEAYSEIGMKGERRRGKGHDGLYQG
LSTATKDTYDALHMQALPPRPGPQCTNYALLKLAGDVESNPGPMR
ISKPHLRSISIQCYLCLLLNSHELTEAGIHVFILGCFSAGLPKTE
ANWVNVISDLKKIEDLIQSMHIDATLYTESDVHPSCKVTAMKCFL
LELQVISLESGDASIHDTVENLIILANNSLSSNGNVTESGCKECE
ELEEKNIKEFLQSFVHIVQMFINTS*

In TCRpp65Zicd15, the corresponding component sequences are as follows, although these particular sequences or others may be utilized in this and/or other constructs:

TCRb-extracellular domain:
(SEQ ID NO: 40)
MLEGVTQTPKFQVLKTGQSMTLQCAQDMNHEYMSWYRQDPGMGLR
LIHYSVGAGITDQGEVPNGYNVSRSTTEDFPLRLLSAAPSQTSVY
FCASSPVTGGIYGYTFGSGTRLTVVEDLNKVFPPEVAVFEPSEAE
ISHTQKATLVCLATGFFPDHVELSWWVNGKEVHSGVSTDPQPLKE
QPALNDSRYCLSSRLRVSATFWQNPRNHFRCQVQFYGLSENDEWT
QDRAKPVTQIVSAEAWGRAD
(SEQ ID NO: 41)
ATGCTCGAGGGAGTGACCCAGACCCCCAAGTTCCAGGTGCTGAAG
ACCGGACAGAGCATGACCCTGCAGTGCGCCCAGGACATGAACCAC
GAGTACATGAGCTGGTACCGGCAGGACCCCGGAATGGGACTGCGG
CTGATCCACTACAGCGTGGGAGCCGGAATCACCGACCAGGGAGAG
GTGCCCAACGGATACAACGTGAGCCGGAGCACCACCGAGGACTTC
CCCCTGCGGCTGCTGAGCGCCGCCCCCAGCCAGACCAGCGTGTAC
TTCTGCGCCAGCAGCCCCGTGACCGGAGGAATCTACGGATACACC
TTCGGAAGCGGAACCCGGCTGACCGTGGTGGAGGACCTGAACAAG
GTGTTCCCCCCCGAGGTGGCCGTGTTCGAGCCCAGCGAGGCCGAG
ATCAGCCACACCCAGAAGGCCACCCTGGTGTGCCTGGCCACCGGA
TTCTTCCCCGACCACGTGGAGCTGAGCTGGTGGGTGAACGGAAAG
GAGGTGCACAGCGGAGTGAGCACCGACCCCCAGCCCCTGAAGGAG
CAGCCCGCCCTGAACGACAGCCGGTACTGCCTGAGCAGCCGGCTG
CGGGTGAGCGCCACCTTCTGGCAGAACCCCCGGAACCACTTCCGG
TGCCAGGTGCAGTTCTACGGACTGAGCGAGAACGACGAGTGGACC
CAGGACCGGGCCAAGCCCGTGACCCAGATCGTGAGCGCCGAGGCC
TGGGGACGGGCCGAC
CD3 zeta intracellular domain (Z-ICD):
(SEQ ID NO: 42)
RVKFSRSADAPAYQQGQNQLYNELNLGRREEYDVLDKRRGRDPEM
GGKPQRRKNPQEGLYNELQKDKMAEAYSEIGMKGERRRGKGHDGL
YQGLSTATKDTYDALHMQALPPRATNFSLLKQAG
DVEENPGP (where P2A sequence is at the C-
terminus)
(SEQ ID NO: 43)
AGAGTGAAGTTCAGCAGGAGCGCAGACGCCCCCGCGTACCAGCAG
GGCCAGAACCAGCTCTATAACGAGCTCAATCTAGGACGAAGAGAG
GAGTACGATGTTTTGGACAAGAGACGTGGCCGGGACCCTGAGATG
GGGGGAAAGCCGCAGAGAAGGAAGAACCCTCAGGAAGGCCTGTAC
AATGAACTGCAGAAAGATAAGATGGCGGAGGCCTACAGTGAGATT
GGGATGAAAGGCGAGCGCCGGAGGGGCAAGGGGCACGATGGCCTT
TACCAGGGTCTCAGTACAGCCACCAAGGACACCTACGACGCCCTT
CACATGCAGGCCCTGCCCCCTCGCgccaccaacttctccctgctg
aagcaggccggcgacgtggaggagaaccccggcccc
(where the lowercase
sequence is P2A sequence)
TCRa-extracellular domain:
(SEQ ID NO: 44)
MILNVEQSPQSLHVQEGDSTNFTCSFPSSNFYALHWYRWETAKSP
EALFVMTLNGDEKKKGRISATLNTKEGYSYLYIKGSQPEDSATYL
CARNTGNQFYFGTGTSLTVIPNIQNPDPAVYQLRDSKSSDKSVCL
FTDFDSQTNVSQSKDSDAYITDKTVLDMRSMDFKSNSAVAWSNKS
DFACANAFNNSIIPEDTFFPSPESS
(SEQ ID NO: 45)
ATGATCCTGAACGTGGAGCAGAGCCCCCAGAGCCTGCACGTGCAG
GAGGGAGACAGCACCAACTTCACCTGCAGCTTCCCCAGCAGCAAC
TTCTACGCCCTGCACTGGTACCGGTGGGAGACCGCCAAGAGCCCC
GAGGCCCTGTTCGTGATGACCCTGAACGGAGACGAGAAGAAGAAG
GGACGGATCAGCGCCACCCTGAACACCAAGGAGGGATACAGCTAC
CTGTACATCAAGGGAAGCCAGCCCGAGGACAGCGCCACCTACCTG
TGCGCCCGGAACACCGGAAACCAGTTCTACTTCGGAACCGGAACC
AGCCTGACCGTGATCCCCAACATCCAGAACCCCGACCCCGCCGTG
TACCAGCTGCGGGACAGCAAGAGCAGCGACAAGAGCGTGTGCCTG
TTCACCGACTTCGACAGCCAGACCAACGTGAGCCAGAGCAAGGAC
AGCGACGCCTACATCACCGACAAGACCGTGCTGGACATGCGGAGC
ATGGACTTCAAGAGCAACAGCGCCGTGGCCTGGAGCAACAAGAGC
GACTTCGCCTGCGCCAACGCCTTCAACAACAGCATCATCCCCGAG
GACACCTTCTTCCCCAGCCCCGAGAGCAGC
CD3 zeta intracellular domain (Z-ICD)
(in specific embodiments, two or more Z-
ICD sequences may be utilized):
(SEQ ID NO: 53)
RVKFSRSADAPAYQQGQNQLYNELNLGRREEYDVLDKRRGRDPEM
GGKPQRRKNPQEGLYNELQKDKMAEAYSEIGMKGERRRGKGHDGL
YQGLSTATKDTYDALHMQALPPRPGPQCTNYALLKLAGDVESNPG
P
(SEQ ID NO: 54)
AGAGTGAAGTTCAGCAGGAGCGCAGACGCCCCCGCGTACCAGCAG
GGCCAGAACCAGCTCTATAACGAGCTCAATCTAGGACGAAGAGAG
GAGTACGATGTTTTGGACAAGAGACGTGGCCGGGACCCTGAGATG
GGGGGAAAGCCGCAGAGAAGGAAGAACCCTCAGGAAGGCCTGTAC
AATGAACTGCAGAAAGATAAGATGGCGGAGGCCTACAGTGAGATT
GGGATGAAAGGCGAGCGCCGGAGGGGCAAGGGGCACGATGGCCTT
TACCAGGGTCTCAGTACAGCCACCAAGGACACCTACGACGCCCTT
CACATGCAGGCCCTGCCCCCTCGCCAGTGCACCAACTACGCCCTG
CTGAAGCTGGCCGGCGACGTGGAGAGCAACCCCGGCCCC
IL-15:
(SEQ ID NO: 48)
MRISKPHLRSISIQCYLCLLLNSHELTEAGIHVFILGCFSAGLPK
TEANWVNVISDLKKIEDLIQSMHIDATLYTESDVHPSCKVTAMKC
FLLELQVISLESGDASIHDTVENLIILANNSLSSNGNVTESGCKE
CEELEEKNIKEFLQSFVHIVQMFINTS*
(SEQ ID NO: 49)
ATGCGCATTAGCAAGCCCCACCTGCGGAGCATCAGCATCCAGTGC
TACCTGTGCCTGCTGCTGAACAGCCACTTCCTGACCGAGGCCGGC
ATCCACGTGTTCATCCTGGGCTGCTTCAGCGCCGGACTGCCCAAG
ACCGAGGCCAACTGGGTGAACGTGATCAGCGACCTGAAGAAGATC
GAGGACCTGATCCAGAGCATGCACATCGACGCCACCCTGTACACC
GAGAGCGACGTGCACCCCAGCTGCAAGGTGACCGCCATGAAGTGC
TTTCTGCTGGAACTGCAGGTGATCAGCCTGGAAAGCGGCGACGCC
AGCATCCACGACACCGTGGAGAACCTGATCATCCTGGCCAACAAC
AGCCTGAGCAGCAACGGCAACGTGACCGAGAGCGGCTGCAAAGAG
TGCGAGGAACTGGAAGAGAAGAACATCAAAGAGTTTCTGCAGAGC
TTCGTGCACATCGTGCAGATGTTCATCAACACCAGC

TCR4: refers to TCRpp65 that also may be referred to as TCRpp65betaalpha, and a representative sequence is as follows:

(SEQ ID NO: 55)
MLEGVTQTPKFQVLKTGQSMTLQCAQDMNHEYMSWYRQDPGMGLR
LIHYSVGAGITDQGEVPNGYNVSRSTTEDFPLRLLSAAPSQTSVY
FCASSPVTGGIYGYTFGSGTRLTVVEDLNKVFPPEVAVFEPSEAE
ISHTQKATLVCLATGFFPDHVELSWWVNGKEVHSGVSTDPQPLKE
QPALNDSRYCLSSRLRVSATFWQNPRNHFRCQVQFYGLSENDEWT
QDRAKPVTQIVSAEAWGRADRVKFSRSADAPAYQQGQNQLYNELN
LGRREEYDVLDKRRGRDPEMGGKPQRRKNPQEGLYNELQKDKMAE
AYSEIGMKGERRRGKGHDGLYQGLSTATKDTYDALHMQALPPRAT
NFSLLKQAGDVEENPGPMILNVEQSPQSLHVQEGDSTNFTCSFPS
SNFYALHWYRWETAKSPEALFVMTLNGDEKKKGRISATLNTKEGY
SYLYIKGSQPEDSATYLCARNTGNQFYFGTGTSLTVIPNIQNPDP
AVYQLRDSKSSDKSVCLFTDFDSQTNVSQSKDSDAYITDKTVLDM
RSMDFKSNSAVAWSNKSDFACANAFNNSIIPEDTFFPSPESSRVK
ESRSADAPAYQQGQNQLYNELNLGRREEYDVLDKRRGRDPEMGGK
PQRRKNPQEGLYNELQKDKMAEAYSEIGMKGERRRGKGHDGLYQG
LSTATKDTYDALHMQALPPRPGPQCTNYALLKLAGDVESNPGPMR
ISKPHLRSISIQCYLCLLLNSHELTEAGIHVFILGCFSAGLPKTE
ANWVNVISDLKKIEDLIQSMHIDATLYTESDVHPSCKVTAMKCFL
LELQVISLESGDASIHDTVENLIILANNSLSSNGNVTESGCKECE
ELEEKNIKEFLQSFVHIVQMFINTS*

For TCRpp65betaalpha, the corresponding component sequences are as follows, although these particular sequences or others may be utilized in this and/or other constructs:

TCRb-extracellular domain:
(SEQ ID NO: 40)
MLEGVTQTPKFQVLKTGQSMTLQCAQDMNHEYMSWYRQDPGMGLR
LIHYSVGAGITDQGEVPNGYNVSRSTTEDFPLRLLSAAPSQTSVY
FCASSPVTGGIYGYTFGSGTRLTVVEDLNKVFPPEVAVFEPSEAE
ISHTQKATLVCLATGFFPDHVELSWWVNGKEVHSGVSTDPQPLKE
QPALNDSRYCLSSRLRVSATFWQNPRNHFRCQVQFYGLSENDEWT
QDRAKPVTQIVSAEAWGRAD
(SEQ ID NO: 41)
ATGCTCGAGGGAGTGACCCAGACCCCCAAGTTCCAGGTGCTGAAG
ACCGGACAGAGCATGACCCTGCAGTGCGCCCAGGACATGAACCAC
GAGTACATGAGCTGGTACCGGCAGGACCCCGGAATGGGACTGCGG
CTGATCCACTACAGCGTGGGAGCCGGAATCACCGACCAGGGAGAG
GTGCCCAACGGATACAACGTGAGCCGGAGCACCACCGAGGACTTC
CCCCTGCGGCTGCTGAGCGCCGCCCCCAGCCAGACCAGCGTGTAC
TTCTGCGCCAGCAGCCCCGTGACCGGAGGAATCTACGGATACACC
TTCGGAAGCGGAACCCGGCTGACCGTGGTGGAGGACCTGAACAAG
GTGTTCCCCCCCGAGGTGGCCGTGTTCGAGCCCAGCGAGGCCGAG
ATCAGCCACACCCAGAAGGCCACCCTGGTGTGCCTGGCCACCGGA
TTCTTCCCCGACCACGTGGAGCTGAGCTGGTGGGTGAACGGAAAG
GAGGTGCACAGCGGAGTGAGCACCGACCCCCAGCCCCTGAAGGAG
CAGCCCGCCCTGAACGACAGCCGGTACTGCCTGAGCAGCCGGCTG
CGGGTGAGCGCCACCTTCTGGCAGAACCCCCGGAACCACTTCCGG
TGCCAGGTGCAGTTCTACGGACTGAGCGAGAACGACGAGTGGACC
CAGGACCGGGCCAAGCCCGTGACCCAGATCGTGAGCGCCGAGGCC
TGGGGACGGGCCGAC
CD3 zeta intracellular domain (Z-ICD):
(SEQ ID NO: 42)
RVKFSRSADAPAYQQGQNQLYNELNLGRREEYDVLDKRRGRDPEM
GGKPQRRKNPQEGLYNELQKDKMAEAYSEIGMKGERRRGKGHDGL
YQGLSTATKDTYDALHMQALPPRATNFSLLKQAGDVEENPGP
(SEQ ID NO: 54)
AGAGTGAAGTTCAGCAGGAGCGCAGACGCCCCCGCGTACCAGCAG
GGCCAGAACCAGCTCTATAACGAGCTCAATCTAGGACGAAGAGAG
GAGTACGATGTTTTGGACAAGAGACGTGGCCGGGACCCTGAGATG
GGGGGAAAGCCGCAGAGAAGGAAGAACCCTCAGGAAGGCCTGTAC
AATGAACTGCAGAAAGATAAGATGGCGGAGGCCTACAGTGAGATT
GGGATGAAAGGCGAGCGCCGGAGGGGCAAGGGGCACGATGGCCTT
TACCAGGGTCTCAGTACAGCCACCAAGGACACCTACGACGCCCTT
CACATGCAGGCCCTGCCCCCTCGCCAGTGCACCAACTACGCCCTG
CTGAAGCTGGCCGGCGACGTGGAGAGCAACCCCGGCCCC
TCRa-extracellular domain:
(SEQ ID NO: 44)
MILNVEQSPQSLHVQEGDSTNFTCSFPSSNFYALHWYRWETAKSP
EALFVMTLNGDEKKKGRISATLNTKEGYSYLYIKGSQPEDSATYL
CARNTGNQFYFGTGTSLTVIPNIQNPDPAVYQLRDSKSSDKSVCL
FTDFDSQTNVSQSKDSDAYITDKTVLDMRSMDFKSNSAVAWSNKS
DFACANAFNNSIIPEDTFFPSPESS
(SEQ ID NO: 45)
ATGATCCTGAACGTGGAGCAGAGCCCCCAGAGCCTGCACGTGCAG
GAGGGAGACAGCACCAACTTCACCTGCAGCTTCCCCAGCAGCAAC
TICTACGCCCTGCACTGGTACCGGTGGGAGACCGCCAAGAGCCCC
GAGGCCCTGTTCGTGATGACCCTGAACGGAGACGAGAAGAAGAAG
GGACGGATCAGCGCCACCCTGAACACCAAGGAGGGATACAGCTAC
CTGTACATCAAGGGAAGCCAGCCCGAGGACAGCGCCACCTACCTG
TGCGCCCGGAACACCGGAAACCAGTTCTACTTCGGAACCGGAACC
AGCCTGACCGTGATCCCCAACATCCAGAACCCCGACCCCGCCGTG
TACCAGCTGCGGGACAGCAAGAGCAGCGACAAGAGCGTGTGCCTG
TTCACCGACTTCGACAGCCAGACCAACGTGAGCCAGAGCAAGGAC
AGCGACGCCTACATCACCGACAAGACCGTGCTGGACATGCGGAGC
ATGGACTTCAAGAGCAACAGCGCCGTGGCCTGGAGCAACAAGAGC
GACTTCGCCTGCGCCAACGCCTTCAACAACAGCATCATCCCCGAG
GACACCTTCTTCCCCAGCCCCGAGAGCAGC
CD3 zeta intracellular domain (Z-ICD):
(SEQ ID NO: 53)
RVKFSRSADAPAYQQGQNQLYNELNLGRREEYDVLDKRRGRDPEM
GGKPQRRKNPQEGLYNELQKDKMAEAYSEIGMKGERRRGKGHDGL
YQGLSTATKDTYDALHMQALPPRPGPQCTNYALLKLAGDVESNPG
P
(SEQ ID NO: 54)
AGAGTGAAGTTCAGCAGGAGCGCAGACGCCCCCGCGTACCAGCAG
GGCCAGAACCAGCTCTATAACGAGCTCAATCTAGGACGAAGAGAG
GAGTACGATGTTTTGGACAAGAGACGTGGCCGGGACCCTGAGATG
GGGGGAAAGCCGCAGAGAAGGAAGAACCCTCAGGAAGGCCTGTAC
AATGAACTGCAGAAAGATAAGATGGCGGAGGCCTACAGTGAGATT
GGGATGAAAGGCGAGCGCCGGAGGGGCAAGGGGCACGATGGCCTT
TACCAGGGTCTCAGTACAGCCACCAAGGACACCTACGACGCCCTT
CACATGCAGGCCCTGCCCCCTCGCCAGTGCACCAACTACGCCCTG
CTGAAGCTGGCCGGCGACGTGGAGAGCAACCCCGGCCCC
IL-15:
(SEQ ID NO: 48)
MRISKPHLRSISIQCYLCLLLNSHELTEAGIHVFILGCFSAGLPK
TEANWVNVISDLKKIEDLIQSMHIDATLYTESDVHPSCKVTAMKC
FLLELQVISLESGDASIHDTVENLIILANNSLSSNGNVTESGCKE
CEELEEKNIKEFLQSFVHIVQMFINTS*
(SEQ ID NO: 49)
ATGCGCATTAGCAAGCCCCACCTGCGGAGCATCAGCATCCAGTGC
TACCTGTGCCTGCTGCTGAACAGCCACTTCCTGACCGAGGCCGGC
ATCCACGTGTTCATCCTGGGCTGCTTCAGCGCCGGACTGCCCAAG
ACCGAGGCCAACTGGGTGAACGTGATCAGCGACCTGAAGAAGATC
GAGGACCTGATCCAGAGCATGCACATCGACGCCACCCTGTACACC
GAGAGCGACGTGCACCCCAGCTGCAAGGTGACCGCCATGAAGTGC
TTTCTGCTGGAACTGCAGGTGATCAGCCTGGAAAGCGGCGACGCC
AGCATCCACGACACCGTGGAGAACCTGATCATCCTGGCCAACAAC
AGCCTGAGCAGCAACGGCAACGTGACCGAGAGCGGCTGCAAAGAG
TGCGAGGAACTGGAAGAGAAGAACATCAAAGAGTTTCTGCAGAGC
TTCGTGCACATCGTGCAGATGTTCATCAACACCAGC

An additional representative sequence for TCRpp65betaalpha is as follows:

(SEQ ID NO: 56)
ATGGACTCCTGGACCTTCTGCTGTGTGTCCCTTTGCATCCTGGTA
GCAAAGCACACAGATGCTGGAGTTATCCAGTCACCCCGGCACGAG
GTGACAGAGATGGGACAAGAAGTGACTCTGAGATGTAAACCAATT
TCAGGACACGACTACCTTTTCTGGTACAGACAGACCATGATGCGG
GGACTGGAGTTGCTCATTTACTTTAACAACAACGTTCCGATAGAT
GATTCAGGGATGCCCGAGGATCGATTCTCAGCTAAGATGCCTAAT
GCATCATTCTCCACTCTGAAGATCCAGCCCTCAGAACCCAGGGAC
TCAGCTGTGTACTTCTGTGCCAGCAGTTCGGCAAACTATGGCTAC
ACCTTCGGTTCGGGGACCAGGTTAACCGTTGTAGAGGACCTGAAC
AAGGTGTTCCCACCCGAGGTCGCTGTGTTTGAGCCATCAGAAGCA
GAGATCTCCCACACCCAAAAGGCCACACTGGTGTGCCTGGCCACA
GGCTTCTTCCCTGACCACGTGGAGCTGAGCTGGTGGGTGAATGGG
AAGGAGGTGCACAGTGGGGTCAGCACGGACCCGCAGCCCCTCAAG
GAGCAGCCCGCCCTCAATGACTCCAGATACTGCCTGAGCAGCCGC
CTGAGGGTCTCGGCCACCTTCTGGCAGAACCCCCGCAACCACTTC
CGCTGTCAAGTCCAGTTCTACGGGCTCTCGGAGAATGACGAGTGG
ACCCAGGATAGGGCCAAACCCGTCACCCAGATCGTCAGCGCCGAG
GCCTGGGGTAGAGCAGACTGTGGCTTTACCTCGGTGTCCTACCAG
CAAGGGGTCCTGTCTGCCACCATCCTCTATGAGATCCTGCTAGGG
AAGGCCACCCTGTATGCTGTGCTGGTCAGCGCCCTTGTGTTGATG
GCCATGGTCAAGAGAAAGGATTTCGAGGGCAGGGGAAGTCTTCTA
ACATGCGGGGACGTGGAGGAAAATCCCGGGCCCATGCTCCTTGAA
CATTTATTAATAATCTTGTGGATGCAGCTGACATGGGTCAGTGGT
CAACAGCTGAATCAGAGTCCTCAATCTATGTTTATCCAGGAAGGA
GAAGATGTCTCCATGAACTGCACTTCTTCAAGCATATTTAACACC
TGGCTATGGTACAAGCAGGACCCTGGGGAAGGTCCTGTCCTCTTG
ATAGCCTTATATAAGGCTGGTGAATTGACCTCAAATGGAAGACTG
ACTGCTCAGTTTGGTATAACCAGAAAGGACAGCTTCCTGAATATC
TCAGCATCCATACCCAGTGATGTAGGCATCTACTTCTGTGCTGGA
CCCATGAAAACCTCCTACGACAAGGTGATATTTGGGCCAGGGACA
AGCTTATCAGTCATTCCAAATATCCAGAACCCTGACCCTGCCGTG
TACCAGCTGAGAGACTCTAAATCCAGTGACAAGTCTGTCTGCCTA
TTCACCGATTTTGATTCTCAAACAAATGTGTCACAAAGTAAGGAT
TCTGATGTGTATATCACAGACAAAACTGTGCTAGACATGAGGTCT
ATGGACTTCAAGAGCAACAGTGCTGTGGCCTGGAGCAACAAATCT
GACTTTGCATGTGCAAACGCCTTCAACAACAGCATTATTCCAGAA
GACACCTTCTTCCCCAGCCCAGAAAGTTCCTGTGATGTCAAGCTG
GTCGAGAAAAGCTTTGAAACAGATACGAACCTAAACTTTCAAAAC
CTGTCAGTGATTGGGTTCCGAATCCTCCTCCTGAAAGTGGCCGGG
TTTAATCTGCTCATGACGCTGCGGCTGTGGTCCAGCTGA
(SEQ ID NO: 57)
MDSWTFCCVSLCILVAKHTDAGVIQSPRHEVTEMGQEVTLRCKPI
SGHDYLFWYRQTMMRGLELLIYFNNNVPIDDSGMPEDRFSAKMPN
ASESTLKIQPSEPRDSAVYFCASSSANYGYTFGSGTRLTVVEDLN
KVEPPEVAVFEPSEAEISHTQKATLVCLATGFFPDHVELSWWVNG
KEVHSGVSTDPQPLKEQPALNDSRYCLSSRLRVSATFWQNPRNHF
RCQVQFYGLSENDEWTQDRAKPVTQIVSAEAWGRADCGFTSVSYQ
QGVLSATILYEILLGKATLYAVLVSALVLMAMVKRKDFEGRGSLL
TCGDVEENPGPMLLEHLLIILWMQLTWVSGQQLNQSPQSMFIQEG
EDVSMNCTSSSIFNTWLWYKQDPGEGPVLLIALYKAGELTSNGRL
TAQFGITRKDSFLNISASIPSDVGIYFCAGPMKTSYDKVIFGPGT
SLSVIPNIQNPDPAVYQLRDSKSSDKSVCLFTDFDSQTNVSQSKD
SDVYITDKTVLDMRSMDFKSNSAVAWSNKSDFACANAFNNSIIPE
DTFFPSPESSCDVKLVEKSFETDTNLNFQNLSVIGFRILLLKVAG
ENLLMTLRLWSS*

Z1: refers to full length CD3zeta, full length CD3 gamma, full length CD3 delta, and full length CD3 epsilon linked to IL15 (see FIGS. 2A and 2B), and it may also be referred to as CD3ZFLGDEFL15, and representative sequences may be as follows:

(SEQ ID NO: 58)
MLEMKWKALFTAAILQAQLPITEAQSFGLLDPKLCYLLDGILFIY
GVILTALFLRVKFSRSADAPAYQQGQNQLYNELNLGRREEYDVLD
KRRGRDPEMGGKPQRRKNPQEGLYNELQKDKMAEAYSEIGMKGER
RRGKGHDGLYQGLSTATKDTYDALHMQALPPRQCTNYALLKLAGD
VESNPGPMEQGKGLAVLILAIILLQGTLAQSIKGNHLVKVYDYQE
DGSVLLTCDAEAKNITWFKDGKMIGFLTEDKKKWNLGSNAKDPRG
MYQCKGSQNKSKPLQVYYRMCQNCIELNAATISGFLFAEIVSIFV
LAVGVYFIAGQDGVRQSRASDKQTLLPNDQLYQPLKDREDDQYSH
LQGNQLRRNVKQTLNFDLLKLAGDVESNPGPMEHSTFLSGLVLAT
LLSQVSPFKIPIEELEDRVFVNCNTSITWVEGTVGTLLSDITRLD
LGKRILDPRGIYRCNGTDIYKDKESTVQVHYRMCQSCVELDPATV
AGIIVTDVIATLLLALGVFCFAGHETGRLSGAADTQALLRNDQVY
QPLRDRDDAQYSHLGGNWARNKEGRGSLLTCGDVEENPGPMQSGT
HWRVLGLCLLSVGVWGQDGNEEMGGITQTPYKVSISGTTVILTCP
QYPGSEILWQHNDKNIGGDEDDKNIGSDEDHLSLKEFSELEQSGY
YVCYPRGSKPEDANFYLYLRARVCENCMEMDVMSVATIVIVDICI
TGGLLLLVYYWSKNRKAKAKPVTRGAGAGGRQRGQNKERPPPVPN
PDYEPIRKGQRDLYSGLNQRRIGPQCTNYALLKLAGDVESNPGPM
RISKPHLRSISIQCYLCLLLNSHELTEAGIHVFILGCFSAGLPKT
EANWVNVISDLKKIEDLIQSMHIDATLYTESDVHPSCKVTAMKCF
LLELQVISLESGDASIHDTVENLIILANNSLSSNGNVTESGCKEC
EELEEKNIKEFLQSFVHIVQMFINTS
(SEQ ID NO: 59)
ATGCTCGAGATGAAGTGGAAGGCGCTTTTCACCGCGGCCATCCTG
CAGGCACAGTTGCCGATTACAGAGGCACAGAGCTTTGGCCTGCTG
GATCCCAAACTCTGCTACCTGCTGGATGGAATCCTCTTCATCTAT
GGTGTCATTCTCACTGCCTTGTTCCTGAGAGTGAAGTTCAGCAGG
AGCGCAGACGCCCCCGCGTACCAGCAGGGCCAGAACCAGCTCTAT
AACGAGCTCAATCTAGGACGAAGAGAGGAGTACGATGTTTTGGAC
AAGAGACGTGGCCGGGACCCTGAGATGGGGGGAAAGCCGCAGAGA
AGGAAGAACCCTCAGGAAGGCCTGTACAATGAACTGCAGAAAGAT
AAGATGGCGGAGGCCTACAGTGAGATTGGGATGAAAGGCGAGCGC
CGGAGGGGCAAGGGGCACGATGGCCTTTACCAGGGTCTCAGTACA
GCCACCAAGGACACCTACGACGCCCTTCACATGCAGGCCCTGCCC
CCTCGCCAGTGCACCAACTACGCCCTGCTGAAGCTGGCCGGCGAC
GTGGAGAGCAACCCCGGCCCCATGGAACAGGGGAAGGGCCTGGCT
GTCCTCATCCTGGCTATCATTCTTCTTCAAGGTACTTTGGCCCAG
TCAATCAAAGGAAACCACTTGGTTAAGGTGTATGACTATCAAGAA
GATGGTTCGGTACTTCTGACTTGTGATGCAGAAGCCAAAAATATC
ACATGGTTTAAAGATGGGAAGATGATCGGCTTCCTAACTGAAGAT
AAAAAAAAATGGAATCTGGGAAGTAATGCCAAGGACCCTCGTGGG
ATGTATCAGTGTAAAGGATCACAGAACAAGTCAAAACCACTCCAA
GTGTATTACAGAATGTGTCAGAACTGCATTGAACTAAATGCAGCC
ACCATATCTGGCTTTCTCTTTGCTGAAATCGTCAGCATTTTCGTC
CTTGCTGTTGGGGTCTACTTCATTGCTGGACAGGATGGAGTTCGC
CAGTCGAGAGCTTCAGACAAGCAGACTCTGTTGCCCAATGACCAG
CTCTACCAGCCCCTCAAGGATCGAGAAGATGACCAGTACAGCCAC
CTTCAAGGAAACCAGTTGAGGAGGAATGTGAAGCAGACCCTGAAC
TTCGACCTGCTGAAGCTGGCCGGCGACGTGGAGAGCAACCCCGGC
CCCATGGAGCACAGCACCTTCCTGAGCGGCCTGGTGCTGGCCACC
CTGCTGAGCCAGGTGAGCCCCTTCAAGATCCCCATCGAGGAGCTG
GAGGACAGAGTGTTCGTGAACTGCAACACCAGCATCACCTGGGTG
GAGGGCACCGTGGGCACCCTGCTGAGCGACATCACCAGACTGGAC
CTGGGCAAGAGAATCCTGGACCCCAGAGGCATCTACAGATGCAAC
GGCACCGACATCTACAAGGACAAGGAGAGCACCGTGCAGGTGCAC
TACAGAATGTGCCAGAGCTGCGTGGAGCTGGACCCCGCCACCGTG
GCCGGCATCATCGTGACCGACGTGATCGCCACCCTGCTGCTGGCC
CTGGGCGTGTTCTGCTTCGCCGGCCACGAGACCGGCAGACTGAGC
GGCGCCGCCGACACCCAGGCCCTGCTGAGAAACGACCAGGTGTAC
CAGCCCCTGAGAGACAGAGACGACGCCCAGTACAGCCACCTGGGC
GGCAACTGGGCCAGAAACAAGGAGGGCAGAGGCAGCCTGCTGACC
TGCGGCGACGTGGAGGAGAACCCCGGCCCCATGCAGAGCGGCACC
CACTGGAGAGTGCTGGGCCTGTGCCTGCTGAGCGTGGGCGTGTGG
GGCCAGGACGGCAACGAGGAGATGGGCGGCATCACCCAGACCCCC
TACAAGGTGAGCATCAGCGGCACCACCGTGATCCTGACCTGCCCC
CAGTACCCCGGCAGCGAGATCCTGTGGCAGCACAACGACAAGAAC
ATCGGCGGCGACGAGGACGACAAGAACATCGGCAGCGACGAGGAC
CACCTGAGCCTGAAGGAGTTCAGCGAGCTGGAGCAGAGCGGCTAC
TACGTGTGCTACCCCAGAGGCAGCAAGCCCGAGGACGCCAACTTC
TACCTGTACCTGAGAGCCAGAGTGTGCGAGAACTGCATGGAGATG
GACGTGATGAGCGTGGCCACCATCGTGATCGTGGACATCTGCATC
ACCGGCGGCCTGCTGCTGCTGGTGTACTACTGGAGCAAGAACAGA
AAGGCCAAGGCCAAGCCCGTGACCAGAGGCGCCGGCGCCGGCGGC
AGACAGAGAGGCCAGAACAAGGAGAGACCCCCCCCCGTGCCCAAC
CCCGACTACGAGCCCATCAGAAAGGGCCAGAGAGACCTGTACAGC
GGCCTGAACCAGAGAAGAATCGGACCGCAGTGTACTAATTATGCT
CTCTTGAAATTGGCTGGAGATGTTGAGAGCAATCCCGGGCCCATG
CGCATTAGCAAGCCCCACCTGCGGAGCATCAGCATCCAGTGCTAC
CTGTGCCTGCTGCTGAACAGCCACTTCCTGACCGAGGCCGGCATC
CACGTGTTCATCCTGGGCTGCTTCAGCGCCGGACTGCCCAAGACC
GAGGCCAACTGGGTGAACGTGATCAGCGACCTGAAGAAGATCGAG
GACCTGATCCAGAGCATGCACATCGACGCCACCCTGTACACCGAG
AGCGACGTGCACCCCAGCTGCAAGGTGACCGCCATGAAGTGCTTT
CTGCTGGAACTGCAGGTGATCAGCCTGGAAAGCGGCGACGCCAGC
ATCCACGACACCGTGGAGAACCTGATCATCCTGGCCAACAACAGC
CTGAGCAGCAACGGCAACGTGACCGAGAGCGGCTGCAAAGAGTGC
GAGGAACTGGAAGAGAAGAACATCAAAGAGTTTCTGCAGAGCTTC
GTGCACATCGTGCAGATGTTCATCAACACCAGC

Z2: refers to full length CD3zeta, full length CD3 gamma, full length CD3 delta, and full length CD3 epsilon linked to membrane bound IL21 (with CD8 transmembrane domain for the membrane bound IL21), and it may also be referred to as CD3ZGDEFLSP821CD28, and a representative sequence is as follows:

(SEQ ID NO: 60)
MLEMKWKALFTAAILQAQLPITEAQSFGLLDPKLCYLLDGILFIY
GVILTALFLRVKESRSADAPAYQQGQNQLYNELNLGRREEYDVLD
KRRGRDPEMGGKPQRRKNPQEGLYNELQKDKMAEAYSEIGMKGER
RRGKGHDGLYQGLSTATKDTYDALHMQALPPRQCTNYALLKLAGD
VESNPGPMEQGKGLAVLILAIILLQGTLAQSIKGNHLVKVYDYQE
DGSVLLTCDAEAKNITWFKDGKMIGFLTEDKKKWNLGSNAKDPRG
MYQCKGSQNKSKPLQVYYRMCQNCIELNAATISGFLFAEIVSIFV
LAVGVYFIAGQDGVRQSRASDKQTLLPNDQLYQPLKDREDDQYSH
LQGNQLRRNVKQTLNFDLLKLAGDVESNPGPMEHSTELSGLVLAT
LLSQVSPEKIPIEELEDRVFVNCNTSITWVEGTVGTLLSDITRLD
LGKRILDPRGIYRCNGTDIYKDKESTVQVHYRMCQSCVELDPATV
AGIIVTDVIATLLLALGVFCFAGHETGRLSGAADTQALLRNDQVY
QPLRDRDDAQYSHLGGNWARNKEGRGSLLTCGDVEENPGPMQSGT
HWRVLGLCLLSVGVWGQDGNEEMGGITQTPYKVSISGTTVILTCP
QYPGSEILWQHNDKNIGGDEDDKNIGSDEDHLSLKEFSELEQSGY
YVCYPRGSKPEDANFYLYLRARVCENCMEMDVMSVATIVIVDICI
TGGLLLLVYYWSKNRKAKAKPVTRGAGAGGRQRGQNKERPPPVPN
PDYEPIRKGQRDLYSGLNQRRIGPQCTNYALLKLAGDVESNPGPM
RICLTSDRLAPAAGLAAPRRQAVHKSSSQGQDRHMIRMRQLIDIV
DQLKNYVNDLVPEFLPAPEDVETNCEWSAFSCFQKAQLKSANTGN
NERIINVSIKKLKRKPPSTNAGRRQKHRLTCPSCDSYEKKPPKEF
LERFKSLLQKMIHQHLSSRTHGSEDSTTTPAPRPPTPAPTIASQP
LSLRPEACRPAAGGAVHTRGLDFACDFWVLVVVGGVLACYSLLVT
VAFIIFWV*

For CD3ZGDEFLSP821CD28, the corresponding component sequences are as follows, although these particular sequences or others may be utilized in this and/or other constructs:

CD3:
(SEQ ID NO: 61)
MLEMKWKALFTAAILQAQLPITEAQSFGLLDPKLCYLLDGILFIY
GVILTALFLRVKFSRSADAPAYQQGQNQLYNELNLGRREEYDVLD
KRRGRDPEMGGKPQRRKNPQEGLYNELQKDKMAEAYSEIGMKGER
RRGKGHDGLYQGLSTATKDTYDALHMQALPPRQCTNYALLKLAGD
VESNPGPMEQGKGLAVLILAIILLQGTLAQSIKGNHLVKVYDYQE
DGSVLLTCDAEAKNITWFKDGKMIGFLTEDKKKWNLGSNAKDPRG
MYQCKGSQNKSKPLQVYYRMCQNCIELNAATISGELFAEIVSIFV
LAVGVYFIAGQDGVRQSRASDKQTLLPNDQLYQPLKDREDDQYSH
LQGNQLRRNVKQTLNFDLLKLAGDVESNPGPMEHSTFLSGLVLAT
LLSQVSPFKIPIEELEDRVFVNCNTSITWVEGTVGTLLSDITRLD
LGKRILDPRGIYRCNGTDIYKDKESTVQVHYRMCQSCVELDPATV
AGIIVTDVIATLLLALGVFCFAGHETGRLSGAADTQALLRNDQVY
QPLRDRDDAQYSHLGGNWARNKEGRGSLLTCGDVEENPGPMQSGT
HWRVLGLCLLSVGVWGQDGNEEMGGITQTPYKVSISGTTVILTCP
QYPGSEILWQHNDKNIGGDEDDKNIGSDEDHLSLKEFSELEQSGY
YVCYPRGSKPEDANFYLYLRARVCENCMEMDVMSVATIVIVDICI
TGGLLLLVYYWSKNRKAKAKPVTRGAGAGGRQRGQNKERPPPVPN
PDYEPIRKGQRDLYSGLNQRRIGPQCTNYALLKLAGDVESNPGP
(SEQ ID NO: 62)
ATGCTCGAGATGAAGTGGAAGGCGCTTTTCACCGCGGCCATCCTG
CAGGCACAGTTGCCGATTACAGAGGCACAGAGCTTTGGCCTGCTG
GATCCCAAACTCTGCTACCTGCTGGATGGAATCCTCTTCATCTAT
GGTGTCATTCTCACTGCCTTGTTCCTGAGAGTGAAGTTCAGCAGG
AGCGCAGACGCCCCCGCGTACCAGCAGGGCCAGAACCAGCTCTAT
AACGAGCTCAATCTAGGACGAAGAGAGGAGTACGATGTTTTGGAC
AAGAGACGTGGCCGGGACCCTGAGATGGGGGGAAAGCCGCAGAGA
AGGAAGAACCCTCAGGAAGGCCTGTACAATGAACTGCAGAAAGAT
AAGATGGCGGAGGCCTACAGTGAGATTGGGATGAAAGGCGAGCGC
CGGAGGGGCAAGGGGCACGATGGCCTTTACCAGGGTCTCAGTACA
GCCACCAAGGACACCTACGACGCCCTTCACATGCAGGCCCTGCCC
CCTCGCCAGTGCACCAACTACGCCCTGCTGAAGCTGGCCGGCGAC
GTGGAGAGCAACCCCGGCCCCATGGAACAGGGGAAGGGCCTGGCT
GTCCTCATCCTGGCTATCATTCTTCTTCAAGGTACTTTGGCCCAG
TCAATCAAAGGAAACCACTTGGTTAAGGTGTATGACTATCAAGAA
GATGGTTCGGTACTTCTGACTTGTGATGCAGAAGCCAAAAATATC
ACATGGTTTAAAGATGGGAAGATGATCGGCTTCCTAACTGAAGAT
AAAAAAAAATGGAATCTGGGAAGTAATGCCAAGGACCCTCGTGGG
ATGTATCAGTGTAAAGGATCACAGAACAAGTCAAAACCACTCCAA
GTGTATTACAGAATGTGTCAGAACTGCATTGAACTAAATGCAGCC
ACCATATCTGGCTTTCTCTTTGCTGAAATCGTCAGCATTTTCGTC
CTTGCTGTTGGGGTCTACTTCATTGCTGGACAGGATGGAGTTCGC
CAGTCGAGAGCTTCAGACAAGCAGACTCTGTTGCCCAATGACCAG
CTCTACCAGCCCCTCAAGGATCGAGAAGATGACCAGTACAGCCAC
CTTCAAGGAAACCAGTTGAGGAGGAATGTGAAGCAGACCCTGAAC
TTCGACCTGCTGAAGCTGGCCGGCGACGTGGAGAGCAACCCCGGC
CCCATGGAGCACAGCACCTTCCTGAGCGGCCTGGTGCTGGCCACC
CTGCTGAGCCAGGTGAGCCCCTTCAAGATCCCCATCGAGGAGCTG
GAGGACAGAGTGTTCGTGAACTGCAACACCAGCATCACCTGGGTG
GAGGGCACCGTGGGCACCCTGCTGAGCGACATCACCAGACTGGAC
CTGGGCAAGAGAATCCTGGACCCCAGAGGCATCTACAGATGCAAC
GGCACCGACATCTACAAGGACAAGGAGAGCACCGTGCAGGTGCAC
TACAGAATGTGCCAGAGCTGCGTGGAGCTGGACCCCGCCACCGTG
GCCGGCATCATCGTGACCGACGTGATCGCCACCCTGCTGCTGGCC
CTGGGCGTGTTCTGCTTCGCCGGCCACGAGACCGGCAGACTGAGC
GGCGCCGCCGACACCCAGGCCCTGCTGAGAAACGACCAGGTGTAC
CAGCCCCTGAGAGACAGAGACGACGCCCAGTACAGCCACCTGGGC
GGCAACTGGGCCAGAAACAAGGAGGGCAGAGGCAGCCTGCTGACC
TGCGGCGACGTGGAGGAGAACCCCGGCCCCATGCAGAGCGGCACC
CACTGGAGAGTGCTGGGCCTGTGCCTGCTGAGCGTGGGCGTGTGG
GGCCAGGACGGCAACGAGGAGATGGGCGGCATCACCCAGACCCCC
TACAAGGTGAGCATCAGCGGCACCACCGTGATCCTGACCTGCCCC
CAGTACCCCGGCAGCGAGATCCTGTGGCAGCACAACGACAAGAAC
ATCGGCGGCGACGAGGACGACAAGAACATCGGCAGCGACGAGGAC
CACCTGAGCCTGAAGGAGTTCAGCGAGCTGGAGCAGAGCGGCTAC
TACGTGTGCTACCCCAGAGGCAGCAAGCCCGAGGACGCCAACTTC
TACCTGTACCTGAGAGCCAGAGTGTGCGAGAACTGCATGGAGATG
GACGTGATGAGCGTGGCCACCATCGTGATCGTGGACATCTGCATC
ACCGGCGGCCTGCTGCTGCTGGTGTACTACTGGAGCAAGAACAGA
AAGGCCAAGGCCAAGCCCGTGACCAGAGGCGCCGGCGCCGGCGGC
AGACAGAGAGGCCAGAACAAGGAGAGACCCCCCCCCGTGCCCAAC
CCCGACTACGAGCCCATCAGAAAGGGCCAGAGAGACCTGTACAGC
GGCCTGAACCAGAGAAGAATCGGACCGCAGTGTACTAATTATGCT
CTCTTGAAATTGGCTGGAGATGTTGAGAGCAATCCCGGGCCC
SP CD8:
(SEQ ID NO: 63)
MRICLTSDRLAPAAGLAAPRRQAV
(SEQ ID NO: 64)
atgcgcatttgcctgaccagcgatcgcctggcgccggcggcgggc
ctggcggcgccgcgccgccaggcggtg
IL-21:
(SEQ ID NO: 65)
HKSSSQGQDRHMIRMRQLIDIVDQLKNYVNDLVPEFLPAPEDVET
NCEWSAFSCFQKAQLKSANTGNNERIINVSIKKLKRKPPSTNAGR
RQKHRLTCPSCDSYEKKPPKEFLERFKSLLQKMIHQHLSSRTHGS
EDS
(SEQ ID NO: 66)
CATAAATCTTCCTCTCAAGGTCAGGACCGCCATATGATTCGAATG
CGGCAGCTGATTGACATAGTCGATCAACTGAAGAACTATGTGAAT
GATCTTGTGCCCGAGTTTTTGCCAGCCCCTGAAGACGTAGAAACT
AATTGTGAGTGGAGTGCCTTTTCCTGCTTTCAAAAGGCACAGCTG
AAATCCGCCAACACGGGCAATAACGAACGGATAATTAACGTATCC
ATTAAGAAGCTGAAGCGGAAGCCGCCCTCAACCAATGCGGGACGG
CGGCAAAAGCATCGCTTGACCTGTCCGTCATGCGACAGCTACGAG
AAAAAGCCCCCGAAGGAGTTCTTGGAACGCTTCAAGAGTCTCCTT
CAGAAAATGATTCACCAGCACCTGTCCTCACGGACGCACGGAAGC
GAGGACAGT
CD8 hinge:
(SEQ ID NO: 67)
TTTPAPRPPTPAPTIASQPLSLRPEACRPAAGGAVHTRGLDFACD
(SEQ ID NO: 68)
ACCACGACGCCAGCGCCGCGACCACCAACACCGGCGCCCACCATC
GCGTCGCAGCCCCTGTCCCTGCGCCCAGAGGCGTGCCGGCCAGCG
GCGGGGGGCGCAGTGCACACGAGGGGGCTGGACTTCGCCTGTGAT
CD28 Transmembrane domain:
(SEQ ID NO: 69)
FWVLVVVGGVLACYSLLVTVAFIIFWV*
(SEQ ID NO: 70)
TTTTGGGTGCTGGTGGTGGTTGGTGGAGTCCTGGCTTGCTATAGC
TTGCTAGTAACAGTGGCCTTTATTATTTTCTGGGTG

Z3: refers to full length CD3zeta, full length CD3 gamma, full length CD3 delta, and full length CD3 epsilon linked to membrane bound IL21 (with CD28 transmembrane domain for the membrane bound IL21), and it may also be referred to as CD3ZGDEFL8SP21CD8 with a representative sequence as follows:

(SEQ ID NO: 71)
MLEMKWKALFTAAILQAQLPITEAQSFGLLDPKLCYLLD
GILFIYGVILTALFLRVKFSRSADAPAYQQGQNQLYNEL
NLGRREEYDVLDKRRGRDPEMGGKPQRRKNPQEGLYNEL
QKDKMAEAYSEIGMKGERRRGKGHDGLYQGLSTATKDTY
DALHMQALPPRQCTNYALLKLAGDVESNPGPMEQGKGLA
VLILAIILLQGTLAQSIKGNHLVKVYDYQEDGSVLLTCD
AEAKNITWFKDGKMIGFLTEDKKKWNLGSNAKDPRGMYQ
CKGSQNKSKPLQVYYRMCQNCIELNAATISGELFAEIVS
IFVLAVGVYFIAGQDGVRQSRASDKQTLLPNDQLYQPLK
DREDDQYSHLQGNQLRRNVKQTLNFDLLKLAGDVESNPG
PMEHSTFLSGLVLATLLSQVSPFKIPIEELEDRVFVNCN
TSITWVEGTVGTLLSDITRLDLGKRILDPRGIYRCNGTD
IYKDKESTVQVHYRMCQSCVELDPATVAGIIVTDVIATL
LLALGVFCFAGHETGRLSGAADTQALLRNDQVYQPLRDR
DDAQYSHLGGNWARNKEGRGSLLTCGDVEENPGPMQSGT
HWRVLGLCLLSVGVWGQDGNEEMGGITQTPYKVSISGTT
VILTCPQYPGSEILWQHNDKNIGGDEDDKNIGSDEDHLS
LKEFSELEQSGYYVCYPRGSKPEDANFYLYLRARVCENC
MEMDVMSVATIVIVDICITGGLLLLVYYWSKNRKAKAKP
VTRGAGAGGRQRGQNKERPPPVPNPDYEPIRKGQRDLYS
GLNQRRIGPQCTNYALLKLAGDVESNPGPMRICLTSDRL
APAAGLAAPRRQAVHKSSSQGQDRHMIRMRQLIDIVDQL
KNYVNDLVPEFLPAPEDVETNCEWSAFSCFQKAQLKSAN
TGNNERIINVSIKKLKRKPPSTNAGRRQKHRLTCPSCDS
YEKKPPKEFLERFKSLLQKMIHQHLSSRTHGSEDSTTTP
APRPPTPAPTIASQPLSLRPEACRPAAGGAVHTRGLDFA
CDIYIWAPLAGTCGVLLLSLVIT.

For CD3ZGDEFL8SP21CD8, the corresponding component sequences are as follows, although these particular sequences or others may be utilized in this and/or other constructs:

CD3:
(SEQ ID NO: 61)
MLEMKWKALFTAAILQAQLPITEAQSFGLLDPKLCYLLD
GILFIYGVILTALFLRVKESRSADAPAYQQGQNQLYNEL
NLGRREEYDVLDKRRGRDPEMGGKPQRRKNPQEGLYNEL
QKDKMAEAYSEIGMKGERRRGKGHDGLYQGLSTATKDTY
DALHMQALPPRQCTNYALLKLAGDVESNPGPMEQGKGLA
VLILAIILLQGTLAQSIKGNHLVKVYDYQEDGSVLLTCD
AEAKNITWFKDGKMIGFLTEDKKKWNLGSNAKDPRGMYQ
CKGSQNKSKPLQVYYRMCQNCIELNAATISGELFAEIVS
IFVLAVGVYFIAGQDGVRQSRASDKQTLLPNDQLYQPLK
DREDDQYSHLQGNQLRRNVKQTLNFDLLKLAGDVESNPG
PMEHSTFLSGLVLATLLSQVSPFKIPIEELEDRVFVNCN
TSITWVEGTVGTLLSDITRLDLGKRILDPRGIYRCNGTD
IYKDKESTVQVHYRMCQSCVELDPATVAGIIVTDVIATL
LLALGVFCFAGHETGRLSGAADTQALLRNDQVYQPLRDR
DDAQYSHLGGNWARNKEGRGSLLTCGDVEENPGPMQSGT
HWRVLGLCLLSVGVWGQDGNEEMGGITQTPYKVSISGTT
VILTCPQYPGSEILWQHNDKNIGGDEDDKNIGSDEDHLS
LKEFSELEQSGYYVCYPRGSKPEDANFYLYLRARVCENC
MEMDVMSVATIVIVDICITGGLLLLVYYWSKNRKAKAKP
VTRGAGAGGRQRGQNKERPPPVPNPDYEPIRKGQRDLYS
GLNQRRIGPQCTNYALLKLAGDVESNPGP
(SEQ ID NO: 62)
ATGCTCGAGATGAAGTGGAAGGCGCTTTTCACCGCGGCC
ATCCTGCAGGCACAGTTGCCGATTACAGAGGCACAGAGC
TTTGGCCTGCTGGATCCCAAACTCTGCTACCTGCTGGAT
GGAATCCTCTTCATCTATGGTGTCATTCTCACTGCCTTG
TTCCTGAGAGTGAAGTTCAGCAGGAGCGCAGACGCCCCC
GCGTACCAGCAGGGCCAGAACCAGCTCTATAACGAGCTC
AATCTAGGACGAAGAGAGGAGTACGATGTTTTGGACAAG
AGACGTGGCCGGGACCCTGAGATGGGGGGAAAGCCGCAG
AGAAGGAAGAACCCTCAGGAAGGCCTGTACAATGAACTG
CAGAAAGATAAGATGGCGGAGGCCTACAGTGAGATTGGG
ATGAAAGGCGAGCGCCGGAGGGGCAAGGGGCACGATGGC
CTTTACCAGGGTCTCAGTACAGCCACCAAGGACACCTAC
GACGCCCTTCACATGCAGGCCCTGCCCCCTCGCCAGTGC
ACCAACTACGCCCTGCTGAAGCTGGCCGGCGACGTGGAG
AGCAACCCCGGCCCCATGGAACAGGGGAAGGGCCTGGCT
GTCCTCATCCTGGCTATCATTCTTCTTCAAGGTACTTTG
GCCCAGTCAATCAAAGGAAACCACTTGGTTAAGGTGTAT
GACTATCAAGAAGATGGTTCGGTACTTCTGACTTGTGAT
GCAGAAGCCAAAAATATCACATGGTTTAAAGATGGGAAG
ATGATCGGCTTCCTAACTGAAGATAAAAAAAAATGGAAT
CTGGGAAGTAATGCCAAGGACCCTCGTGGGATGTATCAG
TGTAAAGGATCACAGAACAAGTCAAAACCACTCCAAGTG
TATTACAGAATGTGTCAGAACTGCATTGAACTAAATGCA
GCCACCATATCTGGCTTTCTCTTTGCTGAAATCGTCAGC
ATTTTCGTCCTTGCTGTTGGGGTCTACTTCATTGCTGGA
CAGGATGGAGTTCGCCAGTCGAGAGCTTCAGACAAGCAG
ACTCTGTTGCCCAATGACCAGCTCTACCAGCCCCTCAAG
GATCGAGAAGATGACCAGTACAGCCACCTTCAAGGAAAC
CAGTTGAGGAGGAATGTGAAGCAGACCCTGAACTTCGAC
CTGCTGAAGCTGGCCGGCGACGTGGAGAGCAACCCCGGC
CCCATGGAGCACAGCACCTTCCTGAGCGGCCTGGTGCTG
GCCACCCTGCTGAGCCAGGTGAGCCCCTTCAAGATCCCC
ATCGAGGAGCTGGAGGACAGAGTGTTCGTGAACTGCAAC
ACCAGCATCACCTGGGTGGAGGGCACCGTGGGCACCCTG
CTGAGCGACATCACCAGACTGGACCTGGGCAAGAGAATC
CTGGACCCCAGAGGCATCTACAGATGCAACGGCACCGAC
ATCTACAAGGACAAGGAGAGCACCGTGCAGGTGCACTAC
AGAATGTGCCAGAGCTGCGTGGAGCTGGACCCCGCCACC
GTGGCCGGCATCATCGTGACCGACGTGATCGCCACCCTG
CTGCTGGCCCTGGGCGTGTTCTGCTTCGCCGGCCACGAG
ACCGGCAGACTGAGCGGCGCCGCCGACACCCAGGCCCTG
CTGAGAAACGACCAGGTGTACCAGCCCCTGAGAGACAGA
GACGACGCCCAGTACAGCCACCTGGGCGGCAACTGGGCC
AGAAACAAGGAGGGCAGAGGCAGCCTGCTGACCTGCGGC
GACGTGGAGGAGAACCCCGGCCCCATGCAGAGCGGCACC
CACTGGAGAGTGCTGGGCCTGTGCCTGCTGAGCGTGGGC
GTGTGGGGCCAGGACGGCAACGAGGAGATGGGCGGCATC
ACCCAGACCCCCTACAAGGTGAGCATCAGCGGCACCACC
GTGATCCTGACCTGCCCCCAGTACCCCGGCAGCGAGATC
CTGTGGCAGCACAACGACAAGAACATCGGCGGCGACGAG
GACGACAAGAACATCGGCAGCGACGAGGACCACCTGAGC
CTGAAGGAGTTCAGCGAGCTGGAGCAGAGCGGCTACTAC
GTGTGCTACCCCAGAGGCAGCAAGCCCGAGGACGCCAAC
TTCTACCTGTACCTGAGAGCCAGAGTGTGCGAGAACTGC
ATGGAGATGGACGTGATGAGCGTGGCCACCATCGTGATC
GTGGACATCTGCATCACCGGCGGCCTGCTGCTGCTGGTG
TACTACTGGAGCAAGAACAGAAAGGCCAAGGCCAAGCCC
GTGACCAGAGGCGCCGGCGCCGGCGGCAGACAGAGAGGC
CAGAACAAGGAGAGACCCCCCCCCGTGCCCAACCCCGAC
TACGAGCCCATCAGAAAGGGCCAGAGAGACCTGTACAGC
GGCCTGAACCAGAGAAGAATCGGACCGCAGTGTACTAAT
TATGCTCTCTTGAAATTGGCTGGAGATGTTGAGAGCAAT
CCCGGGCCC
SP CD8:
(SEQ ID NO: 63)
MRICLTSDRLAPAAGLAAPRRQAV
(SEQ ID NO: 64)
atgcgcatttgcctgaccagcgatcgcctggcgccggcg
ggggcctggcggcgccgcgccgccaggcggtg
IL-21:
(SEQ ID NO: 65)
HKSSSQGQDRHMIRMRQLIDIVDQLKNYVNDLVPEFLPA
PEDVETNCEWSAFSCFQKAQLKSANTGNNERIINVSIKK
LKRKPPSTNAGRRQKHRLTCPSCDSYEKKPPKEFLEREK
SLLQKMIHQHLSSRTHGSEDS
(SEQ ID NO: 65)
cataaatcttcctctcaaggtcaggaccgccatatgatt
cgaatgcggcagctgattgacatagtcgatcaactgaag
aactatgtgaatgatcttgtgcccgagtttttgccagcc
cctgaagacgtagaaactaattgtgagtggagtgccttt
tcctgctttcaaaaggcacagctgaaatccgccaacacg
ggcaataacgaacggataattaacgtatccattaagaag
ctgaagcggaagccgccctcaaccaatgcgggacggcgg
caaaagcatcgcttgacctgtccgtcatgcgacagctac
gagaaaaagcccccgaaggagttcttggaacgcttcaag
agtctccttcagaaaatgattcaccagcacctgtcctca
cggacgcacggaagcgaggacagt
CD8 hinge:
(SEQ ID NO: 67)
TTTPAPRPPTPAPTIASQPLSLRPEACRPAAGGAVHTRGLDFACD
(SEQ ID NO: 68)
ACCACGACGCCAGCGCCGCGACCACCAACACCGGCGCCC
ACCATCGCGTCGCAGCCCCTGTCCCTGCGCCCAGAGGCG
TGCCGGCCAGCGGCGGGGGGCGCAGTGCACACGAGGGGG
CTGGACTTCGCCTGTGAT
CD8 Transmembrane Domain:
(SEQ ID NO: 72)
IYIWAPLAGTCGVLLLSLVIT*
(SEQ ID NO: 73)
ATCTACATCTGGGCGCCCTTGGCCGGGACTTGTGGGGTC
CTTCTCCTGTCACTGGTTATCACC

In certain embodiments, provided herein are CD3 constructs comprising a fusion with an intracellular co-stimulatory domain derived from CD16, NKG2D, DAP10, DAP12, 2B4, 4-1BB, CD2, CD28, DNAM, or any combination thereof. In certain embodiments, an intracellular co-stimulatory domain is fused to CD3δ, CD3ε, CD3γ, and/or CD3ζ. In certain embodiments, such a CD3 fusion construct comprises a CD3ζ fused to a DAP10 intracellular co-stimulatory domain. In certain embodiments, such a CD3 fusion construct comprises a CD3ζ fused to a CD28 intracellular co-stimulatory domain. In certain embodiments, such a CD3 fusion construct comprises a CD3ζ fused to a DAP10 intracellular co-stimulatory domain and a CD28 intracellular co-stimulatory domain. In certain embodiments, a CD3ζ fused to a DAP10 intracellular co-stimulatory domain is represented by a nucleotide sequence that is at least, or exactly, 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 106. In certain embodiments, a CD3ζ fused to a CD28 intracellular co-stimulatory domain is represented by a nucleotide sequence that is at least, or exactly, 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 107. In certain embodiments, a CD3ζ fused to a DAP10 intracellular co-stimulatory domain and a CD28 intracellular co-stimulatory domain is represented by a nucleotide sequence that is at least, or exactly, 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 108. In certain embodiments, a CD3ζ fused to a DAP10 intracellular co-stimulatory domain is represented by an amino acid sequence that is at least, or exactly, 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 109. In certain embodiments, a CD3ζ fused to a CD28 intracellular co-stimulatory domain is represented by an amino acid sequence that is at least, or exactly, 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 110. In certain embodiments, a CD3ζ fused to a DAP10 intracellular co-stimulatory domain and a CD28 intracellular co-stimulatory domain is represented by an amino acid sequence that is at least, or exactly, 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 111. In certain embodiments, a CD3ζ fused to an intracellular domain may not comprise a C terminal 2A domain. In certain embodiments, a CD3ζ fused to an intracellular domain may not comprise an N terminal signal peptide domain.

(SEQ ID NO: 106)
ATGAAGTGGAAGGCGCTTTTCACCGCGGCCATCCTGCAGGCACAG
TTGCCGATTACAGAGGCACAGAGCTTTGGCCTGCTGGATCCCAAA
CTCTGCTACCTGCTGGATGGAATCCTCTTCATCTATGGTGTCATT
CTCACTGCCTTGTTCCTGCTTTGCGCACGCCCACGCCGCAGCCCC
GCCCAAGAAGATGGCAAAGTCTACATCAACATGCCAGGCAGGGGC
AGAGTGAAGTTCAGCAGGAGCGCAGACGCCCCCGCGTACCAGCAG
GGCCAGAACCAGCTCTATAACGAGCTCAATCTAGGACGAAGAGAG
GAGTACGATGTTTTGGACAAGAGACGTGGCCGGGACCCTGAGATG
GGGGGAAAGCCGCAGAGAAGGAAGAACCCTCAGGAAGGCCTGTAC
AATGAACTGCAGAAAGATAAGATGGCGGAGGCCTACAGTGAGATT
GGGATGAAAGGCGAGCGCCGGAGGGGCAAGGGGCACGATGGCCTT
TACCAGGGTCTCAGTACAGCCACCAAGGACACCTACGACGCCCTT
CACATGCAGGCCCTGCCCCCTCGCCAGTGCACCAACTACGCCCTG
CTGAAGCTGGCCGGCGACGTGGAGAGCAACCCCGGCCCC
(SEQ ID NO: 107)
ATGAAGTGGAAGGCGCTTTTCACCGCGGCCATCCTGCAGGCACAG
TTGCCGATTACAGAGGCACAGAGCTTTGGCCTGCTGGATCCCAAA
CTCTGCTACCTGCTGGATGGAATCCTCTTCATCTATGGTGTCATT
CTCACTGCCTTGTTCCTGAGGAGTAAGAGGAGCAGGCTCCTGCAC
AGTGACTACATGAACATGACTCCCCGCCGCCCCGGGCCCACCCGC
AAGCATTACCAGCCCTATGCCCCACCACGCGACTTCGCAGCCTAT
CGCTCAAGAGTGAAGTTCAGCAGGAGCGCAGACGCCCCCGCGTAC
CAGCAGGGCCAGAACCAGCTCTATAACGAGCTCAATCTAGGACGA
AGAGAGGAGTACGATGTTTTGGACAAGAGACGTGGCCGGGACCCT
GAGATGGGGGGAAAGCCGCAGAGAAGGAAGAACCCTCAGGAAGGC
CTGTACAATGAACTGCAGAAAGATAAGATGGCGGAGGCCTACAGT
GAGATTGGGATGAAAGGCGAGCGCCGGAGGGGCAAGGGGCACGAT
GGCCTTTACCAGGGTCTCAGTACAGCCACCAAGGACACCTACGAC
GCCCTTCACATGCAGGCCCTGCCCCCTCGCCAGTGCACCAACTAC
GCCCTGCTGAAGCTGGCCGGCGACGTGGAGAGCAACCCCGGCCCC
(SEQ ID NO: 108)
ATGAAGTGGAAGGCGCTTTTCACCGCGGCCATCCTGCAGGCACAG
TTGCCGATTACAGAGGCACAGAGCTTTGGCCTGCTGGATCCCAAA
CTCTGCTACCTGCTGGATGGAATCCTCTTCATCTATGGTGTCATT
CTCACTGCCTTGTTCCTGAGGAGTAAGAGGAGCAGGCTCCTGCAC
AGTGACTACATGAACATGACTCCCCGCCGCCCCGGGCCCACCCGC
AAGCATTACCAGCCCTATGCCCCACCACGCGACTTCGCAGCCTAT
CGCTCACTTTGCGCACGCCCACGCCGCAGCCCCGCCCAAGAAGAT
GGCAAAGTCTACATCAACATGCCAGGCAGGGGCAGAGTGAAGTTC
AGCAGGAGCGCAGACGCCCCCGCGTACCAGCAGGGCCAGAACCAG
CTCTATAACGAGCTCAATCTAGGACGAAGAGAGGAGTACGATGTT
TTGGACAAGAGACGTGGCCGGGACCCTGAGATGGGGGGAAAGCCG
CAGAGAAGGAAGAACCCTCAGGAAGGCCTGTACAATGAACTGCAG
AAAGATAAGATGGCGGAGGCCTACAGTGAGATTGGGATGAAAGGC
GAGCGCCGGAGGGGCAAGGGGCACGATGGCCTTTACCAGGGTCTC
AGTACAGCCACCAAGGACACCTACGACGCCCTTCACATGCAGGCC
CTGCCCCCTCGCCAGTGCACCAACTACGCCCTGCTGAAGCTGGCC
GGCGACGTGGAGAGCAACCCCGGCCCC
(SEQ ID NO: 109)
MKWKALFTAAILQAQLPITEAQSFGLLDPKLCYLLDGILFIYGVI
LTALFLLCARPRRSPAQEDGKVYINMPGRGRVKFSRSADAPAYQQ
GQNQLYNELNLGRREEYDVLDKRRGRDPEMGGKPQRRKNPQEGLY
NELQKDKMAEAYSEIGMKGERRRGKGHDGLYQGLSTATKDTYDAL
HMQALPPRQCTNYALLKLAGDVESNPGP
(SEQ ID NO: 110)
MKWKALFTAAILQAQLPITEAQSFGLLDPKLCYLLDGILFIYGVI
LTALFLRSKRSRLLHSDYMNMTPRRPGPTRKHYQPYAPPRDFAAY
RSRVKESRSADAPAYQQGQNQLYNELNLGRREEYDVLDKRRGRDP
EMGGKPQRRKNPQEGLYNELQKDKMAEAYSEIGMKGERRRGKGHD
GLYQGLSTATKDTYDALHMQALPPRQCTNYALLKLAGDVESNPGP
(SEQ ID NO: 111)
MKWKALFTAAILQAQLPITEAQSFGLLDPKLCYLLDGILFIYGVI
LTALFLRSKRSRLLHSDYMNMTPRRPGPTRKHYQPYAPPRDFAAY
RSLCARPRRSPAQEDGKVYINMPGRGRVKFSRSADAPAYQQGQNQ
LYNELNLGRREEYDVLDKRRGRDPEMGGKPQRRKNPQEGLYNELQ
KDKMAEAYSEIGMKGERRRGKGHDGLYQGLSTATKDTYDALHMQA
LPPRQCTNYALLKLAGDVESNPGP

In certain embodiments, a DAP10 intracellular co-stimulatory domain is represented by a nucleotide sequence that is at least, or exactly, 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 112. In certain embodiments, a CD28 intracellular co-stimulatory domain is represented by a nucleotide sequence that is at least, or exactly, 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 113. In certain embodiments, a DAP10 intracellular co-stimulatory domain and CD28 intracellular co-stimulatory domain is represented by a nucleotide sequence that is at least, or exactly, 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 114. In certain embodiments, a DAP10 intracellular co-stimulatory domain is represented by an amino acid sequence that is at least, or exactly, 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 115. In certain embodiments, a CD28 intracellular co-stimulatory domain is represented by an amino acid sequence that is at least, or exactly, 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 116. In certain embodiments, a DAP10 intracellular co-stimulatory domain and CD28 intracellular co-stimulatory domain is represented by an amino acid sequence that is at least, or exactly, 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 117.

(SEQ ID NO: 112)
CTTTGCGCACGCCCACGCCGCAGCCCCGCCCAAGAAGATGGCAAA
GTCTACATCAACATGCCAGGCAGGGGC
(SEQ ID NO: 113)
AGGAGTAAGAGGAGCAGGCTCCTGCACAGTGACTACATGAACATG
ACTCCCCGCCGCCCCGGGCCCACCCGCAAGCATTACCAGCCCTAT
GCCCCACCACGCGACTTCGCAGCCTATCGCTCA
(SEQ ID NO: 114)
AGGAGTAAGAGGAGCAGGCTCCTGCACAGTGACTACATGAACATG
ACTCCCCGCCGCCCCGGGCCCACCCGCAAGCATTACCAGCCCTAT
GCCCCACCACGCGACTTCGCAGCCTATCGCTCACTTTGCGCACGC
CCACGCCGCAGCCCCGCCCAAGAAGATGGCAAAGTCTACATCAAC
ATGCCAGGCAGGGGC
(SEQ ID NO: 115)
LCARPRRSPAQEDGKVYINMPGRG
(SEQ ID NO: 116)
RSKRSRLLHSDYMNMTPRRPGPTRKHYQPYAPPRDFAAYRS
(SEQ ID NO: 117)
RSKRSRLLHSDYMNMTPRRPGPTRKHYQPYAPPRDFAAYRSLCAR
PRRSPAQEDGKVYINMPGRG

UTNK15-DAP10: refers to full length CD3zeta comprising a fusion with an intracellular co-stimulatory domain derived from DAP10, full length CD3 gamma, full length CD3 delta, and full length CD3 epsilon linked to I15, it may be represented by a nucleotide sequence that is at least, or exactly, 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 118. In certain embodiments, a UTNK15-DAP10 amino acid sequence may be represented by an amino acid sequence that is at least, or exactly, 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 119.

(SEQ ID NO: 118)
ATGAAGTGGAAGGCGCTTTTCACCGCGGCCATCCTGCAGGCACAG
TTGCCGATTACAGAGGCACAGAGCTTTGGCCTGCTGGATCCCAAA
CTCTGCTACCTGCTGGATGGAATCCTCTTCATCTATGGTGTCATT
CTCACTGCCTTGTTCCTGCTTTGCGCACGCCCACGCCGCAGCCCC
GCCCAAGAAGATGGCAAAGTCTACATCAACATGCCAGGCAGGGGC
AGAGTGAAGTTCAGCAGGAGCGCAGACGCCCCCGCGTACCAGCAG
GGCCAGAACCAGCTCTATAACGAGCTCAATCTAGGACGAAGAGAG
GAGTACGATGTTTTGGACAAGAGACGTGGCCGGGACCCTGAGATG
GGGGGAAAGCCGCAGAGAAGGAAGAACCCTCAGGAAGGCCTGTAC
AATGAACTGCAGAAAGATAAGATGGCGGAGGCCTACAGTGAGATT
GGGATGAAAGGCGAGCGCCGGAGGGGCAAGGGGCACGATGGCCTT
TACCAGGGTCTCAGTACAGCCACCAAGGACACCTACGACGCCCTT
CACATGCAGGCCCTGCCCCCTCGCCAGTGCACCAACTACGCCCTG
CTGAAGCTGGCCGGCGACGTGGAGAGCAACCCCGGCCCCATGGAA
CAGGGGAAGGGCCTGGCTGTCCTCATCCTGGCTATCATTCTTCTT
CAAGGTACTTTGGCCCAGTCAATCAAAGGAAACCACTTGGTTAAG
GTGTATGACTATCAAGAAGATGGTTCGGTACTTCTGACTTGTGAT
GCAGAAGCCAAAAATATCACATGGTTTAAAGATGGGAAGATGATC
GGCTTCCTAACTGAAGATAAAAAAAAATGGAATCTGGGAAGTAAT
GCCAAGGACCCTCGTGGGATGTATCAGTGTAAAGGATCACAGAAC
AAGTCAAAACCACTCCAAGTGTATTACAGAATGTGTCAGAACTGC
ATTGAACTAAATGCAGCCACCATATCTGGCTTTCTCTTTGCTGAA
ATCGTCAGCATTTTCGTCCTTGCTGTTGGGGTCTACTTCATTGCT
GGACAGGATGGAGTTCGCCAGTCGAGAGCTTCAGACAAGCAGACT
CTGTTGCCCAATGACCAGCTCTACCAGCCCCTCAAGGATCGAGAA
GATGACCAGTACAGCCACCTTCAAGGAAACCAGTTGAGGAGGAAT
GTGAAGCAGACCCTGAACTTCGACCTGCTGAAGCTGGCCGGCGAC
GTGGAGAGCAACCCCGGCCCCATGGAGCACAGCACCTTCCTGAGC
GGCCTGGTGCTGGCCACCCTGCTGAGCCAGGTGAGCCCCTTCAAG
ATCCCCATCGAGGAGCTGGAGGACAGAGTGTTCGTGAACTGCAAC
ACCAGCATCACCTGGGTGGAGGGCACCGTGGGCACCCTGCTGAGC
GACATCACCAGACTGGACCTGGGCAAGAGAATCCTGGACCCCAGA
GGCATCTACAGATGCAACGGCACCGACATCTACAAGGACAAGGAG
AGCACCGTGCAGGTGCACTACAGAATGTGCCAGAGCTGCGTGGAG
CTGGACCCCGCCACCGTGGCCGGCATCATCGTGACCGACGTGATC
GCCACCCTGCTGCTGGCCCTGGGCGTGTTCTGCTTCGCCGGCCAC
GAGACCGGCAGACTGAGCGGCGCCGCCGACACCCAGGCCCTGCTG
AGAAACGACCAGGTGTACCAGCCCCTGAGAGACAGAGACGACGCC
CAGTACAGCCACCTGGGCGGCAACTGGGCCAGAAACAAGGAGGGC
AGAGGCAGCCTGCTGACCTGCGGCGACGTGGAGGAGAACCCCGGC
CCCATGCAGAGCGGCACCCACTGGAGAGTGCTGGGCCTGTGCCTG
CTGAGCGTGGGCGTGTGGGGCCAGGACGGCAACGAGGAGATGGGC
GGCATCACCCAGACCCCCTACAAGGTGAGCATCAGCGGCACCACC
GTGATCCTGACCTGCCCCCAGTACCCCGGCAGCGAGATCCTGTGG
CAGCACAACGACAAGAACATCGGCGGCGACGAGGACGACAAGAAC
ATCGGCAGCGACGAGGACCACCTGAGCCTGAAGGAGTTCAGCGAG
CTGGAGCAGAGCGGCTACTACGTGTGCTACCCCAGAGGCAGCAAG
CCCGAGGACGCCAACTTCTACCTGTACCTGAGAGCCAGAGTGTGC
GAGAACTGCATGGAGATGGACGTGATGAGCGTGGCCACCATCGTG
ATCGTGGACATCTGCATCACCGGCGGCCTGCTGCTGCTGGTGTAC
TACTGGAGCAAGAACAGAAAGGCCAAGGCCAAGCCCGTGACCAGA
GGCGCCGGCGCCGGCGGCAGACAGAGAGGCCAGAACAAGGAGAGA
CCCCCCCCCGTGCCCAACCCCGACTACGAGCCCATCAGAAAGGGC
CAGAGAGACCTGTACAGCGGCCTGAACCAGAGAAGAATCGGACCG
CAGTGTACTAATTATGCTCTCTTGAAATTGGCTGGAGATGTTGAG
AGCAATCCCGGGCCCATGCGCATTAGCAAGCCCCACCTGCGGAGC
ATCAGCATCCAGTGCTACCTGTGCCTGCTGCTGAACAGCCACTTC
CTGACCGAGGCCGGCATCCACGTGTTCATCCTGGGCTGCTTCAGC
GCCGGACTGCCCAAGACCGAGGCCAACTGGGTGAACGTGATCAGC
GACCTGAAGAAGATCGAGGACCTGATCCAGAGCATGCACATCGAC
GCCACCCTGTACACCGAGAGCGACGTGCACCCCAGCTGCAAGGTG
ACCGCCATGAAGTGCTTTCTGCTGGAACTGCAGGTGATCAGCCTG
GAAAGCGGCGACGCCAGCATCCACGACACCGTGGAGAACCTGATC
ATCCTGGCCAACAACAGCCTGAGCAGCAACGGCAACGTGACCGAG
AGCGGCTGCAAAGAGTGCGAGGAACTGGAAGAGAAGAACATCAAA
GAGTTTCTGCAGAGCTTCGTGCACATCGTGCAGATGTTCATCAAC
ACCAGC
(SEQ ID NO: 119)
MKWKALFTAAILQAQLPITEAQSFGLLDPKLCYLLDGILFIYGVI
LTALFLLCARPRRSPAQEDGKVYINMPGRGRVKFSRSADAPAYQQ
GQNQLYNELNLGRREEYDVLDKRRGRDPEMGGKPQRRKNPQEGLY
NELQKDKMAEAYSEIGMKGERRRGKGHDGLYQGLSTATKDTYDAL
HMQALPPRQCTNYALLKLAGDVESNPGPMEQGKGLAVLILAIILL
QGTLAQSIKGNHLVKVYDYQEDGSVLLTCDAEAKNITWFKDGKMI
GFLTEDKKKWNLGSNAKDPRGMYQCKGSQNKSKPLQVYYRMCQNC
IELNAATISGELFAEIVSIFVLAVGVYFIAGQDGVRQSRASDKQT
LLPNDQLYQPLKDREDDQYSHLQGNQLRRNVKQTLNFDLLKLAGD
VESNPGPMEHSTFLSGLVLATLLSQVSPFKIPIEELEDRVFVNCN
TSITWVEGTVGTLLSDITRLDLGKRILDPRGIYRCNGTDIYKDKE
STVQVHYRMCQSCVELDPATVAGIIVTDVIATLLLALGVFCFAGH
ETGRLSGAADTQALLRNDQVYQPLRDRDDAQYSHLGGNWARNKEG
RGSLLTCGDVEENPGPMQSGTHWRVLGLCLLSVGVWGQ.DGNEEM
GGITQTPYKVSISGTTVILTCPQYPGSEILWQHNDKNIGGDEDDK
NIGSDEDHLSLKEFSELEQSGYYVCYPRGSKPEDANFYLYLRARV
CENCMEMDVMSVATIVIVDICITGGLLLLVYYWSKNRKAKAKPVT
RGAGAGGRQRGQNKERPPPVPNPDYEPIRKGQRDLYSGLNQRRIG
PQCTNYALLKLAGDVESNPGPMRISKPHLRSISIQCYLCLLLNSH
ELTEAGIHVFILGCFSAGLPKTEANWVNVISDLKKIEDLIQSMHI
DATLYTESDVHPSCKVTAMKCFLLELQVISLESGDASIHDTVENL
IILANNSLSSNGNVTESGCKECEELEEKNIKEFLQSFVHIVQMFI
NTS

UTNK15-28: refers to full length CD3zeta comprising a fusion with an intracellular co-stimulatory domain derived from CD28, full length CD3 gamma, full length CD3 delta, and full length CD3 epsilon linked to IL15, it may be represented by a nucleotide sequence that is at least, or exactly, 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 120. In certain embodiments, a UTNK15-28 amino acid sequence may be represented by an amino acid sequence that is at least, or exactly, 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 121.

(SEQ ID NO: 120)
ATGAAGTGGAAGGCGCTTTTCACCGCGGCCATCCTGCAG
GCACAGTTGCCGATTACAGAGGCACAGAGCTTTGGCCTG
CTGGATCCCAAACTCTGCTACCTGCTGGATGGAATCCTC
TTCATCTATGGTGTCATTCTCACTGCCTTGTTCCTGAGG
AGTAAGAGGAGCAGGCTCCTGCACAGTGACTACATGAAC
ATGACTCCCCGCCGCCCCGGGCCCACCCGCAAGCATTAC
CAGCCCTATGCCCCACCACGCGACTTCGCAGCCTATCGC
TCAAGAGTGAAGTTCAGCAGGAGCGCAGACGCCCCCGCG
TACCAGCAGGGCCAGAACCAGCTCTATAACGAGCTCAAT
CTAGGACGAAGAGAGGAGTACGATGTTTTGGACAAGAGA
CGTGGCCGGGACCCTGAGATGGGGGGAAAGCCGCAGAGA
AGGAAGAACCCTCAGGAAGGCCTGTACAATGAACTGCAG
AAAGATAAGATGGCGGAGGCCTACAGTGAGATTGGGATG
AAAGGCGAGCGCCGGAGGGGCAAGGGGCACGATGGCCTT
TACCAGGGTCTCAGTACAGCCACCAAGGACACCTACGAC
GCCCTTCACATGCAGGCCCTGCCCCCTCGCCAGTGCACC
AACTACGCCCTGCTGAAGCTGGCCGGCGACGTGGAGAGC
AACCCCGGCCCCATGGAACAGGGGAAGGGCCTGGCTGTC
CTCATCCTGGCTATCATTCTTCTTCAAGGTACTTTGGCC
CAGTCAATCAAAGGAAACCACTTGGTTAAGGTGTATGAC
TATCAAGAAGATGGTTCGGTACTTCTGACTTGTGATGCA
GAAGCCAAAAATATCACATGGTTTAAAGATGGGAAGATG
ATCGGCTTCCTAACTGAAGATAAAAAAAAATGGAATCTG
GGAAGTAATGCCAAGGACCCTCGTGGGATGTATCAGTGT
AAAGGATCACAGAACAAGTCAAAACCACTCCAAGTGTAT
TACAGAATGTGTCAGAACTGCATTGAACTAAATGCAGCC
ACCATATCTGGCTTTCTCTTTGCTGAAATCGTCAGCATT
TTCGTCCTTGCTGTTGGGGTCTACTTCATTGCTGGACAG
GATGGAGTTCGCCAGTCGAGAGCTTCAGACAAGCAGACT
CTGTTGCCCAATGACCAGCTCTACCAGCCCCTCAAGGAT
CGAGAAGATGACCAGTACAGCCACCTTCAAGGAAACCAG
TTGAGGAGGAATGTGAAGCAGACCCTGAACTTCGACCTG
CTGAAGCTGGCCGGCGACGTGGAGAGCAACCCCGGCCCC
ATGGAGCACAGCACCTTCCTGAGCGGCCTGGTGCTGGCC
ACCCTGCTGAGCCAGGTGAGCCCCTTCAAGATCCCCATC
GAGGAGCTGGAGGACAGAGTGTTCGTGAACTGCAACACC
AGCATCACCTGGGTGGAGGGCACCGTGGGCACCCTGCTG
AGCGACATCACCAGACTGGACCTGGGCAAGAGAATCCTG
GACCCCAGAGGCATCTACAGATGCAACGGCACCGACATC
TACAAGGACAAGGAGAGCACCGTGCAGGTGCACTACAGA
ATGTGCCAGAGCTGCGTGGAGCTGGACCCCGCCACCGTG
GCCGGCATCATCGTGACCGACGTGATCGCCACCCTGCTG
CTGGCCCTGGGCGTGTTCTGCTTCGCCGGCCACGAGACC
GGCAGACTGAGCGGCGCCGCCGACACCCAGGCCCTGCTG
AGAAACGACCAGGTGTACCAGCCCCTGAGAGACAGAGAC
GACGCCCAGTACAGCCACCTGGGCGGCAACTGGGCCAGA
AACAAGGAGGGCAGAGGCAGCCTGCTGACCTGCGGCGAC
GTGGAGGAGAACCCCGGCCCCATGCAGAGCGGCACCCAC
TGGAGAGTGCTGGGCCTGTGCCTGCTGAGCGTGGGCGTG
TGGGGCCAGGACGGCAACGAGGAGATGGGCGGCATCACC
CAGACCCCCTACAAGGTGAGCATCAGCGGCACCACCGTG
ATCCTGACCTGCCCCCAGTACCCCGGCAGCGAGATCCTG
TGGCAGCACAACGACAAGAACATCGGCGGCGACGAGGAC
GACAAGAACATCGGCAGCGACGAGGACCACCTGAGCCTG
AAGGAGTTCAGCGAGCTGGAGCAGAGCGGCTACTACGTG
TGCTACCCCAGAGGCAGCAAGCCCGAGGACGCCAACTTC
TACCTGTACCTGAGAGCCAGAGTGTGCGAGAACTGCATG
GAGATGGACGTGATGAGCGTGGCCACCATCGTGATCGTG
GACATCTGCATCACCGGCGGCCTGCTGCTGCTGGTGTAC
TACTGGAGCAAGAACAGAAAGGCCAAGGCCAAGCCCGTG
ACCAGAGGCGCCGGCGCCGGCGGCAGACAGAGAGGCCAG
AACAAGGAGAGACCCCCCCCCGTGCCCAACCCCGACTAC
GAGCCCATCAGAAAGGGCCAGAGAGACCTGTACAGCGGC
CTGAACCAGAGAAGAATCGGACCGCAGTGTACTAATTAT
GCTCTCTTGAAATTGGCTGGAGATGTTGAGAGCAATCCC
GGGCCCATGCGCATTAGCAAGCCCCACCTGCGGAGCATC
AGCATCCAGTGCTACCTGTGCCTGCTGCTGAACAGCCAC
TTCCTGACCGAGGCCGGCATCCACGTGTTCATCCTGGGC
TGCTTCAGCGCCGGACTGCCCAAGACCGAGGCCAACTGG
GTGAACGTGATCAGCGACCTGAAGAAGATCGAGGACCTG
ATCCAGAGCATGCACATCGACGCCACCCTGTACACCGAG
AGCGACGTGCACCCCAGCTGCAAGGTGACCGCCATGAAG
TGCTTTCTGCTGGAACTGCAGGTGATCAGCCTGGAAAGC
GGCGACGCCAGCATCCACGACACCGTGGAGAACCTGATC
ATCCTGGCCAACAACAGCCTGAGCAGCAACGGCAACGTG
ACCGAGAGCGGCTGCAAAGAGTGCGAGGAACTGGAAGAG
AAGAACATCAAAGAGTTTCTGCAGAGCTTCGTGCACATC
GTGCAGATGTTCATCAACACCAGC
(SEQ ID NO: 121)
MKWKALFTAAILQAQLPITEAQSFGLLDPKLCYLLDGIL
FIYGVILTALFLRSKRSRLLHSDYMNMTPRRPGPTRKHY
QPYAPPRDFAAYRSRVKFSRSADAPAYQQGQNQLYNELN
LGRREEYDVLDKRRGRDPEMGGKPQRRKNPQEGLYNELQ
KDKMAEAYSEIGMKGERRRGKGHDGLYQGLSTATKDTYD
ALHMQALPPRQCTNYALLKLAGDVESNPGPMEQGKGLAV
LILAIILLQGTLAQSIKGNHLVKVYDYQEDGSVLLTCDA
EAKNITWFKDGKMIGFLTEDKKKWNLGSNAKDPRGMYQC
KGSQNKSKPLQVYYRMCQNCIELNAATISGFLFAEIVSI
FVLAVGVYFIAGQDGVRQSRASDKQTLLPNDQLYQPLKD
REDDQYSHLQGNQLRRNVKQTLNFDLLKLAGDVESNPGP
MEHSTFLSGLVLATLLSQVSPFKIPIEELEDRVFVNCNT
SITWVEGTVGTLLSDITRLDLGKRILDPRGIYRCNGTDI
YKDKESTVQVHYRMCQSCVELDPATVAGIIVTDVIATLL
LALGVFCFAGHETGRLSGAADTQALLRNDQVYQPLRDRD
DAQYSHLGGNWARNKEGRGSLLTCGDVEENPGPMQSGTH
WRVLGLCLLSVGVWGQ.DGNEEMGGITQTPYKVSISGTT
VILTCPQYPGSEILWQHNDKNIGGDEDDKNIGSDEDHLS
LKEFSELEQSGYYVCYPRGSKPEDANFYLYLRARVCENC
MEMDVMSVATIVIVDICITGGLLLLVYYWSKNRKAKAKP
VTRGAGAGGRQRGQNKERPPPVPNPDYEPIRKGQRDLYS
GLNQRRIGPQCTNYALLKLAGDVESNPGPMRISKPHLRS
ISIQCYLCLLLNSHFLTEAGIHVFILGCFSAGLPKTEAN
WVNVISDLKKIEDLIQSMHIDATLYTESDVHPSCKVTAM
KCFLLELQVISLESGDASIHDTVENLIILANNSLSSNGN
VTESGCKECEELEEKNIKEFLQSFVHIVQMFINTS

UTNK15-28-DAP10: refers to full length CD3zeta comprising a fusion with an intracellular co-stimulatory domain derived from DAP10 and an intracellular co-stimulatory domain derived from CD28, full length CD3 gamma, full length CD3 delta, and full length CD3 epsilon linked to I15, it may be represented by a nucleotide sequence that is at least, or exactly, 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 122. In certain embodiments, a UTNK15-28-DAP10 amino acid sequence may be represented by an amino acid sequence that is at least, or exactly, 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 123.

(SEQ ID NO: 122)
ATGAAGTGGAAGGCGCTTTTCACCGCGGCCATCCTGCAG
GCACAGTTGCCGATTACAGAGGCACAGAGCTTTGGCCTG
CTGGATCCCAAACTCTGCTACCTGCTGGATGGAATCCTC
TTCATCTATGGTGTCATTCTCACTGCCTTGTTCCTGAGG
AGTAAGAGGAGCAGGCTCCTGCACAGTGACTACATGAAC
ATGACTCCCCGCCGCCCCGGGCCCACCCGCAAGCATTAC
CAGCCCTATGCCCCACCACGCGACTTCGCAGCCTATCGC
TCACTTTGCGCACGCCCACGCCGCAGCCCCGCCCAAGAA
GATGGCAAAGTCTACATCAACATGCCAGGCAGGGGCAGA
GTGAAGTTCAGCAGGAGCGCAGACGCCCCCGCGTACCAG
CAGGGCCAGAACCAGCTCTATAACGAGCTCAATCTAGGA
CGAAGAGAGGAGTACGATGTTTTGGACAAGAGACGTGGC
CGGGACCCTGAGATGGGGGGAAAGCCGCAGAGAAGGAAG
AACCCTCAGGAAGGCCTGTACAATGAACTGCAGAAAGAT
AAGATGGCGGAGGCCTACAGTGAGATTGGGATGAAAGGC
GAGCGCCGGAGGGGCAAGGGGCACGATGGCCTTTACCAG
GGTCTCAGTACAGCCACCAAGGACACCTACGACGCCCTT
CACATGCAGGCCCTGCCCCCTCGCCAGTGCACCAACTAC
GCCCTGCTGAAGCTGGCCGGCGACGTGGAGAGCAACCCC
GGCCCCATGGAACAGGGGAAGGGCCTGGCTGTCCTCATC
CTGGCTATCATTCTTCTTCAAGGTACTTTGGCCCAGTCA
ATCAAAGGAAACCACTTGGTTAAGGTGTATGACTATCAA
GAAGATGGTTCGGTACTTCTGACTTGTGATGCAGAAGCC
AAAAATATCACATGGTTTAAAGATGGGAAGATGATCGGC
TTCCTAACTGAAGATAAAAAAAAATGGAATCTGGGAAGT
AATGCCAAGGACCCTCGTGGGATGTATCAGTGTAAAGGA
TCACAGAACAAGTCAAAACCACTCCAAGTGTATTACAGA
ATGTGTCAGAACTGCATTGAACTAAATGCAGCCACCATA
TCTGGCTTTCTCTTTGCTGAAATCGTCAGCATTTTCGTC
CTTGCTGTTGGGGTCTACTTCATTGCTGGACAGGATGGA
GTTCGCCAGTCGAGAGCTTCAGACAAGCAGACTCTGTTG
CCCAATGACCAGCTCTACCAGCCCCTCAAGGATCGAGAA
GATGACCAGTACAGCCACCTTCAAGGAAACCAGTTGAGG
AGGAATGTGAAGCAGACCCTGAACTTCGACCTGCTGAAG
CTGGCCGGCGACGTGGAGAGCAACCCCGGCCCCATGGAG
CACAGCACCTTCCTGAGCGGCCTGGTGCTGGCCACCCTG
CTGAGCCAGGTGAGCCCCTTCAAGATCCCCATCGAGGAG
CTGGAGGACAGAGTGTTCGTGAACTGCAACACCAGCATC
ACCTGGGTGGAGGGCACCGTGGGCACCCTGCTGAGCGAC
ATCACCAGACTGGACCTGGGCAAGAGAATCCTGGACCCC
AGAGGCATCTACAGATGCAACGGCACCGACATCTACAAG
GACAAGGAGAGCACCGTGCAGGTGCACTACAGAATGTGC
CAGAGCTGCGTGGAGCTGGACCCCGCCACCGTGGCCGGC
ATCATCGTGACCGACGTGATCGCCACCCTGCTGCTGGCC
CTGGGCGTGTTCTGCTTCGCCGGCCACGAGACCGGCAGA
CTGAGCGGCGCCGCCGACACCCAGGCCCTGCTGAGAAAC
GACCAGGTGTACCAGCCCCTGAGAGACAGAGACGACGCC
CAGTACAGCCACCTGGGCGGCAACTGGGCCAGAAACAAG
GAGGGCAGAGGCAGCCTGCTGACCTGCGGCGACGTGGAG
GAGAACCCCGGCCCCATGCAGAGCGGCACCCACTGGAGA
GTGCTGGGCCTGTGCCTGCTGAGCGTGGGCGTGTGGGGC
CAGGACGGCAACGAGGAGATGGGCGGCATCACCCAGACC
CCCTACAAGGTGAGCATCAGCGGCACCACCGTGATCCTG
ACCTGCCCCCAGTACCCCGGCAGCGAGATCCTGTGGCAG
CACAACGACAAGAACATCGGCGGCGACGAGGACGACAAG
AACATCGGCAGCGACGAGGACCACCTGAGCCTGAAGGAG
TTCAGCGAGCTGGAGCAGAGCGGCTACTACGTGTGCTAC
CCCAGAGGCAGCAAGCCCGAGGACGCCAACTTCTACCTG
TACCTGAGAGCCAGAGTGTGCGAGAACTGCATGGAGATG
GACGTGATGAGCGTGGCCACCATCGTGATCGTGGACATC
TGCATCACCGGCGGCCTGCTGCTGCTGGTGTACTACTGG
AGCAAGAACAGAAAGGCCAAGGCCAAGCCCGTGACCAGA
GGCGCCGGCGCCGGCGGCAGACAGAGAGGCCAGAACAAG
GAGAGACCCCCCCCCGTGCCCAACCCCGACTACGAGCCC
ATCAGAAAGGGCCAGAGAGACCTGTACAGCGGCCTGAAC
CAGAGAAGAATCGGACCGCAGTGTACTAATTATGCTCTC
TTGAAATTGGCTGGAGATGTTGAGAGCAATCCCGGGCCC
ATGCGCATTAGCAAGCCCCACCTGCGGAGCATCAGCATC
CAGTGCTACCTGTGCCTGCTGCTGAACAGCCACTTCCTG
ACCGAGGCCGGCATCCACGTGTTCATCCTGGGCTGCTTC
AGCGCCGGACTGCCCAAGACCGAGGCCAACTGGGTGAAC
GTGATCAGCGACCTGAAGAAGATCGAGGACCTGATCCAG
AGCATGCACATCGACGCCACCCTGTACACCGAGAGCGAC
GTGCACCCCAGCTGCAAGGTGACCGCCATGAAGTGCTTT
CTGCTGGAACTGCAGGTGATCAGCCTGGAAAGCGGCGAC
GCCAGCATCCACGACACCGTGGAGAACCTGATCATCCTG
GCCAACAACAGCCTGAGCAGCAACGGCAACGTGACCGAG
AGCGGCTGCAAAGAGTGCGAGGAACTGGAAGAGAAGAAC
ATCAAAGAGTTTCTGCAGAGCTTCGTGCACATCGTGCAG
ATGTTCATCAACACCAGC
(SEQ ID NO: 123)
MKWKALFTAAILQAQLPITEAQSFGLLDPKLCYLLDGIL
FIYGVILTALFLRSKRSRLLHSDYMNMTPRRPGPTRKHY
QPYAPPRDFAAYRSLCARPRRSPAQEDGKVYINMPGRGR
VKFSRSADAPAYQQGQNQLYNELNLGRREEYDVLDKRRG
RDPEMGGKPQRRKNPQEGLYNELQKDKMAEAYSEIGMKG
ERRRGKGHDGLYQGLSTATKDTYDALHMQALPPRQCTNY
ALLKLAGDVESNPGPMEQGKGLAVLILAIILLQGTLAQS
IKGNHLVKVYDYQEDGSVLLTCDAEAKNITWFKDGKMIG
FLTEDKKKWNLGSNAKDPRGMYQCKGSQNKSKPLQVYYR
MCQNCIELNAATISGFLFAEIVSIFVLAVGVYFIAGQDG
VRQSRASDKQTLLPNDQLYQPLKDREDDQYSHLQGNQLR
RNVKQTLNFDLLKLAGDVESNPGPMEHSTFLSGLVLATL
LSQVSPFKIPIEELEDRVFVNCNTSITWVEGTVGTLLSD
ITRLDLGKRILDPRGIYRCNGTDIYKDKESTVQVHYRMC
QSCVELDPATVAGIIVTDVIATLLLALGVFCFAGHETGR
LSGAADTQALLRNDQVYQPLRDRDDAQYSHLGGNWARNK
EGRGSLLTCGDVEENPGPMQSGTHWRVLGLCLLSVGVWG
Q.DGNEEMGGITQTPYKVSISGTTVILTCPQYPGSEILW
QHNDKNIGGDEDDKNIGSDEDHLSLKEFSELEQSGYYVC
YPRGSKPEDANFYLYLRARVCENCMEMDVMSVATIVIVD
ICITGGLLLLVYYWSKNRKAKAKPVTRGAGAGGRQRGQN
KERPPPVPNPDYEPIRKGQRDLYSGLNQRRIGPQCTNYA
LLKLAGDVESNPGPMRISKPHLRSISIQCYLCLLLNSHE
LTEAGIHVFILGCFSAGLPKTEANWVNVISDLKKIEDLI
QSMHIDATLYTESDVHPSCKVTAMKCELLELQVISLESG
DASIHDTVENLIILANNSLSSNGNVTESGCKECEELEEK
NIKEFLQSFVHIVQMFINTS

As depicted in FIG. 3 and described above, the term “linked” refers to being present on the same polynucleotide vector and does not necessarily mean that the two polypeptides are expressed as one polypeptide. For example, a cytokine produced from a vector of the disclosure may ultimately be produced as a separate molecule from any one or more TCR/CD3 receptor complex components. Whereas, the term “fused” or “fusion” refers to two polypeptides that comprise a peptide bond conjoining the two molecules, i.e. that the two polypeptides are covalently bound by an amide bond and are not separated by a splitting element, such as a 2A element.

One specific example of a TCR that may be utilized in the cells is NY-ESO TCR, and specific examples of sequences include at least the following:

TCRα:
(SEQ ID NO: 25)
XQEVTQIPAALSVPEGENLVLNCSFTDSAIYNLQWFRQD
PGKGLTSLLLIQSSQREQTSGRLNASLDKSSGRSTLYIA
ASQPGDSATYLCAVRPLYGGSYIPTFGRGTSLIVHPYIQ
NPDPAVYQLRDSKSSDKSVCLFTDFDSQTNVSQSKDSDV
YITDKTVLDMRSMDFKSNSAVAWSNKSDFACANAFNNSI
IPEDTFFPSPESSCDVKLVEKSFETDTNLNFQNLSVIGF
RILLLKVAGENLLMTLRLWSS
TCRβ:
(SEQ ID NO: 26)
GVTQTPKFQVLKTGQSMTLQCAQDMNHEYMSWYRQDPGM
GLRLIHYSVGAGITDQGEVPNGYNVSRSTTEDFPLRLLS
AAPSQTSVYFCASSYVGNTGELFFGEGSRLTVLEDLKNV
FPPKVAVFEPSEAEISHTQKATLVCLATGFYPDHVELSW
WVNGKEVHSGVSTDPQPLKEQPALNDSRYCLSSRLRVSA
TFWQNPRNHFRCQVQFYGLSENDEWTQDRAKPVTQIVSA
EAWGRADCGFTSESYQQGVLSATILYEILLGKATLYAVL
VSALVLMAMVKRKDSRG

In certain embodiments, a TCR may comprise a TCR alpha chain variable region encoded by a nucleotide sequence that is at least, or exactly, 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 85.

(SEQ ID NO: 85)
aaacaggaggtgacacagattcctgcagctctgagtgtc
ccagaaggagaaaacttggttctcaactgcagtttcact
gatagcgctatttacaacctccagtggtttaggcaggac
cctgggaaaggtctcacatctctgttgcttattcagtca
agtcagagagagcaaacaagtggaagacttaatgcctcg
ctggataaatcatcaggacgtagtactttatacattgca
gcttctcagcctggtgactcagccacctacctctgtgct
gtgaggcccctttatggaggaagctacatacctacattt
ggaagaggaaccagccttattgttcatccgtat

In certain embodiments, a TCR may comprise a TCR alpha chain constant region encoded by a nucleotide sequence that is at least, or exactly, 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 86.

(SEQ ID NO: 86)
atccagaaccctgaccctgccgtgtaccagctgagagac
tctaaatccagtgacaagtctgtctgcctattcaccgat
tttgattctcaaacaaatgtgtcacaaagtaaggattct
gatgtgtatatcacagacaaaactgtgctagacatgagg
tctatggacttcaagagcaacagtgctgtggcctggagc
aacaaatctgactttgcatgtgcaaacgccttcaacaac
agcattattccagaagacaccttcttccccagcccagaa
agttcctgtgatgtcaagctggtcgagaaaagctttgaa
acagatacgaacctaaactttcaaaacctgtcagtgatt
gggttccgaatcctcctcctgaaagtggccgggtttaat
ctgctcatgacgctgcggctgtggtccagc

In certain embodiments, a TCR may comprise a TCR alpha chain encoded by a nucleotide sequence that is at least, or exactly, 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 87.

(SEQ ID NO: 87)
atggagaccctcttgggcctgcttatcctttggctgcag
ctgcaatgggtgagcagcaaacaggaggtgacacagatt
cctgcagctctgagtgtcccagaaggagaaaacttggtt
ctcaactgcagtttcactgatagcgctatttacaacctc
cagtggtttaggcaggaccctgggaaaggtctcacatct
ctgttgcttattcagtcaagtcagagagagcaaacaagt
ggaagacttaatgcctcgctggataaatcatcaggacgt
agtactttatacattgcagcttctcagcctggtgactca
gccacctacctctgtgctgtgaggcccctttatggagga
agctacatacctacatttggaagaggaaccagccttatt
gttcatccgtatatccagaaccctgaccctgccgtgtac
cagctgagagactctaaatccagtgacaagtctgtctgc
ctattcaccgattttgattctcaaacaaatgtgtcacaa
agtaaggattctgatgtgtatatcacagacaaaactgtg
ctagacatgaggtctatggacttcaagagcaacagtgct
gtggcctggagcaacaaatctgactttgcatgtgcaaac
gccttcaacaacagcattattccagaagacaccttcttc
cccagcccagaaagttcctgtgatgtcaagctggtcgag
aaaagctttgaaacagatacgaacctaaactttcaaaac
ctgtcagtgattgggttccgaatcctcctcctgaaagtg
gccgggtttaatctgctcatgacgctgcggctgtggtcc
agc

In certain embodiments, a TCR may comprise a TCR alpha chain variable region amino acid sequence that is at least, or exactly, 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 88.

(SEQ ID NO: 88)
XQEVTQIPAALSVPEGENLVLNCSFTDSAIYNLQWFRQ
DPGKGLTSLLLIQSSQREQTSGRLNASLDKSSGRSTLY
IAASQPGDSATYLCAVRPLYGGSYIPTFGRGTSLIVHP
Y

In certain embodiments, a TCR may comprise a TCR alpha chain constant region amino acid sequence that is at least, or exactly, 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 89.

(SEQ ID NO: 89)
IQNPDPAVYQLRDSKSSDKSVCLFTDFDSQTNVSQSKDS
DVYITDKTVLDMRSMDFKSNSAVAWSNKSDFACANAFNN
SIIPEDTFFPSPESSCDVKLVEKSFETDINLNFQNLSVI
GFRILLLKVAGFNLLMTLRLWSS

In certain embodiments, a TCR may comprise an alpha chain CDR1 amino acid sequence that is at least, or exactly, 80% or 100% identical to SEQ ID NO: 90. DSAIYN (SEQ ID NO: 90)

In certain embodiments, a TCR may comprise an alpha chain CDR2 amino acid sequence that is at least, or exactly, 80% or 100% identical to SEQ ID NO: 91. IQSSQRE (SEQ ID NO: 91)

In certain embodiments, a TCR may comprise an alpha chain CDR3 amino acid sequence that is at least, or exactly, 80% or 100% identical to SEQ ID NO: 92. CAVRPLYGGSYIPTF (SEQ ID NO: 92)

In certain embodiments, a TCR may comprise a TCR beta chain variable encoded by a nucleotide sequence that is at least, or exactly, 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 93.

(SEQ ID NO: 93)
ggtgtcactcagaccccaaaattccaggtcctgaagaca
ggacagagcatgacactgcagtgtgcccaggatatgaac
catgaatacatgtcctggtatcgacaagacccaggcatg
gggctgaggctgattcattactcagttggtgctggtatc
actgaccaaggagaagtccccaatggctacaatgtctcc
agatcaaccacagaggatttcccgctcaggctgctgtcg
gctgctccctcccagacatctgtgtacttctgtgccagc
agttacgtcgggaacaccggggagctgttttttggagaa
ggctctaggctgaccgtactggag

In certain embodiments, a TCR may comprise a TCR beta chain constant region encoded by a nucleotide sequence that is at least, or exactly, 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 94.

(SEQ ID NO: 94)
Gacctgaaaaacgtgttcccacccaaggtcgctgtgttt
gagccatcagaagcagagatctcccacacccaaaaggcc
acactggtatgcctggccacaggcttctaccccgaccac
gtggagctgagctggtgggtgaatgggaaggaggtgcac
agtggggtcagcacagacccgcagcccctcaaggagcag
cccgccctcaatgactccagatactgcctgagcagccgc
ctgagggtctcggccaccttctggcagaacccccgcaac
cacttccgctgtcaagtccagttctacgggctctcggag
aatgacgagtggacccaggatagggccaaacccgtcacc
cagatcgtcagcgccgaggcctggggtagagcagactgt
ggcttcacctccgagtcttaccagcaaggggtcctgtct
gccaccatcctctatgagatcttgctagggaaggccacc
ttgtatgccgtgctggtcagtgccctcgtgctgatggcc
atggtcaagagaaaggattccagaggc

In certain embodiments, a TCR may comprise a TCR beta chain encoded by a nucleotide sequence that is at least, or exactly, 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 95.

(SEQ ID NO: 95)
Atgagcatcggcctcctgtgctgtgcagccttgtctctc
ctgtgggcaggtccagtgaatgctggtgtcactcagacc
ccaaaattccaggtcctgaagacaggacagagcatgaca
ctgcagtgtgcccaggatatgaaccatgaatacatgtcc
tggtatcgacaagacccaggcatggggctgaggctgatt
cattactcagttggtgctggtatcactgaccaaggagaa
gtccccaatggctacaatgtctccagatcaaccacagag
gatttcccgctcaggctgctgtcggctgctccctcccag
acatctgtgtacttctgtgccagcagttacgtcgggaac
accggggagctgttttttggagaaggctctaggctgacc
gtactggaggacctgaaaaacgtgttcccacccAaggtc
gctgtgtttgagccatcagaagcagagatctcccacacc
caaaaggccacactggtatgcctggccacaggcttctac
cccgaccacgtggagctgagctggtgggtgaatgggaag
gaggtgcacagtggggtcagcacagacccgcagcccctc
aaggagcagcccgccctcaatgactccagatactgcctg
agcagccgcctgagggtctcggccaccttctggcagaac
ccccgcaaccacttccgctgtcaagtccagttctacggg
ctctcggagaatgacgagtggacccaggatagggccaaa
cccgtcacccagatcgtcagcgccgaggcctggggtaga
gcagactgtggcttcacctccgagtcttaccagcaaggg
gtcctgtctgccaccatcctctatgagatcttgctaggg
aaggccaccttgtatgccgtgctggtcagtgccctcgtg
ctgatggccatggtcaagagaaaggattccagaggc

In certain embodiments, a TCR may comprise a TCR beta chain variable region amino acid sequence that is at least, or exactly, 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 96.

(SEQ ID NO: 96)
GVTQTPKFQVLKTGQSMTLQCAQDMNHEYMSWYRQDPGM
GLRLIHYSVGAGITDQGEVPNGYNVSRSTTEDFPLRLLS
AAPSQTSVYFCASSYVGNTGELFFGEGSRLTVLE

In certain embodiments, a TCR may comprise a TCR beta chain constant region amino acid sequence that is at least, or exactly, 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 97.

DLKNVFPPKVAVFEPSEAEISHTQKATLVCLATGFYPDH
VELSWWVNGKEVHSGVSTDPQPLKEQPALNDSRYCLSSR
LRVSATFWQNPRNHFRCQVQFYGLSENDEWTQDRAKPVT
QIVSAEAWGRADCGFTSESYQQGVLSATILYEILLGKAT
LYAVLVSALVLMAMVKRKDSRG

In certain embodiments, a TCR may comprise a beta chain CDR1 amino acid sequence that is at least, or exactly, 80% or 100% identical to SEQ ID NO: 98.

(SEQ ID NO: 98)
MNHEY

In certain embodiments, a TCR may comprise a beta chain CDR2 amino acid sequence that is at least, or exactly, 80% or 100% identical to SEQ ID NO: 99.

(SEQ ID NO: 99)
SVGAGI

In certain embodiments, a TCR may comprise a beta chain CDR3 amino acid sequence that is at least, or exactly, 80% or 100% identical to SEQ ID NO: 100. CASSYVGNTGELFF (SEQ ID NO: 100)

In certain embodiments, a TCR (e.g., a TCR alpha, beta, delta, and/or gamma) chain may comprise a signal peptide. In certain embodiments, a signal peptide is encoded by a nucleic acid that is at least, or exactly 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 101 or SEQ ID NO: 102. In certain embodiments, a signal peptide is at least, or exactly, 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 103 or SEQ ID NO: 104.

(SEQ ID NO: 101)
atggagaccctcttgggcctgcttatcctttggc
tgcagctgcaatgggtgagcagc
(SEQ ID NO: 102)
atgagcatcggcctcctgtgctgtgcagccttgtc
tctcctgtgggcaggtccagtgaatgct
(SEQ ID NO: 103)
METLLGLLILWLQLQWVSS
(SEQ ID NO: 104)
MSIGLLCCAALSLLWAGPVNA

In certain embodiments, a TCR recognizes a peptide corresponding to amino acid residues 157-165 of the human cancer testis Ag NY-ESO-1 in the context of the HLA-A*02 class I allele. In certain embodiments, a TCR may target an epitope characterized by the amino acid sequence according to SEQ ID NO: 105.
SLLMWITQC (SEQ ID NO: 105)

One specific example of a TCR that may be utilized in the cells is TCRpp65alpha, and specific examples of sequences include at least the following (underlining refers to signal peptide sequence):

(SEQ ID NO: 27)
ATGGACTCCTGGACCTTCTGCTGTGTGTCCCTTTGCATCCTGGTA
GCAAAGCACACAGATGCTGGACAACAGCTGAATCAGAGTCCTCAA
TCTATGTTTATCCAGGAAGGAGAAGATGTCTCCATGAACTGCACT
TCTTCAAGCATATTTAACACCTGGCTATGGTACAAGCAGGACCCT
GGGGAAGGTCCTGTCCTCTTGATAGCCTTATATAAGGCTGGTGAA
TTGACCTCAAATGGAAGACTGACTGCTCAGTTTGGTATAACCAGA
AAGGACAGCTTCCTGAATATCTCAGCATCCATACCCAGTGATGTA
GGCATCTACTTCTGTGCTGGACCCATGAAAACCTCCTACGACAAG
GTGATATTTGGGCCAGGGACAAGCTTATCAGTCATTCCAAATATC
CAGAACCCTGACCCTGCCGTGTACCAGCTGAGAGACTCTAAATCC
AGTGACAAGTCTGTCTGCCTATTCACCGATTTTGATTCTCAAACA
AATGTGTCACAAAGTAAGGATTCTGATGTGTATATCACAGACAAA
ACTGTGCTAGACATGAGGTCTATGGACTTCAAGAGCAACAGTGCT
GTGGCCTGGAGCAACAAATCTGACTTTGCATGTGCAAACGCCTTC
AACAACAGCATTATTCCAGAAGACACCTTCTTCCCCAGCCCAGAA
AGTTCCTGTGATGTCAAGCTGGTCGAGAAAAGCTTTGAAACAGAT
ACGAACCTAAACTTTCAAAACCTGTCAGTGATTGGGTTCCGAATC
CTCCTCCTGAAAGTGGCCGGGTTTAATCTGCTCATGACGCTGCGG
CTGTGGTCCAGC
(SEQ ID NO: 28)
MDSWTFCCVSLCILVAKHTDAGQQLNQSPQSMFIQEGEDVSMNCT
SSSIFNTWLWYKQDPGEGPVLLIALYKAGELTSNGRLTAQFGITR
KDSFLNISASIPSDVGIYFCAGPMKTSYDKVIFGPGTSLSVIPNI
QNPDPAVYQLRDSKSSDKSVCLFTDFDSQTNVSQSKDSDVYITDK
TVLDMRSMDFKSNSAVAWSNKSDFACANAFNNSIIPEDTFFPSPE
SSCDVKLVEKSFETDINLNFQNLSVIGFRILLLKVAGENLLMTLR
LWSS

One specific example of a TCR that may be utilized in the cells is TCRpp65beta, and specific examples of sequences include at least the following (underlining refers to signal peptide sequence):

(SEQ ID NO: 29)
ATGGACTCCTGGACCTTCTGCTGTGTGTCCCTTTGCATCCTGGTA
GCAAAGCACACAGATGCTGGAGTTATCCAGTCACCCCGGCACGAG
GTGACAGAGATGGGACAAGAAGTGACTCTGAGATGTAAACCAATT
TCAGGACACGACTACCTTTTCTGGTACAGACAGACCATGATGCGG
GGACTGGAGTTGCTCATTTACTTTAACAACAACGTTCCGATAGAT
GATTCAGGGATGCCCGAGGATCGATTCTCAGCTAAGATGCCTAAT
GCATCATTCTCCACTCTGAAGATCCAGCCCTCAGAACCCAGGGAC
TCAGCTGTGTACTTCTGTGCCAGCAGTTCGGCAAACTATGGCTAC
ACCTTCGGTTCGGGGACCAGGTTAACCGTTGTAGAGGACCTGAAC
AAGGTGTTCCCACCCGAGGTCGCTGTGTTTGAGCCATCAGAAGCA
GAGATCTCCCACACCCAAAAGGCCACACTGGTGTGCCTGGCCACA
GGCTTCTTCCCTGACCACGTGGAGCTGAGCTGGTGGGTGAATGGG
AAGGAGGTGCACAGTGGGGTCAGCACGGACCCGCAGCCCCTCAAG
GAGCAGCCCGCCCTCAATGACTCCAGATACTGCCTGAGCAGCCGC
CTGAGGGTCTCGGCCACCTTCTGGCAGAACCCCCGCAACCACTTC
CGCTGTCAAGTCCAGTTCTACGGGCTCTCGGAGAATGACGAGTGG
ACCCAGGATAGGGCCAAACCCGTCACCCAGATCGTCAGCGCCGAG
GCCTGGGGTAGAGCAGACTGTGGCTTTACCTCGGTGTCCTACCAG
CAAGGGGTCCTGTCTGCCACCATCCTCTATGAGATCCTGCTAGGG
AAGGCCACCCTGTATGCTGTGCTGGTCAGCGCCCTTGTGTTGATG
GCCATGGTCAAGAGAAAGGATTTC
(SEQ ID NO: 30)
MDSWTFCCVSLCILVAKHTDAGVIQSPRHEVTEMGQEVTLRCKPI
SGHDYLFWYRQTMMRGLELLIYFNNNVPIDDSGMPEDRESAKMPN
ASFSTLKIQPSEPRDSAVYFCASSSANYGYTFGSGTRLTVVEDLN
KVFPPEVAVFEPSEAEISHTQKATLVCLATGFFPDHVELSWWVNG
KEVHSGVSTDPQPLKEQPALNDSRYCLSSRLRVSATFWQNPRNHF
RCQVQFYGLSENDEWTQDRAKPVTQIVSAEAWGRADCGFTSVSYQ
QGVLSATILYEILLGKATLYAVLVSALVLMAMVKRKDF

TCRpp65ZFLGDEFL15

In certain embodiments, one may utilize a construct in which TCRpp65 is linked to full length CD3zeta, full length CD3 gamma, full length CD3 delta, full length CD3 epsilon, and also linked to IL-15 (and may be referred to as TCRpp65ZFLGDEFL15). One representative sequence for such a construct is as follows:

(SEQ ID NO: 74)
MLEGVTQTPKFQVLKTGQSMTLQCAQDMNHEYMSWYRQDPGMGLR
LIHYSVGAGITDQGEVPNGYNVSRSTTEDFPLRLLSAAPSQTSVY
FCASSPVTGGIYGYTFGSGTRLTVVEDLNKVFPPEVAVFEPSEAE
ISHTQKATLVCLATGFFPDHVELSWWVNGKEVHSGVSTDPQPLKE
QPALNDSRYCLSSRLRVSATFWQNPRNHFRCQVQFYGLSENDEWT
QDRAKPVTQIVSAEAWGRADATNFSLLKQAGDVEENPGPMILNVE
QSPQSLHVQEGDSTNFTCSFPSSNFYALHWYRWETAKSPEALFVM
TLNGDEKKKGRISATLNTKEGYSYLYIKGSQPEDSATYLCARNTG
NQFYFGTGTSLTVIPNIQNPDPAVYQLRDSKSSDKSVCLFTDFDS
QTNVSQSKDSDAYITDKTVLDMRSMDFKSNSAVAWSNKSDFACAN
AFNNSIIPEDTFFPSPESSEGRGSLLTCGDVEENPGPMKWKALFT
AAILQAQLPITEAQSFGLLDPKLCYLLDGILFIYGVILTALFLRV
KFSRSADAPAYQQGQNQLYNELNLGRREEYDVLDKRRGRDPEMGG
KPQRRKNPQEGLYNELQKDKMAEAYSEIGMKGERRRGKGHDGLYQ
GLSTATKDTYDALHMQALPPRQCTNYALLKLAGDVESNPGPMEQG
KGLAVLILAIILLQGTLAQSIKGNHLVKVYDYQEDGSVLLTCDAE
AKNITWFKDGKMIGFLTEDKKKWNLGSNAKDPRGMYQCKGSQNKS
KPLQVYYRMCQNCIELNAATISGFLFAEIVSIFVLAVGVYFIAGQ
DGVRQSRASDKQTLLPNDQLYQPLKDREDDQYSHLQGNQLRRNVK
QTLNFDLLKLAGDVESNPGPMEHSTFLSGLVLATLLSQVSPFKIP
IEELEDRVFVNCNTSITWVEGTVGTLLSDITRLDLGKRILDPRGI
YRCNGTDIYKDKESTVQVHYRMCQSCVELDPATVAGIIVTDVIAT
LLLALGVFCFAGHETGRLSGAADTQALLRNDQVYQPLRDRDDAQY
SHLGGNWARNKEGRGSLLTCGDVEENPGPMQSGTHWRVLGLCLLS
VGVWGQDGNEEMGGITQTPYKVSISGTTVILTCPQYPGSEILWQH
NDKNIGGDEDDKNIGSDEDHLSLKEFSELEQSGYYVCYPRGSKPE
DANFYLYLRARVCENCMEMDVMSVATIVIVDICITGGLLLLVYYW
SKNRKAKAKPVTRGAGAGGRQRGQNKERPPPVPNPDYEPIRKGQR
DLYSGLNQRRIGPQCTNYALLKLAGDVESNPGPMRISKPHLRSIS
IQCYLCLLLNSHFLTEAGIHVFILGCFSAGLPKTEANWVNVISDL
KKIEDLIQSMHIDATLYTESDVHPSCKVTAMKCFLLELQVISLES
GDASIHDTVENLIILANNSLSSNGNVTESGCKECEELEEKNIKEF
LQSFVHIVQMFINTS*.

In TCRpp65ZFLGDEFL15, the corresponding component sequences are as follows, although these particular sequences or others may be utilized in this and/or other constructs:

TCRb-extracellular domain:
(SEQ ID NO: 75)
MLEGVTQTPKFQVLKTGQSMTLQCAQDMNHEYMSWYRQDPGMGLR
LIHYSVGAGITDQGEVPNGYNVSRSTTEDFPLRLLSAAPSQTSVY
FCASSPVTGGIYGYTFGSGTRLTVVEDLNKVFPPEVAVFEPSEAE
ISHTQKATLVCLATGFFPDHVELSWWVNGKEVHSGVSTDPQPLKE
QPALNDSRYCLSSRLRVSATFWQNPRNHFRCQVQFYGLSENDEWT
QDRAKPVTQIVSAEAWGRADATNFSLLKQAGDVEENPGP
(and that includes the P2A sequence
at its C-terminus)
(SEQ ID NO: 76)
ATGCTCGAGGGAGTGACCCAGACCCCCAAGTTCCAGGTGCTGAAG
ACCGGACAGAGCATGACCCTGCAGTGCGCCCAGGACATGAACCAC
GAGTACATGAGCTGGTACCGGCAGGACCCCGGAATGGGACTGCGG
CTGATCCACTACAGCGTGGGAGCCGGAATCACCGACCAGGGAGAG
GTGCCCAACGGATACAACGTGAGCCGGAGCACCACCGAGGACTTC
CCCCTGCGGCTGCTGAGCGCCGCCCCCAGCCAGACCAGCGTGTAC
TTCTGCGCCAGCAGCCCCGTGACCGGAGGAATCTACGGATACACC
TTCGGAAGCGGAACCCGGCTGACCGTGGTGGAGGACCTGAACAAG
GTGTTCCCCCCCGAGGTGGCCGTGTTCGAGCCCAGCGAGGCCGAG
ATCAGCCACACCCAGAAGGCCACCCTGGTGTGCCTGGCCACCGGA
TTCTTCCCCGACCACGTGGAGCTGAGCTGGTGGGTGAACGGAAAG
GAGGTGCACAGCGGAGTGAGCACCGACCCCCAGCCCCTGAAGGAG
CAGCCCGCCCTGAACGACAGCCGGTACTGCCTGAGCAGCCGGCTG
CGGGTGAGCGCCACCTTCTGGCAGAACCCCCGGAACCACTTCCGG
TGCCAGGTGCAGTTCTACGGACTGAGCGAGAACGACGAGTGGACC
CAGGACCGGGCCAAGCCCGTGACCCAGATCGTGAGCGCCGAGGCC
TGGGGACGGGCCGAC
TCRa-extracellular domain:
(SEQ ID NO: 77)
MILNVEQSPQSLHVQEGDSTNFTCSFPSSNFYALHWYRWETAKSP
EALFVMTLNGDEKKKGRISATLNTKEGYSYLYIKGSQPEDSATYL
CARNTGNQFYFGTGTSLTVIPNIQNPDPAVYQLRDSKSSDKSVCL
FTDFDSQTNVSQSKDSDAYITDKTVLDMRSMDFKSNSAVAWSNKS
DFACANAFNNSIIPEDTFFPSPESSEGRGSLLTCGDVEENPGP
(and that includes the T2A sequence
at its C-terminus)
(SEQ ID NO: 78)
ATGATCCTGAACGTGGAGCAGAGCCCCCAGAGCCTGCACGTGCAG
GAGGGAGACAGCACCAACTTCACCTGCAGCTTCCCCAGCAGCAAC
TTCTACGCCCTGCACTGGTACCGGTGGGAGACCGCCAAGAGCCCC
GAGGCCCTGTTCGTGATGACCCTGAACGGAGACGAGAAGAAGAAG
GGACGGATCAGCGCCACCCTGAACACCAAGGAGGGATACAGCTAC
CTGTACATCAAGGGAAGCCAGCCCGAGGACAGCGCCACCTACCTG
TGCGCCCGGAACACCGGAAACCAGTTCTACTTCGGAACCGGAACC
AGCCTGACCGTGATCCCCAACATCCAGAACCCCGACCCCGCCGTG
TACCAGCTGCGGGACAGCAAGAGCAGCGACAAGAGCGTGTGCCTG
TTCACCGACTTCGACAGCCAGACCAACGTGAGCCAGAGCAAGGAC
AGCGACGCCTACATCACCGACAAGACCGTGCTGGACATGCGGAGC
ATGGACTTCAAGAGCAACAGCGCCGTGGCCTGGAGCAACAAGAGC
GACTTCGCCTGCGCCAACGCCTTCAACAACAGCATCATCCCCGAG
GACACCTTCTTCCCCAGCCCCGAGAGCAGCGCCACCAACTTCTCC
CTGCTGAAGCAGGCCGGCGACGTGGAGGAGAACCCCGGCCCC

TCR5: referred to TCRCgdZFLGDEFL15, is the constant region of TCR gamma and delta, linked to full length CD3zeta, full length CD3 gamma, full length CD3 delta, and full length CD3 epsilon; and IL-15. Representative sequences are as follows:

TCR constant gamma-delta
(TCRCgd)
(SEQ ID NO: 81)
ATGCGGTGGGCCCTACTGGTGCTTCTAGCTTTCCTGTCTCCTGCC
AGTCAGGATAAACAACTTGATGCAGATGTTTCCCCCAAGCCCACT
ATTTTTCTTCCTTCGATTGCTGAAACAAAACTCCAGAAGGCTGGA
ACATACCTTTGTCTTCTTGAGAAATTTTTCCCAGATATTATTAAG
ATACATTGGCAAGAAAAGAAGAGCAACACGATTCTGGGATCCCAG
GAGGGGAACACCATGAAGACTAACGACACATACATGAAATTTAGC
TGGTTAACGGTGCCAGAAGAGTCACTGGACAAAGAACACAGATGT
ATCGTCAGACATGAGAATAATAAAAACGGAATTGATCAAGAAATT
ATCTTTCCTCCAATAAAGACAGATGTCACCACAGTGGATCCCAAA
TACAATTATTCAAAGGATGCAAATGATGTCATCACAATGGATCCC
AAAGACAATTGGTCAAAAGATGCAAATGATACACTACTGCTGCAG
CTCACAAACACCTCTGCATATTACACGTACCTCCTCCTGCTCCTC
AAGAGTGTGGTCTATTTTGCCATCATCACCTGCTGTCTGCTTAGA
AGAACGGCTTTCTGCTGCAATGGAGAGAAATCAGGAAGCGGAGCT
ACTAACTTTAGCCTGCTGAAGCAGGCTGGAGATGTGGAGGAGAAC
CCTGGACCTATGATTCTTACTGTGGGCTTTAGCTTTTTGTTTTTC
TACAGGGGCACGCTGTGTAGTCAGCCTCATACCAAACCATCCGTT
TTTGTCATGAAAAATGGAACAAATGTCGCTTGTCTGGTGAAGGAA
TTCTACCCCAAGGATATAAGAATAAATCTCGTGTCATCCAAGAAG
ATAACAGAGTTTGATCCTGCTATTGTCATCTCTCCCAGTGGGAAG
TACAATGCTGTCAAGCTTGGTAAATATGAAGATTCAAATTCAGTG
ACATGTTCAGTTCAACACGACAATAAAACTGTGCACTCCACTGAC
TTTGAAGTGAAGACAGATTCTACAGATCACGTAAAACCAAAGGAA
ACTGAAAACACAAAGCAACCTTCAAAGAGCTGCCATAAACCCAAA
GCCATAGTTCATACCGAGAAGGTGAACATGATGTCCCTCACAGTG
CTTGGGCTACGAATGCTGTTTGCAAAGACTGTTGCCGTCAATTTT
CTCTTGACTGCCAAGTTATTTTTCTTGTAA
(SEQ ID NO: 82)
MRWALLVLLAFLSPA
SQDKQLDADVSPKPTIFLPSIAETKLQKAGTYLCLLEKFFPDIIK
IHWQEKKSNTILGSQEGNTMKINDTYMKESWLTVPEESLDKEHRC
IVRHENNKNGIDQEIIFPPIKTDVTTVDPKYNYSKDANDVITMDP
KDNWSKDANDTLLLQLTNTSAYYTYLLLLLKSVVYFAIITCCLLR
RTAFCCNGEKSGSGATNFSLLKQAGDVEENPGPMILTVGFSFLFF
YRGTLCSQPHTKPSVFVMKNGTNVACLVKEFYPKDIRINLVSSKK
ITEFDPAIVISPSGKYNAVKLGKYEDSNSVTCSVQHDNKTVHSTD
FEVKTDSTDHVKPKETENTKQPSKSCHKPKAIVHTEKVNMMSLTV
LGLRMLFAKTVAVNELLTAKLFFL
CD3:
(SEQ ID NO: 79)
MKWKALFTAAILQAQLPITEAQSFGLLDPKLCYLLDGILFIYGVI
LTALFLRVKFSRSADAPAYQQGQNQLYNELNLGRREEYDVLDKRR
GRDPEMGGKPQRRKNPQEGLYNELQKDKMAEAYSEIGMKGERRRG
KGHDGLYQGLSTATKDTYDALHMQALPPRQCTNYALLKLAGDVES
NPGPMEQGKGLAVLILAIILLQGTLAQSIKGNHLVKVYDYQEDGS
VLLTCDAEAKNITWFKDGKMIGFLTEDKKKWNLGSNAKDPRGMYQ
CKGSQNKSKPLQVYYRMCQNCIELNAATISGELFAEIVSIFVLAV
GVYFIAGQDGVRQSRASDKQTLLPNDQLYQPLKDREDDQYSHLQG
NQLRRNVKQTLNFDLLKLAGDVESNPGPMEHSTFLSGLVLATLLS
QVSPFKIPIEELEDRVFVNCNTSITWVEGTVGTLLSDITRLDLGK
RILDPRGIYRCNGTDIYKDKESTVQVHYRMCQSCVELDPATVAGI
IVTDVIATLLLALGVFCFAGHETGRLSGAADTQALLRNDQVYQPL
RDRDDAQYSHLGGNWARNKEGRGSLLTCGDVEENPGPMQSGTHWR
VLGLCLLSVGVWGQDGNEEMGGITQTPYKVSISGTTVILTCPQYP
GSEILWQHNDKNIGGDEDDKNIGSDEDHLSLKEFSELEQSGYYVC
YPRGSKPEDANFYLYLRARVCENCMEMDVMSVATIVIVDICITGG
LLLLVYYWSKNRKAKAKPVTRGAGAGGRQRGQNKERPPPVPNPDY
EPIRKGQRDLYSGLNQRRIGPQCTNYALLKLAGDVESNPGP
(SEQ ID NO: 80)
ATGAAGTGGAAGGCGCTTTTCACCGCGGCCATCCTGCAGGCACAG
TTGCCGATTACAGAGGCACAGAGCTTTGGCCTGCTGGATCCCAAA
CTCTGCTACCTGCTGGATGGAATCCTCTTCATCTATGGTGTCATT
CTCACTGCCTTGTTCCTGAGAGTGAAGTTCAGCAGGAGCGCAGAC
GCCCCCGCGTACCAGCAGGGCCAGAACCAGCTCTATAACGAGCTC
AATCTAGGACGAAGAGAGGAGTACGATGTTTTGGACAAGAGACGT
GGCCGGGACCCTGAGATGGGGGGAAAGCCGCAGAGAAGGAAGAAC
CCTCAGGAAGGCCTGTACAATGAACTGCAGAAAGATAAGATGGCG
GAGGCCTACAGTGAGATTGGGATGAAAGGCGAGCGCCGGAGGGGC
AAGGGGCACGATGGCCTTTACCAGGGTCTCAGTACAGCCACCAAG
GACACCTACGACGCCCTTCACATGCAGGCCCTGCCCCCTCGCCAG
TGCACCAACTACGCCCTGCTGAAGCTGGCCGGCGACGTGGAGAGC
AACCCCGGCCCCATGGAACAGGGGAAGGGCCTGGCTGTCCTCATC
CTGGCTATCATTCTTCTTCAAGGTACTTTGGCCCAGTCAATCAAA
GGAAACCACTTGGTTAAGGTGTATGACTATCAAGAAGATGGTTCG
GTACTTCTGACTTGTGATGCAGAAGCCAAAAATATCACATGGTTT
AAAGATGGGAAGATGATCGGCTTCCTAACTGAAGATAAAAAAAAA
TGGAATCTGGGAAGTAATGCCAAGGACCCTCGTGGGATGTATCAG
TGTAAAGGATCACAGAACAAGTCAAAACCACTCCAAGTGTATTAC
AGAATGTGTCAGAACTGCATTGAACTAAATGCAGCCACCATATCT
GGCTTTCTCTTTGCTGAAATCGTCAGCATTTTCGTCCTTGCTGTT
GGGGTCTACTTCATTGCTGGACAGGATGGAGTTCGCCAGTCGAGA
GCTTCAGACAAGCAGACTCTGTTGCCCAATGACCAGCTCTACCAG
CCCCTCAAGGATCGAGAAGATGACCAGTACAGCCACCTTCAAGGA
AACCAGTTGAGGAGGAATGTGAAGCAGACCCTGAACTTCGACCTG
CTGAAGCTGGCCGGCGACGTGGAGAGCAACCCCGGCCCCATGGAG
CACAGCACCTTCCTGAGCGGCCTGGTGCTGGCCACCCTGCTGAGC
CAGGTGAGCCCCTTCAAGATCCCCATCGAGGAGCTGGAGGACAGA
GTGTTCGTGAACTGCAACACCAGCATCACCTGGGTGGAGGGCACC
GTGGGCACCCTGCTGAGCGACATCACCAGACTGGACCTGGGCAAG
AGAATCCTGGACCCCAGAGGCATCTACAGATGCAACGGCACCGAC
ATCTACAAGGACAAGGAGAGCACCGTGCAGGTGCACTACAGAATG
TGCCAGAGCTGCGTGGAGCTGGACCCCGCCACCGTGGCCGGCATC
ATCGTGACCGACGTGATCGCCACCCTGCTGCTGGCCCTGGGCGTG
TTCTGCTTCGCCGGCCACGAGACCGGCAGACTGAGCGGCGCCGCC
GACACCCAGGCCCTGCTGAGAAACGACCAGGTGTACCAGCCCCTG
AGAGACAGAGACGACGCCCAGTACAGCCACCTGGGCGGCAACTGG
GCCAGAAACAAGGAGGGCAGAGGCAGCCTGCTGACCTGCGGCGAC
GTGGAGGAGAACCCCGGCCCCATGCAGAGCGGCACCCACTGGAGA
GTGCTGGGCCTGTGCCTGCTGAGCGTGGGCGTGTGGGGCCAGGAC
GGCAACGAGGAGATGGGCGGCATCACCCAGACCCCCTACAAGGTG
AGCATCAGCGGCACCACCGTGATCCTGACCTGCCCCCAGTACCCC
GGCAGCGAGATCCTGTGGCAGCACAACGACAAGAACATCGGCGGC
GACGAGGACGACAAGAACATCGGCAGCGACGAGGACCACCTGAGC
CTGAAGGAGTTCAGCGAGCTGGAGCAGAGCGGCTACTACGTGTGC
TACCCCAGAGGCAGCAAGCCCGAGGACGCCAACTTCTACCTGTAC
CTGAGAGCCAGAGTGTGCGAGAACTGCATGGAGATGGACGTGATG
AGCGTGGCCACCATCGTGATCGTGGACATCTGCATCACCGGCGGC
CTGCTGCTGCTGGTGTACTACTGGAGCAAGAACAGAAAGGCCAAG
GCCAAGCCCGTGACCAGAGGCGCCGGCGCCGGCGGCAGACAGAGA
GGCCAGAACAAGGAGAGACCCCCCCCCGTGCCCAACCCCGACTAC
GAGCCCATCAGAAAGGGCCAGAGAGACCTGTACAGCGGCCTGAAC
CAGAGAAGAATCGGACCGCAGTGTACTAATTATGCTCTCTTGAAA
TTGGCTGGAGATGTTGAGAGCAATCCCGGGCCC
IL-15:
(SEQ ID NO: 48)
MRISKPHLRSISIQCYLCLLLNSHELTEAGIHVFILGCFSAGLPK
TEANWVNVISDLKKIEDLIQSMHIDATLYTESDVHPSCKVTAMKC
FLLELQVISLESGDASIHDTVENLIILANNSLSSNGNVTESGCKE
CEELEEKNIKEFLQSFVHIVQMFINTS*
(SEQ ID NO: 49)
ATGCGCATTAGCAAGCCCCACCTGCGGAGCATCAGCATCCAGTGC
TACCTGTGCCTGCTGCTGAACAGCCACTTCCTGACCGAGGCCGGC
ATCCACGTGTTCATCCTGGGCTGCTTCAGCGCCGGACTGCCCAAG
ACCGAGGCCAACTGGGTGAACGTGATCAGCGACCTGAAGAAGATC
GAGGACCTGATCCAGAGCATGCACATCGACGCCACCCTGTACACC
GAGAGCGACGTGCACCCCAGCTGCAAGGTGACCGCCATGAAGTGC
TTTCTGCTGGAACTGCAGGTGATCAGCCTGGAAAGCGGCGACGCC
AGCATCCACGACACCGTGGAGAACCTGATCATCCTGGCCAACAAC
AGCCTGAGCAGCAACGGCAACGTGACCGAGAGCGGCTGCAAAGAG
TGCGAGGAACTGGAAGAGAAGAACATCAAAGAGTTTCTGCAGAGC
TTCGTGCACATCGTGCAGATGTTCATCAACACCAGC

TCR6: also referred to TCRCabZFLGDEFL 15, is the constant region of TCR alpha and beta, linked to full length CD3zeta, full length CD3 gamma, full length CD3 delta, and full length CD3 epsilon; and IL-15. Representative sequences are as follows:

TCR constant alpha-beta
(TCRCab)
(SEQ ID NO: 83)
METLLGLLILWLQLQWVSSIQNPDPAVYQLRDSKSSDKSVCLFTD
FDSQTNVSQSKDSDVYITDKTVLDMRSMDFKSNSAVAWSNKSDFA
CANAFNNSIIPEDTFFPSPESSCDVKLVEKSFETDTNLNFQNLSV
IGFRILLLKVAGFNLLMTLRLWSSGSGATNFSLLKQAGDVEENPG
PMSIGLLCCAALSLLWAGPVNADLKNVFPPKVAVFEPSEAEISHT
QKATLVCLATGFYPDHVELSWWVNGKEVHSGVSTDPQPLKEQPAL
NDSRYCLSSRLRVSATFWQNPRNHFRCQVQFYGLSENDEWTQDRA
KPVTQIVSAEAWGRADCGFTSESYQQGVLSATILYEILLGKATLY
AVLVSALVLMAMVKRKDSRG
(SEQ ID NO: 84)
ATGGAGACCCTCTTGGGCCTGCTTATCCTTTGGCTGCAGCTGCAA
TGGGTGAGCAGCATCCAGAACCCTGACCCTGCCGTGTACCAGCTG
AGAGACTCTAAATCCAGTGACAAGTCTGTCTGCCTATTCACCGAT
TTTGATTCTCAAACAAATGTGTCACAAAGTAAGGATTCTGATGTG
TATATCACAGACAAAACTGTGCTAGACATGAGGTCTATGGACTTC
AAGAGCAACAGTGCTGTGGCCTGGAGCAACAAATCTGACTTTGCA
TGTGCAAACGCCTTCAACAACAGCATTATTCCAGAAGACACCTTC
TTCCCCAGCCCAGAAAGTTCCTGTGATGTCAAGCTGGTCGAGAAA
AGCTTTGAAACAGATACGAACCTAAACTTTCAAAACCTGTCAGTG
ATTGGGTTCCGAATCCTCCTCCTGAAAGTGGCCGGGTTTAATCTG
CTCATGACGCTGCGGCTGTGGTCCAGCGGAAGCGGAGCTACTAAC
TTTAGCCTGCTGAAGCAGGCTGGAGATGTGGAGGAGAACCCTGGA
CCTATGAGCATCGGCCTCCTGTGCTGTGCAGCCTTGTCTCTCCTG
TGGGCAGGTCCAGTGAATGCTGACCTGAAAAACGTGTTCCCACCC
AAGGTCGCTGTGTTTGAGCCATCAGAAGCAGAGATCTCCCACACC
CAAAAGGCCACACTGGTATGCCTGGCCACAGGCTTCTACCCCGAC
CACGTGGAGCTGAGCTGGTGGGTGAATGGGAAGGAGGTGCACAGT
GGGGTCAGCACAGACCCGCAGCCCCTCAAGGAGCAGCCCGCCCTC
AATGACTCCAGATACTGCCTGAGCAGCCGCCTGAGGGTCTCGGCC
ACCTTCTGGCAGAACCCCCGCAACCACTTCCGCTGTCAAGTCCAG
TTCTACGGGCTCTCGGAGAATGACGAGTGGACCCAGGATAGGGCC
AAACCCGTCACCCAGATCGTCAGCGCCGAGGCCTGGGGTAGAGCA
GACTGTGGCTTCACCTCCGAGTCTTACCAGCAAGGGGTCCTGTCT
GCCACCATCCTCTATGAGATCTTGCTAGGGAAGGCCACCTTGTAT
GCCGTGCTGGTCAGTGCCCTCGTGCTGATGGCCATGGTCAAGAGA
AAGGATTCCAGAGGCTAA
CD3:
(SEQ ID NO: 79)
MKWKALFTAAILQAQLPITEAQSFGLLDPKLCYLLDGILFIYGVI
LTALFLRVKESRSADAPAYQQGQNQLYNELNLGRREEYDVLDKRR
GRDPEMGGKPQRRKNPQEGLYNELQKDKMAEAYSEIGMKGERRRG
KGHDGLYQGLSTATKDTYDALHMQALPPRQCTNYALLKLAGDVES
NPGPMEQGKGLAVLILAIILLQGTLAQSIKGNHLVKVYDYQEDGS
VLLTCDAEAKNITWFKDGKMIGFLTEDKKKWNLGSNAKDPRGMYQ
CKGSQNKSKPLQVYYRMCQNCIELNAATISGELFAEIVSIFVLAV
GVYFIAGQDGVRQSRASDKQTLLPNDQLYQPLKDREDDQYSHLQG
NQLRRNVKQTLNFDLLKLAGDVESNPGPMEHSTFLSGLVLATLLS
QVSPFKIPIEELEDRVFVNCNTSITWVEGTVGTLLSDITRLDLGK
RILDPRGIYRCNGTDIYKDKESTVQVHYRMCQSCVELDPATVAGI
IVTDVIATLLLALGVFCFAGHETGRLSGAADTQALLRNDQVYQPL
RDRDDAQYSHLGGNWARNKEGRGSLLTCGDVEENPGPMQSGTHWR
VLGLCLLSVGVWGQDGNEEMGGITQTPYKVSISGTTVILTCPQYP
GSEILWQHNDKNIGGDEDDKNIGSDEDHLSLKEFSELEQSGYYVC
YPRGSKPEDANFYLYLRARVCENCMEMDVMSVATIVIVDICITGG
LLLLVYYWSKNRKAKAKPVTRGAGAGGRQRGQNKERPPPVPNPDY
EPIRKGQRDLYSGLNQRRIGPQCTNYALLKLAGDVESNPGP
(SEQ ID NO: 80)
ATGAAGTGGAAGGCGCTTTTCACCGCGGCCATCCTGCAGGCACAG
TTGCCGATTACAGAGGCACAGAGCTTTGGCCTGCTGGATCCCAAA
CTCTGCTACCTGCTGGATGGAATCCTCTTCATCTATGGTGTCATT
CTCACTGCCTTGTTCCTGAGAGTGAAGTTCAGCAGGAGCGCAGAC
GCCCCCGCGTACCAGCAGGGCCAGAACCAGCTCTATAACGAGCTC
AATCTAGGACGAAGAGAGGAGTACGATGTTTTGGACAAGAGACGT
GGCCGGGACCCTGAGATGGGGGGAAAGCCGCAGAGAAGGAAGAAC
CCTCAGGAAGGCCTGTACAATGAACTGCAGAAAGATAAGATGGCG
GAGGCCTACAGTGAGATTGGGATGAAAGGCGAGCGCCGGAGGGGC
AAGGGGCACGATGGCCTTTACCAGGGTCTCAGTACAGCCACCAAG
GACACCTACGACGCCCTTCACATGCAGGCCCTGCCCCCTCGCCAG
TGCACCAACTACGCCCTGCTGAAGCTGGCCGGCGACGTGGAGAGC
AACCCCGGCCCCATGGAACAGGGGAAGGGCCTGGCTGTCCTCATC
CTGGCTATCATTCTTCTTCAAGGTACTTTGGCCCAGTCAATCAAA
GGAAACCACTTGGTTAAGGTGTATGACTATCAAGAAGATGGTTCG
GTACTTCTGACTTGTGATGCAGAAGCCAAAAATATCACATGGTTT
AAAGATGGGAAGATGATCGGCTTCCTAACTGAAGATAAAAAAAAA
TGGAATCTGGGAAGTAATGCCAAGGACCCTCGTGGGATGTATCAG
TGTAAAGGATCACAGAACAAGTCAAAACCACTCCAAGTGTATTAC
AGAATGTGTCAGAACTGCATTGAACTAAATGCAGCCACCATATCT
GGCTTTCTCTTTGCTGAAATCGTCAGCATTTTCGTCCTTGCTGTT
GGGGTCTACTTCATTGCTGGACAGGATGGAGTTCGCCAGTCGAGA
GCTTCAGACAAGCAGACTCTGTTGCCCAATGACCAGCTCTACCAG
CCCCTCAAGGATCGAGAAGATGACCAGTACAGCCACCTTCAAGGA
AACCAGTTGAGGAGGAATGTGAAGCAGACCCTGAACTTCGACCTG
CTGAAGCTGGCCGGCGACGTGGAGAGCAACCCCGGCCCCATGGAG
CACAGCACCTTCCTGAGCGGCCTGGTGCTGGCCACCCTGCTGAGC
CAGGTGAGCCCCTTCAAGATCCCCATCGAGGAGCTGGAGGACAGA
GTGTTCGTGAACTGCAACACCAGCATCACCTGGGTGGAGGGCACC
GTGGGCACCCTGCTGAGCGACATCACCAGACTGGACCTGGGCAAG
AGAATCCTGGACCCCAGAGGCATCTACAGATGCAACGGCACCGAC
ATCTACAAGGACAAGGAGAGCACCGTGCAGGTGCACTACAGAATG
TGCCAGAGCTGCGTGGAGCTGGACCCCGCCACCGTGGCCGGCATC
ATCGTGACCGACGTGATCGCCACCCTGCTGCTGGCCCTGGGCGTG
TTCTGCTTCGCCGGCCACGAGACCGGCAGACTGAGCGGCGCCGCC
GACACCCAGGCCCTGCTGAGAAACGACCAGGTGTACCAGCCCCTG
AGAGACAGAGACGACGCCCAGTACAGCCACCTGGGCGGCAACTGG
GCCAGAAACAAGGAGGGCAGAGGCAGCCTGCTGACCTGCGGCGAC
GTGGAGGAGAACCCCGGCCCCATGCAGAGCGGCACCCACTGGAGA
GTGCTGGGCCTGTGCCTGCTGAGCGTGGGCGTGTGGGGCCAGGAC
GGCAACGAGGAGATGGGCGGCATCACCCAGACCCCCTACAAGGTG
AGCATCAGCGGCACCACCGTGATCCTGACCTGCCCCCAGTACCCC
GGCAGCGAGATCCTGTGGCAGCACAACGACAAGAACATCGGCGGC
GACGAGGACGACAAGAACATCGGCAGCGACGAGGACCACCTGAGC
CTGAAGGAGTTCAGCGAGCTGGAGCAGAGCGGCTACTACGTGTGC
TACCCCAGAGGCAGCAAGCCCGAGGACGCCAACTTCTACCTGTAC
CTGAGAGCCAGAGTGTGCGAGAACTGCATGGAGATGGACGTGATG
AGCGTGGCCACCATCGTGATCGTGGACATCTGCATCACCGGCGGC
CTGCTGCTGCTGGTGTACTACTGGAGCAAGAACAGAAAGGCCAAG
GCCAAGCCCGTGACCAGAGGCGCCGGCGCCGGCGGCAGACAGAGA
GGCCAGAACAAGGAGAGACCCCCCCCCGTGCCCAACCCCGACTAC
GAGCCCATCAGAAAGGGCCAGAGAGACCTGTACAGCGGCCTGAAC
CAGAGAAGAATCGGACCGCAGTGTACTAATTATGCTCTCTTGAAA
TTGGCTGGAGATGTTGAGAGCAATCCCGGGCCC
IL-15:
(SEQ ID NO: 48)
MRISKPHLRSISIQCYLCLLLNSHELTEAGIHVFILGCFSAGLPK
TEANWVNVISDLKKIEDLIQSMHIDATLYTESDVHPSCKVTAMKC
FLLELQVISLESGDASIHDTVENLIILANNSLSSNGNVTESGCKE
CEELEEKNIKEFLQSFVHIVQMFINTS*
(SEQ ID NO: 49)
ATGCGCATTAGCAAGCCCCACCTGCGGAGCATCAGCATCCAGTGC
TACCTGTGCCTGCTGCTGAACAGCCACTTCCTGACCGAGGCCGGC
ATCCACGTGTTCATCCTGGGCTGCTTCAGCGCCGGACTGCCCAAG
ACCGAGGCCAACTGGGTGAACGTGATCAGCGACCTGAAGAAGATC
GAGGACCTGATCCAGAGCATGCACATCGACGCCACCCTGTACACC
GAGAGCGACGTGCACCCCAGCTGCAAGGTGACCGCCATGAAGTGC
TTTCTGCTGGAACTGCAGGTGATCAGCCTGGAAAGCGGCGACGCC
AGCATCCACGACACCGTGGAGAACCTGATCATCCTGGCCAACAAC
AGCCTGAGCAGCAACGGCAACGTGACCGAGAGCGGCTGCAAAGAG
TGCGAGGAACTGGAAGAGAAGAACATCAAAGAGTTTCTGCAGAGC
TTCGTGCACATCGTGCAGATGTTCATCAACACCAGC

In some embodiments, a TCR construct comprises an NY-ESO-specific TCR and a CD8 alpha/beta co-receptor molecule. In some embodiments, such a construct can comprise a TCR alpha chain variable region signal peptide, a TCR alpha chain variable region, a TCR alpha chain constant region, a 2A element (e.g., P2A element), a TCR beta chain variable region signal peptide, a TCR beta chain variable region, a TCR beta chain constant region, a 2A element (e.g., a E2A element), a CD8-beta polypeptide, a 2A element (e.g., a T2A element), and a CD8-alpha polypeptide. In some embodiments, a TCR construct comprising an NY-ESO-specific TCR and a CD8 alpha/beta co-receptor molecule nucleotide coding sequence is at least, or exactly, 85%, 86%, 87%, 88%, 89%, 90%, 91%, 92%, 93%, 94%, 95%, 96%, 97%, 98%, 99%, or 100%, identical to SEQ ID NO: 124. In some embodiments, a TCR construct comprising an NY-ESO-specific TCR and a CD8 alpha/beta co-receptor molecule amino acid sequence is at least, or exactly, 85%, 86%, 87%, 88%, 89%, 90%, 91%, 92%, 93%, 94%, 95%, 96%, 97%, 98%, 99%, or 100%, identical to SEQ ID NO: 125.

In some embodiments, a CD8 alpha co-receptor molecule is transcriptionally linked to any TCR molecule disclosed herein. In some embodiments, a CD8 alpha co-receptor molecule nucleotide coding sequence is at least, or exactly, 85%, 86%, 87%, 88%, 89%, 90%, 91%, 92%, 93%, 94%, 95%, 96%, 97%, 98%, 99%, or 100%, identical to SEQ ID NO: 126. In some embodiments, a CD8 beta co-receptor molecule nucleotide coding sequence is at least, or exactly, 85%, 86%, 87%, 88%, 89%, 90%, 91%, 92%, 93%, 94%, 95%, 96%, 97%, 98%, 99%, or 100%, identical to SEQ ID NO: 127. In some embodiments, a CD8 alpha co-receptor amino acid sequence is at least, or exactly, 85%, 86%, 87%, 88%, 89%, 90%, 91%, 92%, 93%, 94%, 95%, 96%, 97%, 98%, 99%, or 100%, identical to SEQ ID NO: 128. In some embodiments, a CD8 beta co-receptor amino acid sequence is at least, or exactly, 85%, 86%, 87%, 88%, 89%, 90%, 91%, 92%, 93%, 94%, 95%, 96%, 97%, 98%, 99%, or 100%, identical to SEQ ID NO: 129.

(SEQ ID NO: 124)
ATGGAGACCCTCTTGGGCCTGCTTATCCTTTGGCTGCAGCTGCAA
TGGGTGAGCAGCAAACAGGAGGTGACACAGATTCCTGCAGCTCTG
AGTGTCCCAGAAGGAGAAAACTTGGTTCTCAACTGCAGTTTCACT
GATAGCGCTATTTACAACCTCCAGTGGTTTAGGCAGGACCCTGGG
AAAGGTCTCACATCTCTGTTGCTTATTCAGTCAAGTCAGAGAGAG
CAAACAAGTGGAAGACTTAATGCCTCGCTGGATAAATCATCAGGA
CGTAGTACTTTATACATTGCAGCTTCTCAGCCTGGTGACTCAGCC
ACCTACCTCTGTGCTGTGAGGCCCCTTTATGGAGGAAGCTACATA
CCTACATTTGGAAGAGGAACCAGCCTTATTGTTCATCCGTATATC
CAGAACCCTGACCCTGCCGTGTACCAGCTGAGAGACTCTAAATCC
AGTGACAAGTCTGTCTGCCTATTCACCGATTTTGATTCTCAAACA
AATGTGTCACAAAGTAAGGATTCTGATGTGTATATCACAGACAAA
ACTGTGCTAGACATGAGGTCTATGGACTTCAAGAGCAACAGTGCT
GTGGCCTGGAGCAACAAATCTGACTTTGCATGTGCAAACGCCTTC
AACAACAGCATTATTCCAGAAGACACCTTCTTCCCCAGCCCAGAA
AGTTCCTGTGATGTCAAGCTGGTCGAGAAAAGCTTTGAAACAGAT
ACGAACCTAAACTTTCAAAACCTGTCAGTGATTGGGTTCCGAATC
CTCCTCCTGAAAGTGGCCGGGTTTAATCTGCTCATGACGCTGCGG
CTGTGGTCCAGCGGAAGCGGAGCTACTAACTTTAGCCTGCTGAAG
CAGGCTGGAGATGTGGAGGAGAACCCTGGACCTATGAGCATCGGC
CTCCTGTGCTGTGCAGCCTTGTCTCTCCTGTGGGCAGGTCCAGTG
AATGCTGGTGTCACTCAGACCCCAAAATTCCAGGTCCTGAAGACA
GGACAGAGCATGACACTGCAGTGTGCCCAGGATATGAACCATGAA
TACATGTCCTGGTATCGACAAGACCCAGGCATGGGGCTGAGGCTG
ATTCATTACTCAGTTGGTGCTGGTATCACTGACCAAGGAGAAGTC
CCCAATGGCTACAATGTCTCCAGATCAACCACAGAGGATTTCCCG
CTCAGGCTGCTGTCGGCTGCTCCCTCCCAGACATCTGTGTACTTC
TGTGCCAGCAGTTACGTCGGGAACACCGGGGAGCTGTTTTTTGGA
GAAGGCTCTAGGCTGACCGTACTGGAGGACCTGAAAAACGTGTTC
CCACCCAAGGTCGCTGTGTTTGAGCCATCAGAAGCAGAGATCTCC
CACACCCAAAAGGCCACACTGGTATGCCTGGCCACAGGCTTCTAC
CCCGACCACGTGGAGCTGAGCTGGTGGGTGAATGGGAAGGAGGTG
CACAGTGGGGTCAGCACAGACCCGCAGCCCCTCAAGGAGCAGCCC
GCCCTCAATGACTCCAGATACTGCCTGAGCAGCCGCCTGAGGGTC
TCGGCCACCTTCTGGCAGAACCCCCGCAACCACTTCCGCTGTCAA
GTCCAGTTCTACGGGCTCTCGGAGAATGACGAGTGGACCCAGGAT
AGGGCCAAACCCGTCACCCAGATCGTCAGCGCCGAGGCCTGGGGT
AGAGCAGACTGTGGCTTCACCTCCGAGTCTTACCAGCAAGGGGTC
CTGTCTGCCACCATCCTCTATGAGATCTTGCTAGGGAAGGCCACC
TTGTATGCCGTGCTGGTCAGTGCCCTCGTGCTGATGGCCATGGTC
AAGAGAAAGGATTCCAGAGGCAGTGGACAGTGCACCAACTACGCC
CTGCTGAAGCTGGCCGGCGACGTGGAGAGCAACCCCGGCCCCATG
GCCTTGCCCGTCACTGCGCTTTTGCTCCCGCTCGCTCTTCTCCTG
CATGCAGCCCGACCATCTCAATTTAGAGTTTCTCCACTCGACAGG
ACGTGGAACCTCGGCGAAACCGTCGAACTTAAATGTCAAGTACTT
CTCTCAAATCCGACTTCTGGTTGCTCATGGCTCTTTCAGCCGAGA
GGAGCAGCTGCCAGCCCCACCTTCCTGCTGTATCTCTCCCAGAAC
AAGCCGAAGGCCGCCGAAGGGCTCGATACTCAACGATTTAGCGGG
AAGCGACTCGGGGACACGTTCGTTCTTACTCTCAGCGATTTTAGA
AGAGAGAACGAGGGATATTATTTTTGTTCCGCACTCTCTAACAGC
ATCATGTACTICAGTCATTTTGTACCAGTCTTTCTCCCTGCAAAA
CCAACGACTACTCCAGCACCAAGACCGCCCACTCCCGCACCTACT
ATTGCAAGCCAACCTTTGAGTCTCCGACCAGAGGCATGCAGACCT
GCTGCTGGAGGTGCAGTACATACGCGAGGGTTGGATTTTGCCTGC
GATATCTATATCTGGGCCCCCTTGGCCGGCACGTGCGGGGTGCTC
CTGCTGAGTCTCGTAATTACTCTTTATTGTAATCATAGAAACCGC
AGAAGGGTGTGTAAGTGTCCCCGGCCTGTCGTGAAAAGCGGGGAT
AAGCCCAGTTTGTCTGCTCGGTACGTCGGAAGCGGTGAGGGCAGG
GGAAGTCTTCTAACATGCGGGGACGTGGAGGAAAATCCCGGACCC
ATGAGGCCACGACTTTGGCTGCTGCTCGCTGCACAGTTGACTGTA
CTGCATGGCAATAGTGTGTTGCAGCAGACACCTGCATACATCAAG
GTTCAGACAAATAAGATGGTTATGCTGAGTTGCGAGGCAAAAATT
AGTTTGAGCAATATGCGGATCTACTGGTTGCGACAGAGACAGGCT
CCCAGTAGTGATAGTCACCACGAATTCCTGGCTCTTTGGGATTCC
GCAAAAGGAACGATTCATGGGGAAGAAGTAGAGCAGGAGAAGATT
GCGGTTTTCCGCGATGCATCTCGCTTTATCCTTAATCTTACATCC
GTTAAGCCTGAGGACAGTGGGATCTATTTTTGTATGATTGTAGGG
TCCCCCGAATTGACATTTGGGAAGGGTACGCAGCTCTCCGTAGTT
GACTTTCTGCCCACAACGGCACAACCCACTAAGAAGTCCACCCTG
AAGAAGCGCGTCTGTCGCTTGCCCAGACCTGAAACCCAAAAGGGT
CCACTCTGTTCCCCTATAACCCTGGGGTTGTTGGTGGCGGGCGTC
TTGGTCCTGCTTGTTAGCTTGGGCGTAGCCATTCATCTGTGTTGC
CGAAGACGCAGAGCCCGACTTAGATTTATGAAGCAATTCTATAAG
TGA
(SEQ ID NO: 125)
METLLGLLILWLQLQWVSSKQEVTQIPAALSVPEGENLVLNCSFT
DSAIYNLQWFRQDPGKGLTSLLLIQSSQREQTSGRLNASLDKSSG
RSTLYIAASQPGDSATYLCAVRPLYGGSYIPTFGRGTSLIVHPYI
QNPDPAVYQLRDSKSSDKSVCLFTDFDSQTNVSQSKDSDVYITDK
TVLDMRSMDFKSNSAVAWSNKSDFACANAFNNSIIPEDTFFPSPE
SSCDVKLVEKSFETDINLNFQNLSVIGFRILLLKVAGFNLLMTLR
LWSSGSGATNFSLLKQAGDVEENPGPMSIGLLCCAALSLLWAGPV
NAGVTQTPKFQVLKTGQSMTLQCAQDMNHEYMSWYRQDPGMGLRL
IHYSVGAGITDQGEVPNGYNVSRSTTEDEPLRLLSAAPSQTSVYF
CASSYVGNTGELFFGEGSRLTVLEDLKNVFPPKVAVFEPSEAEIS
HTQKATLVCLATGFYPDHVELSWWVNGKEVHSGVSTDPQPLKEQP
ALNDSRYCLSSRLRVSATFWQNPRNHFRCQVQFYGLSENDEWTQD
RAKPVTQIVSAEAWGRADCGFTSESYQQGVLSATILYEILLGKAT
LYAVLVSALVLMAMVKRKDSRGSGQCTNYALLKLAGDVESNPGPM
ALPVTALLLPLALLLHAARPSQFRVSPLDRTWNLGETVELKCQVL
LSNPTSGCSWLFQPRGAAASPTELLYLSQNKPKAAEGLDTQRFSG
KRLGDTFVLTLSDFRRENEGYYFCSALSNSIMYFSHFVPVFLPAK
PTTTPAPRPPTPAPTIASQPLSLRPEACRPAAGGAVHTRGLDFAC
DIYIWAPLAGTCGVLLLSLVITLYCNHRNRRRVCKCPRPVVKSGD
KPSLSARYVGSGEGRGSLLTCGDVEENPGPMRPRLWLLLAAQLTV
LHGNSVLQQTPAYIKVQTNKMVMLSCEAKISLSNMRIYWLRQRQA
PSSDSHHEFLALWDSAKGTIHGEEVEQEKIAVERDASRFILNLTS
VKPEDSGIYFCMIVGSPELTFGKGTQLSVVDFLPTTAQPTKKSTL
KKRVCRLPRPETQKGPLCSPITLGLLVAGVLVLLVSLGVAIHLCC
RRRRARLREMKQFYK*
(SEQ ID NO: 126)
ATGAGGCCACGACTTTGGCTGCTGCTCGCTGCACAGTTGACTGTA
CTGCATGGCAATAGTGTGTTGCAGCAGACACCTGCATACATCAAG
GTTCAGACAAATAAGATGGTTATGCTGAGTTGCGAGGCAAAAATT
AGTTTGAGCAATATGCGGATCTACTGGTTGCGACAGAGACAGGCT
CCCAGTAGTGATAGTCACCACGAATTCCTGGCTCTTTGGGATTCC
GCAAAAGGAACGATTCATGGGGAAGAAGTAGAGCAGGAGAAGATT
GCGGTTTTCCGCGATGCATCTCGCTTTATCCTTAATCTTACATCC
GTTAAGCCTGAGGACAGTGGGATCTATTTTTGTATGATTGTAGGG
TCCCCCGAATTGACATTTGGGAAGGGTACGCAGCTCTCCGTAGTT
GACTTTCTGCCCACAACGGCACAACCCACTAAGAAGTCCACCCTG
AAGAAGCGCGTCTGTCGCTTGCCCAGACCTGAAACCCAAAAGGGT
CCACTCTGTTCCCCTATAACCCTGGGGTTGTTGGTGGCGGGCGTC
TTGGTCCTGCTTGTTAGCTTGGGCGTAGCCATTCATCTGTGTTGC
CGAAGACGCAGAGCCCGACTTAGATTTATGAAGCAATTCTATAAG
TGA
(SEQ ID NO: 127)
ATGGCCTTGCCCGTCACTGCGCTTTTGCTCCCGCTCGCTCTTCTC
CTGCATGCAGCCCGACCATCTCAATTTAGAGTTTCTCCACTCGAC
AGGACGTGGAACCTCGGCGAAACCGTCGAACTTAAATGTCAAGTA
CTTCTCTCAAATCCGACTTCTGGTTGCTCATGGCTCTTTCAGCCG
AGAGGAGCAGCTGCCAGCCCCACCTTCCTGCTGTATCTCTCCCAG
AACAAGCCGAAGGCCGCCGAAGGGCTCGATACTCAACGATTTAGC
GGGAAGCGACTCGGGGACACGTTCGTTCTTACTCTCAGCGATTTT
AGAAGAGAGAACGAGGGATATTATTTTTGTTCCGCACTCTCTAAC
AGCATCATGTACTTCAGTCATTTTGTACCAGTCTTTCTCCCTGCA
AAACCAACGACTACTCCAGCACCAAGACCGCCCACTCCCGCACCT
ACTATTGCAAGCCAACCTTTGAGTCTCCGACCAGAGGCATGCAGA
CCTGCTGCTGGAGGTGCAGTACATACGCGAGGGTTGGATTTTGCC
TGCGATATCTATATCTGGGCCCCCTTGGCCGGCACGTGCGGGGTG
CTCCTGCTGAGTCTCGTAATTACTCTTTATTGTAATCATAGAAAC
CGCAGAAGGGTGTGTAAGTGTCCCCGGCCTGTCGTGAAAAGCGGG
GATAAGCCCAGTTTGTCTGCTCGGTACGTC
(SEQ ID NO: 128)
MRPRLWLLLAAQLTVLHGNSVLQQTPAYIKVQTNKMVMLSCEAKI
SLSNMRIYWLRQRQAPSSDSHHEFLALWDSAKGTIHGEEVEQEKI
AVFRDASRFILNLTSVKPEDSGIYFCMIVGSPELTFGKGTQLSVV
DFLPTTAQPTKKSTLKKRVCRLPRPETQKGPLCSPITLGLLVAGV
LVLLVSLGVAIHLCCRRRRARLREMKQFYK
(SEQ ID NO: 129)
MALPVTALLLPLALLLHAARPSQFRVSPLDRTWNLGETVELKCQV
LLSNPTSGCSWLFQPRGAAASPTELLYLSQNKPKAAEGLDTQRFS
GKRLGDTFVLTLSDERRENEGYYFCSALSNSIMYFSHFVPVELPA
KPTTTPAPRPPTPAPTIASQPLSLRPEACRPAAGGAVHTRGLDFA
CDIYIWAPLAGTCGVLLLSLVITLYCNHRNRRRVCKCPRPVVKSG
DKPSLSARYV

In some embodiments, a TCR construct comprises PRAME-specific TCR chains. In some embodiments, a TCR construct comprising PRAME-specific TCR chains comprises TCR alpha and TCR beta chains found in PRAME-specific TCR clone 46, clone 54, and/or clone DSK3. In some embodiments, a TCR construct comprising PRAME-specific TCR chains comprises TCR alpha and TCR beta chains that target PRAME epitopes SLLQHLIGL (SEQ ID NO: 131) and/or QLLALLPSL (SEQ ID NO: 132).

In some embodiments, a TCR construct comprising PRAME-specific TCR chains comprises a nucleotide coding sequence that is at least, or exactly, 85%, 86%, 87%, 88%, 89%, 90%, 91%, 92%, 93%, 94%, 95%, 96%, 97%, 98%, 99%, or 100%, identical to SEQ ID NO: 133 (e.g., TCR clone 46 TCR alpha) and/or 134 (e.g., TCR clone 46 TCR beta). In some embodiments, a TCR construct comprising PRAME-specific TCR chains comprises an amino acid sequence that is at least, or exactly, 85%, 86%, 87%, 88%, 89%, 90%, 91%, 92%, 93%, 94%, 95%, 96%, 97%, 98%, 99%, or 100%, identical to SEQ ID NO: 135 (e.g., TCR clone 46 TCR alpha) and/or 136 (e.g., TCR clone 46 TCR beta).

(SEQ ID NO: 133)
ATGCTTCTGGAACACCTGCTGATTATCCTGTGGATGCAACTCACG
TGGGTCTCCGGGCAACAACTGAATCAAAGCCCCCAATCCATGTTT
ATACAGGAGGGAGAGGACGTAAGTATGAATTGCACATCTTCATCT
ATCTTTAACACCTGGCTGTGGTACAAACAAGACCCCGGAGAAGGT
CCTGTACTTCTCATCGCACTTTACAAAGCAGGTGAGCTTACCAGT
AACGGGAGACTCACCGCACAGTTCGGTATTACAAGAAAGGATTCC
TTTCTCAACATCTCCGCTTCTATCCCTTCAGACGTCGGAATTTAT
TTTTGTGCTGGTATCCCTCGAGACAATTACGGTCAAAACTTTGTA
TTTGGGCCTGGGACTCGGCTGTCAGTTTTGCCGTATATCCAGAAC
CCCGACCCCGCCGTGTACCAGCTGCGGGACAGCAAGAGCAGCGAC
AAGAGCGTGTGCCTGTTCACCGACTTCGACAGCCAGACCAACGTG
TCCCAGAGCAAGGACAGCGACGTGTACATCACCGATAAGTGCGTG
CTGGACATGCGGAGCATGGACTTCAAGAGCAACAGCGCCGTGGCC
TGGTCCAACAAGAGCGACTTCGCCTGCGCCAACGCCTTCAACAAC
AGCATCATCCCCGAGGACACATTCTTCCCAAGCCCCGAGAGCAGC
TGCGACGTGAAGCTGGTGGAGAAGTCCTTCGAGACAGACACCAAC
CTGAACTTCCAGAACCTGTCCGTGATCGGCTTCAGAATCCTGCTG
CTGAAAGTGGCCGGCTTCAACCTGCTGATGACCCTGCGGCTGTGG
TCCAGC
(SEQ ID NO: 134)
ATGGGCATTAGGCTGCTGTGCAGAGTAGCATTTTGCTTTCTGGCA
GTAGGATTGGTCGATGTAAAGGTTACACAGTCCTCACGGTACTTG
GTAAAGCGCACTGGTGAAAAGGTCTTTCTGGAATGTGTACAAGAT
ATGGATCACGAAAATATGTTTTGGTACAGGCAAGATCCCGGCCTT
GGACTTAGACTGATATATTTCTCCTACGATGTTAAAATGAAGGAG
AAGGGCGATATTCCAGAAGGATATTCCGTGAGCCGCGAAAAGAAG
GAGCGATTCAGTTTGATACTCGAAAGTGCCTCCACAAACCAAACC
TCTATGTACCTTTGCGCGTCAACGCCGTGGCTGGCCGGTGGCAAT
GAACAATTCTTCGGGCCGGGTACGCGCCTCACTGTCCTGGAGGAC
CTCAAGAATGTGTTTCCGCCCGAAGTCGCGGTTTTTGAACCATCA
GAAGCCGAGATCTCTCATACACAAAAGGCGACGCTCGTATGCCTT
GCGACGGGATTTTATCCGGACCACGTCGAGCTTTCCTGGTGGGTT
AATGGAAAGGAGGTGCATTCCGGAGTTTGCACGGACCCTCAGCCA
TTGAAGGAACAGCCCGCACTGAACGACAGTAGGTATTGCCTTTCA
TCTCGCCTGCGCGTGTCTGCGACATTCTGGCAAAACCCAAGAAAT
CACTTCAGATGTCAAGTTCAGTTCTACGGTCTCAGCGAGAATGAT
GAGTGGACACAAGATAGGGCTAAACCCGTGACTCAAATAGTCTCT
GCCGAGGCCTGGGGGAGGGCGGATTGCGGCTTCACATCAGAATCA
TACCAACAAGGAGTATTGAGCGCGACAATTCTTTACGAAATTCTG
CTTGGGAAAGCGACTCTGTACGCGGTGCTCGTGTCCGCTTTGGTT
CTTATGGCAATGGTTAAACGAAAGGATAGTAGGGGC
(SEQ ID NO: 135)
MLLEHLLIILWMQLTWVSGQQLNQSPQSMFIQEGEDVSMNCTSSS
IFNTWLWYKQDPGEGPVLLIALYKAGELTSNGRLTAQFGITRKDS
FLNISASIPSDVGIYFCAGIPRDNYGQNFVFGPGTRLSVLPYIQN
PDPAVYQLRDSKSSDKSVCLFTDFDSQTNVSQSKDSDVYITDKCV
LDMRSMDFKSNSAVAWSNKSDFACANAFNNSIIPEDTFFPSPESS
CDVKLVEKSFETDINLNFQNLSVIGFRILLLKVAGENLLMTLRLW
SS
(SEQ ID NO: 136)
MGIRLLCRVAFCFLAVGLVDVKVTQSSRYLVKRTGEKVFLECVQD
MDHENMFWYRQDPGLGLRLIYFSYDVKMKEKGDIPEGYSVSREKK
ERFSLILESASTNQTSMYLCASTPWLAGGNEQFFGPGTRLTVLED
LKNVFPPEVAVFEPSEAEISHTQKATLVCLATGFYPDHVELSWWV
NGKEVHSGVCTDPQPLKEQPALNDSRYCLSSRLRVSATFWQNPRN
HFRCQVQFYGLSENDEWTQDRAKPVTQIVSAEAWGRADCGFTSES
YQQGVLSATILYEILLGKATLYAVLVSALVLMAMVKRKDSRG

In some embodiments, a TCR construct comprising PRAME-specific TCR chains comprises a nucleotide coding sequence that is at least, or exactly, 85%, 86%, 87%, 88%, 89%, 90%, 91%, 92%, 93%, 94%, 95%, 96%, 97%, 98%, 99%, or 100%, identical to SEQ ID NO: 137 (e.g., TCR clone 54 TCR alpha) and/or 138 (e.g., TCR clone 54 TCR beta). In some embodiments, a TCR construct comprising PRAME-specific TCR chains comprises an amino acid sequence that is at least, or exactly, 85%, 86%, 87%, 88%, 89%, 90%, 91%, 92%, 93%, 94%, 95%, 96%, 97%, 98%, 99%, or 100%, identical to SEQ ID NO: 139 (e.g., TCR clone 54 TCR alpha) and/or 140 (e.g., TCR clone 54 TCR beta).

(SEQ ID NO: 137)
ATGCTGCTGCTGCTGGTGCCCGTGCTGGAAGTGATCTTCACCCTG
GGCGGCACCAGAGCCCAGAGCGTGACACAGCTGGGCAGCCACGTG
TCCGTGTCTGAGAGGGCCCTGGTGCTGCTGAGATGCAACTACTCT
TCTAGCGTGCCCCCCTACCTGTTTTGGTACGTGCAGTACCCCAAC
CAGGGGCTGCAGCTGCTCCTGAAGTACACCAGCGCCGCCACACTG
GTGAAGGGCATCAACGGCTTCGAGGCCGAGTTCAAGAAGTCCGAG
ACAAGCTTCCACCTGACCAAGCCCAGCGCCCACATGTCTGACGCC
GCCGAGTACTTCTGTGCCGTGAGCGGCCAGACCGGCGCCAACAAC
CTGTTCTTCGGCACCGGCACCCGGCTGACAGTGATCCCTTACATC
CAGAACCCCGACCCCGCCGTGTACCAGCTGCGGGACAGCAAGAGC
AGCGACAAGAGCGTGTGCCTGTTCACCGACTTCGACAGCCAGACC
AACGTGTCCCAGAGCAAGGACAGCGACGTGTACATCACCGATAAG
TGCGTGCTGGACATGCGGAGCATGGACTTCAAGAGCAACAGCGCC
GTGGCCTGGTCCAACAAGAGCGACTTCGCCTGCGCCAACGCCTTC
AACAACAGCATCATCCCCGAGGACACATTCTTCCCAAGCCCCGAG
AGCAGCTGCGACGTGAAGCTGGTGGAGAAGTCCTTCGAGACAGAC
ACCAACCTGAACTTCCAGAACCTGTCCGTGATCGGCTTCAGAATC
CTGCTGCTGAAAGTGGCCGGCTTCAACCTGCTGATGACCCTGCGG
CTGTGGTCCAGC
(SEQ ID NO: 138)
ATGGGCTTCCGGCTGCTGTGCTGCGTGGCCTTTTGTCTGCTGGGA
GCCGGACCTGTGGATAGCGGCGTGACCCAGACCCCCAAGCACCTG
ATCACCGCCACCGGCCAGAGAGTGACCCTGCGCTGCAGCCCTAGA
AGCGGCGACCTGAGCGTGTACTGGTATCAGCAGAGCCTCGACCAG
GGCCTGCAGTTCCTGATCCAGTACTACAACGGCGAGGAACGGGCC
AAGGGCAACATCCTGGAACGGTTCAGCGCCCAGCAGTTCCCCGAT
CTGCACAGCGAGCTGAACCTGAGCAGCCTGGAACTGGGCGACAGC
GCCCTGTACTTCTGCGCCAGCGCCAGATGGGATAGAGGCGGCGAG
CAGTACTTCGGCCCTGGCACCAGACTGACCGTGACCGAGGACCTC
AAGAATGTGTTTCCGCCCGAAGTCGCGGTTTTTGAACCATCAGAA
GCCGAGATCTCTCATACACAAAAGGCGACGCTCGTATGCCTTGCG
ACGGGATTTTATCCGGACCACGTCGAGCTTTCCTGGTGGGTTAAT
GGAAAGGAGGTGCATTCCGGAGTTTGCACGGACCCTCAGCCATTG
AAGGAACAGCCCGCACTGAACGACAGTAGGTATTGCCTTTCATCT
CGCCTGCGCGTGTCTGCGACATTCTGGCAAAACCCAAGAAATCAC
TTCAGATGTCAAGTTCAGTTCTACGGTCTCAGCGAGAATGATGAG
TGGACACAAGATAGGGCTAAACCCGTGACTCAAATAGTCTCTGCC
GAGGCCTGGGGGAGGGCGGATTGCGGCTTCACATCAGAATCATAC
CAACAAGGAGTATTGAGCGCGACAATTCTTTACGAAATTCTGCTT
GGGAAAGCGACTCTGTACGCGGTGCTCGTGTCCGCTTTGGTTCTT
ATGGCAATGGTTAAACGAAAGGATAGTAGGGGC
(SEQ ID NO: 139)
MLLLLVPVLEVIFTLGGTRAQSVTQLGSHVSVSERALVLLRCNYS
SSVPPYLFWYVQYPNQGLQLLLKYTSAATLVKGINGFEAEFKKSE
TSFHLTKPSAHMSDAAEYFCAVSGQTGANNLFFGTGTRLTVIPYI
QNPDPAVYQLRDSKSSDKSVCLFTDFDSQTNVSQSKDSDVYITDK
CVLDMRSMDFKSNSAVAWSNKSDFACANAFNNSIIPEDTFFPSPE
SSCDVKLVEKSFETDINLNFQNLSVIGFRILLLKVAGENLLMTLR
LWSS
(SEQ ID NO: 140)
MGFRLLCCVAFCLLGAGPVDSGVTQTPKHLITATGQRVTLRCSPR
SGDLSVYWYQQSLDQGLQFLIQYYNGEERAKGNILERFSAQQFPD
LHSELNLSSLELGDSALYFCASARWDRGGEQYFGPGTRLTVTEDL
KNVFPPEVAVFEPSEAEISHTQKATLVCLATGFYPDHVELSWWVN
GKEVHSGVCTDPQPLKEQPALNDSRYCLSSRLRVSATFWQNPRNH
FRCQVQFYGLSENDEWTQDRAKPVTQIVSAEAWGRADCGFTSESY
QQGVLSATILYEILLGKATLYAVLVSALVLMAMVKRKDSRG

In some embodiments, a TCR construct comprising PRAME-specific TCR chains comprises a nucleotide coding sequence that is at least, or exactly, 85%, 86%, 87%, 88%, 89%, 90%, 91%, 92%, 93%, 94%, 95%, 96%, 97%, 98%, 99%, or 100%, identical to SEQ ID NO: 141 (e.g., TCR clone DSK3 TCR alpha) and/or 142 (e.g., TCR clone DSK3 TCR beta). In some embodiments, a TCR construct comprising PRAME-specific TCR chains comprises an amino acid sequence that is at least, or exactly, 85%, 86%, 87%, 88%, 89%, 90%, 91%, 92%, 93%, 94%, 95%, 96%, 97%, 98%, 99%, or 100%, identical to SEQ ID NO: 143 (e.g., TCR clone DSK3 TCR alpha) and/or 144 (e.g., TCR clone DSK3 TCR beta).

(SEQ ID NO: 141)
ATGAAGAGCCTGAGGGTACTGCTGGTGATATTGTGGCTTCAGCTT
AGTTGGGTCTGGTCACAACAAAAGGAAGTTGAGCAAAACTCAGGA
CCACTGAGTGTACCCGAGGGCGCTATAGCATCACTGAACTGTACC
TACTCAGATCGGGGAAGCCAATCCTTTTTCTGGTACAGACAGTAT
TCCGGGAAGAGTCCTGAGTTGATCATGTTTATATACTCCAATGGC
GATAAGGAGGATGGACGCTTCACCGCTCAGCTTAATAAAGCGTCA
CAGTATGTATCCCTCCTGATTCGGGACTCACAACCATCTGACTCT
GCAACATACCTTTGTGCCGTAAAGGACAACGCCGGGAACATGCTC
ACTTTTGGAGGAGGTACCCGGCTTATGGTAAAACCACATATCCAG
AACCCCGACCCCGCCGTGTACCAGCTGCGGGACAGCAAGAGCAGC
GACAAGAGCGTGTGCCTGTTCACCGACTTCGACAGCCAGACCAAC
GTGTCCCAGAGCAAGGACAGCGACGTGTACATCACCGATAAGTGC
GTGCTGGACATGCGGAGCATGGACTTCAAGAGCAACAGCGCCGTG
GCCTGGTCCAACAAGAGCGACTTCGCCTGCGCCAACGCCTTCAAC
AACAGCATCATCCCCGAGGACACATTCTTCCCAAGCCCCGAGAGC
AGCTGCGACGTGAAGCTGGTGGAGAAGTCCTTCGAGACAGACACC
AACCTGAACTTCCAGAACCTGTCCGTGATCGGCTTCAGAATCCTG
CTGCTGAAAGTGGCCGGCTTCAACCTGCTGATGACCCTGCGGCTG
TGGTCCAGC
(SEQ ID NO: 142)
MKSLRVLLVILWLQLSWVWSQQKEVEQNSGPLSVPEGAIASLNCT
YSDRGSQSFFWYRQYSGKSPELIMFIYSNGDKEDGRFTAQLNKAS
QYVSLLIRDSQPSDSATYLCAVKDNAGNMLTEGGGTRLMVKPHIQ
NPDPAVYQLRDSKSSDKSVCLFTDFDSQTNVSQSKDSDVYITDKC
VLDMRSMDFKSNSAVAWSNKSDFACANAFNNSIIPEDTFFPSPES
SCDVKLVEKSFETDINLNFQNLSVIGFRILLLKVAGENLLMTLRL
WSS
(SEQ ID NO: 143)
ATGGGATTCCGGCTTCTTTGTTGTGTGGCATTTTGTCTGTTGGGT
GCGGGTCCAGTCGATAGTGGTGTAACTCAGACACCAAAACACCTT
ATCACGGCAACTGGGCAACGAGTGACGCTCCGCTGTAGCCCGAGG
TCCGGTGATTTGAGTGTGTACTGGTACCAGCAATCTTTGGACCAG
GGCTTGCAGTTCCTCATACAGTATTACAATGGTGAAGAAAGAGCG
AAGGGTAATATCCTGGAAAGATTCTCCGCACAACAGTTTCCTGAT
CTCCACAGCGAACTGAACCTGAGTTCTCTCGAGCTCGGGGATAGT
GCTTTGTACTTCTGCGCGTCATCCGACGGTGGCGGAGTCTATGAA
CAATATTTCGGCCCAGGGACTAGGCTTACGGTGACGGAGGACCTC
AAGAATGTGTTTCCGCCCGAAGTCGCGGTTTTTGAACCATCAGAA
GCCGAGATCTCTCATACACAAAAGGCGACGCTCGTATGCCTTGCG
ACGGGATTTTATCCGGACCACGTCGAGCTTTCCTGGTGGGTTAAT
GGAAAGGAGGTGCATTCCGGAGTTTGCACGGACCCTCAGCCATTG
AAGGAACAGCCCGCACTGAACGACAGTAGGTATTGCCTTTCATCT
CGCCTGCGCGTGTCTGCGACATTCTGGCAAAACCCAAGAAATCAC
TTCAGATGTCAAGTTCAGTTCTACGGTCTCAGCGAGAATGATGAG
TGGACACAAGATAGGGCTAAACCCGTGACTCAAATAGTCTCTGCC
GAGGCCTGGGGGAGGGCGGATTGCGGCTTCACATCAGAATCATAC
CAACAAGGAGTATTGAGCGCGACAATTCTTTACGAAATTCTGCTT
GGGAAAGCGACTCTGTACGCGGTGCTCGTGTCCGCTTTGGTTCTT
ATGGCAATGGTTAAACGAAAGGATAGTAGGGGC
(SEQ ID NO: 144)
MGFRLLCCVAFCLLGAGPVDSGVTQTPKHLITATGQRVTLRCSPR
SGDLSVYWYQQSLDQGLQFLIQYYNGEERAKGNILERFSAQQFPD
LHSELNLSSLELGDSALYFCASSDGGGVYEQYFGPGTRLTVTEDL
KNVFPPEVAVFEPSEAEISHTQKATLVCLATGFYPDHVELSWWVN
GKEVHSGVCTDPQPLKEQPALNDSRYCLSSRLRVSATFWQNPRNH
FRCQVQFYGLSENDEWTQDRAKPVTQIVSAEAWGRADCGFTSESY
QQGVLSATILYEILLGKATLYAVLVSALVLMAMVKRKDSRG

In some embodiments, a TCR construct comprising PRAME-specific TCR chains comprises a nucleotide coding sequence that is at least, or exactly, 85%, 86%, 87%, 88%, 89%, 90%, 91%, 92%, 93%, 94%, 95%, 96%, 97%, 98%, 99%, or 100%, identical to one or more of SEQ ID NOs: 145-152. In some embodiments, a TCR construct comprising PRAME-specific TCR chains comprises TCR alpha and TCR beta chains found in PRAME-specific TCR clone T116-49 and/or T402-93 and/or modified versions thereof. In some embodiments, a TCR construct comprising PRAME-specific TCR chains comprises TCR alpha and TCR beta chains that target PRAME epitope LYVDSLFFL (SEQ ID NO: 167). In some embodiments, PRAME-specific TCR sequences, TCR variable domain sequences, CDR sequences, and/or TCR constant domain sequences, are described in international patent application publication WO 2022/063966 A1, which is incorporated herein by reference for the purpose described herein. In some embodiments, a TCR construct comprising PRAME-specific TCR chains comprises an amino acid sequence that is at least, or exactly, 85%, 86%, 87%, 88%, 89%, 90%, 91%, 92%, 93%, 94%, 95%, 96%, 97%, 98%, 99%, or 100%, identical to one or more of SEQ ID NOs: 153-166.

(SEQ ID NO: 145)
ATGGAGACACTGCTGAAGGTGCTGTCTGGCACACTGCTGTGGCAG
CTGACCTGGGTCCGATCTCAGCAGCCTGTTCAGTCTCCTCAGGCC
GTGATCCTGAGAGAAGGCGAGGACGCCGTGATCAACTGCAGCAGC
TCTAAGGCCCTGTACAGCGTGCACTGGTACAGACAGAAGCACGGC
GAGGCCCCTGTGTTCCTGATGATCCTGCTGAAAGGCGGCGAGCAG
AAGGGCCACGAGAAGATCAGCGCCAGCTTCAACGAGAAGAAGCAG
CAGTCCAGCCTGTACCTGACAGCCAGCCAGCTGAGCTACAGCGGC
ACCTACTTTTGCGGCACAGCCAATAGCGGCGGCAGCAACTACAAG
CTGACCTTCGGCAAGGGCACCCTGCTGACCGTGAATCCCAAT
(SEQ ID NO: 146)
ATGCTGCTGATCACCTCCATGCTGGTGCTGTGGATGCAGCTGAGC
CAAGTGAACGGCCAGCAAGTGATGCAGATCCCTCAGTACCAGCAC
GTGCAAGAAGGCGAGGACTTCACCACCTACTGCAACAGCAGCACC
ACACTGAGCAACATCCAGTGGTACAAGCAGCGGCCTGGCGGACAC
CCTGTGTTTCTGATCCAGCTGGTCAAGTCCGGCGAAGTGAAGAAG
CAGAAGCGGCTGACCTTCCAGTTCGGCGAGGCCAAGAAGAACAGC
AGCCTGCACATCACCGCCACACAGACCACCGATGTGGGCACCTAC
TTTTGTGCTGGCGCCCTGCCTAGAGCCGGCAGCTATCAACTGACA
TTCGGCAAGGGCACCAAGCTGAGCGTGATCCCCAAC
(SEQ ID NO: 147)
ATGGAGACA
CTGCTGAAGGTGCTGTCTGGCACACTGCTGTGGCAGCTGACCTGG
GTCCGATCTCAGCAGCCTGTTCAGTCTCCTCAGGCCGTGATCCTG
AGAGAAGGCGAGGACGCCGTGATCAACTGCAGCAGCTCTAAGGCC
CTGTACAGCGTGCACTGGTACAGACAGAAGCACGGCGAGGCCCCT
GTGTTCCTGATGATCCTGCTGAAAGGCGGCGAGCAGAAGGGCCAC
GAGAAGATCAGCGCCAGCTTCAACGAGAAGAAGCAGCAGTCCAGC
CTGTACCTGACAGCCAGCCAGCTGAGCTACAGCGGCACCTACTTT
TGCGGCACAGCCAATAGCGGCGGCAGCAACTACAAGCTGACCTTC
GGCAAGGGCACCCTGCTGACCGTGAATCCCAATATCCAGAATCCG
GAGCCCGCCGTATACCAGCTGAAGGACCCTAGAAGCCAGGACAGC
ACCCTGTGCCTGTTCACCGACTTCGACAGCCAGATCAACGTGCCC
AAGACCATGGAAAGCGGCACCTTCATCACCGACAAGACAGTGCTG
GACATGAAGGCCATGGACAGCAAGTCCAACGGCGCAATCGCCTGG
TCCAACCAGACCAGCTTCACATGCCAGGACATCTTCAAAGAGACA
AACGCCACATACCCCAGCAGCGACGTGCCCTGTGATGCCACCCTG
ACAGAGAAGTCCTTCGAGACAGACATGAACCTGAACTTCCAGAAT
CTGTCCGTGATGGGCCTGAGAATCCTGCTGCTGAAGGTGGCCGGC
TTCAATCTGCTGATGACCCTGCGGCTGTGGTCCAGC
(SEQ ID NO: 148)
ATGCTGCTGATCACCTCCATGCTGGTGCTGTGGATGCAGCTGAGC
CAAGTGAACGGCCAGCAAGTGATGCAGATCCCTCAGTACCAGCAC
GTGCAAGAAGGCGAGGACTTCACCACCTACTGCAACAGCAGCACC
ACACTGAGCAACATCCAGTGGTACAAGCAGCGGCCTGGCGGACAC
CCTGTGTTTCTGATCCAGCTGGTCAAGTCCGGCGAAGTGAAGAAG
CAGAAGCGGCTGACCTTCCAGTTCGGCGAGGCCAAGAAGAACAGC
AGCCTGCACATCACCGCCACACAGACCACCGATGTGGGCACCTAC
TTTTGTGCTGGCGCCCTGCCTAGAGCCGGCAGCTATCAACTGACA
TTCGGCAAGGGCACCAAGCTGAGCGTGATCCCCAACATCCAGAAT
CCGGAGCCCGCCGTATACCAGCTGAAGGACCCTAGAAGCCAGGAC
AGCACCCTGTGCCTGTTCACCGACTTCGACAGCCAGATCAACGTG
CCCAAGACCATGGAAAGCGGCACCTTCATCACCGACAAGACAGTG
CTGGACATGAAGGCCATGGACAGCAAGTCCAACGGCGCAATCGCC
TGGTCCAACCAGACCAGCTTCACATGCCAGGACATCTTCAAAGAG
ACAAACGCCACATACCCCAGCAGCGACGTGCCCTGTGATGCCACC
CTGACAGAGAAGTCCTTCGAGACAGACATGAACCTGAACTTCCAG
AATCTGTCCGTGATGGGCCTGAGAATCCTGCTGCTGAAGGTGGCC
GGCTTCAATCTGCTGATGACCCTGCGGCTGTGGTCCAGC
(SEQ ID NO: 149)
ATGGGCACCAGACTGTTCTTCTACGTGGCCCTGTGTCTGCTGTGG
ACAGGCCATGTGGATGCCGGAATCACACAGAGCCCCAGACACAAA
GTGACCGAGACAGGCACCCCTGTGACACTGAGATGTCACCAGACC
GAGAACCATCGGTACATGTATTGGTACAGACAGGACCCCGGCCAC
GGCCTGAGACTGATCCACTATAGCTACGGCGTGAAGGACACCGAC
AAGGGCGAAGTGTCTGACGGCTACAGCGTGTCCAGAAGCAAGACC
GAGGACTTCCTGCTGACCCTGGAAAGCGCCACAAGCAGCCAGACC
AGCGTGTACTTCTGCGCCATCAGCGACTACGAGGGCACCGAGGCC
TTTTTTGGCCAAGGCACAAGACTGACCGTGGTG
(SEQ ID NO: 150)
ATGCTGTGTTCTCTGCTGGCTCTGCTGCTGGGCACCTTTTTTGGC
GTCAGAAGCCAGACCATCCACCAGTGGCCTGCTACACTGGTGCAG
CCTGTTGGAAGCCCTCTGAGCCTGGAATGTACCGTGGAAGGCACC
AGCAATCCCAACCTGTACTGGTACAGACAGGCCGCTGGAAGAGGA
CTGCAGCTGCTGTTTTACAGCGTCGGCATCGGCCAGATCAGCAGC
GAGGTTCCACAGAATCTGAGCGCCAGCAGACCCCAGGACAGACAG
TTTATCCTGAGCAGCAAGAAGCTGCTGCTGAGCGACAGCGGCTTC
TACCTGTGTGCTTGGAGCCTCGGAGCCGGCTACACCGACACACAG
TATTTTGGCCCTGGCACCAGACTGACCGTGCTG
(SEQ ID NO: 151)
ATGGGCACCAGACTGTTCTTCTACGTGGCCCTGTGTCTGCTGTGG
ACAGGCCATGTGGATGCCGGAATCACACAGAGCCCCAGACACAAA
GTGACCGAGACAGGCACCCCTGTGACACTGAGATGTCACCAGACC
GAGAACCATCGGTACATGTATTGGTACAGACAGGACCCCGGCCAC
GGCCTGAGACTGATCCACTATAGCTACGGCGTGAAGGACACCGAC
AAGGGCGAAGTGTCTGACGGCTACAGCGTGTCCAGAAGCAAGACC
GAGGACTTCCTGCTGACCCTGGAAAGCGCCACAAGCAGCCAGACC
AGCGTGTACTTCTGCGCCATCAGCGACTACGAGGGCACCGAGGCC
TTTTTTGGCCAAGGCACAAGACTGACCGTGGTGGAAGATCTCCGG
AACGTGACCCCCCCTAAAGTGACCCTGTTCGAACCCAGCAAGGCC
GAGATCGCCAACAAGCAGAAAGCCACCCTCGTGTGCCTGGCCAGA
GGCTTCTTCCCCGACCATGTGGAACTGTCTTGGTGGGTCAACGGC
AAAGAGGTGCACAGCGGAGTGTCCACCGACCCTCAGGCCTACAAA
GAGAGCAACTACAGCTACTGCCTGAGCAGCAGACTGCGGGTGTCC
GCCACCTTCTGGCACAACCCCCGGAACCACTTCAGATGCCAGGTG
CAGTTTCACGGCCTGAGCGAAGAGGACAAGTGGCCCGAAGGCTCC
CCCAAGCCCGTGACCCAGAATATCTCTGCCGAGGCCTGGGGCAGA
GCCGACTGTGGAATTACCAGCGCCAGCTACCACCAGGGCGTGCTG
TCTGCCACCATCCTGTACGAGATCCTGCTGGGCAAGGCCACCCTG
TACGCCGTGCTGGTGTCTGGCCTGGTGCTGATGGCCATGGTCAAG
AAGAAGAACAGC
(SEQ ID NO: 152])
ATGCTGTGTTCTCTGCTGGCTCTGCTGCTGGGCACCTTTTTTGGC
GTCAGAAGCCAGACCATCCACCAGTGGCCTGCTACACTGGTGCAG
CCTGTTGGAAGCCCTCTGAGCCTGGAATGTACCGTGGAAGGCACC
AGCAATCCCAACCTGTACTGGTACAGACAGGCCGCTGGAAGAGGA
CTGCAGCTGCTGTTTTACAGCGTCGGCATCGGCCAGATCAGCAGC
GAGGTTCCACAGAATCTGAGCGCCAGCAGACCCCAGGACAGACAG
TTTATCCTGAGCAGCAAGAAGCTGCTGCTGAGCGACAGCGGCTTC
TACCTGTGTGCTTGGAGCCTCGGAGCCGGCTACACCGACACACAG
TATTTTGGCCCTGGCACCAGACTGACCGTGCTGGAAGATCTCCGG
AACGTGACCCCCCCTAAAGTGACCCTGTTCGAACCCAGCAAGGCC
GAGATCGCCAACAAGCAGAAAGCCACCCTCGTGTGCCTGGCCAGA
GGCTTCTTCCCCGACCATGTGGAACTGTCTTGGTGGGTCAACGGC
AAAGAGGTGCACAGCGGAGTGTCCACCGACCCTCAGGCCTACAAA
GAGAGCAACTACAGCTACTGCCTGAGCAGCAGACTGCGGGTGTCC
GCCACCTTCTGGCACAACCCCCGGAACCACTTCAGATGCCAGGTG
CAGTTTCACGGCCTGAGCGAAGAGGACAAGTGGCCCGAAGGCTCC
CCCAAGCCCGTGACCCAGAATATCTCTGCCGAGGCCTGGGGCAGA
GCCGACTGTGGAATTACCAGCGCCAGCTACCACCAGGGCGTGCTG
TCTGCCACCATCCTGTACGAGATCCTGCTGGGCAAGGCCACCCTG
TACGCCGTGCTGGTGTCTGGCCTGGTGCTGATGGCCATGGTCAAG
AAGAAGAACAGC
(SEQ ID NO: 153)
METLLKVLSGTLLWQLTWVRSQQPVQSPQAVILREGEDAVINCSS
SKALYSVHWYRQKHGEAPVFLMILLKGGEQKGHEKISASFNEKKQ
QSSLYLTASQLSYSGTYFCGTANSGGSNYKLTFGKGTLLTVNPN
(SEQ ID NO: 154)
MLLITSMLVLWMQLSQVNGQQVMQIPQYQHVQEGEDETTYCNSST
TLSNIQWYKQRPGGHPVFLIQLVKSGEVKKQKRLTFQFGEAKKNS
SLHITATQTTDVGTYFCAGALPRAGSYQLTFGKGTKLSVIPN
(SEQ ID NO: 155)
IQNPEPAVYQLKDPRSQDSTLCLFTDFDSQINVPKTMESGTFITD
KTVLDMKAMDSKSNGAIAWSNQTSFTCQDIFKETNATYPSSDVPC
DATLTEKSFETDMNLNFQNLSVMGLRILLLKVAGFNLLMTLRLWS
S
(SEQ ID NO: 156)
IQNPDPAVYQLRDSKSSDKSVCLFTDFDSQTNVSQSKDSDVYITD
KTVLDMRSMDFKSNSAVAWSNKSDFACANAFNNSIIPEDTFFPSP
ESSCDVKLVEKSFETDTNLNFQNLSVIGFRILLLKVAGFNLLMTL
RLWSS
(SEQ ID NO: 157)
IQNPDPAVYQLRDSKSSDKSVCLFTDFDSQTNVSQSKDSDVYITD
KTVLDMRSMDFKSNSAVAWSNKSDFACANAENNSIIPEDTFFPSS
DVPCDVKLVEKSFETDTNLNFQNLSVIGFRILLLKVAGFNLLMTL
RLWSS
(SEQ ID NO: 158)
METLLKVLSGTLLWQLTWVRSQQPVQSPQAVILREGEDAVINCSS
SKALYSVHWYRQKHGEAPVFLMILLKGGEQKGHEKISASFNEKKQ
QSSLYLTASQLSYSGTYFCGTANSGGSNYKLTFGKGTLLTVNPNI
QNPEPAVYQLKDPRSQDSTLCLFTDFDSQINVPKTMESGTFITDK
TVLDMKAMDSKSNGAIAWSNQTSFTCQDIFKETNATYPSSDVPCD
ATLTEKSFETDMNLNFQNLSVMGLRILLLKVAGFNLLMTLRLWSS
(SEQ ID NO: 159)
MLLITSMLVLWMQLSQVNGQQVMQIPQYQHVQEGEDFTTYCNSST
TLSNIQWYKQRPGGHPVFLIQLVKSGEVKKQKRLTFQFGEAKKNS
SLHITATQTTDVGTYFCAGALPRAGSYQLTFGKGTKLSVIPNIQN
PEPAVYQLKDPRSQDSTLCLFTDFDSQINVPKTMESGTFITDKTV
LDMKAMDSKSNGAIAWSNQTSFTCQDIFKETNATYPSSDVPCDAT
LTEKSFETDMNLNFQNLSVMGLRILLLKVAGENLLMTLRLWSS
(SEQ ID NO: 160)
MGTRLFFYVALCLLWTGHVDAGITQSPRHKVTETGTPVTLRCHQT
ENHRYMYWYRQDPGHGLRLIHYSYGVKDTDKGEVSDGYSVSRSKT
EDFLLTLESATSSQTSVYFCAISDYEGTEAFFGQGTRLTVV
(SEQ ID NO: 161)
MLCSLLALLLGTFFGVRSQTIHQWPATLVQPVGSPLSLECTVEGT
SNPNLYWYRQAAGRGLQLLFYSVGIGQISSEVPQNLSASRPQDRQ
FILSSKKLLLSDSGFYLCAWSLGAGYTDTQYFGPGTRLTVL
(SEQ ID NO: 162)
EDLRNVTPPKVTLFEPSKAEIANKQKATLVCLARGFFPDHVELSW
WVNGKEVHSGVSTDPQAYKESNYSYCLSSRLRVSATFWHNPRNHF
RCQVQFHGLSEEDKWPEGSPKPVTQNISAEAWGRADCGITSASYH
QGVLSATILYEILLGKATLYAVLVSGLVLMAMVKKKNS
(SEQ ID NO: 163)
DLNKVFPPEVAVFEPSEAEISHTQKATLVCLATGFFPDHVELSWW
VNGKEVHSGVSTDPQPLKEQPALNDSRYCLSSRLRVSATFWQNPR
NHFRCQVQFYGLSENDEWTQDRAKPVTQIVSAEAWGRADCGFTSV
SYQQGVLSATILYEILLGKATLYAVLVSALVLMAMVKRKDF
(SEQ ID NO: 164)
EDLNKVFPPEVAVFEPSKAEIAHTQKATLVCLATGFFPDHVELSW
WVNGKEVHSGVSTDPQPLKEQPALNDSRYCLSSRLRVSATFWQNP
RNHERCQVQFYGLSENDEWTQDRAKPVTQIVSAEAWGRADCGITS
ASYHQGVLSATILYEILLGKATLYAVLVSALVLMAMVKRKDF
(SEQ ID NO: 165)
MGTRLFFYVALCLLWTGHVDAGITQSPRHKVTETGTPVTLRCHQT
ENHRYMYWYRQDPGHGLRLIHYSYGVKDTDKGEVSDGYSVSRSKT
EDFLLTLESATSSQTSVYFCAISDYEGTEAFFGQGTRLTVVEDLR
NVTPPKVTLFEPSKAEIANKQKATLVCLARGFFPDHVELSWWVNG
KEVHSGVSTDPQAYKESNYSYCLSSRLRVSATFWHNPRNHFRCQV
QFHGLSEEDKWPEGSPKPVTQNISAEAWGRADCGITSASYHQGVL
SATILYEILLGKATLYAVLVSGLVLMAMVKKKNS
(SEQ ID NO: 166)
MLCSLLALLLGTFFGVRSQTIHQWPATLVQPVGSPLSLECTVEGT
SNPNLYWYRQAAGRGLQLLFYSVGIGQISSEVPQNLSASRPQDRQ
FILSSKKLLLSDSGFYLCAWSLGAGYTDTQYFGPGTRLTVLEDLR
NVTPPKVTLFEPSKAEIANKQKATLVCLARGFFPDHVELSWWVNG
KEVHSGVSTDPQAYKESNYSYCLSSRLRVSATFWHNPRNHFRCQV
QFHGLSEEDKWPEGSPKPVTQNISAEAWGRADCGITSASYHQGVL
SATILYEILLGKATLYAVLVSGLVLMAMVKKKNS

In some embodiments, a TCR construct comprises gp100-specific TCR chains. In some embodiments, a TCR construct comprising gp100-specific TCR chains comprises TCR alpha and TCR beta chains found in gp100-specific TCR clone Sp(0.01)A and/or modified versions thereof. In some embodiments, a TCR construct comprising gp100-specific TCR chains comprises TCR alpha and TCR beta chains that target gp100 epitope KTWGQYWQV (SEQ ID NO: 168). In some embodiments, gp100-specific TCR sequences, TCR variable domain sequences, CDR sequences, and/or TCR constant domain sequences, are described in patent publication U.S. Pat. No. 8,216,565 B2, which is incorporated herein by reference for the purpose described herein.

In some embodiments, a TCR construct comprising gp100-specific TCR chains comprises a nucleotide coding sequence that is at least, or exactly, 85%, 86%, 87%, 88%, 89%, 90%, 91%, 92%, 93%, 94%, 95%, 96%, 97%, 98%, 99%, or 100%, identical to one or more of SEQ ID NOs: 169 and/or 170. In some embodiments, a TCR construct comprising gp100-specific TCR chains comprises an amino acid sequence that is at least, or exactly, 85%, 86%, 87%, 88%, 89%, 90%, 91%, 92%, 93%, 94%, 95%, 96%, 97%, 98%, 99%, or 100%, identical to one or more of SEQ ID NOs: 171-174.

(SEQ ID NO: 169)
ATGAAATCCTTGAGTGTTTCCCTAGTGGTCCTGTGGCTCCAGTTA
AACTGGGTGAACAGCCAGCAGAAGGTGCAGCAGAGCCCAGAATCC
CTCATTGTCCCAGAGGGAGCCATGACCTCTCTCAACTGCACTTTC
AGCGACAGTGCTTCTCAGTATTTTGCATGGTACAGACAGCATTCT
GGGAAAGCCCCCAAGGCACTGATGTCCATCTTCTCCAATGGTGAA
AAAGAAGAAGGCAGATTCACAATTCACCTCAATAAAGCCAGTCTG
CATTTCTCGCTACACATCAGAGACTCCCAGCCCAGTGACTCTGCT
CTCTACCTCTGTGCAGCCAATAACTATGCCCAGGGATTAACCTTC
GGTCTTGGCACCAGAGTATCTGTGTTTCCCTACATCCAGAACCCA
GAACCTGCTGTGTACCAGTTAAAAGATCCTCGGTCTCAGGACAGC
ACCCTCTGCCTGTTCACCGACTTTGACTCCCAAATCAATGTGCCG
AAAACCATGGAATCTGGAACGTTCATCACTGACAAAACTGTGCTG
GACATGAAAGCTATGGATTCCAAGAGCAATGGGGCCATTGCCTGG
AGCAACCAGACAAGCTTCACCTGCCAAGATATCTTCAAAGAGACC
AACGCCACCTACCCCAGTTCAGACGTTCCCTGTGATGCCACGTTG
ACTGAGAAAAGCTTTGAAACAGATATGAACCTAAACTTTCAAAAC
CTGTCAGTTATGGGACTCCGAATCCTCCTGCTGAAAGTAGCCGGA
TTTAACCTGCTCATGACGCTGAGGCTGTGGTCCAGTTGA
(SEQ ID NO: 170)
ATGGGCTCCAGACTCTTCTTTGTGGTTTTGATTCTCCTGTGTGCA
AAACACATGGAGGCTGCAGTCACCCAAAGTCCAAGAAGCAAGGTG
GCAGTAACAGGAGGAAAGGTGACATTGAGCTGTCACCAGACTAAT
AACCATGACTATATGTACTGGTATCGGCAGGACACGGGGCATGGG
CTGAGGCTGATCCATTACTCATATGTCGCTGACAGCACGGAGAAA
GGAGATATCCCTGATGGGTACAAGGCCTCCAGACCAAGCCAAGAG
AATTTCTCTCTCATTCTGGAGTTGGCTTCCCTTTCTCAGACAGCT
GTATATTTCTGTGCCAGCAGCCCTGGGGGGGGGGGGGAACAGTAC
TTCGGTCCCGGCACCAGGCTCACGGTTTTAGAGGATCTGAGAAAT
GTGACTCCACCCAAGGTCTCCTTGTTTGAGCCATCAAAAGCAGAG
ATTGCAAACAAACGAAAGGCTACCCTCGTGTGCTTGGCCAGGGGC
TTCTTCCCTGACCACGTGGAGCTGAGCTGGTGGGTGAATGGCAAG
GAGGTCCACAGTGGGGTCAGCACGGACCCTCAGGCCTACAAGGAG
AGCAATTATAGCTACTGCCTGAGCAGCCGCCTGAGGGTCTCTGCT
ACCTTCTGGCACAATCCTCGAAACCACTTCCGCTGCCAAGTGCAG
TTCCATGGGCTTTCAGAGGAGGACAAGTGGCCAGAGGGCTCACCC
AAACCTGTCACACAGAACATCAGTGCAGAGGCCTGGGGCCGAGCA
GACTGTGGGATTACCTCAGCATCCTATCAACAAGGGGTCTTGTCT
GCCACCATCCTCTATGAGATCCTGCTAGGGAAAGCCACCCTGTAT
GCTGTGCTTGTCAGTACACTGGTGGTGATGGCTATGGTCAAAAGA
AAGAATTCATGA
(SEQ ID NO: 171)
MKSLSVSLVVLWLQLNWVNSQQKVQQSPESLIV
PEGAMTSLNCTFSDSASQYFAWYRQHSGKAPKALMSIFSNGEKEE
GRFTIHLNKASLHESLHIRDSQPSDSALYLCAANNYAQGLTFGLG
TRVSVFPYIQNPEPAVYQLKDPRSQDSTLCLFTDFDSQINVPKTM
ESGTFITDKTVLDMKAMDSKSNGAIAWSNQTSFTCQDIFKETNAT
YPSSDVPCDATLTEKSFETDMNLNFQNLSVMGLRILLLKVAGFNL
LMTLRLWSS
(SEQ ID NO: 172)
MGSRLFFVVLILLCAKHMEAAVTQSPRSKVAVTGGKVTLSCHQTN
NHDYMYWYRQDTGHGLRLIHYSYVADSTEKGDIPDGYKASRPSQE
NFSLILELASLSQTAVYFCASSPGGGGEQYFGPGTRLTVLEDLRN
VTPPKVSLFEPSKAEIANKRKATLVCLARGFFPDHVELSWWVNGK
EVHSGVSTDPQAYKESNYSYCLSSRLRVSATFWHNPRNHERCQVQ
FHGLSEEDKWPEGSPKPVTQNISAEAWGRADCGITSASYQQGVLS
ATILYEILLGKATLYAVLVSTLVVMAMVKRKNS
(SEQ ID NO: 173)
QQKVQQSPESLIVPEGAMTSLNCTFSDSASQYFAWYRQHSGKAPK
ALMSIFSNGEKEEGRFTIHLNKASLHESLHIRDSQPSDSALYLCA
ANNYAQGLTFGLGTRVSVFPY
(SEQ ID NO: 174)
EAAVTQSPRSKVAVTGGKVTLSCHQTNNHDYMYWYRQDTGHGLRL
IHYSYVADSTEKGDIPDGYKASRPSQENFSLILELASLSQTAVYF
CASSPGGGGEQYFGPGTRLTVL

In some embodiments, a TCR construct comprises MART-1-specific TCR chains. In some embodiments, a TCR construct comprising MART-1-specific TCR chains comprises TCR alpha and TCR beta chains found in MART-1-specific TCR clones F4 and/or F5 and/or modified versions thereof. In some embodiments, a TCR construct comprising MART-1-specific TCR chains comprises TCR alpha and TCR beta chains that target MART-1 epitope AAGIGILTV (SEQ ID NO: 175). In some embodiments, MART-1-specific TCR sequences, TCR variable domain sequences, CDR sequences, and/or TCR constant domain sequences, are described in patent publication U.S. Pat. No. 9,128,080 B2, which is incorporated herein by reference for the purpose described herein.

In some embodiments, a TCR construct comprising MART-1-specific TCR chains comprises a nucleotide coding sequence that is at least, or exactly, 85%, 86%, 87%, 88%, 89%, 90%, 91%, 92%, 93%, 94%, 95%, 96%, 97%, 98%, 99%, or 100%, identical to one or more of SEQ ID NOs: 176-179. In some embodiments, a TCR construct comprising MART-1-specific TCR chains comprises an amino acid sequence that is at least, or exactly, 85%, 86%, 87%, 88%, 89%, 90%, 91%, 92%, 93%, 94%, 95%, 96%, 97%, 98%, 99%, or 100%, identical to one or more of SEQ ID NOs: 180-183.

(SEQ ID NO: 176)
ATGTTGCTTGAACATTTATTAATAATCTTGTGGATGCAGCTGACA
TGGGTCAGTGGTCAACAGCTGAATCAGAGTCCTCAATCTATGTTT
ATCCAGGAAGGAGAAGATGTCTCCATGAACTGCACTTCTTCAAGC
ATATTTAACACCTGGCTATGGTACAAGCAGGACCCTGGGGAAGGT
CCTGTCCTCTTGATAGCCTTATATAAGGCTGGTGAATTGACCTCA
AATGGAAGACTGACTGCTCAGTTTGGTATAACCAGAAAGGACAGC
TTCCTGAATATCTCAGCATCCATACCTAGTGATGTAGGCATCTAC
TTCTGTGCTGGTGGGACCGGTAACCAGTTCTATTTTGGGACAGGG
ACAAGTTTGACGGTCATTCCAAATATCCAGAACCCTGACCCTGCC
GTGTACCAGCTGAGAGACTCTAAATCCAGTGACAAGTCTGTCTGC
CTATTCACCGATTTTGATTCTCAAACAAATGTGTCACAAAGTAAG
GATTCTGATGTGTATATCACAGACAAAACTGTGCTAGACATGAGG
TCTATGGACTTCAAGAGCAACAGTGCTGTGGCCTGGAGCAACAAA
TCTGACTTTGCATGTGCAAACGCCTTCAACAACAGCATTATTCCA
GAAGACACCTTCTTCCCCAGCCCAGAAAGTTCCTGTGATGTCAAG
CTGGTCGAGAAAAGCTTTGAAACAGATACGAACCTAAACTTTCAA
AACCTGTCAGTGATTGGGTTCCGAATCCTCCTCCTGAAGGTGGCC
GGGTTTAATCTGCTCATGACGCTGCGGCTGTGGTCCAGC
(SEQ ID NO: 177)
ATGGGCACAAGGTTGTTCTTCTATGTGGCCCTTTGTCTCCTGTGG
ACAGGACACATGGATGCTGGAATCACCCAGAGCCCAAGACACAAG
GTCACAGAGACAGGAACACCAGTGACTCTGAGATGTCACCAGACT
GAGAACCACCGCTATATGTACTGGTATCGACAAGACCCGGGGCAT
GGGCTGAGGCTGATCCATTACTCATATGGTGTTAAAGATACTGAC
AAAGGAGAAGTCTCAGATGGCTATAGTGTCTCTAGATCAAAGACA
GAGGATTTCCTCCTCACTCTGGAGTCCGCTACCAGCTCCCAGACA
TCTGTGTACTTCTGTGCCATCAGTGAGGTAGGGGTTGGGCAGCCC
CAGCATTTTGGTGATGGGACTCGACTCTCCATCCTAGAGGACCTG
AACAAGGTGTTCCCACCCGAGGTCGCTGTGTTTGAGCCATCAGAA
GCAGAGATCTCCCACACCCAAAAGGCCACACTGGTGTGCCTGGCC
ACAGGCTTCTTCCCCGACCACGTGGAGCTGAGCTGGTGGGTGAAT
GGGAAGGAGGTGCACAGTGGGGTCAGCACGGACCCGCAGCCCCTC
AAGGAGCAGCCCGCCCTCAATGACTCCAGATACTGCCTGAGCAGC
CGCCTGAGGGTCTCGGCCACCTTCTGGCAGAACCCCCGCAACCAC
TTCCGCTGTCAAGTCCAGTTCTACGGGCTCTCGGAGAATGACGAG
TGGACCCAGGATAGGGCCAAACCCGTCACCCAGATCGTCAGCGCC
GAGGCCTGGGGTAGAGCATGTGGCTTTACCTCGTCCTACCAGCAA
GGGGTCCTGTCTGCCACCATCCTCTATGAGATCCTGCTAGGGAAG
GCCACCCTGTATGCTGTGCTGGTCAGCGCCCTTGTGTTGATGGCC
ATGGTCAAGAGAAAGGATTTC
(SEQ ID NO: 178)
ATGATGAAATCCTTGAGAGTTTTACTAGTGATCCTGTGGCTTCAG
TTGAGCTGGGTTTGGAGCCAACAGAAGGAGGTGGAGCAGAATTCT
GGACCCCTCAGTGTTCCAGAGGGAGCCATTGCCTCTCTCAACTGC
ACTTACAGTGACCGAGGTTCCCAGTCCTTCTTCTGGTACAGACAA
TATTCTGGGAAAAGCCCTGAGTTGATAATGTTCATATACTCCAAT
GGTGACAAAGAAGATGGAAGGTTTACAGCACAGCTCAATAAAGCC
AGCCAGTATGTTTCTCTGCTCATCAGAGACTCCCAGCCCAGTGAT
TCAGCCACCTACCTCTGTGCCGTGAACTTCGGAGGAGGAAAGCTT
ATCTTCGGACAGGGAACGGAGTTATCTGTGAAACCCAATATCCAG
AACCCTGACCCTGCCGTGTACCAGCTGAGAGACTCTAAATCCAGT
GACAAGTCTGTCTGCCTATTCACCGATTTTGATTCTCAAACAAAT
GTGTCACAAAGTAAGGATTCTGATGTGTATATCACAGACAAAACT
GTGCTAGACATGAGGTCTATGGACTTCAAGAGCAACAGTGCTGTG
GCCTGGAGCAACAAATCTGACTTTGCATGTGCAAACGCCTTCAAC
AACAGCATTATTCCAGAAGACACCTTCTTCCCCAGCCCAGAAAGT
TCCTGTGATGTCAAGCTGGTCGAGAAAAGCTTTGAAACAGATACG
AACCTAAACTTTCAAAACCTGTCAGTGATTGGGTTCCGAATCCTC
CTCCTGAAAGTGGCCGGGTTTAATCTGCTCATGACGCTGCGGCTG
TGGTCCAGCTGA
(SEQ ID NO: 179)
ATGAGAATCAGGCTCCTGTGCTGTGTGGCCTTTTCTCTCCTGTGG
GCAGGTCCAGTGATTGCTGGGATCACCCAGGCACCAACATCTCAG
ATCCTGGCAGCAGGACGGCGCATGACACTGAGATGTACCCAGGAT
ATGAGACATAATGCCATGTACTGGTATAGACAAGATCTAGGACTG
GGGCTAAGGCTCATCCATTATTCAAATACTGCAGGTACCACTGGC
AAAGGAGAAGTCCCTGATGGTTATAGTGTCTCCAGAGCAAACACA
GATGATTTCCCCCTCACGTTGGCGTCTGCTGTACCCTCTCAGACA
TCTGTGTACTTCTGTGCCAGCAGCCTAAGTTTCGGCACTGAAGCT
TTCTTTGGACAAGGCACCAGACTCACAGTTGTAGAGGACCTGAAC
AAGGTGTTCCCACCCGAGGTCGCTGTGTTTGAGCCATCAGAAGCA
GAGATCTCCCACACCCAAAAGGCCACACTGGTGTGCCTGGCCACA
GGCTTCTTCCCCGACCACGTGGAGCTGAGCTGGTGGGTGAATGGG
AAGGAGGTGCACAGTGGGGTCAGCACGGACCCGCAGCCCCTCAAG
GAGCAGCCCGCCCTCAATGACTCCAGATACTGCCTGAGCAGCCGC
CTGAGGGTCTCGGCCACCTTCTGGCAGAACCCCCGCAACCACTTC
CGCTGTCAAGTCCAGTTCTACGGGCTCTCGGAGAATGACGAGTGG
ACCCAGGATAGGGCCAAACCCGTCACCCAGATCGTCAGCGCCGAG
GCCTGGGGTAGAGCATGTGGCTTTACCTCGTCCTACCAGCAAGGG
GTCCTGTCTGCCACCATCCTCTATGAGATCCTGCTAGGGAAGGCC
ACCCTGTATGCTGTGCTGGTCAGCGCCCTTGTGTTGATGGCCATG
GTCAAGAGAAAGGATTTC
(SEQ ID NO: 180)
GQQLNQSPQSMFIQEGEDVSMNCTSSSIFNTWLWYKQDPGEGPVL
LIALYKAGELTSNGRLTAQFGITRKDSFLNISASIPSDVGIYFCA
GGTGNQFYFGTGTSLTVIPNIQNPDPAVYQLRDSKSSDKSVCLFT
DFDSQTNVSQSKDSDVYITDKTVLDMRSMDFKSNSAVAWSNKSDF
ACANAFNNSIIPEDTFFPSPESSCDVKLVEKSFETDTNLNFQNLS
VIGFRILLLKVAGENLLMTLRLWSS
(SEQ ID NO: 181)
DAGITQSPRHKVTETGTPVTLRCHQTENHRYMYWYRQDPGHGLRL
IHYSYGVKDTDKGEVSDGYSVSRSKTEDFLLTLESATSSQTSVYF
CAISEVGVGQPQHFGDGTRLSILEDLNKVFPPEVAVFEPSEAEIS
HTQKATLVCLATGFFPDHVELSWWVNGKEVHSGVSTDPQPLKEQP
ALNDSRYCLSSRLRVSATFWQNPRNHFRCQVQFYGLSENDEWTQD
RAKPVTQIVSAEAWGRACGFTSSYQQGVLSATILYEILLGKATLY
AVLVSALVLMAMVKRKDF
(SEQ ID NO: 182)
QKEVEQNSGPLSVPEGAIASLNCTYSDRGSQSFFWYRQYSGKSPE
LIMFIYSNGDKEDGRFTAQLNKASQYVSLLIRDSQPSDSATYLCA
VNFGGGKLIFGQGTELSVKPNIQNPDPAVYQLRDSKSSDKSVCLF
TDFDSQTNVSQSKDSDVYITDKTVLDMRSMDFKSNSAVAWSNKSD
FACANAFNNSIIPEDTFFPSPESSCDVKLVEKSFETDINLNFQNL
SVIGFRILLLKVAGENLLMTLRLWSS
(SEQ ID NO: 183)
IAGITQAPTSQILAAGRRMTLRCTQDMRHNAMYWYRQDLGLGLRL
IHYSNTAGTTGKGEVPDGYSVSRANTDDFPLTLASAVPSQTSVYF
CASSLSFGTEAFFGQGTRLTVVEDLNKVEPPEVAVFEPSEAEISH
TQKATLVCLATGFFPDHVELSWWVNGKEVHSGVSTDPQPLKEQPA
LNDSRYCLSSRLRVSATFWQNPRNHFRCQVQFYGLSENDEWTQDR
AKPVTQIVSAEAWGRACGFTSSYQQGVLSATILYEILLGKATLYA
VLVSALVLMAMVKRKDF

In some embodiments, a TCR construct comprises Tyrosinase-specific TCR chains. In some embodiments, a TCR construct comprising Tyrosinase-specific TCR chains comprises TCR alpha and TCR beta chains found in Tyrosinase-specific TCR clone TIL 1383I and/or modified versions thereof. In some embodiments, a TCR construct comprising Tyrosinase-specific TCR chains comprises TCR alpha and TCR beta chains that target Tyrosinase epitope represented by amino acids 368-376 of tyrosinase (reactive against a class I MHC (HLA-A2)-restricted epitope (368-376) of tyrosinase). In some embodiments, Tyrosinase-specific TCR sequences, TCR variable domain sequences, CDR sequences, and/or TCR constant domain sequences, are described in publication Roszkowski et al, Cancer Res. 65(4): 1570-6 (2005), which is incorporated herein by reference for the purpose described herein.

In some embodiments, a TCR construct comprises MAGE-A3-specific TCR chains. In some embodiments, a TCR construct comprising MAGE-A3-specific TCR chains comprises TCR alpha and TCR beta chains that target amino acids 271-279 of MAGE-A3, e.g., the epitope FLWGPRALV (SEQ ID NO: 184). In some embodiments, a TCR construct comprising MAGE-A3-specific TCR chains comprises TCR alpha and TCR beta chains that target amino acids 112-120 of MAGE-A3, e.g., the epitope KVAELVHFL (SEQ ID NO: 185). In some embodiments, MAGE-A3-specific TCR sequences, TCR variable domain sequences, CDR sequences, and/or TCR constant domain sequences, are described in international patent application publication WO 2012/054825 A1, which is incorporated herein by reference for the purpose described herein. In certain embodiments, an anti-MAGE-A3 112-120 TCR comprise an A118T substitution relative to wild type (wherein the 118 position in the alpha chain is threonine). In certain embodiments, an anti-MAGE-A3 112-120 TCR comprises an A118V substitution relative to wild type (wherein the 118 position in the alpha chain is valine).

In some embodiments, a TCR construct comprising MAGE-A3-specific TCR chains comprises a nucleotide coding sequence that is at least, or exactly, 85%, 86%, 87%, 88%, 89%, 90%, 91%, 92%, 93%, 94%, 95%, 96%, 97%, 98%, 99%, or 100%, identical to one or more of SEQ ID NOs: 186-193. In some embodiments, a TCR construct comprising MAGE-A3-specific TCR chains comprises an amino acid sequence that is at least, or exactly, 85%, 86%, 87%, 88%, 89%, 90%, 91%, 92%, 93%, 94%, 95%, 96%, 97%, 98%, 99%, or 100%, identical to one or more of SEQ ID NOs: 194-201.

(SEQ ID NO: 186)
ATGGGTCCTGTCACCTGCTCAGTTCTTGTGCTCCTCCTAATGCTC
AGGAGGAGCAATGGCGATGGAGACTCCGTGACCCAGACAGAAGGC
CTGGTCACTCTCACAGAAGGGTTGCCTGTGATGCTGAACTGCACC
TATCAGACTATTTACTCAAATCCTTTCCTTTTCTGGTATGTGCAA
CATCTCAATGAATCCCCTCGGCTACTCCTGAAGAGCTTCACAGAC
AACAAGAGGACCGAGCACCAAGGGTTCCACGCCACTCTCCATAAG
AGCAGCAGCTCCTTCCATCTGCAGAAGTCCTCAGCGCAGCTGTCA
GACTCTGCCCTGTACTACTGTGCTTTCGACACAAATGCTTACAAA
GTCATCTTT
(SEQ ID NO: 187)
ATGAGAGTTAGGCTCATCTCTGCTGTGGTGCTGTGTTCCCTAGGA
ACAGGCCTTGTGGACATGAAAGTAACCCAGATGCCAAGATACCTG
ATCAAAAGAATGGGAGAGAATGTTTTGCTGGAATGTGGACAGGAC
ATGAGCCATGAAACAATGTACTGGTATCGACAAGACCCTGGTCTG
GGGCTACAGCTGATTTATATCTCATACGATGTTGATAGTAACAGC
GAAGGAGACATCCCTAAAGGATACAGGGTCTCACGGAAGAAGCGG
GAGCATTTCTCCCTGATTCTGGATTCTGCTAAAACAAACCAGACA
TCTGTGTACTTCTGTGCTAGCAGTTCAACAAACACAGAAGTCTTC
TTT
(SEQ ID NO: 188)
ATGGGTCCTGTCACCTGCTCAGTTCTTGTGCTCCTCCTAATGCTC
AGGAGGAGCAATGGCGATGGAGACTCCGTGACCCAGACAGAAGGC
CTGGTCACTCTCACAGAAGGGTTGCCTGTGATGCTGAACTGCACC
TATCAGACTATTTACTCAAATCCTTTCCTTTTCTGGTATGTGCAA
CATCTCAATGAATCCCCTCGGCTACTCCTGAAGAGCTTCACAGAC
AACAAGAGGACCGAGCACCAAGGGTTCCACGCCACTCTCCATAAG
AGCAGCAGCTCCTTCCATCTGCAGAAGTCCTCAGCGCAGCTGTCA
GACTCTGCCCTGTACTACTGTGCTTTCGACACAAATGCTTACAAA
GTCATCTTTGGAAAAGGGACACATCTTCATGTTCTCCCTAACATC
CAGAACCCAGAACCTGCTGTGTACCAGTTAAAAGATCCTCGGTCT
CAGGACAGCACCCTCTGCCTGTTCACCGACTTTGACTCCCAAATC
AATGTGCCGAAAACCATGGAATCTGGAACGTTCATCACTGACAAA
ACTGTGCTGGACATGAAAGCTATGGATTCCAAGAGCAATGGGGCC
ATTGCCTGGAGCAACCAGACAAGCTTCACCTGCCAAGATATCTTC
AAAGAGACCAACACCACCTACCCCAGTTCAGACGTTCCCTGTGAT
GCCACGTTGACTGAGAAAAGCTTTGAAACAGATATGAACCTAAAC
TTTCAAAACCTGTCAGTTATGGGACTCCGAATCCTCCTGCTGAAA
GTAGCCGGATTTAACCTGCTCATGACGCTGAGGCTGTGGTCCAGT
TGA
(SEQ ID NO: 189)
ATGAGAGTTAGGCTCATCTCTGCTGTGGTGCTGTGTTCCCTAGGA
ACAGGCCTTGTGGACATGAAAGTAACCCAGATGCCAAGATACCTG
ATCAAAAGAATGGGAGAGAATGTTTTGCTGGAATGTGGACAGGAC
ATGAGCCATGAAACAATGTACTGGTATCGACAAGACCCTGGTCTG
GGGCTACAGCTGATTTATATCTCATACGATGTTGATAGTAACAGC
GAAGGAGACATCCCTAAAGGATACAGGGTCTCACGGAAGAAGCGG
GAGCATTTCTCCCTGATTCTGGATTCTGCTAAAACAAACCAGACA
TCTGTGTACTTCTGTGCTAGCAGTTCAACAAACACAGAAGTCTTC
TTTGGTAAAGGAACCAGACTCACAGTTGTAGAGGATCTGAGAAAT
GTGACTCCACCCAAGGTCTCCTTGTTTGAGCCATCAAAAGCAGAG
ATTGCAAACAAACAAAAGGCTACCCTCGTGTGCTTGGCCAGGGGC
TTCTTCCCTGACCACGTGGAGCTGAGCTGGTGGGTGAATGGCAAG
GAGGTCCACAGTGGGGTCAGCACGGACCCTCAGGCCTACAAGGAG
AGCAATTATAGCTACTGCCTGAGCAGCCGCCTGAGGGTCTCTGCT
ACCTTCTGGCACAATCCTCGCAACCACTTCCGCTGCCAAGTGCAG
TTCCATGGGCTTTCAGAGGAGGACAAGTGGCCAGAGGGCTCACCC
AAACCTGTCACACAGAACATCAGTGCAGAGGCCTGGGGCCGAGCA
GACTGTGGGATTACCTCAGCATCCTATCAACAAGGGGTCTTGTCT
GCCACCATCCTCTATGAGATCCTGCTAGGGAAAGCCACCCTGTAT
GCTGTGCTTGTCAGTACACTGGTGGTGATGGCTATGGTCAAAAGA
AAGAACTCGTGA
(SEQ ID NO: 190)
ATGGTCCTAGTGACCATTCTGCTGCTCAGCGCGTTCTTCTCACTG
AGAGGAAACAGTGCCCAGTCCGTGGACCAGCCTGATGCTCATGTC
ACGCTCTCTGAAGGAGCCTCCCTGGAGCTCAGATGCAGTTATTCA
TACAGTGCAGCACCTTACCTCTTCTGGTACGTGCAGTATCCTGGC
CAGAGCCTCCAGTTTCTCCTCAAATACATCACAGGAGACACCGTT
GTTAAAGGCACCAAGGGCTTTGAGGCCGAGTTTAGGAAGAGTAAC
TCCTCTTTCAACCTGAAGAAATCCCCAGCCCATTGGAGCGACTCA
GCCAAGTACTTCTGTGCACTGGAGGGCCCGGATACAGGAAACTAC
AAATACGTCTT
(SEQ ID NO: 191)
ATGGGCATCCAGACCCTCTGTTGTGTGATCTTTTATGTTCTGATA
GCAAATCACACAGATGCTGGAGTTACCCAGACACCCAGACATGAG
GTGGCAGAGAAAGGACAAACAATAATCCTGAAGTGTGAGCCAGTT
TCAGGCCACAATGACCTTTTCTGGTACAGACAGACCAAGATACAG
GGACTAGAGTTGCTGAGCTACTTCCGCAGCAAGTCTCTTATGGAA
GATGGTGGGGCTTTCAAGGATCGATTCAAAGCTGAGATGCTAAAT
TCATCCTTCTCCACTCTGAAGATTCAACCTACAGAACCCAGGGAC
TCAGCTGTGTATCTGTGTGCCAGCAGTTTTGGGACAGCTAGTGCA
GAAACGCTGTATTTT
(SEQ ID NO: 192)
ATGGTCCTAGTGACCATTCTGCTGCTCAGCGCGTTCTTCTCACTG
AGAGGAAACAGTGCCCAGTCCGTGGACCAGCCTGATGCTCATGTC
ACGCTCTCTGAAGGAGCCTCCCTGGAGCTCAGATGCAGTTATTCA
TACAGTGCAGCACCTTACCTCTTCTGGTACGTGCAGTATCCTGGC
CAGAGCCTCCAGTTTCTCCTCAAATACATCACAGGAGACACCGTT
GTTAAAGGCACCAAGGGCTTTGAGGCCGAGTTTAGGAAGAGTAAC
TCCTCTTTCAACCTGAAGAAATCCCCAGCCCATTGGAGCGACTCA
GCCAAGTACTTCTGTGCACTGGAGGGCCCGGATACAGGAAACTAC
AAATACGTCTTTGGAGCAGGTACCAGACTGAAGGTTATAGCACAC
ATCCAGAACCCAGAACCTGCTGTGTACCAGTTAAAAGATCCTCGG
TCTCAGGACAGCACCCTCTGCCTGTTCACCGACTTTGACTCCCAA
ATCAATGTGCCGAAAACCATGGAATCTGGAACGTTCATCACTGAC
AAAACTGTGCTGGACATGAAAGCTATGGATTCCAAGAGCAATGGG
GCCATTGCCTGGAGCAACCAGACAAGCTTCACCTGCCAAGATATC
TTCAAAGAGACCAACGCCACCTACCCCAGTTCAGACGTTCCCTGT
GATGCCACGTTGACTGAGAAAAGCTTTGAAACAGATATGAACCTA
AACTTCCAAAACCTGTCAGTTATGGGACTCCGAATCCTCCTGCTG
AAAGTAGCCGGATTTAACCTGCTCATGACGCTGAGGCTGTGGTCC
AGTTGA
(SEQ ID NO: 193)
ATGGGCATCCAGACCCTCTGTTGTGTGATCTTTTATGTTCTGATA
GCAAATCACACAGATGCTGGAGTTACCCAGACACCCAGACATGAG
GTGGCAGAGAAAGGACAAACAATAATCCTGAAGTGTGAGCCAGTT
TCAGGCCACAATGACCTTTTCTGGTACAGACAGACCAAGATACAG
GGACTAGAGTTGCTGAGCTACTTCCGCAGCAAGTCTCTTATGGAA
GATGGTGGGGCTTTCAAGGATCGATTCAAAGCTGAGATGCTAAAT
TCATCCTTCTCCACTCTGAAGATTCAACCTACAGAACCCAGGGAC
TCAGCTGTGTATCTGTGTGCCAGCAGTTTTGGGACAGCTAGTGCA
GAAACGCTGTATTTTGGCTCAGGAACCAGACTGACTGTTCTCGAG
GATCTGAGAAATGTGACTCCACCCAAGGTCTCCTTGTTTGAGCCA
TCAAAAGCAGAGATTGCAAACAAACAAAAGGCTACCCTCGTGTGC
TTGGCCAGGGGCTTCTTCCCCTGACACGTGGAGCTGAGCTGGTGG
GTGAATGGCAAGGAGGTCCACAGTGGGGTCAGCACGGACCCTCAG
GCCTACAAGGAGAGCAATTATAGCTACTGCCTGAGCAGCCGCCTG
AGGGTCTCTGCTACCTTCTGGCACAATCCTCGAAACCACTTCCGC
TGTCAAGTGCAGTTCCATGGGCTTTCAGAGGAGGACAAGTGGCCA
GAGGGCTCACCCAAACCTGTCACACAGAACATCAGTGCAGAGGCC
TGGGGCCGAGCAGACTGTGGAATCACTTCAGCATCCTATCATCAG
GGGGTTCTGTCTGCAACCATCCTCTATGAGATCCTACTGGGGAAG
GCCACCCTATATGCTGTGCTGGTCAGTGGCCTGGTGCTGATGGCC
ATGGTCAAGAAAAAAAATTCCTGA
(SEQ ID NO: 194)
MGPVTCSVLVLLLMLRRSNGDGDSVTQTEGLVTLTEGLPVMLNCT
YQTIYSNPFLEWYVQHLNESPRLLLKSFTDNKRTEHQGFHATLHK
SSSSFHLQKSSAQLSDSALYYCAFDTNAYKVIF
(SEQ ID NO: 195)
MRVRLISAVVLCSLGTGLVDMKVTQMPRYLIKRMGENVLLECGQD
MSHETMYWYRQDPGLGLQLIYISYDVDSNSEGDIPKGYRVSRKKR
EHFSLILDSAKTNQTSVYFCASSSTNTEVE
(SEQ ID NO: 196)
MGPVTCSVLVLLLMLRRSNGDGDSVTQTEGLVTLTEGLPVMLNCT
YQTIYSNPFLFWYVQHLNESPRLLLKSFTDNKRTEHQGFHATLHK
SSSSFHLQKSSAQLSDSALYYCAFDTNAYKVIFGKGTHLHVLPNI
QNPEPAVYQLKDPRSQDSTLCLFTDFDSQINVPKTMESGTFITDK
TVLDMKAMDSKSNGAIAWSNQTSFTCQDIFKETNTTYPSSDVPCD
ATLTEKSFETDMNLNFQNLSVMGLRILLLKVAGFNLLMTLRLWSS
L
(SEQ ID NO: 197)
MRVRLISAVVLCSLGTGLVDMKVTQMPRYLIKRMGENVLLECGQD
MSHETMYWYRQDPGLGLQLIYISYDVDSNSEGDIPKGYRVSRKKR
EHFSLILDSAKTNQTSVYFCASSSTNTEVFFGKGTRLTVVEDLRN
VTPPKVSLFEPSKAEIANKQKATLVCLARGFFPDHVELSWWVNGK
EVHSGVSTDPQAYKESNYSYCLSSRLRVSATFWHNPRNHFRCQVQ
FHGLSEEDKWPEGSPKPVTQNISAEAWGRADCGITSASYQQGVLS
ATILYEILLGKATLYAVLVSTLVVM
(SEQ ID NO: 198)
MVLVTILLLSAFFSLRGNSAQSVDQPDAHVTLSEGASLELRCSYS
YSAAPYLFWYVQYPGQSLQFLLKYITGDTVVKGTKGFEAEFRKSN
SSFNLKKSPAHWSDSAKYFCALEGPDTGNYKYV
(SEQ ID NO: 199)
MGIQTLCCVIFYVLIANHTDAGVTQTPRHEVAEKGQTIILKCEPV
SGHNDLFWYRQTKIQGLELLSYFRSKSLMEDGGAFKDRFKAEMLN
SSFSTLKIQPTEPRDSAVYLCASSEGTASAETLY
(SEQ ID NO: 200)
MVLVTILLLSAFFSLRGNSAQSVDQPDAHVTLSEGASLELRCSYS
YSAAPYLFWYVQYPGQSLQFLLKYITGDTVVKGTKGFEAEFRKSN
SSFNLKKSPAHWSDSAKYFCALEGPDTGNYKYVFGAGTRLKVIAH
IQNPEPAVYQLKDPRSQDSTLCLFTDFDSQINVPKTMESGTFITD
KTVLDMKAMDSKSNGAIAWSNQTSFTCQDIFKETNATYPSSDVPC
DATLTEKSFETDMNLNFQNLSVMGLRILLLKVAGENLLMTLRLWS
S
(SEQ ID NO: 201)
MGIQTLCCVIFYVLIANHTDAGVTQTPRHEVAEKGQTIILKCEPV
SGHNDLFWYRQTKIQGLELLSYFRSKSLMEDGGAFKDRFKAEMLN
SSFSTLKIQPTEPRDSAVYLCASSFGTASAETLYFGSGTRLTVLE
DLRNVTPPKVSLFEPSKAEIANKQKATLVCLARGFFPHVELSWWV
NGKEVHSGVSTDPQAYKESNYSYCLSSRLRVSATFWHNPRNHFRC
QVQFHGLSEEDKWPEGSPKPVTQNISAEAWGRADCGITSASYHQG
VLSATILYEILLGKATLYAVLVSGLVLMAMVKKKNS

In some embodiments, a TCR construct comprises MAGE-A4-specific TCR chains. In some embodiments, a TCR construct comprising MAGE-A4-specific TCR chains comprises TCR alpha and TCR beta chains that target the epitope GVYDGREHTV (SEQ ID NO: 202). In some embodiments, a TCR construct comprising MAGE-A4-specific TCR chains comprises TCR alpha and TCR beta chains that target the epitope FMNKFIYEI (SEQ ID NO: 203). In some embodiments, MAGE-A4-specific TCR sequences, TCR variable domain sequences, CDR sequences, and/or TCR constant domain sequences, are described in international patent application publications WO 2017/174824 A1 and WO 2021/229212 A1, each of which are incorporated herein by reference for the purpose described herein. In certain embodiments, an anti-MAGE-A4 TCR alpha chain variable domain may have an M4V or an M4L amino acid substitution. In certain embodiments, an anti-MAGE-A4 TCR beta chain variable domain may have a N10E amino acid substitution.

In some embodiments, a TCR construct comprising MAGE-A4-specific TCR chains comprises a nucleotide coding sequence that is at least, or exactly, 85%, 86%, 87%, 88%, 89%, 90%, 91%, 92%, 93%, 94%, 95%, 96%, 97%, 98%, 99%, or 100%, identical to one or more of SEQ ID NOs: 204-205. In some embodiments, a TCR construct comprising MAGE-A4-specific TCR chains comprises an amino acid sequence that is at least, or exactly, 85%, 86%, 87%, 88%, 89%, 90%, 91%, 92%, 93%, 94%, 95%, 96%, 97%, 98%, 99%, or 100%, identical to one or more of SEQ ID NOs: 206-214.

(SEQ ID NO: 204)
ATGAAGAAGCACCTGACCACCTTTCTCGTGATCCTGTGGCTGTAC
TTCTACCGGGGCAACGGCAAGAACCAGGTGGAACAGAGCCCCCAG
AGCCTGATCATCCTGGAAGGCAAGAACTGCACCCTGCAGTGCAAC
TACACCGTGTCCCCCTTCAGCAACCTGCGGTGGTACAAGCAGGAC
ACCGGCAGAGGCCCTGTGTCCCTGACCATCCTGACCTTCAGCGAG
AACACCAAGAGCAACGGCCGGTACACCGCCACCCTGGACGCCGAT
ACAAAGCAGAGCAGCCTGCACATCACCGCCAGCCAGCTGAGCGAT
AGCGCCAGCTACATCTGCGTGGTGTCCGGCGGCACAGACAGCTGG
GGCAAGCTGCAGTTTGGCGCCGGAACACAGGTGGTCGTGACCCCC
GACATCCAGAACCCTGACCCTGCCGTGTACCAGCTGCGGGACAGC
AAGAGCAGCGACAAGAGCGTGTGCCTGTTCACCGACTTCGACAGC
CAGACCAACGTGTCCCAGAGCAAGGACAGCGACGTGTACATCACC
GACAAGACCGTGCTGGACATGCGGAGCATGGACTTCAAGAGCAAT
AGCGCCGTGGCCTGGTCCAACAAGAGCGACTTCGCCTGCGCCAAC
GCCTTCAACAACAGCATTATCCCCGAGGACACATTCTTCCCAAGC
CCCGAGAGCAGCTGCGACGTCAAGCTGGTGGAAAAGAGCTTCGAG
ACAGACACCAACCTGAACTTCCAGAACCTGAGCGTGATCGGCTTC
AGAATCCTGCTGCTGAAGGTGGCCGGCTTCAACCTGCTGATGACC
CTGAGACTGTGGTCCAGCGGCAGCCGGGCCAAGAGA
(SEQ ID NO: 205)
ATGGCCAGCCTGCTGTTCTTCTGCGGCGCCTTCTACCTGCTGGGC
ACCGGCTCTATGGATGCCGACGTGACCCAGACCCCCCGGAACAGA
ATCACCAAGACCGGCAAGCGGATCATGCTGGAATGCTCCCAGACC
AAGGGCCACGACCGGATGTACTGGTACAGACAGGACCCTGGCCTG
GGCCTGCGGCTGATCTACTACAGCTTCGACGTGAAGGACATCAAC
AAGGGCGAGATCAGCGACGGCTACAGCGTGTCCAGACAGGCTCAG
GCCAAGTTCAGCCTGTCCCTGGAAAGCGCCATCCCCAACCAGACC
GCCCTGTACTTTTGTGCCACAAGCGGCCAGGGCGCCTACGAGGAG
CAGTTCTTTGGCCCTGGCACCCGGCTGACAGTGCTGGAAGATCTG
AAGAACGTGTTCCCCCCAGAGGTGGCCGTGTTCGAGCCTTCTGAG
GCCGAAATCAGCCACACCCAGAAAGCCACACTCGTGTGTCTGGCC
ACCGGCTTCTACCCCGACCACGTGGAACTGTCTTGGTGGGTCAAC
GGCAAAGAGGTGCACAGCGGCGTGTCCACCGATCCCCAGCCTCTG
AAAGAACAGCCCGCCCTGAACGACAGCCGGTACTGCCTGAGCAGC
AGACTGAGAGTGTCCGCCACCTTCTGGCAGAACCCCAGAAACCAC
TTCAGATGCCAGGTGCAGTTTTACGGCCTGAGCGAGAACGACGAG
TGGACCCAGGACAGAGCCAAGCCCGTGACACAGATCGTGTCTGCC
GAAGCTTGGGGGCGCGCCGATTGTGGCTTTACCAGCGAGAGCTAC
CAGCAGGGCGTGCTGAGCGCCACCATCCTGTACGAGATCCTGCTG
GGAAAGGCCACACTGTACGCCGTGCTGGTGTCTGCCCTGGTGCTG
ATGGCCATGGTCAAGCGGAAGGACAGCCGGGGC
(SEQ ID NO: 206)
MKKHLTTFLVILWLYFYRGNGKNQVEQSPQSLIILEGKNCTLQCN
YTVSPFSNLRWYKQDTGRGPVSLTILTFSENTKSNGRYTATLDAD
TKQSSLHITASQLSDSASYICVVSGGTDSWGKLQFGAGTQVVVTP
DIQNPDPAVYQLRDSKSSDKSVCLFTDFDSQTNVSQSKDSDVYIT
DKTVLDMRSMDFKSNSAVAWSNKSDFACANAFNNSIIPEDTFFPS
PESSCDVKLVEKSFETDINLNFQNLSVIGFRILLLKVAGFNLLMT
LRLWSSGSRAKR
(SEQ ID NO: 207)
MKKHLTTFLVILWLYFYRGNGKNQVEQSPQSLIILEGKNCTLQCN
YTVSPFSNLRWYKQDTGRGPVSLTILTFSENTKSNGRYTATLDAD
TKQSSLHITASQLSDSASYICVVSGGTDSWGKLQFGAGTQVVVTP
D
(SEQ ID NO: 208)
MASLLFFCGAFYLLGTGSMDADVTQTPRNRITKTGKRIMLECSQT
KGHDRMYWYRQDPGLGLRLIYYSFDVKDINKGEISDGYSVSRQAQ
AKFSLSLESAIPNQTALYFCATSGQGAYEEQFFGPGTRLTVLEDL
KNVFPPEVAVFEPSEAEISHTQKATLVCLATGFYPDHVELSWWVN
GKEVHSGVSTDPQPLKEQPALNDSRYCLSSRLRVSATFWQNPRNH
ERCQVQFYGLSENDEWTQDRAKPVTQIVSAEAWGRADCGFTSESY
QQGVLSATILYEILLGKATLYAVLVSALVLMAMVKRKDSRG
(SEQ ID NO: 209)
MASLLFFCGAFYLLGTGSMDADVTQTPRNRITKTGKRIMLECSQT
KGHDRMYWYRQDPGLGLRLIYYSFDVKDINKGEISDGYSVSRQAQ
AKFSLSLESAIPNQTALYFCATSGQGAYEEQFFGPGTRLTVLE
(SEQ ID NO: 210)
MKNQVEQSPQSLIILEGKNCTLQCNYTVSPFSNLRWYKQDTGRGP
VSLTIMTFSENTKSNGRYTATLDADTKQSSLHITASQLSDSASYI
CVVSGGTDSWGKLQF
(SEQ ID NO: 211)
MKNQVEQSPQSLIILEGKNCTLQCNYTVSPFSNLRWYKQDTGRGP
VSLTIVTFSENTKSNGRYTATLDADTKQSSLHITASQLSDSASYI
CVVSGGTDSWGKLQF
(SEQ ID NO: 212)
MKNQVEQSPQSLIILEGKNCTLQCNYTVSPFSNLRWYKQDTGRGP
VSLTILTFSENTKSNGRYTATLDADTKQSSLHITASQLSDSASYI
CVVSGGTDSWGKLQF
(SEQ ID NO: 213)
MASLLFFCGAFYLLGTGSMDADVTQTPRNRITKTGKRIMLECSQT
KGHDRMYWYRQDPGLGLRLIYYSFDVKDINKGEISDGYSVSRQAQ
AKFSLSLESAIPNQTALYFCATSGQGAYNEQFF
(SEQ ID NO: 214)
MASLLFFCGAFYLLGTGSMDADVTQTPRNRITKTGKRIMLECSQT
KGHDRMYWYRQDPGLGLRLIYYSFDVKDINKGEISDGYSVSRQAQ
AKFSLSLESAIPNQTALYFCATSGQGAYEEQFF

In some embodiments, a TCR construct comprises Wilms' tumor antigen (WT1) WT1-specific TCR chains. In some embodiments, a TCR construct comprising WT1-specific TCR chains comprises TCR alpha and TCR beta chains that target the epitope VLDFAPPGA (SEQ ID NO: 215). In some embodiments, a TCR construct comprising WT1-specific TCR chains comprises TCR alpha and TCR beta chains that target the epitope RMFPNAPYL (SEQ ID NO: 216). In some embodiments, WT1-specific TCR sequences, TCR variable domain sequences, CDR sequences, and/or TCR constant domain sequences, are described in international patent application publications WO 2020/185796 A1 and WO 2021/034976 A1, each of which are incorporated herein by reference for the purpose described herein. In some embodiments, a leader sequence and/or signal peptide may be removed from a TCR amino acid sequence, and percentage sequence identity may be calculated based on the TCR amino acid sequence without the leader sequence and/or signal peptide.

In some embodiments, a TCR construct comprising WT1-specific TCR chains comprises a nucleotide coding sequence that is at least, or exactly, 85%, 86%, 87%, 88%, 89%, 90%, 91%, 92%, 93%, 94%, 95%, 96%, 97%, 98%, 99%, or 100%, identical to one or more of SEQ ID NOs: 217-256. In some embodiments, a TCR construct comprising WT1-specific TCR chains comprises an amino acid sequence that is at least, or exactly, 85%, 86%, 87%, 88%, 89%, 90%, 91%, 92%, 93%, 94%, 95%, 96%, 97%, 98%, 99%, or 100%, identical to one or more of SEQ ID NOs: 257-291.

(SEQ ID NO: 217)
ATGGAGACACTGCTGGGACTACTGATTCTGTGGCTGCAACTGCAA
TGGGTGAGCAGCAAACAGGAGGTTACCCAGATTCCTGCTGCTCTG
TCTGTTCCTGAAGGCGAGAATCTGGTGCTGAACTGCAGCTTCACA
GATAGCGCCATCTACAACCTGCAGTGGTTCAGACAGGATCCTGGA
AAAGGCCTGACAAGCCTGCTGCTGATTCAGAGCTCTCAGAGAGAG
CAGACATCTGGAAGACTGAATGCTAGCCTGGACAAGTCTAGCGGC
AGAAGCACCCTGTATATTGCCGCCTCTCAACCTGGAGATTCTGCC
ACATACCTGTGTGCTGTGAAGGAGACATCTGGCTCTAGACTGACC
TTTGGCGAGGGAACACAACTGACCGTGAATCCTGAC
(SEQ ID NO: 218)
ATGACCAGAGTTAGCCTGTTATGGGCTGTGGTGGTGAGCACATGT
CTGGAATCTGGAATGGCCCAGACAGTGACACAGTCTCAGCCTGAA
ATGTCTGTGCAGGAAGCCGAAACCGTTACACTGAGCTGCACCTAC
GATACAAGCGAGAACAACTACTACCTGTTCTGGTACAAGCAGCCC
CCCTCTAGGCAGATGATCCTGGTGATCAGACAGGAGGCCTATAAA
CAGCAGAATGCCACAGAGAACCGGTTCAGCGTGAACTTCCAGAAA
GCCGCCAAGAGCTTCAGCCTGAAGATCTCTGATTCTCAGCTGGGC
GATACAGCCATGTACTTTTGCGCCTTCATCTACCCCAGCTACACA
AGCGGCACATACAAGTACATCTTCGGCACCGGCACAAGACTGAAG
GTTCTGGCCAAC
(SEQ ID NO: 219)
ATGGCCATGTTACTAGGAGCGAGCGTGCTGATTCTGTGGTTACAG
CCTGATTGGGTGAACTCTCAGCAGAAGAACGATGATCAGCAGGTG
AAGCAGAACAGCCCCTCTCTGTCTGTGCAGGAAGGCAGAATCAGC
ATCCTGAATTGCGATTACACCAACAGCATGTTCGACTACTTCCTG
TGGTACAAGAAGTACCCCGCCGAGGGCCCTACCTTTCTGATCAGC
ATCTCTAGCATCAAGGACAAGAACGAAGATGGCAGATTCACCGTG
TTCCTGAACAAGAGCGCCAAGCACCTGAGCCTGCACATTGTGCCT
TCTCAACCTGGAGATTCTGCCGTGTACTTTTGTGCTGCCTCTGGA
ACAGGCGGAAGCTATATCCCCACATTTGGAAGAGGAACAAGCCTG
ATCGTGCACCCTTAC
(SEQ ID NO: 220)
ATGGCCATGTTACTAGGAGCGAGCGTGCTGATTCTGTGGTTACAG
CCTGATTGGGTGAACTCTCAGCAGAAGAACGATGATCAGCAGGTG
AAGCAGAACAGCCCCTCTCTGTCTGTGCAGGAAGGCAGAATCAGC
ATCCTGAATTGCGATTACACCAACAGCATGTTCGACTACTTCCTG
TGGTACAAGAAGTACCCCGCCGAGGGCCCTACCTTTCTGATCAGC
ATCTCTAGCATCAAGGACAAGAACGAAGATGGCAGATTCACCGTG
TTCCTGAACAAGAGCGCCAAGCACCTGAGCCTGCACATTGTGCCT
TCTCAACCTGGAGATTCTGCCGTGTACTTTTGTGCTGCCTCTGGC
ATTGGCGACTACAAACTGAGCTTTGGAGCCGGCACAACAGTGACC
GTTAGAGCCAAT
(SEQ ID NO: 221)
ATGGTGAAGATCCGGCAGTTCCTCCTGGCTATTCTGTGGCTGCAA
CTGTCTTGTGTGTCTGCTGCCAAGAATGAAGTGGAGCAGTCTCCC
CAGAACCTTACAGCCCAGGAAGGCGAGTTTATCACCATCAACTGC
AGCTATTCTGTGGGCATTAGCGCCCTGCATTGGCTGCAGCAACAC
CCTGGAGGAGGAATTGTGTCTCTGTTTATGCTGTCTTCTGGCAAG
AAGAAGCACGGCCGGCTGATTGCCACCATCAACATCCAGGAGAAG
CACTCTTCTCTGCACATTACAGCCTCTCATCCCAGGGATTCTGCC
GTGTACATCTGTGCCGTGAGAACCAGCTACGATAAGGTGATTTTC
GGACCAGGCACCTCTCTGAGCGTGATCCCCAAT
(SEQ ID NO: 222)
ATGAAGAGCCTGAGAGTCCTGCTGGTGATTTTGTGGCTGCAGCTG
TCTTGGGTTTGGTCTCAGCAGAAAGAAGTGGAGCAGAATAGCGGC
CCTCTGTCTGTTCCTGAAGGCGCTATTGCTAGCCTGAATTGCACA
TACAGCGATAGAGGATCTCAGAGCTTCTTCTGGTACCGGCAGTAC
AGCGGCAAGAGCCCAGAACTGATCATGTTCATCTACAGCAATGGC
GACAAGGAGGATGGCAGGTTTACAGCCCAGCTGAACAAGGCCAGC
CAGTATGTTTCTCTGCTGATCAGAGATAGCCAGCCTAGCGATTCT
GCCACCTACCTGTGTGCCGTGAACTTACTTGGAGCTACAGGATAC
TCTACACTGACCTTCGGCAAAGGCACCATGCTGCTGGTGAGCCCT
GAT
(SEQ ID NO: 223)
ATGTGGGGCGTTTTCCTTCTGTATGTGAGCATGAAGATGGGCGGC
ACAACAGGCCAGAACATCGATCAGCCTACCGAGATGACAGCCACA
GAAGGAGCTATTGTTCAGATCAACTGCACCTACCAGACAAGCGGC
TTCAACGGCCTGTTCTGGTACCAGCAGCATGCTGGAGAAGCTCCT
ACATTTCTGAGCTACAATGTGCTGGATGGCCTGGAGGAGAAAGGC
AGGTTTAGCAGCTTCCTGAGCAGGTCTAAGGGCTATTCTTATCTG
CTGCTGAAGGAGCTGCAGATGAAGGATTCCGCCAGCTACCTGTGT
GCCGTTAGGGGCATCAATGATTACAAGCTGAGCTTTGGAGCCGGA
ACAACAGTGACCGTGAGAGCCAAC
(SEQ ID NO: 224)
ATGGAGAAGATGCTGGAGTGTGCGTTCATCGTTCTGTGGCTGCAA
CTTGGATGGCTGTCTGGAGAGGATCAGGTTACACAGTCTCCTGAA
GCCCTGAGACTGCAAGAAGGAGAAAGCTCTAGCCTGAACTGCAGC
TACACAGTGTCTGGACTGAGAGGCCTGTTCTGGTACAGACAGGAT
CCTGGAAAAGGCCCAGAGTTCCTGTTTACCCTGTATTCTGCCGGC
GAGGAGAAGGAGAAAGAGAGACTGAAAGCTACCCTGACCAAGAAG
GAGAGCTTCCTGCACATTACCGCCCCCAAACCTGAGGATTCTGCC
ACATATCTGTGTGCCGTGATTACCGGCTTTCAGAAGCTGGTGTTT
GGCACAGGCACCAGACTGCTGGTTTCTCCCAAT
(SEQ ID NO: 225)
ATGAGACTGGTGGCACGCGTAACTGTGTTTCTGACCTTTGGCACC
ATCATCGATGCCAAGACAACCCAGCCTACAAGCATGGACTGTGCC
GAGGGAAGAGCTGCTAATCTGCCATGTAATCACAGCACAATCAGC
GGCAACGAGTACGTGTACTGGTACCGGCAGATCCACTCTCAAGGA
CCTCAGTACATCATTCATGGCCTGAAGAACAACGAGACCAACGAG
ATGGCCAGCCTGATCATCACCGAGGACAGGAAGTCTTCTACCCTG
ATTCTGCCTCATGCTACACTGAGAGATACCGCCGTGTACTACTGC
ATTGCCGGAGTGGGAAGAGGCCAGAATTTCGTGTTTGGACCTGGA
ACAAGACTGAGCGTTCTGCCCTAT
(SEQ ID NO: 226)
ATGGAGAAGAACCCCTTGGCAGCACCTCTGCTTATTCTGTGGTTC
CACCTGGATTGTGTGAGCAGCATCCTGAATGTGGAGCAGTCTCCT
CAGAGCCTGCATGTGCAAGAAGGCGATAGCACCAATTTCACCTGC
AGCTTTCCAAGCAGCAACTTCTACGCCCTGCACTGGTACAGATGG
GAAACCGCCAAATCTCCTGAAGCCCTGTTTGTGATGACCCTGAAT
GGCGACGAGAAGAAGAAGGGCAGAATTAGCGCCACCCTGAATACC
AAGGAGGGCTACAGCTACCTGTACATCAAGGGCTCTCAACCTGAG
GATTCTGCCACCTACCTTTGCGCCTTTCACCCCAATTTCGGCAAC
GAGAAACTGACCTTTGGAACCGGAACAAGGCTGACCATCATCCCC
AAC
(SEQ ID NO: 227)
ATGGAGAAGATGCTGGAGTGTGCGTTCATCGTTCTGTGGCTGCAA
CTTGGATGGCTGTCTGGAGAGGATCAGGTTACACAGTCTCCTGAA
GCCCTGAGACTGCAAGAAGGAGAAAGCTCTAGCCTGAACTGCAGC
TACACAGTGTCTGGACTGAGAGGCCTGTTCTGGTACAGACAGGAT
CCTGGAAAAGGCCCAGAGTTCCTGTTTACCCTGTATTCTGCCGGC
GAGGAGAAGGAGAAAGAGAGACTGAAAGCTACCCTGACCAAGAAG
GAGAGCTTCCTGCACATTACCGCCCCCAAACCTGAGGATTCTGCC
ACATATCTGTGTGCTGTTCAGCCTAGAGGAGATGGCTCTAGCAAT
ACCGGCAAGCTGATCTTTGGCCAGGGAACAACACTGCAGGTGAAG
CCTGAT
(SEQ ID NO: 228)
ATCCAGAATCCCGATCCTGCTGTGTACCAGCTGCGGGACAGCAAG
AGCAGCGACAAGAGCGTGTGCCTGTTCACCGACTTCGACAGCCAG
ACCAACGTGTCCCAGAGCAAGGACAGCGACGTGTACATCACCGAT
AAGTGCGTGCTGGACATGCGGAGCATGGACTTCAAGAGCAACAGC
GCCGTGGCCTGGTCCAACAAGAGCGACTTCGCCTGCGCCAACGCC
TTCAACAACAGCATTATCCCCGAGGACACATTCTTCCCAAGCCCC
GAGAGCAGCTGCGACGTGAAGCTGGTGGAAAAGAGCTTCGAGACA
GACACCAACCTGAACTTCCAGAACCTCAGCGTGATCGGCTTCCGG
ATCCTGCTGCTGAAGGTGGCCGGCTTCAACCTGCTGATGACCCTG
CGGCTGTGGTCCAGCTGA
(SEQ ID NO: 229)
CTCAATAAAAGAGCCCACAACCCCTCACTCGGCGCGCCACCATGG
GCACATCTCTTCTCTGTTGGGTGGTTCTGGGCTTTCTGGGCACAG
ATCATACAGGAGCTGGAGTTAGCCAGTCTCCTAGGTATAAGGTGA
CCAAGAGGGGACAGGATGTGGCTCTGAGATGTGACCCTATTAGCG
GACATGTGAGCCTGTACTGGTACAGACAAGCTCTGGGACAAGGAC
CCGAGTTTCTGACCTACTTCAACTATGAGGCCCAGCAGGACAAAT
CTGGACTGCCCAACGACAGATTCAGCGCCGAAAGACCAGAAGGCT
CTATTAGCACACTGACCATCCAGAGAACAGAGCAGAGGGATTCTG
CCATGTACAGATGCGCCAGCAGCTTAACAGGCTCTTACGAGCAGT
ACTTTGGACCTGGCACAAGACTGACAGTGACAGAG
(SEQ ID NO: 230)
CTCAATAAAAGAGCCCACAACCCCTCACTCGGCGCGCCACCATGCT
GCTTCTTCTCCTCCTTCTCGGACCTGCTGGATCTGGATTAGGAGC
TGTTGTGTCTCAGCACCCTTCTTGGGTGATCTGTAAAAGCGGCAC
AAGCGTGAAGATCGAGTGCAGAAGCCTGGACTTTCAGGCCACAAC
CATGTTCTGGTATAGGCAGTTCCCCAAGCAGTCTCTGATGCTGAT
GGCCACCTCTAATGAGGGCTCTAAGGCCACATATGAACAGGGAGT
GGAGAAGGACAAGTTCCTGATCAACCACGCCTCTCTGACCCTGTC
TACCCTGACAGTTACATCTGCCCACCCTGAGGATAGCAGCTTTTA
CATCTGTAGCGCCACACCTGAAGCCTCTAGCCCATATGAGCAGTA
CTTTGGCCCTGGCACCAGATTAACAGTGACAGAG
(SEQ ID NO: 231)
CTCAATAAAAGAGCCCACAACCCCTCACTCGGCGCGCCACCATGG
GACCTGGACTGCTTCATTGGATGGCTCTGTGTTTGCTGGGAACAG
GACATGGAGATGCTATGGTGATCCAGAACCCCAGGTATCAGGTGA
CCCAGTTTGGCAAACCAGTGACACTGAGCTGTTCTCAGACCCTGA
ACCACAACGTGATGTACTGGTACCAGCAGAAGTCTTCTCAGGCCC
CTAAGCTGCTGTTCCACTACTACGACAAGGACTTCAACAACGAGG
CCGATACCCCTGACAATTTCCAGAGCAGGAGGCCCAATACCAGCT
TCTGTTTCCTGGACATTAGAAGCCCTGGACTGGGAGATGCTGCCA
TGTACCTGTGTGCCACCAGCAATTTACAGGGAAGACAACCTCAGC
ACTTTGGCGATGGCACAAGGCTGTCTATCCTGGAG
(SEQ ID NO: 232)
CTCAATAAAAGAGCCCACAACCCCTCACTCGGCGCGCCACCATGC
TGAGCCCTGATCTCCCTGATTCTGCCTGGAATACCAGACTGCTGT
GTCATGTGATGCTGTGTCTGCTTGGAGCCGTTTCTGTGGCTGCTG
GCGTGATTCAATCTCCTAGACACCTGATCAAGGAGAAGAGAGAAA
CAGCCACCCTGAAGTGCTACCCCATCCCCAGACACGATACAGTGT
ACTGGTATCAGCAAGGACCTGGACAAGATCCCCAGTTCCTGATCA
GCTTCTACGAGAAGATGCAGAGCGACAAAGGCAGCATCCCAGACA
GATTTAGCGCCCAGCAGTTTAGCGACTATCACTCTGAGCTGAACA
TGAGCAGCCTGGAACTGGGCGATTCTGCTCTGTACTTCTGTGCCT
CTTCTCTGAGACTGGGAAGAGAAACCCAGTACTTTGGACCCGGCA
CAAGACTGCTGGTTCTTGAG
(SEQ ID NO: 233)
CTCAATAAAAGAGCCCACAACCCCTCACTCGGCGCGCCACCATGG
GCACAAGACTTCTCTGCTGGGTGGTGCTTGGATTTCTGGGCACAG
ATCATACAGGAGCTGGAGTTAGCCAGTCTCCTAGGTACAAAGTGG
CCAAGAGAGGACAGGATGTGGCTCTGAGATGTGACCCTATTAGCG
GACATGTGAGCCTGTTTTGGTACCAGCAAGCTCTGGGACAAGGAC
CCGAGTTTCTGACCTACTTCCAGAATGAAGCCCAGCTGGATAAAT
CTGGACTGCCTAGCGACCGGTTCTTCGCCGAAAGACCTGAAGGAT
CTGTTAGCACCCTGAAGATTCAGAGAACACAGCAGGAGGACTCTG
CCGTGTACCTGTGTGCCTCTTCTTTAGGACAGGCCTATGAGCAGT
ATTTTGGACCTGGCACCAGACTGACCGTGACAGAG
(SEQ ID NO: 234)
CTCAATAAAAGAGCCCACAACCCCTCACTCGGCGCGCCACCATGG
GCACAAGACTTCTCTGCTGGGTGGCCTTTTGTCTGCTGGTGGAAG
AGCTGATTGAAGCTGGAGTTGTGCAGTCTCCTAGGTACAAGATCA
TCGAGAAGAAGCAGCCCGTGGCCTTCTGGTGTAATCCCATTTCTG
GCCACAACACCCTGTACTGGTATCTGCAGAATCTGGGACAGGGCC
CTGAACTGCTGATCAGATACGAGAACGAAGAAGCCGTGGACGATT
CTCAACTGCCTAAGGACCGCTTTTCTGCCGAGAGGCTGAAAGGAG
TGGATTCTACCCTGAAGATCCAACCTGCTGAACTGGGCGATTCTG
CTGTGTACCTGTGCGCTTCTAGCCTGACAAGAGGAGCTGAAGCCT
TTTTTGGACAGGGCACAAGACTGACAGTGGTGGAG
(SEQ ID NO: 235)
CTCAATAAAAGAGCCCACAACCCCTCACTCGGCGCGCCACCATGG
GACCTCAGCTTCTTGGATACGTTGTGCTGTGTCTGCTTGGAGCTG
GACCTCTTGAAGCTCAGGTTACCCAGAACCCCAGATACCTGATTA
CCGTGACAGGCAAAAAGCTGACCGTGACATGTAGCCAGAACATGA
ACCACGAGTACATGAGCTGGTACCGGCAGGATCCTGGATTAGGCC
TGAGACAGATCTACTACAGCATGAACGTGGAGGTGACCGATAAAG
GCGACGTGCCTGAGGGATACAAGGTGAGCAGAAAGGAGAAGAGGA
ATTTCCCCCTGATCCTGGAAAGCCCAAGCCCCAATCAGACAAGCC
TGTACTTTTGTGCCAGCAGCTTTTCTGGCGGCACATATGAGCAGT
ACTTCGGCCCTGGCACAAGACTGACAGTTACAGAG
(SEQ ID NO: 236)
CTCAATAAAAGAGCCCACAACCCCTCACTCGGCGCGCCACCATGC
TGAGCCCTGATCTCCCTGATTCTGCCTGGAATACCAGACTGCTGT
GTCATGTGATGCTGTGTCTGCTTGGAGCCGTTTCTGTGGCTGCTG
GCGTGATTCAATCTCCTAGACACCTGATCAAGGAGAAGAGAGAAA
CAGCCACCCTGAAGTGCTACCCCATCCCCAGACACGATACAGTGT
ACTGGTATCAGCAAGGACCTGGACAAGATCCCCAGTTCCTGATCA
GCTTCTACGAGAAGATGCAGAGCGACAAAGGCAGCATCCCAGACA
GATTTAGCGCCCAGCAGTTTAGCGACTATCACTCTGAGCTGAACA
TGAGCAGCCTGGAACTGGGCGATTCTGCTCTGTACTTCTGTGCCA
GCAGCTATAGAGGAGGCAGCACATATGAGCAGTACTTTGGCCCTG
GCACAAGACTGACAGTGACAGAG
(SEQ ID NO: 237)
CTCAATAAAAGAGCCCACAACCCCTCACTCGGCGCGCCACCATGA
GCACCAGACTCCTTTGCTGGATGGCTTTGTGTCTGCTTGGAGCTG
AGCTGTCTGAAGCTGAAGTTGCCCAGTCTCCCAGATACAAGATCA
CCGAGAAATCTCAGGCTGTGGCCTTCTGGTGTGACCCTATTTCTG
GACACGCCACCCTGTACTGGTATAGGCAAATTCTGGGACAAGGCC
CTGAACTGCTGGTGCAATTTCAGGATGAGAGCGTGGTGGACGATT
CTCAACTGCCTAAGGACAGGTTTTCTGCCGAGCGGCTGAAAGGAG
TTGATAGCACCCTGAAGATCCAACCTGCTGAACTGGGCGATTCTG
CTATGTACCTGTGCGCCTCTTCTCAGAGAGATAGCCCTAACGAGA
AGCTGTTCTTTGGCTCTGGAACCCAGCTGTCTGTGCTGGAG
(SEQ ID NO: 238)
CTCAATAAAAGAGCCCACAACCCCTCACTCGGCGCGCCACCATGG
GCTGTAGACTGTTGTGTTGTGCTGTGCTGTGTCTGTTGGGAGCTG
TGCCTATGGAAACAGGCGTTACCCAGACACCTAGACATCTGGTTA
TGGGCATGACCAACAAGAAGAGCCTGAAGTGCGAGCAGCATCTGG
GCCATAACGCCATGTACTGGTATAAGCAGAGCGCCAAGAAACCAC
TGGAACTGATGTTCGTGTACAGCCTGGAGGAGAGGGTGGAGAATA
ATAGCGTGCCCAGCAGATTTAGCCCTGAGTGCCCAAATTCTTCTC
ACCTGTTCCTGCACCTGCACACATTACAGCCCGAGGATTCTGCCC
TGTACCTGTGTGCTTCTTCTCAAGACCCTTACAAGCTGAGCGGCA
ATACCATCTACTTCGGCGAAGGCTCTTGGCTGACAGTGGTTGAA
(SEQ ID NO: 239)
GATCTGAACAAGGTGTTCCCCCCAGAGGTGGCCGTGTTCGAGCCT
TCTGAGGCCGAGATCTCCCACACCCAGAAAGCCACCCTCGTGTGC
CTGGCCACCGGCTTTTTCCCCGACCACGTGGAACTGTCTTGGTGG
GTCAACGGCAAAGAGGTGCACTCCGGCGTGTGCACCGATCCCCAG
CCTCTGAAAGAACAGCCCGCCCTGAACGACAGCCGGTACTGCCTG
AGCAGCAGACTGAGAGTGTCCGCCACCTTCTGGCAGAACCCCCGG
AACCACTTCAGATGCCAGGTGCAGTTCTACGGCCTGAGCGAGAAC
GACGAGTGGACCCAGGACAGAGCCAAGCCCGTGACACAGATCGTG
TCTGCCGAAGCCTGGGGCAGAGCCGATTGCGGCTTTACCTCCGTG
TCCTATCAGCAGGGCGTGCTGAGCGCCACAATCCTGTACGAGATC
CTGCTGGGCAAGGCCACCCTGTACGCCGTGCTGGTGTCTGCCCTG
GTGCTGATGGCCATGGTCAAGCGGAAGGACTTC
(SEQ ID NO: 240)
GACCTGAAGAACGTGTTCCCCCCAGAGGTGGCCGTGTTCGAGCCT
AGCGAGGCCGAGATCAGCCACACCCAGAAAGCCACCCTCGTGTGC
CTGGCCACCGGCTTTTACCCCGACCACGTGGAACTGTCTTGGTGG
GTCAACGGCAAAGAGGTGCACAGCGGCGTCTGCACCGACCCCCAG
CCCCTGAAAGAGCAGCCCGCCCTGAACGACAGCCGGTACTGTCTG
AGCAGCAGACTGAGAGTGTCCGCCACCTTCTGGCAGAACCCCCGG
AACCACTTCAGATGCCAGGTGCAGTTCTACGGCCTGAGCGAGAAC
GACGAGTGGACCCAGGACCGGGCCAAGCCCGTGACCCAGATCGTG
TCTGCTGAGGCCTGGGGCAGAGCCGATTGCGGCTTCACCAGCGAG
AGCTACCAGCAGGGCGTGCTGAGCGCCACCATCCTGTACGAGATC
CTGCTGGGCAAGGCCACCCTGTACGCCGTGCTGGTGTCCGCCCTG
GTGCTGATGGCCATGGTCAAGCGGAAGGACAGCCGGGGC
(SEQ ID NO: 241)
ATGAAATCCTTGAGAGTTTTACTAGTGATCCTGTGGCTTCAGTTG
AGCTGGGTTTGGAGCCAACAGAAGGAGGTGGAGCAGAATTCTGGA
CCCCTCAGTGTTCCAGAGGGAGCCATTGCCTCTCTCAACTGCACT
TACAGTGACCGAGGTTCCCAGTCCTTCTTCTGGTACAGACAATAT
TCTGGGAAAAGCCCTGAGTTGATAATGTTCATATACTCCAATGGT
GACAAAGAAGATGGAAGGTTTACAGCACAGCTCAATAAAGCCAGC
CAGTATGTTTCTCTGCTCATCAGAGACTCCCAGCCCAGTGATTCA
GCCACCTACCTCTGTGCCGTGAACATAGGAAACCATGACATGCGC
TTTGGAGCAGGGACCAGACTGACAGTAAAACCAAATATCCAGAAC
CCTGACCCTGCCGTGTACCAGCTGAGAGACTCTAAATCCAGTGAC
AAGTCTGTCTGCCTATTCACCGATTTTGATTCTCAAACAAATGTG
TCACAAAGTAAGGATTCTGATGTGTATATCACAGACAAAACTGTG
CTAGACATGAGGTCTATGGACTTCAAGAGCAACAGTGCTGTGGCC
TGGAGCAACAAATCTGACTTTGCATGTGCAAACGCCTTCAACAAC
AGCATTATTCCAGAAGACACCTTCTTCCCCAGCCCAGAAAGTTCC
TGTGATGTCAAGCTGGTCGAGAAAAGCTTTGAAACAGATACGAAC
CTAAACTTTCAAAACCTGTCAGTGATTGGGTTCCGAATCCTCCTC
CTGAAAGTGGCCGGGTTTAATCTGCTCATGACGCTGCGGCTGTGG
TCCAGCTGA
(SEQ ID NO: 242)
ATGGAGAAAATGTTGGAGTGTGCATTCATAGTCTTGTGGCTTCAG
CTTGGCTGGTTGAGTGGAGAAGACCAGGTGACGCAGAGTCCCGAG
GCCCTGAGACTCCAGGAGGGAGAGAGTAGCAGTCTCAACTGCAGT
TACACAGTCAGCGGTTTAAGAGGGCTGTTCTGGTATAGGCAAGAT
CCTGGGAAAGGCCCTGAATTCCTCTTCACCCTGTATTCAGCTGGG
GAAGAAAAGGAGAAAGAAAGGCTAAAAGCCACATTAACAAAGAAG
GAAAGCTTTCTGCACATCACAGCCCCTAAACCTGAAGACTCAGCC
ACTTATCTCTGTGCTGTGCAGACCATGGACGGTAACCAGTTCTAT
TTTGGGACAGGGACAAGTTTGACGGTCATTCCAAATATCCAGAAC
CCTGACCCTGCCGTGTACCAGCTGAGAGACTCTAAATCCAGTGAC
AAGTCTGTCTGCCTATTCACCGATTTTGATTCTCAAACAAATGTG
TCACAAAGTAAGGATTCTGATGTGTATATCACAGACAAAACTGTG
CTAGACATGAGGTCTATGGACTTCAAGAGCAACAGTGCTGTGGCC
TGGAGCAACAAATCTGACTTTGCATGTGCAAACGCCTTCAACAAC
AGCATTATTCCAGAAGACACCTTCTTCCCCAGCCCAGAAAGTTCC
TGTGATGTCAAGCTGGTCGAGAAAAGCTTTGAAACAGATACGAAC
CTAAACTTTCAAAACCTGTCAGTGATTGGGTTCCGAATCCTCCTC
CTGAAAGTGGCCGGGTTTAATCTGCTCATGACGCTGCGGCTGTGG
TCCAGCTGA
(SEQ ID NO: 243)
ATGGCATGCCCTGGCTTCCTGTGGGCACTTGTGATCTCCACCTGT
CTTGAATTTAGCATGGCTCAGACAGTCACTCAGTCTCAACCAGAG
ATGTCTGTGCAGGAGGCAGAGACCGTGACCCTGAGCTGCACATAT
GACACCAGTGAGAGTGATTATTATTTATTCTGGTACAAGCAGCCT
CCCAGCAGGCAGATGATTCTCGTTATTCGCCAAGAAGCTTATAAG
CAACAGAATGCAACAGAGAATCGTTTCTCTGTGAACTTCCAGAAA
GCAGCCAAATCCTTCAGTCTCAAGATCTCAGACTCACAGCTGGGG
GATGCCGCGATGTATTTCTGTGCTTCCAGTCCAGGAACCTACAAA
TACATCTTTGGAACAGGCACCAGGCTGAAGGTTTTAGCAAATATC
CAGAACCCTGACCCTGCCGTGTACCAGCTGAGAGACTCTAAATCC
AGTGACAAGTCTGTCTGCCTATTCACCGATTTTGATTCTCAAACA
AATGTGTCACAAAGTAAGGATTCTGATGTGTATATCACAGACAAA
ACTGTGCTAGACATGAGGTCTATGGACTTCAAGAGCAACAGTGCT
GTGGCCTGGAGCAACAAATCTGACTTTGCATGTGCAAACGCCTTC
AACAACAGCATTATTCCAGAAGACACCTTCTTCCCCAGCCCAGAA
AGTTCCTGTGATGTCAAGCTGGTCGAGAAAAGCTTTGAAACAGAT
ACGAACCTAAACTTTCAAAACCTGTCAGTGATTGGGTTCCGAATC
CTCCTCCTGAAAGTGGCCGGGTTTAATCTGCTCATGACGCTGCGG
CTGTGGTCCAGCTGA
(SEQ ID NO: 244)
ATGACACGAGTTAGCTTGCTGTGGGCAGTCGTGGTCTCCACCTGT
CTTGAATCCGGCATGGCCCAGACAGTCACTCAGTCTCAACCAGAG
ATGTCTGTGCAGGAGGCAGAGACTGTGACCCTGAGTTGCACATAT
GACACCAGTGAGAGTAATTATTATTTGTTCTGGTACAAACAGCCT
CCCAGCAGGCAGATGATTCTCGTTATTCGCCAAGAAGCTTATAAG
CAACAGAATGCAACGGAGAATCGTTTCTCTGTGAACTTCCAGAAA
GCAGCCAAATCCTTCAGTCTCAAGATCTCAGACTCACAGCTGGGG
GACACTGCGATGTATTTCTGTGCTTTCAACCCTTGGGAGAACTAT
GGTCAGAATTTTGTCTTTGGTCCCGGAACCAGATTGTCCGTGCTG
CCCTATATCCAGAACCCTGACCCTGCCGTGTACCAGCTGAGAGAC
TCTAAATCCAGTGACAAGTCTGTCTGCCTATTCACCGATTTTGAT
TCTCAAACAAATGTGTCACAAAGTAAGGATTCTGATGTGTATATC
ACAGACAAAACTGTGCTAGACATGAGGTCTATGGACTTCAAGAGC
AACAGTGCTGTGGCCTGGAGCAACAAATCTGACTTTGCATGTGCA
AACGCCTTCAACAACAGCATTATTCCAGAAGACACCTTCTTCCCC
AGCCCAGAAAGTTCCTGTGATGTCAAGCTGGTCGAGAAAAGCTTT
GAAACAGATACGAACCTAAACTTTCAAAACCTGTCAGTGATTGGG
TTCCGAATCCTCCTCCTGAAAGTGGCCGGGTTTAATCTGCTCATG
ACGCTGCGGCTGTGGTCCAGCTGA
(SEQ ID NO: 245)
ATGAAGAGCCTGAGAGTCCTGCTGGTGATTTTGTGGCTGCAGCTG
TCTTGGGTTTGGTCTCAGCAGAAAGAAGTGGAGCAGAATAGCGGC
CCTCTGTCTGTTCCTGAAGGCGCTATTGCTAGCCTGAATTGCACA
TACAGCGATAGAGGATCTCAGAGCTTCTTCTGGTACCGGCAGTAC
AGCGGCAAGAGCCCAGAACTGATCATGTTCATCTACAGCAATGGC
GACAAGGAGGATGGCAGGTTTACAGCCCAGCTGAACAAGGCCAGC
CAGTATGTTTCTCTGCTGATCAGAGATAGCCAGCCTAGCGATTCT
GCCACCTACCTGTGTGCCGTGAACATCGGAAATCACGACATGAGA
TTTGGAGCCGGCACAAGACTGACCGTGAAGCCCAATATCCAGAAC
CCTGATCCTGCTGTGTACCAGCTGCGGGACAGCAAGAGCAGCGAC
AAGAGCGTGTGCCTGTTCACCGACTTCGACAGCCAGACCAACGTG
TCCCAGAGCAAGGACAGCGACGTGTACATCACCGATAAGTGCGTG
CTGGACATGCGGAGCATGGACTTCAAGAGCAACAGCGCCGTGGCC
TGGTCCAACAAGAGCGACTTCGCCTGCGCCAACGCCTTCAACAAC
AGCATTATCCCCGAGGACACATTCTTCCCAAGCCCCGAGAGCAGC
TGCGACGTGAAGCTGGTGGAAAAGAGCTTCGAGACAGACACCAAC
CTGAACTTCCAGAACCTCAGCGTGATCGGCTTCCGGATCCTGCTG
CTGAAGGTGGCCGGCTTCAACCTGCTGATGACCCTGCGGCTGTGG
TCCAGCTGA
(SEQ ID NO: 246)
ATGGAGAAGATGCTGGAGTGTGCGTTCATCGTTCTGTGGCTGCAA
CTTGGATGGCTGTCTGGAGAGGATCAGGTTACACAGTCTCCTGAA
GCCCTGAGACTGCAAGAAGGAGAAAGCTCTAGCCTGAACTGCAGC
TACACAGTGTCTGGACTGAGAGGCCTGTTCTGGTACAGACAGGAT
CCTGGAAAAGGCCCAGAGTTCCTGTTTACCCTGTATTCTGCCGGC
GAGGAGAAGGAGAAAGAGAGACTGAAAGCTACCCTGACCAAGAAG
GAGAGCTTCCTGCACATTACCGCCCCCAAACCTGAGGATTCTGCC
ACATATCTGTGTGCTGTGCAGACCATGGATGGCAACCAGTTCTAC
TTCGGCACAGGCACATCTCTGACCGTTATCCCCAATATCCAGAAC
CCTGATCCTGCCGTGTACCAGCTGCGGGACAGCAAGAGCAGCGAC
AAGAGCGTGTGCCTGTTCACCGACTTCGACAGCCAGACCAACGTG
TCCCAGAGCAAGGACAGCGACGTGTACATCACCGATAAGTGCGTG
CTGGACATGCGGAGCATGGACTTCAAGAGCAACAGCGCCGTGGCC
TGGTCCAACAAGAGCGACTTCGCCTGCGCCAACGCCTTCAACAAC
AGCATTATCCCCGAGGACACATTCTTCCCAAGCCCCGAGAGCAGC
TGCGACGTGAAGCTGGTGGAAAAGAGCTTCGAGACAGACACCAAC
CTGAACTTCCAGAACCTCAGCGTGATCGGCTTCCGGATCCTGCTG
CTGAAGGTGGCCGGCTTCAACCTGCTGATGACCCTGCGGCTGTGG
TCCAGCTGA
(SEQ ID NO: 247)
ATGGCTTGTCCTGGATTCTTATGGGCTCTGGTGATCAGCACCTGT
CTGGAGTTCTCTATGGCCCAGACAGTGACACAGTCTCAGCCTGAA
ATGTCTGTGCAGGAAGCCGAAACCGTGACACTGTCTTGCACCTAC
GATACAAGCGAGAGCGACTACTACCTGTTCTGGTACAAGCAGCCT
CCCTCTAGGCAGATGATCCTGGTGATTAGACAGGAGGCCTACAAA
CAGCAGAATGCCACCGAGAACCGGTTTAGCGTGAACTTCCAGAAA
GCCGCCAAGAGCTTCAGCCTGAAAATCTCTGACAGCCAGCTGGGA
GATGCTGCCATGTACTTTTGTGCCAGCTCTCCAGGCACCTACAAG
TACATTTTTGGCACCGGCACCAGACTGAAGGTGCTGGCCAATATC
CAGAATCCCGATCCTGCCGTGTACCAGCTGCGGGACAGCAAGAGC
AGCGACAAGAGCGTGTGCCTGTTCACCGACTTCGACAGCCAGACC
AACGTGTCCCAGAGCAAGGACAGCGACGTGTACATCACCGATAAG
TGCGTGCTGGACATGCGGAGCATGGACTTCAAGAGCAACAGCGCC
GTGGCCTGGTCCAACAAGAGCGACTTCGCCTGCGCCAACGCCTTC
AACAACAGCATTATCCCCGAGGACACATTCTTCCCAAGCCCCGAG
AGCAGCTGCGACGTGAAGCTGGTGGAAAAGAGCTTCGAGACAGAC
ACCAACCTGAACTTCCAGAACCTCAGCGTGATCGGCTTCCGGATC
CTGCTGCTGAAGGTGGCCGGCTTCAACCTGCTGATGACCCTGCGG
CTGTGGTCCAGCTGA
(SEQ ID NO: 248)
ATGACCAGAGTTAGCCTGTTATGGGCTGTGGTGGTGAGCACATGT
CTGGAATCTGGAATGGCCCAGACAGTGACACAGTCTCAGCCTGAA
ATGTCTGTGCAGGAAGCCGAAACCGTTACACTGAGCTGCACCTAC
GATACAAGCGAGAGCAACTACTACCTGTTCTGGTACAAGCAGCCC
CCTTCTAGGCAGATGATCCTGGTGATCAGACAGGAGGCCTATAAA
CAGCAGAATGCCACCGAGAACCGGTTTAGCGTGAACTTCCAGAAA
GCCGCCAAGAGCTTCAGCCTGAAAATCTCTGACAGCCAGCTGGGC
GATACAGCCATGTACTTTTGTGCCTTCAACCCCTGGGAGAACTAT
GGCCAGAATTTCGTGTTCGGCCCTGGCACCAGACTGTCTGTTCTG
CCTTATATCCAGAACCCCGATCCTGCTGTGTACCAGCTGCGGGAC
AGCAAGAGCAGCGACAAGAGCGTGTGCCTGTTCACCGACTTCGAC
AGCCAGACCAACGTGTCCCAGAGCAAGGACAGCGACGTGTACATC
ACCGATAAGTGCGTGCTGGACATGCGGAGCATGGACTTCAAGAGC
AACAGCGCCGTGGCCTGGTCCAACAAGAGCGACTTCGCCTGCGCC
AACGCCTTCAACAACAGCATTATCCCCGAGGACACATTCTTCCCA
AGCCCCGAGAGCAGCTGCGACGTGAAGCTGGTGGAAAAGAGCTTC
GAGACAGACACCAACCTGAACTTCCAGAACCTCAGCGTGATCGGC
TTCCGGATCCTGCTGCTGAAGGTGGCCGGCTTCAACCTGCTGATG
ACCCTGCGGCTGTGGTCCAGCTGA
(SEQ ID NO: 249)
ATGGGCTGCAGGCTGCTCTGCTGTGCGGTTCTCTGTCTCCTGGGA
GCAGTTCCCATAGACACTGAAGTTACCCAGACACCAAAACACCTG
GTCATGGGAATGACAAATAAGAAGTCTTTGAAATGTGAACAACAT
ATGGGGCACAGGGCTATGTATTGGTACAAGCAGAAAGCTAAGAAG
CCACCGGAGCTCATGTTTGTCTACAGCTATGAGAAACTCTCTATA
AATGAAAGTGTGCCAAGTCGCTTCTCACCTGAATGCCCCAACAGC
TCTCTCTTAAACCTTCACCTACACGCCCTGCAGCCAGAAGACTCA
GCCCTGTATCTCTGCGCCAGCAGCCAAGGGACTAGCGGGGCAGAT
ACGCAGTATTTTGGCCCAGGCACCCGGCTGACAGTGCTCGAGGAC
CTGAAAAACGTGTTCCCACCCGAGGTCGCTGTGTTTGAGCCATCA
GAAGCAGAGATCTCCCACACCCAAAAGGCCACACTGGTGTGCCTG
GCCACAGGCTTCTACCCCGACCACGTGGAGCTGAGCTGGTGGGTG
AATGGGAAGGAGGTGCACAGTGGGGTCAGCACAGACCCGCAGCCC
CTCAAGGAGCAGCCCGCCCTCAATGACTCCAGATACTGCCTGAGC
AGCCGCCTGAGGGTCTCGGCCACCTTCTGGCAGAACCCCCGCAAC
CACTTCCGCTGTCAAGTCCAGTTCTACGGGCTCTCGGAGAATGAC
GAGTGGACCCAGGATAGGGCCAAACCTGTCACCCAGATCGTCAGC
GCCGAGGCCTGGGGTAGAGCAGACTGTGGCTTCACCTCCGAGTCT
TACCAGCAAGGGGTCCTGTCTGCCACCATCCTCTATGAGATCTTG
CTAGGGAAGGCCACCTTGTATGCCGTGCTGGTCAGTGCCCTCGTG
CTGATGGCCATGGTCAAGAGAAAGGATTCCAGAGGCTAG
(SEQ ID NO: 250)
ATGAGCATCGGCCTCCTGTGCTGTGCAGCCTTGTCTCTCCTGTGG
GCAGGTCCAGTGAATGCTGGTGTCACTCAGACCCCAAAATTCCAG
GTCCTGAAGACAGGACAGAGCATGACACTGCAGTGTGCCCAGGAT
ATGAACCATGAATACATGTCCTGGTATCGACAAGACCCAGGCATG
GGGCTGAGGCTGATTCATTACTCAGTTGGTGCTGGTATCACTGAC
CAAGGAGAAGTCCCCAATGGCTACAATGTCTCCAGATCAACCACA
GAGGATTTCCCGCTCAGGCTGCTGTCGGCTGCTCCCTCCCAGACA
TCTGTGTACTTCTGTGCCAGCAGTTACTCTCTTTGGGACCTTCAA
GAGACCCAGTACTTCGGGCCAGGCACGCGGCTCCTGGTGCTCGAG
GACCTGAAAAACGTGTTCCCACCCGAGGTCGCTGTGTTTGAGCCA
TCAGAAGCAGAGATCTCCCACACCCAAAAGGCCACACTGGTGTGC
CTGGCCACAGGCTTCTACCCCGACCACGTGGAGCTGAGCTGGTGG
GTGAATGGGAAGGAGGTGCACAGTGGGGTCAGCACAGACCCGCAG
CCCCTCAAGGAGCAGCCCGCCCTCAATGACTCCAGATACTGCCTG
AGCAGCCGCCTGAGGGTCTCGGCCACCTTCTGGCAGAACCCCCGC
AACCACTTCCGCTGTCAAGTCCAGTTCTACGGGCTCTCGGAGAAT
GACGAGTGGACCCAGGATAGGGCCAAACCTGTCACCCAGATCGTC
AGCGCCGAGGCCTGGGGTAGAGCAGACTGTGGCTTCACCTCCGAG
TCTTACCAGCAAGGGGTCCTGTCTGCCACCATCCTCTATGAGATC
TTGCTAGGGAAGGCCACCTTGTATGCCGTGCTGGTCAGTGCCCTC
GTGCTGATGGCCATGGTCAAGAGAAAGGATTCCAGAGGCTAG
(SEQ ID NO: 251)
ATGGGCACCAGCCTCCTCTGCTGGATGGCCCTGTGTCTCCTGGGG
GCAGATCACGCAGATACTGGAGTCTCCCAGGACCCCAGACACAAG
ATCACAAAGAGGGGACAGAATGTAACTTTCAGGTGTGATCCAATT
TCTGAACACAACCGCCTTTATTGGTACCGACAGACCCTGGGGCAG
GGCCCAGAGTTTCTGACTTACTTCCAGAATGAAGCTCAACTAGAA
AAATCAAGGCTGCTCAGTGATCGGTTCTCTGCAGAGAGGCCTAAG
GGATCTTTCTCCACCTTGGAGATCCAGCGCACAGAGCAGGGGGAC
TCGGCCATGTATCTCTGTGCCAGCAGCTTTTCAGACGGGGGGGCT
ACAGATACGCAGTATTTTGGCCCAGGCACCCGGCTGACAGTGCTC
GAGGACCTGAAAAACGTGTTCCCACCCGAGGTCGCTGTGTTTGAG
CCATCAGAAGCAGAGATCTCCCACACCCAAAAGGCCACACTGGTG
TGCCTGGCCACAGGCTTCTACCCCGACCACGTGGAGCTGAGCTGG
TGGGTGAATGGGAAGGAGGTGCACAGTGGGGTCAGCACAGACCCG
CAGCCCCTCAAGGAGCAGCCCGCCCTCAATGACTCCAGATACTGC
CTGAGCAGCCGCCTGAGGGTCTCGGCCACCTTCTGGCAGAACCCC
CGCAACCACTTCCGCTGTCAAGTCCAGTTCTACGGGCTCTCGGAG
AATGACGAGTGGACCCAGGATAGGGCCAAACCTGTCACCCAGATC
GTCAGCGCCGAGGCCTGGGGTAGAGCAGACTGTGGCTTCACCTCC
GAGTCTTACCAGCAAGGGGTCCTGTCTGCCACCATCCTCTATGAG
ATCTTGCTAGGGAAGGCCACCTTGTATGCCGTGCTGGTCAGTGCC
CTCGTGCTGATGGCCATGGTCAAGAGAAAGGATTCCAGAGGCTAG
(SEQ ID NO: 252)
ATGCTGCTGCTTCTGCTGCTTCTGGGGCCAGCAGGCTCCGGGCTT
GGTGCTGTCGTCTCTCAACATCCGAGCTGGGTTATCTGTAAGAGT
GGAACCTCTGTGAAGATCGAGTGCCGTTCCCTGGACTTTCAGGCC
ACAACTATGTTTTGGTATCGTCAGTTCCCGAAACAGAGTCTCATG
CTGATGGCAACTTCCAATGAGGGCTCCAAGGCCACATACGAGCAA
GGCGTCGAGAAGGACAAGTTTCTCATCAACCATGCAAGCCTGACC
TTGTCCACTCTGACAGTGACCAGTGCCCATCCTGAAGACAGCAGC
TTCTACATCTGCAGTGCTAGACCCCATTCTCTCACAGATACGCAG
TATTTTGGCCCAGGCACCCGGCTGACAGTGCTCGAGGACCTGAAA
AACGTGTTCCCACCCGAGGTCGCTGTGTTTGAGCCATCAGAAGCA
GAGATCTCCCACACCCAAAAGGCCACACTGGTGTGCCTGGCCACA
GGCTTCTACCCCGACCACGTGGAGCTGAGCTGGTGGGTGAATGGG
AAGGAGGTGCACAGTGGGGTCAGCACAGACCCGCAGCCCCTCAAG
GAGCAGCCCGCCCTCAATGACTCCAGATACTGCCTGAGCAGCCGC
CTGAGGGTCTCGGCCACCTTCTGGCAGAACCCCCGCAACCACTTC
CGCTGTCAAGTCCAGTTCTACGGGCTCTCGGAGAATGACGAGTGG
ACCCAGGATAGGGCCAAACCTGTCACCCAGATCGTCAGCGCCGAG
GCCTGGGGTAGAGCAGACTGTGGCTTCACCTCCGAGTCTTACCAG
CAAGGGGTCCTGTCTGCCACCATCCTCTATGAGATCTTGCTAGGG
AAGGCCACCTTGTATGCCGTGCTGGTCAGTGCCCTCGTGCTGATG
GCCATGGTCAAGAGAAAGGATTCCAGAGGCTAG
(SEQ ID NO: 253)
ATGGGCTGTAGACTGTTGTGTTGTGCTGTGCTGTGTCTGTTGGGA
GCTGTGCCTATCGATACAGAGGTGACCCAGACCCCTAAACATCTG
GTTATGGGCATGACCAACAAGAAGAGCCTGAAGTGCGAGCAGCAC
ATGGGCCATAGGGCCATGTATTGGTATAAGCAGAAGGCCAAGAAA
CCTCCTGAGCTGATGTTCGTGTACAGCTACGAGAAGCTGAGCATC
AACGAGAGCGTGCCCAGCAGATTTTCTCCTGAGTGCCCTAATTCT
AGCCTGCTGAATCTGCACCTGCATGCTCTGCAGCCTGAGGATTCT
GCTCTGTACCTGTGTGCTTCTTCTCAGGGCACATCTGGAGCTGAT
ACACAGTACTTCGGACCTGGCACAAGACTGACAGTGCTGGAAGAC
CTGAAGAACGTGTTCCCCCCAGAGGTGGCCGTGTTCGAGCCTAGC
GAGGCCGAGATCAGCCACACCCAGAAAGCCACCCTCGTGTGCCTG
GCCACCGGCTTTTACCCCGACCACGTGGAACTGTCTTGGTGGGTC
AACGGCAAAGAGGTGCACAGCGGCGTCTGCACCGACCCCCAGCCC
CTGAAAGAGCAGCCCGCCCTGAACGACAGCCGGTACTGTCTGAGC
AGCAGACTGAGAGTGTCCGCCACCTTCTGGCAGAACCCCCGGAAC
CACTTCAGATGCCAGGTGCAGTTCTACGGCCTGAGCGAGAACGAC
GAGTGGACCCAGGACCGGGCCAAGCCCGTGACCCAGATCGTGTCT
GCTGAGGCCTGGGGCAGAGCCGATTGCGGCTTCACCAGCGAGAGC
TACCAGCAGGGCGTGCTGAGCGCCACCATCCTGTACGAGATCCTG
CTGGGCAAGGCCACCCTGTACGCCGTGCTGGTGTCCGCCCTGGTG
CTGATGGCCATGGTCAAGCGGAAGGACAGCCGGGGC
(SEQ ID NO: 254)
ATGTCTATCGGTCTGCTGTGCTGTGCTGCTCTTTCTCTGCTTTGG
GCTGGACCTGTGAATGCTGGAGTTACACAAACCCCCAAGTTCCAA
GTGCTGAAGACAGGACAGAGCATGACCCTGCAGTGTGCTCAGGAC
ATGAATCACGAGTACATGAGCTGGTACAGACAGGATCCTGGAATG
GGCCTGAGGCTGATCCACTACTCTGTTGGAGCCGGAATTACAGAT
CAGGGAGAAGTGCCAAATGGCTACAACGTGAGCAGGAGCACAACC
GAGGACTTCCCCTTAAGACTGTTGTCTGCTGCTCCATCTCAGACA
AGCGTGTACTTTTGCGCCAGCTCCTACTCTCTGTGGGATCTGCAG
GAAACCCAGTACTTTGGACCAGGCACAAGACTGTTAGTGCTGGAG
GACCTGAAGAACGTGTTCCCCCCAGAGGTGGCCGTGTTCGAGCCT
AGCGAGGCCGAGATCAGCCACACCCAGAAAGCCACCCTCGTGTGC
CTGGCCACCGGCTTTTACCCCGACCACGTGGAACTGTCTTGGTGG
GTCAACGGCAAAGAGGTGCACAGCGGCGTCTGCACCGACCCCCAG
CCCCTGAAAGAGCAGCCCGCCCTGAACGACAGCCGGTACTGTCTG
AGCAGCAGACTGAGAGTGTCCGCCACCTTCTGGCAGAACCCCCGG
AACCACTTCAGATGCCAGGTGCAGTTCTACGGCCTGAGCGAGAAC
GACGAGTGGACCCAGGACCGGGCCAAGCCCGTGACCCAGATCGTG
TCTGCTGAGGCCTGGGGCAGAGCCGATTGCGGCTTCACCAGCGAG
AGCTACCAGCAGGGCGTGCTGAGCGCCACCATCCTGTACGAGATC
CTGCTGGGCAAGGCCACCCTGTACGCCGTGCTGGTGTCCGCCCTG
GTGCTGATGGCCATGGTCAAGCGGAAGGACAGCCGGGGC
(SEQ ID NO: 255)
ATGGGCACATCTCTTCTCTGCTGGATGGCTCTTTGTCTGCTTGGA
GCCGATCATGCCGATACAGGAGTTAGCCAGGATCCTAGACACAAG
ATCACCAAGAGAGGCCAGAATGTGACCTTCCGGTGCGATCCTATC
TCTGAGCACAACAGGCTGTACTGGTACAGACAAACACTGGGACAA
GGACCTGAGTTCCTGACCTACTTCCAGAACGAAGCCCAGCTGGAG
AAGTCTAGACTTCTGAGCGACAGATTTAGCGCCGAGAGACCTAAA
GGCAGCTTTAGCACCCTGGAGATCCAGAGAACAGAACAGGGCGAT
TCTGCCATGTACCTGTGTGCTAGCAGCTTTTCTGATGGAGGCGCC
ACCGATACACAGTATTTCGGACCTGGCACAAGACTGACAGTGCTG
GAGGACCTGAAGAACGTGTTCCCCCCAGAGGTGGCCGTGTTCGAG
CCTAGCGAGGCCGAGATCAGCCACACCCAGAAAGCCACCCTCGTG
TGCCTGGCCACCGGCTTTTACCCCGACCACGTGGAACTGTCTTGG
TGGGTCAACGGCAAAGAGGTGCACAGCGGCGTCTGCACCGACCCC
CAGCCCCTGAAAGAGCAGCCCGCCCTGAACGACAGCCGGTACTGT
CTGAGCAGCAGACTGAGAGTGTCCGCCACCTTCTGGCAGAACCCC
CGGAACCACTTCAGATGCCAGGTGCAGTTCTACGGCCTGAGCGAG
AACGACGAGTGGACCCAGGACCGGGCCAAGCCCGTGACCCAGATC
GTGTCTGCTGAGGCCTGGGGCAGAGCCGATTGCGGCTTCACCAGC
GAGAGCTACCAGCAGGGCGTGCTGAGCGCCACCATCCTGTACGAG
ATCCTGCTGGGCAAGGCCACCCTGTACGCCGTGCTGGTGTCCGCC
CTGGTGCTGATGGCCATGGTCAAGCGGAAGGACAGCCGGGGC
(SEQ ID NO: 256)
ATGCTGCTTCTTCTCCTCCTTCTCGGACCTGCTGGATCTGGATTA
GGAGCTGTTGTGTCTCAGCACCCTTCTTGGGTGATCTGTAAAAGC
GGCACAAGCGTGAAGATCGAGTGCAGAAGCCTGGACTTTCAGGCC
ACAACCATGTTCTGGTATAGGCAGTTCCCCAAGCAGTCTCTGATG
CTGATGGCCACCTCTAATGAGGGCTCTAAGGCCACATATGAACAG
GGAGTGGAGAAGGACAAGTTCCTGATCAACCACGCCTCTCTGACC
CTGTCTACCCTGACAGTTACATCTGCCCACCCTGAGGATAGCAGC
TTTTACATCTGTAGCGCCAGACCTCACAGCCTGACCGATACACAG
TACTTTGGCCCTGGCACAAGACTGACAGTGTTAGAAGACCTGAAG
AACGTGTTCCCCCCAGAGGTGGCCGTGTTCGAGCCTAGCGAGGCC
GAGATCAGCCACACCCAGAAAGCCACCCTCGTGTGCCTGGCCACC
GGCTTTTACCCCGACCACGTGGAACTGTCTTGGTGGGTCAACGGC
AAAGAGGTGCACAGCGGCGTCTGCACCGACCCCCAGCCCCTGAAA
GAGCAGCCCGCCCTGAACGACAGCCGGTACTGTCTGAGCAGCAGA
CTGAGAGTGTCCGCCACCTTCTGGCAGAACCCCCGGAACCACTTC
AGATGCCAGGTGCAGTTCTACGGCCTGAGCGAGAACGACGAGTGG
ACCCAGGACCGGGCCAAGCCCGTGACCCAGATCGTGTCTGCTGAG
GCCTGGGGCAGAGCCGATTGCGGCTTCACCAGCGAGAGCTACCAG
CAGGGCGTGCTGAGCGCCACCATCCTGTACGAGATCCTGCTGGGC
AAGGCCACCCTGTACGCCGTGCTGGTGTCCGCCCTGGTGCTGATG
GCCATGGTCAAGCGGAAGGACAGCCGGGGC
(SEQ ID NO: 257)
METLLGLLILWLQLQWVSSKQEVTQIPAALSVPEGENLVLNCSFT
DSAIYNLQWFRQDPGKGLTSLLLIQSSQREQTSGRLNASLDKSSG
RSTLYIAASQPGDSATYLCAVKETSGSRLTFGEGTQLTVNP
(SEQ ID NO: 258)
MTRVSLLWAVVVSTCLESGMAQTVTQSQPEMSVQEAETVTLSCTY
DTSENNYYLFWYKQPPSRQMILVIRQEAYKQQNATENRFSVNFQK
AAKSFSLKISDSQLGDTAMYFCAFIYPSYTSGTYKYIFGTGTRLK
VLAN
(SEQ ID NO: 259)
MAMLLGASVLILWLQPDWVNSQQKNDDQQVKQNSPSLSVQEGRIS
ILNCDYTNSMFDYFLWYKKYPAEGPTFLISISSIKDKNEDGRFTV
FLNKSAKHLSLHIVPSQPGDSAVYFCAASGIGGSYIPTFGRGTSL
IVHPY
(SEQ ID NO: 260)
MAMLLGASVLILWLQPDWVNSQQKNDDQQVKQNSPSLSVQEGRIS
ILNCDYTNSMEDYFLWYKKYPAEGPTFLISISSIKDKNEDGRFTV
FLNKSAKHLSLHIVPSQPGDSAVYFCAASGIGDYKLSFGAGTTVT
VRAN
(SEQ ID NO: 261)
MVKIRQFLLAILWLQLSCVSAAKNEVEQSPQNLTAQEGEFITINC
SYSVGISALHWLQQHPGGGIVSLEMLSSGKKKHGRLIATINIQEK
HSSLHITASHPRDSAVYICAVRTSYDKVIFGPGTSLSVIPN
(SEQ ID NO: 262)
MKSLRVLLVILWLQLSWVWSQQKEVEQNSGPLSVPEGAIASLNCT
YSDRGSQSFFWYRQYSGKSPELIMFIYSNGDKEDGRFTAQLNKAS
QYVSLLIRDSQPSDSATYLCAVNLLGATGYSTLTFGKGTMLLVSP
(SEQ ID NO: 263)
MWGVFLLYVSMKMGGTTGQNIDQPTEMTATEGAIVQINCTYQTSG
ENGLFWYQQHAGEAPTELSYNVLDGLEEKGRESSFLSRSKGYSYL
LLKELQMKDSASYLCAVRGINDYKLSFGAGTTVTVRAN
(SEQ ID NO: 264)
MEKMLECAFIVLWLQLGWLSGEDQVTQSPEALRLQEGESSSLNCS
YTVSGLRGLFWYRQDPGKGPEFLFTLYSAGEEKEKERLKATLTKK
ESFLHITAPKPEDSATYLCAVITGFQKLVFGTGTRLLVSPN
(SEQ ID NO: 265)
MRLVARVTVFLTFGTIIDAKTTQPTSMDCAEGRAANLPCNHSTIS
GNEYVYWYRQIHSQGPQYIIHGLKNNETNEMASLIITEDRKSSTL
ILPHATLRDTAVYYCIAGVGRGQNFVFGPGTRLSVLPY
(SEQ ID NO: 266)
MEKNPLAAPLLILWFHLDCVSSILNVEQSPQSLHVQEGDSTNFTC
SFPSSNFYALHWYRWETAKSPEALFVMTLNGDEKKKGRISATLNT
KEGYSYLYIKGSQPEDSATYLCAFHPNFGNEKLTFGTGTRLTIIP
N
(SEQ ID NO: 267)
MEKMLECAFIVLWLQLGWLSGEDQVTQSPEALRLQEGESSSLNCS
YTVSGLRGLFWYRQDPGKGPEFLFTLYSAGEEKEKERLKATLTKK
ESFLHITAPKPEDSATYLCAVQPRGDGSSNTGKLIFGQGTTLQVK
P
(SEQ ID NO: 268)
IQNPDPAVYQLRDSKSSDKSVCLFTDFDSQTNVSQSKDSDVYITD
KCVLDMRSMDFKSNSAVAWSNKSDFACANAFNNSIIPEDTFFPSP
ESSCDVKLVEKSFETDTNLNFQNLSVIGFRILLLKVAGFNLLMTL
RLWSS
(SEQ ID NO: 269)
MGTSLLCWVVLGFLGTDHTGAGVSQSPRYKVTKRGQDVALRCDPI
SGHVSLYWYRQALGQGPEFLTYFNYEAQQDKSGLPNDRFSAERPE
GSISTLTIQRTEQRDSAMYRCASSLTGSYEQYFGPGTRLTVTE
(SEQ ID NO: 270)
MLLLLLLLGPAGSGLGAVVSQHPSWVICKSGTSVKIECRSLDFQA
TTMFWYRQFPKQSLMLMATSNEGSKATYEQGVEKDKFLINHASLT
LSTLTVTSAHPEDSSFYICSATPEASSPYEQYFGPGTRLTVTE
(SEQ ID NO: 271)
MGPGLLHWMALCLLGTGHGDAMVIQNPRYQVTQFGKPVTLSCSQT
LNHNVMYWYQQKSSQAPKLLFHYYDKDENNEADTPDNFQSRRPNT
SFCFLDIRSPGLGDAAMYLCATSNLQGRQPQHFGDGTRLSILE
(SEQ ID NO: 272)
MLSPDLPDSAWNTRLLCHVMLCLLGAVSVAAGVIQSPRHLIKEKR
ETATLKCYPIPRHDTVYWYQQGPGQDPQFLISFYEKMQSDKGSIP
DRFSAQQFSDYHSELNMSSLELGDSALYFCASSLRLGRETQYFGP
GTRLLVLE
(SEQ ID NO: 273)
MGTRLLCWVVLGFLGTDHTGAGVSQSPRYKVAKRGQDVALRCDPI
SGHVSLFWYQQALGQGPEFLTYFQNEAQLDKSGLPSDRFFAERPE
GSVSTLKIQRTQQEDSAVYLCASSLGQAYEQYFGPGTRLTVTE
(SEQ ID NO: 274)
MGTRLLCWVAFCLLVEELIEAGVVQSPRYKIIEKKQPVAFWCNPI
SGHNTLYWYLQNLGQGPELLIRYENEEAVDDSQLPKDRESAERLK
GVDSTLKIQPAELGDSAVYLCASSLTRGAEAFFGQGTRLTVVE
(SEQ ID NO: 275)
MSNQVLCCVVLCFLGANTVDGGITQSPKYLFRKEGQNVTLSCEQN
LNHDAMYWYRQDPGQGLRLIYYSQIVNDFQKGDIAEGYSVSREKK
ESFPLTVTSAQKNPTAFYLCASSRDREQESPLHEGNGTRLTVTE
(SEQ ID NO: 276)
MGPQLLGYVVLCLLGAGPLEAQVTQNPRYLITVTGKKLTVTCSQN
MNHEYMSWYRQDPGLGLRQIYYSMNVEVTDKGDVPEGYKVSRKEK
RNFPLILESPSPNQTSLYFCASSESGGTYEQYFGPGTRLTVTE
(SEQ ID NO: 277)
MLSPDLPDSAWNTRLLCHVMLCLLGAVSVAAGVIQSPRHLIKEKR
ETATLKCYPIPRHDTVYWYQQGPGQDPQFLISFYEKMQSDKGSIP
DRFSAQQFSDYHSELNMSSLELGDSALYFCASSYRGGSTYEQYFG
PGTRLTVTE
(SEQ ID NO: 278)
MSTRLLCWMALCLLGAELSEAEVAQSPRYKITEKSQAVAFWCDPI
SGHATLYWYRQILGQGPELLVQFQDESVVDDSQLPKDRFSAERLK
GVDSTLKIQPAELGDSAMYLCASSQRDSPNEKLEFGSGTQLSVLE
(SEQ ID NO: 279)
MGCRLLCCAVLCLLGAVPMETGVTQTPRHLVMGMTNKKSLKCEQH
LGHNAMYWYKQSAKKPLELMFVYSLEERVENNSVPSRESPECPNS
SHLFLHLHTLQPEDSALYLCASSQDPYKLSGNTIYFGEGSWLTVV
E
(SEQ ID NO: 280)
DLNKVFPPEVAVFEPSEAEISHTQKATLVCLATGFFPDHVELSWW
VNGKEVHSGVCTDPQPLKEQPALNDSRYCLSSRLRVSATFWQNPR
NHFRCQVQFYGLSENDEWTQDRAKPVTQIVSAEAWGRADCGFTSV
SYQQGVLSATILYEILLGKATLYAVLVSALVLMAMVKRKDF
(SEQ ID NO: 281)
DLKNVFPPEVAVFEPSEAEISHTQKATLVCLATGFYPDHVELSWW
VNGKEVHSGVCTDPQPLKEQPALNDSRYCLSSRLRVSATFWQNPR
NHERCQVQFYGLSENDEWTQDRAKPVTQIVSAEAWGRADCGFTSE
SYQQGVLSATILYEILLGKATLYAVLVSALVLMAMVKRKDSRG
(SEQ ID NO: 282)
MKSLRVLLVILWLQLSWVWSQQKEVEQNSGPLSVPEGAIASLNCT
YSDRGSQSFFWYRQYSGKSPELIMFIYSNGDKEDGRFTAQLNKAS
QYVSLLIRDSQPSDSATYLCAVNIGNHDMRFGAGTRLTVKPN
(SEQ ID NO: 283)
MEKMLECAFIVLWLQLGWLSGEDQVTQSPEALRLQEGESSSLNCS
YTVSGLRGLFWYRQDPGKGPEFLFTLYSAGEEKEKERLKATLTKK
ESFLHITAPKPEDSATYLCAVQTMDGNQFYFGTGTSLTVIPN
(SEQ ID NO: 284)
MACPGFLWALVISTCLEFSMAQTVTQSQPEMSVQEAETVTLSCTY
DTSESDYYLFWYKQPPSRQMILVIRQEAYKQQNATENRFSVNFQK
AAKSFSLKISDSQLGDAAMYFCASSPGTYKYIFGTGTRLKVLAN
(SEQ ID NO: 285)
MTRVSLLWAVVVSTCLESGMAQTVTQSQPEMSVQEAETVTLSCTY
DTSESNYYLFWYKQPPSRQMILVIRQEAYKQQNATENRFSVNFQK
AAKSFSLKISDSQLGDTAMYFCAFNPWENYGQNEVFGPGIRLSVL
PY
(SEQ ID NO: 286)
IQNPDPAVYQLRDSKSSDKSVCLFTDFDSQTNVSQSKDSDVYITD
KCVLDMRSMDFKSNSAVAWSNKSDFACANAFNNSIIPEDTFFPSP
ESSCDVKLVEKSFETDTNLNFQNLSVIGFRILLLKVAGFNLLMTL
RLWSS
(SEQ ID NO: 287)
MGCRLLCCAVLCLLGAVPIDTEVTQTPKHLVMGMTNKKSLKCEQH
MGHRAMYWYKQKAKKPPELMFVYSYEKLSINESVPSRESPECPNS
SLLNLHLHALQPEDSALYLCASSQGTSGADTQYFGPGTRLTVLE
(SEQ ID NO: 288)
MSIGLLCCAALSLLWAGPVNAGVTQTPKFQVLKTGQSMTLQCAQD
MNHEYMSWYRQDPGMGLRLIHYSVGAGITDQGEVPNGYNVSRSTT
EDFPLRLLSAAPSQTSVYFCASSYSLWDLQETQYFGPGTRLLVLE
(SEQ ID NO: 289)
MGTSLLCWMALCLLGADHADTGVSQDPRHKITKRGQNVTFRCDPI
SEHNRLYWYRQTLGQGPEFLTYFQNEAQLEKSRLLSDRESAERPK
GSESTLEIQRTEQGDSAMYLCASSESDGGATDTQYFGPGTRLTVL
E
(SEQ ID NO: 290)
MLLLLLLLGPAGSGLGAVVSQHPSWVICKSGTSVKIECRSLDFQA
TTMFWYRQFPKQSLMLMATSNEGSKATYEQGVEKDKFLINHASLT
LSTLTVTSAHPEDSSFYICSARPHSLTDTQYFGPGTRLTVLE
(SEQ ID NO: 291)
DLKNVFPPEVAVFEPSEAEISHTQKATLVCLATGFYPDHVELSWW
VNGKEVHSGVCTDPQPLKEQPALNDSRYCLSSRLRVSATFWQNPR
NHFRCQVQFYGLSENDEWTQDRAKPVTQIVSAEAWGRADCGFTSE
SYQQGVLSATILYEILLGKATLYAVLVSALVLMAMVKRKDSRG

In some embodiments, a TCR construct comprises Human papilloma virus (HPV)-specific TCR chains. In some embodiments, a TCR construct comprising an HPV-specific TCR chains comprises TCR alpha and TCR beta chains that target the HPV 18 E6 protein, and/or HPV 18 E7 protein. In some embodiments, an HPV 18 E6 epitope is amino acids 121-135 and/or amino acids 77-91 of the HPV 18 E6 protein. In some embodiments, a TCR construct comprising an HPV-specific TCR chains comprises TCR alpha and TCR beta chains that target the HPV 18 E7 protein. In some embodiments, an HPV 18 E7 epitope is amino acids 11-19. In some embodiments, HPV-specific TCR sequences, TCR variable domain sequences, CDR sequences, and/or TCR constant domain sequences, are described in international patent application publications WO 2015/009604 A1, which is incorporated herein by reference for the purpose described herein.

### B. NK Cells

The NK cells that are modified to express the TCR/CD3 receptor complex may be obtained from any suitable source, including fresh or frozen. In certain embodiments, NK cells are derived from human peripheral blood mononuclear cells (PBMC), unstimulated leukapheresis products (PBSC), NK cell lines (e.g., NK-92), human embryonic stem cells (hESCs), induced pluripotent stem cells (iPSCs), bone marrow, or umbilical cord blood by methods well known in the art. Specifically, the NK cells may be isolated from cord blood (CB), peripheral blood (PB), bone marrow, stem cells, NK cell lines, or a mixture thereof. In particular embodiments, the NK cells are isolated from pooled CB. The CB may be pooled from 2, 3, 4, 5, 6, 7, 8, 9, 10, or more units. The NK cells may be autologous or allogeneic with respect to a recipient individual. The isolated NK cells may or may not be haplotype matched for the subject to be administered the cell therapy. NK cells can be detected by specific surface markers, such as CD16 and CD56 in humans, for example. In some cases, the source of the NK cells is cord blood and the NK cells may be in the cord blood in a heterogeneous mixture of cells and may be depleted of certain cells expressing CD3. In other methods, umbilical CB is used to derive NK cells by the isolation of CD34+ cells.

The NK cells may be pre-activated with one or more inflammatory cytokines, and they may be expanded or non-expanded. In some cases, the NK cells are pre-activated either prior to modification to express CD3±TCR or following modification to express CD3±TCR complex. In specific embodiments, pre-activation of the NK cells may comprise culturing the isolated NK cells in the presence of one or more cytokines. The NK cells may be stimulated with IL-2, or other cytokines that bind the common gamma-chain (e.g., IL-7, IL-12, IL-15, IL-18, IL-21, and others). In particular embodiments, the pre-activation cytokines may be selected from the group consisting of IL-12, IL-15, IL-18, and a combination thereof. One or more additional cytokines may be used for the pre-activation step. The pre-activation may be for a short period of time such as 5-72 hours, such as 10-50 hours, particularly 10-20 hours, such as 12, 13, 14, 15, 16, 17, 18, 19, or 20 hours, specifically about 16 hours. The pre-activation culture may comprise IL-12 at a concentration of 0.1-150 ng/mL, such as 0.5-50 ng/mL, particularly 1-20 ng/mL, such as 2, 3, 4, 5, 6, 7, 8, 9, 10, 11, 12, 13, 14, or 15 ng/mL, specifically about 10 ng/mL. The pre-activation culture may comprise IL-18 and/or IL-15 at a concentration of 10-100 ng/mL, such as 40-60 ng/mL, particular 45, 46, 47, 48, 49, 50, 51, 52, 53, 54, or 55 ng/mL, specifically about 50 ng/mL.

In some cases, the NK cells are expanded either prior to modification to express CD3±TCR complex or following modification to express CD3±TCR complex. Pre-activated NK cells may be expanded in the presence of artificial antigen presenting cells (aAPCs). The pre-activated NK cells may be washed prior to expansion, such as 2, 3, 4, or 5 times, specifically 3 times. The aAPCs may be engineered to express CD137 ligand and/or a membrane-bound cytokine. The membrane-bound cytokine may be membrane-bound IL-21 (mIL-21) or membrane-bound IL-15 (mIL-15). In particular embodiments, the aAPCs are engineered to express CD137 ligand and mIL-21. The aAPCs may be derived from cancer cells, such as leukemia cells. The aAPCs may not express endogenous HLA class I, II, or CD1d molecules. They may express ICAM-1 (CD54) and LFA-3 (CD58). In particular, the aAPCs may be K562 cells, such as K562 cells engineered to express CD137 ligand and mIL-21. The aAPCs may be irradiated. The engineering may be by any method known in the art, such as retroviral transduction. The expansion may be for about 2-30 days, such as 3-20 days, particularly 12-16 days, such as 12, 13, 14, 15, 16, 17, 18, or 19 days, specifically about 14 days. The pre-activated NK cells and aAPCs may be present at a ratio of about 3:1-1:3, such as 2:1, 1:1, 1:2, specifically about 1:2. The expansion culture may further comprise cytokines to promote expansion, such as IL-2. The IL-2 may be present at a concentration of about 10-500 U/mL, such as 100-300 U/mL, particularly about 200 U/mL. The IL-2 may be replenished in the expansion culture, such as every 2-3 days. The aAPCs may be added to the culture at least a second time, such as at about 7 days of expansion.

In particular embodiments, the NK cells are transfected or transduced with one or more membrane bound cytokines, including IL-21, IL-12, IL-18, IL-23, IL-7, or IL-15, either secreted by NK cells or tethered to the NK cell membrane. In such cases, the membrane bound cytokine may be tethered to the NK cell membrane with a particular transmembrane domain, such as the transmembrane domain of CD8, CD28, CD27, B7H3, IgG1, IgG4, CD4, DAP10, DAP12, for example.

Following preparation, the modified NK cells may be immediately infused (including with an effective amount of one or more bispecific or multi-specific antibodies, or the NK cells may be stored, such as by cryopreservation. In certain aspects, the cells may be propagated for days, weeks, or months ex vivo as a bulk population within about 1, 2, 3, 4, or 5 days.

### III. HETEROLOGOUS PROTEINS

In specific embodiments, the NK cells are modified not only to express one or more components of the TCR/CD3 complex, but they are also modified to express one or more other heterologous proteins. The heterologous proteins may facilitate activity of the NK cells in any manner, including at least their activation, persistence, expansion, homing, and/or cytotoxicity.

### A. Bispecific or Multi-Specific Antibodies

In some embodiments, the NK cells are modified to express one or more bispecific or multi-specific antibodies, although in other cases the NK cells do not express the antibodies but the antibodies are utilized in conjunction with the NK cells.

In cases wherein the NK cells are modified to express the antibodies, the antibodies may be engagers that bridge a particular immune effector cell with a particular target cell for destruction of the target cell. The present disclosure allows the modified NK cells to be used with standard T-cell engagers (BiTEs) because they have been modified to express CD3 that in many cases is the T cell antigen to which the BiTE engager binds. In such cases, the BiTE used in the invention may also target a cancer or viral antigen that may be tailored to the medical condition of an intended recipient individual. For example, the BiTE may be tailored to bind a cancer antigen that is characteristic of the cancer cells of a cancer of the individual. The anti-CD3 antibody of the BiTE may target the CD3γ chain, CD3δ chain, CD3ε chain, or CD3ζ chain.

In some cases, in addition to expressing the CD3 complex (with or without TCR) that allows the NK cells to be utilized as a therapy with BiTEs, the NK cells may be modified to express (or not to express but instead used in conjunction with) one or more bispecific NK engagers (BiKEs). The BiKE comprises an antibody that binds a surface protein on the NK cell, including a naturally expressed surface protein on NK cells, and also comprises an antibody that binds a desired target antigen. The BiKE may target the NK cells through an antibody an NK surface protein such as CD16, CS1, CD56, NKG2D, NKG2C, DNAM, 2B4, CD2, an NCR, or KIR, for example. In such cases, the BiKE used in the invention may also target a cancer or viral antigen that may be tailored to the medical condition of an intended recipient individual. For example, the BiKE may be tailored to bind a cancer antigen that is characteristic of the cancer cells of a cancer of the individual.

In embodiments wherein an NK cell expresses the CD3 complex (with or without TCR) and one or more BiKEs, one or more vectors may be utilized to transfect or transduce the cells with the CD3 complex components (with or without TCR) and one or more BiKEs. In some cases, one or more of the CD3 complex components (with or without TCR) and the BiKE may or may not be on the same multicistronic vector.

### B. Engineered Receptors

In specific embodiments, the NK cells are engineered to express one or more engineered receptors. In some cases, the engineered receptors are engineered antigen receptors that target a cancer or viral antigen of any kind. The receptor may be tailored to target a desired antigen based on a medical condition of an intended recipient individual.

In some embodiments, the engineered antigen receptor is a chimeric antigen receptor (CAR). The NK cells may be modified to encode at least one CAR, and the CAR may be first generation, second generation, or third or a subsequent generation, for example. The CAR may or may not be bispecific for two or more different antigens. The CAR may comprise one or more costimulatory domains. Each costimulatory domain may comprise the costimulatory domain of any one or more of, for example, members of the TNFR superfamily, CD28, CD137 (4-1BB), CD134 (OX40), DAP10, DAP12, CD27, CD2, CD5, ICAM-1, LFA-1 (CD11a/CD18), Lck, TNFR-I, TNFR-II, Fas, CD30, CD27, NKG2D, 2B4M, CD40 or combinations thereof, for example. In specific embodiments, the CAR comprises CD3zeta. In certain embodiments, the CAR lacks one or more specific costimulatory domains; for example, the CAR may lack 4-1BB and/or lack CD28.

In particular embodiments, the CAR polypeptide in the cells comprises an extracellular spacer domain that links the antigen binding domain and the transmembrane domain, and this may be referred to as a hinge. Extracellular spacer domains may include, but are not limited to, Fc fragments of antibodies or fragments or derivatives thereof, hinge regions of antibodies or fragments or derivatives thereof, CH2 regions of antibodies, CH3 regions antibodies, artificial spacer sequences or combinations thereof. Examples of extracellular spacer domains include but are not limited to CD8-alpha hinge, CD28, artificial spacers made of polypeptides such as Gly3, or CH1, CH3 domains of IgGs (such as human IgG1 or IgG4). In specific cases, the extracellular spacer domain may comprise (i) a hinge, CH2 and CH3 regions of IgG4, (ii) a hinge region of IgG4, (iii) a hinge and CH2 of IgG4, (iv) a hinge region of CD8-alpha or CD4, (v) a hinge, CH2 and CH3 regions of IgG1, (vi) a hinge region of IgG1 or (vii) a hinge and CH2 of IgG1, (viii) a hinge region of CD28, or a combination thereof. In specific embodiments, the hinge is from IgG1 and in certain aspects the CAR polypeptide comprises a particular IgG1 hinge amino acid sequence or is encoded by a particular IgG1 hinge nucleic acid sequence.

The transmembrane domain in the CAR may be derived either from a natural or from a synthetic source. Where the source is natural, the domain in some aspects is derived from any membrane-bound or transmembrane protein. Transmembrane regions include those derived from (i.e., comprise at least the transmembrane region(s) of) the alpha, beta or zeta chain of the T− cell receptor, CD28, CD3 zeta, CD3 epsilon, CD3 gamma, CD3 delta, CD45, CD4, CD5, CD8, CD9, CD 16, CD22, CD33, CD37, CD64, CD80, CD86, CD 134, CD137, CD154, ICOS/CD278, GITR/CD357, NKG2D, and DAP molecules, such as DAP10 or DAP12. Alternatively the transmembrane domain in some embodiments is synthetic. In some aspects, the synthetic transmembrane domain comprises predominantly hydrophobic residues such as leucine and valine. In some aspects, a triplet of phenylalanine, tryptophan and valine may be found at each end of a synthetic transmembrane domain.

In some embodiments, the engineered receptors utilize one or more homing receptors (that can home to a target not necessarily because of a signal release, such as in the event that they utilize adhesion molecules) and/or one or more chemokine receptors. Examples of chemokine receptors include CXC chemokine receptors, CC chemokine receptors, CX3C chemokine receptors and XC chemokine receptors. In specific cases, the chemokine receptor is a receptor for CCR2, CCR3, CCR5, CCR8, CCR7, CXCR3, L-selectin (CD62L) CXCR1, CXCR2, or CX3CR1.

### C. Cytokines

In some embodiments, the cells expressing the NK cells are engineered to express one or more heterologous cytokines and/or are engineered to upregulate normal expression of one or more heterologous cytokines. The cells may or may not be transduced or transfected for one or more cytokines on the same vector as other genes.

One or more cytokines may be co-expressed from a vector, including as a separate polypeptide from any component of the TCR/CD3 complex. Interleukin-15 (IL-15), for example, is tissue restricted and only under pathologic conditions is it observed at any level in the serum, or systemically. IL-15 possesses several attributes that are desirable for adoptive therapy. IL-15 is a homeostatic cytokine that induces development and cell proliferation of natural killer cells, promotes the eradication of established tumors via alleviating functional suppression of tumor-resident cells, and inhibits activation-induced cell death (AICD). In addition to IL-15, other cytokines are envisioned. These include, but are not limited to, cytokines, chemokines, and other molecules that contribute to the activation and proliferation of cells used for human application. NK cells expressing IL-15 are capable of continued supportive cytokine signaling, which is useful for their survival post-infusion.

In specific embodiments, the cells express one or more exogenously provided cytokines. As one example, the cytokine is IL-15, IL-12, IL-2, IL-18, IL-21, IL-23, GMCSF, or a combination thereof. The cytokine may be exogenously provided to the NK cells because it is expressed from an expression vector within the cell. In an alternative case, an endogenous cytokine in the cell is upregulated upon manipulation of regulation of expression of the endogenous cytokine, such as genetic recombination at the promoter site(s) of the cytokine. In cases wherein the cytokine is provided on an expression construct to the cell, the cytokine may be encoded from the same vector as one or more components of the CD3 complex with or without the TCR complex.

In some embodiments, a specific sequence of IL-15 is utilized, such as those that follow (underlining refers to signal peptide sequence):

(SEQ ID NO: 49)
ATGCGCATTAGCAAGCCCCACCTGCGGAGCATCAGCATCCAGTGC
TACCTGTGCCTGCTGCTGAACAGCCACTTCCTGACCGAGGCCGGC
ATCCACGTGTTCATCCTGGGCTGCTTCAGCGCCGGACTGCCCAAG
ACCGAGGCCAACTGGGTGAACGTGATCAGCGACCTGAAGAAGATC
GAGGACCTGATCCAGAGCATGCACATCGACGCCACCCTGTACACC
GAGAGCGACGTGCACCCCAGCTGCAAGGTGACCGCCATGAAGTGC
TTTCTGCTGGAACTGCAGGTGATCAGCCTGGAAAGCGGCGACGCC
AGCATCCACGACACCGTGGAGAACCTGATCATCCTGGCCAACAAC
AGCCTGAGCAGCAACGGCAACGTGACCGAGAGCGGCTGCAAAGAG
TGCGAGGAACTGGAAGAGAAGAACATCAAAGAGTTTCTGCAGAGC
TTCGTGCACATCGTGCAGATGTTCATCAACACCAGC
(SEQ ID NO: 48)
MRISKPHLRSISIQCYLCLLLNSHELTEAGIHVFILGCFSAGLPK
TEANWVNVISDLKKIEDLIQSMHIDATLYTESDVHPSCKVTAMKC
FLLELQVISLESGDASIHDTVENLIILANNSLSSNGNVTESGCKE
CEELEEKNIKEFLQSFVHIVOMFINTS

### D. Antigens

The modified NK cells of the disclosure are utilized with bispecific or multi-specific antibodies that target one or more particular antigens. In addition, the NK cells may be modified with engineered antigen receptors that target one or more particular antigens. In cases wherein the NK cells are modified with one or more engineered antigen receptors, the antigen targeted by the bispecific or multi-specific antibody, and the antigen targeted by the one or more engineered antigen receptors may or may not be the same antigen. In some cases, the antigen targeted by the bispecific or multi-specific antibody, and the antigen targeted by the one or more engineered antigen receptors are different antigens but are associated with the same type of cancer.

Among the antigens targeted by the antibodies and/or engineered antigen receptors are those expressed in the context of a disease, condition, or cell type to be targeted via the adoptive cell therapy. Among the diseases and conditions are proliferative, neoplastic, and malignant diseases and disorders, including cancers and tumors, including hematologic cancers, cancers of the immune system, such as lymphomas, leukemias, and/or myelomas, such as B, T, and myeloid leukemias, lymphomas, and multiple myelomas. In some embodiments, the antigen is selectively expressed or overexpressed on cells of the disease or condition, e.g., the tumor or pathogenic cells, as compared to normal or non-targeted cells or tissues. In other embodiments, the antigen is expressed on normal cells and/or is expressed on the engineered cells.

Any suitable antigen may be targeted in the present method. The antigen may be associated with certain cancer cells but not associated with non-cancerous cells, in some cases. Exemplary antigens include, but are not limited to, antigenic molecules from infectious agents, auto-/self-antigens, tumor-/cancer-associated antigens, and tumor neoantigens (Linnemann et al., 2015). In particular aspects, the antigens include NY-ESO, CD19, EBNA, CD123, HER2, CA-125, TRAIL/DR4, CD20, CD22, CD70, CD38, CD123, CLL1, carcinoembryonic antigen, alphafetoprotein, CD56, AKT, Her3, epithelial tumor antigen, CD319 (CS1), ROR1, folate binding protein, HIV-1 envelope glycoprotein gp120, HIV-1 envelope glycoprotein gp41, CD5, CD23, CD30, HERV-K, IL-11Ralpha, kappa chain, lambda chain, CSPG4, CD33, CD47, CLL-1, U5snRNP200, CD200, BAFF-R, BCMA, CD99, p53, mutated p53, Ras, mutated ras, c-Myc, cytoplasmic serine/threonine kinases (e.g., A-Raf, B-Raf, and C-Raf, cyclin-dependent kinases), MAGE-A1, MAGE-A2, MAGE-A3, MAGE-A4, MAGE-A6, MAGE-A10, MAGE-A12, MART-1, melanoma-associated antigen, BAGE, DAM-6, -10, GAGE-1, -2, -8, GAGE-3, -4, -5, -6, -7B, NA88-A, MC1R, mda-7, gp75, Gp100, PSA, PSM, Tyrosinase, tyrosinase-related protein, TRP-1, TRP-2, ART-4, CAMEL, CEA, Cyp-B, hTERT, hTRT, iCE, MUC1, MUC2, Phosphoinositide 3-kinases (PI3Ks), TRK receptors, PRAME, P15, RU1, RU2, SART-1, SART-3, Wilms' tumor antigen (WT1), AFP, -catenin/m, Caspase-8/m, CDK-4/m, ELF2M, GnT-V, G250, HAGE, HSP70-2M, HST-2, KIAA0205, MUM-1, MUM-2, MUM-3, Myosin/m, RAGE, SART-2, TRP-2/INT2, 707-AP, Annexin II, CDC27/m, TPI/mbcr-abl, BCR-ABL, interferon regulatory factor 4 (IRF4), ETV6/AML, LDLR/FUT, Pml/RAR, Tumor-associated calcium signal transducer 1 (TACSTD1) TACSTD2, receptor tyrosine kinases (e.g., Epidermal Growth Factor receptor (EGFR) (in particular, EGFRvIII), platelet derived growth factor receptor (PDGFR), vascular endothelial growth factor receptor (VEGFR)), VEGFR2, cytoplasmic tyrosine kinases (e.g., src-family, syk-ZAP70 family), integrin-linked kinase (TLK), signal transducers and activators of transcription STAT3, STATS, and STATE, hypoxia inducible factors (e.g., HIF-1 and HIF-2), Nuclear Factor-Kappa B (NF-B), Notch receptors (e.g., Notch1-4), NY ESO 1, c-Met, mammalian targets of rapamycin (mTOR), WNT, extracellular signal-regulated kinases (ERKs), and their regulatory subunits, PMSA, PR-3, MDM2, Mesothelin, renal cell carcinoma-5T4, SM22-alpha, carbonic anhydrases I (CAI) and IX (CAIX) (also known as G250), STEAD, TEL/AML1, GD2, proteinase3, hTERT, sarcoma translocation breakpoints, EphA2, ML-IAP, EpCAM, ERG (TMPRSS2 ETS fusion gene), NA17, PAX3, ALK, androgen receptor, cyclin B1, polysialic acid, MYCN, RhoC, GD3, fucosyl GM1, mesothelian, PSCA, sLe, PLAC1, GM3, BORIS, Tn, GLoboH, NY-BR-1, RGsS, SAGE, SART3, STn, PAX5, OY-TES1, sperm protein 17, LCK, HMWMAA, AKAP-4, SSX2, XAGE 1, B7H3, legumain, TIE2, Page4, MAD-CT-1, FAP, MAD-CT-2, fos related antigen 1, CBX2, CLDN6, SPANX, TPTE, ACTL8, ANKRD30A, CDKN2A, MAD2L1, CTAGIB, SUNC1, and LRRN1. Examples of sequences for antigens are known in the art, for example, in the GENBANK® database: CD19 (Accession No. NG_007275.1), EBNA (Accession No. NG_002392.2), WT1 (Accession No. NG_009272.1), CD123 (Accession No. NC_000023.11), NY-ESO (Accession No. NC_000023.11), EGFRvIII (Accession No. NG_007726.3), MUC1 (Accession No. NG_029383.1), HER2 (Accession No. NG_007503.1), CA-125 (Accession No. NG_055257.1), WT1 (Accession No. NG_009272.1), Mage-A3 (Accession No. NG_013244.1), Mage-A4 (Accession No. NG_013245.1), Mage-A10 (Accession No. NC_000023.11), TRAIL/DR4 (Accession No. NC_000003.12), and/or CEA (Accession No. NC_000019.10).

Tumor-associated antigens may be derived from prostate, breast, colorectal, lung, pancreatic, renal, mesothelioma, ovarian, liver, brain, bone, stomach, spleen, testicular, cervical, anal, gall bladder, thyroid, or melanoma cancers, as examples. Exemplary tumor-associated antigens or tumor cell-derived antigens include MAGE 1, 3, and MAGE 4 (or other MAGE antigens such as those disclosed in International Patent Publication No. WO 99/40188); PRAME; BAGE; RAGE, Lage (also known as NY ESO 1); SAGE; and HAGE or GAGE. These non-limiting examples of tumor antigens are expressed in a wide range of tumor types such as melanoma, lung carcinoma, sarcoma, and bladder carcinoma. See, e.g., U.S. Pat. No. 6,544,518. Prostate cancer tumor-associated antigens include, for example, prostate specific membrane antigen (PSMA), prostate-specific antigen (PSA), prostatic acid phosphates, NKX3.1, and six-transmembrane epithelial antigen of the prostate (STEAP).

Other tumor associated antigens include Plu-1, HASH-1, HasH-2, Cripto and Criptin. Additionally, a tumor antigen may be a self-peptide hormone, such as whole length gonadotrophin hormone releasing hormone (GnRH), a short 10 amino acid long peptide, useful in the treatment of many cancers.

Antigens may include epitopic regions or epitopic peptides derived from genes mutated in tumor cells or from genes transcribed at different levels in tumor cells compared to normal cells, such as telomerase enzyme, survivin, mesothelin, mutated ras, bcr/abl rearrangement, Her2/neu, mutated or wild-type p53, cytochrome P450 1B1, and abnormally expressed intron sequences such as N-acetylglucosaminyltransferase-V; clonal rearrangements of immunoglobulin genes generating unique idiotypes in myeloma and B-cell lymphomas; tumor antigens that include epitopic regions or epitopic peptides derived from oncoviral processes, such as human papilloma virus proteins E6 and E7; Epstein bar virus protein LMP2; nonmutated oncofetal proteins with a tumor-selective expression, such as carcinoembryonic antigen and alpha-fetoprotein.

### E. Suicide Gene

In particular embodiments, a suicide gene is utilized in conjunction with the NK cell therapy to control its use and allow for termination of the cell therapy at a desired event and/or time. The suicide gene is employed in transduced cells for the purpose of eliciting death for the transduced cells when needed. The cells of the present disclosure that have been modified to harbor one or more vectors encompassed by the disclosure that may comprise one or more suicide genes. In some embodiments, the term “suicide gene” as used herein is defined as a gene which, upon administration of a prodrug or other agent, effects transition of a gene product to a compound which kills its host cell. In other embodiments, a suicide gene encodes a gene product that is, when desired, targeted by an agent (such as an antibody) that targets the suicide gene product.

In some cases, the cell therapy may be subject to utilization of one or more suicide genes of any kind when an individual receiving the cell therapy and/or having received the cell therapy shows one or more symptoms of one or more adverse events, such as cytokine release syndrome, neurotoxicity, anaphylaxis/allergy, and/or on-target/off tumor toxicities (as examples) or is considered at risk for having the one or more symptoms, including imminently. The use of the suicide gene may be part of a planned protocol for a therapy or may be used only upon a recognized need for its use. In some cases the cell therapy is terminated by use of agent(s) that targets the suicide gene or a gene product therefrom because the therapy is no longer required.

Utilization of the suicide gene may be instigated upon onset of at least one adverse event for the individual, and that adverse event may be recognized by any means, including upon routine monitoring that may or may not be continuous from the beginning of the cell therapy. The adverse event(s) may be detected upon examination and/or testing. In cases wherein the individual has cytokine release syndrome (which may also be referred to as cytokine storm), the individual may have elevated inflammatory cytokine(s) (merely as examples: interferon-gamma, granulocyte macrophage colony-stimulating factor, IL-10, IL-6 and TNF-alpha); fever; fatigue; hypotension; hypoxia, tachycardia; nausea; capillary leak; cardiac/renal/hepatic dysfunction; or a combination thereof, for example. In cases wherein the individual has neurotoxicity, the individual may have confusion, delirium, aplasia, and/or seizures. In some cases, the individual is tested for a marker associated with onset and/or severity of cytokine release syndrome, such as C-reactive protein, IL-6, TNF-alpha, and/or ferritin.

Examples of suicide genes include engineered nonsecretable (including membrane bound) tumor necrosis factor (TNF)-alpha mutant polypeptides (see PCT/US19/62009, which is incorporated by reference herein in its entirety), and they may be affected by delivery of an antibody that binds the TNF-alpha mutant. Examples of suicide gene/prodrug combinations that may be used are Herpes Simplex Virus-thymidine kinase (HSV-tk) and ganciclovir, acyclovir, or FIAU; oxidoreductase and cycloheximide; cytosine deaminase and 5-fluorocytosine; thymidine kinase thymidylate kinase (Tdk::Tmk) and AZT; and deoxycytidine kinase and cytosine arabinoside. The E. coli purine nucleoside phosphorylase, a so-called suicide gene that converts the prodrug 6-methylpurine deoxyriboside to toxic purine 6-methylpurine, may be utilized. Other suicide genes include CD20, CD52, inducible caspase 9, purine nucleoside phosphorylase (PNP), Cytochrome p450 enzymes (CYP), Carboxypeptidases (CP), Carboxylesterase (CE), Nitroreductase (NTR), Guanine Ribosyltransferase (XGRTP), Glycosidase enzymes, Methionine-α,γ-lyase (MET), EGFRv3, and Thymidine phosphorylase (TP), as examples.

### IV. ADMINISTRATION OF THERAPEUTIC COMPOSITIONS

The CD3-expressing NK cells and the bispecific or multi-specific antibodies are administered to an individual in need thereof, including in such a way as to be in proximity for the anti-CD3 antibody of the bispecific or multi-specific antibody to be able to bind CD3 on the CD3-expressing NK cells. In some cases, the two components are administered separately to an individual, whereas in other cases the two components are complexed together prior to administration, such as in an ex vivo manner. In another embodiment, the NK cells express the antibodies. In some cases, the two components are not pre-complexed prior to administration, but are co-administered by any suitable route of administration, such as by co-infusion to the patient.

Embodiments of the present disclosure concern methods for the use of the compositions comprising NK cells and antibodies provided herein for treating or preventing a medical disease or disorder. The method includes administering to the subject a therapeutically effective amount of the CD3 (±TCR)-modified NK cells with the antibodies, thereby treating or preventing the disease in the subject, including reducing the risk of, reducing the severity of, and/or delaying the onset of the disease. In certain embodiments of the present disclosure, cancer or infection is treated by transfer of a composition comprising the NK cell population and corresponding antibodies. In at least some cases, because of their release of pro-inflammatory cytokines, NK cells may reverse the anti-inflammatory tumor microenvironment and increase adaptive immune responses by promoting differentiation, activation, and/or recruitment of accessory immune cell to sites of malignancy.

Cancers for which the present treatment methods are useful include any malignant cell type, such as those found in a solid tumor or a hematological tumor. Exemplary solid tumors can include, but are not limited to, a tumor of an organ selected from the group consisting of pancreas, colon, cecum, stomach, brain, head, neck, ovary, kidney, larynx, sarcoma, lung, bladder, melanoma, prostate, and breast. Exemplary hematological tumors include tumors of the bone marrow, T or B cell malignancies, leukemias, lymphomas, blastomas, myelomas, and the like. Further examples of cancers that may be treated using the methods provided herein include, but are not limited to, lung cancer (including small-cell lung cancer, non-small cell lung cancer, adenocarcinoma of the lung, and squamous carcinoma of the lung), cancer of the peritoneum, gastric or stomach cancer (including gastrointestinal cancer and gastrointestinal stromal cancer), pancreatic cancer, cervical cancer, ovarian cancer, liver cancer, bladder cancer, breast cancer, colon cancer, colorectal cancer, endometrial or uterine carcinoma, salivary gland carcinoma, kidney or renal cancer, prostate cancer, vulval cancer, thyroid cancer, various types of head and neck cancer, and melanoma.

The cancer may specifically be of the following histological type, though it is not limited to these: neoplasm, malignant; carcinoma; carcinoma, undifferentiated; giant and spindle cell carcinoma; small cell carcinoma; papillary carcinoma; squamous cell carcinoma; lymphoepithelial carcinoma; basal cell carcinoma; pilomatrix carcinoma; transitional cell carcinoma; papillary transitional cell carcinoma; adenocarcinoma; gastrinoma, malignant; cholangiocarcinoma; hepatocellular carcinoma; combined hepatocellular carcinoma and cholangiocarcinoma; trabecular adenocarcinoma; adenoid cystic carcinoma; adenocarcinoma in adenomatous polyp; adenocarcinoma, familial polyposis coli; solid carcinoma; carcinoid tumor, malignant; branchiolo-alveolar adenocarcinoma; papillary adenocarcinoma; chromophobe carcinoma; acidophil carcinoma; oxyphilic adenocarcinoma; basophil carcinoma; clear cell adenocarcinoma; granular cell carcinoma; follicular adenocarcinoma; papillary and follicular adenocarcinoma; nonencapsulating sclerosing carcinoma; adrenal cortical carcinoma; endometroid carcinoma; skin appendage carcinoma; apocrine adenocarcinoma; sebaceous adenocarcinoma; ceruminous adenocarcinoma; mucoepidermoid carcinoma; cystadenocarcinoma; papillary cystadenocarcinoma; papillary serous cystadenocarcinoma; mucinous cystadenocarcinoma; mucinous adenocarcinoma; signet ring cell carcinoma; infiltrating duct carcinoma; medullary carcinoma; lobular carcinoma; inflammatory carcinoma; paget's disease, mammary; acinar cell carcinoma; adenosquamous carcinoma; adenocarcinoma w/squamous metaplasia; thymoma, malignant; ovarian stromal tumor, malignant; thecoma, malignant; granulosa cell tumor, malignant; androblastoma, malignant; sertoli cell carcinoma; leydig cell tumor, malignant; lipid cell tumor, malignant; paraganglioma, malignant; extra-mammary paraganglioma, malignant; pheochromocytoma; glomangiosarcoma; malignant melanoma; amelanotic melanoma; superficial spreading melanoma; lentigo malignant melanoma; acral lentiginous melanomas; nodular melanomas; malignant melanoma in giant pigmented nevus; epithelioid cell melanoma; blue nevus, malignant; sarcoma; fibrosarcoma; fibrous histiocytoma, malignant; myxosarcoma; liposarcoma; leiomyosarcoma; rhabdomyosarcoma; embryonal rhabdomyosarcoma; alveolar rhabdomyosarcoma; stromal sarcoma; mixed tumor, malignant; mullerian mixed tumor; nephroblastoma; hepatoblastoma; carcinosarcoma; mesenchymoma, malignant; brenner tumor, malignant; phyllodes tumor, malignant; synovial sarcoma; mesothelioma, malignant; dysgerminoma; embryonal carcinoma; teratoma, malignant; struma ovarii, malignant; choriocarcinoma; mesonephroma, malignant; hemangiosarcoma; hemangioendothelioma, malignant; kaposi's sarcoma; hemangiopericytoma, malignant; lymphangiosarcoma; osteosarcoma; juxtacortical osteosarcoma; chondrosarcoma; chondroblastoma, malignant; mesenchymal chondrosarcoma; giant cell tumor of bone; ewing's sarcoma; odontogenic tumor, malignant; ameloblastic odontosarcoma; ameloblastoma, malignant; ameloblastic fibrosarcoma; pinealoma, malignant; chordoma; glioma, malignant; ependymoma; astrocytoma; protoplasmic astrocytoma; fibrillary astrocytoma; astroblastoma; glioblastoma; oligodendroglioma; oligodendroblastoma; primitive neuroectodermal; cerebellar sarcoma; ganglioneuroblastoma; neuroblastoma; retinoblastoma; olfactory neurogenic tumor; meningioma, malignant; neurofibrosarcoma; neurilemmoma, malignant; granular cell tumor, malignant; malignant lymphoma; hodgkin's disease; hodgkin's; paragranuloma; malignant lymphoma, small lymphocytic; malignant lymphoma, large cell, diffuse; malignant lymphoma, follicular; mycosis fungoides; other specified non-hodgkin's lymphomas; B-cell lymphoma; low grade/follicular non-Hodgkin's lymphoma (NHL); small lymphocytic (SL) NHL; intermediate grade/follicular NHL; intermediate grade diffuse NHL; high grade immunoblastic NHL; high grade lymphoblastic NHL; high grade small non-cleaved cell NHL; bulky disease NHL; mantle cell lymphoma; AIDS-related lymphoma; Waldenstrom's macroglobulinemia; malignant histiocytosis; multiple myeloma; mast cell sarcoma; immunoproliferative small intestinal disease; leukemia; lymphoid leukemia; plasma cell leukemia; erythroleukemia; lymphosarcoma cell leukemia; myeloid leukemia; basophilic leukemia; eosinophilic leukemia; monocytic leukemia; mast cell leukemia; megakaryoblastic leukemia; myeloid sarcoma; hairy cell leukemia; chronic lymphocytic leukemia (CLL); acute lymphoblastic leukemia (ALL); acute myeloid leukemia (AMIL); and chronic myeloblastic leukemia.

The therapy provided herein may comprise administration of a combination of therapeutic agents, such as a first cancer therapy and a second cancer therapy. The therapies may be administered in any suitable manner known in the art. For example, the first and second cancer treatment may be administered sequentially (at different times) or concurrently (at the same time). In some embodiments, the first and second cancer treatments are administered in a separate composition. In some embodiments, the first and second cancer treatments are in the same composition. Embodiments of the disclosure relate to compositions and methods comprising therapeutic compositions. The different therapies may be administered in one composition or in more than one composition, such as 2 compositions, 3 compositions, or 4 compositions. Various combinations of the agents may be employed. Examples of therapies other than those of the present disclosure include surgery, chemotherapy, drug therapy, radiation, hormone therapy, immunotherapy (other than that of the present disclosure), or a combination thereof.

The therapeutic agents of the disclosure may be administered by the same route of administration or by different routes of administration. In some embodiments, the cancer therapy is administered intravenously, intramuscularly, subcutaneously, topically, orally, transdermally, intraperitoneally, intraorbitally, by implantation, by inhalation, intrathecally, intraventricularly, or intranasally. In some embodiments, the antibiotic is administered intravenously, intramuscularly, subcutaneously, topically, orally, transdermally, intraperitoneally, intraorbitally, by implantation, by inhalation, intrathecally, intraventricularly, or intranasally. The appropriate dosage may be determined based on the type of disease to be treated, severity and course of the disease, the clinical condition of the individual, the individual's clinical history and response to the treatment, and the discretion of the attending physician.

The treatments may include various “unit doses.” Unit dose is defined as containing a predetermined-quantity of the therapeutic composition. The quantity to be administered, and the particular route and formulation, is within the skill of determination of those in the clinical arts. A unit dose need not be administered as a single injection but may comprise continuous infusion over a set period of time. In some embodiments, a unit dose comprises a single administrable dose.

The quantity to be administered, both according to number of treatments and unit dose, depends on the treatment effect desired. An effective dose is understood to refer to an amount necessary to achieve a particular effect. In the practice in certain embodiments, it is contemplated that doses in the range from 10 mg/kg to 200 mg/kg can affect the protective capability of these agents. Thus, it is contemplated that doses include doses of about 0.1, 0.5, 1, 5, 10, 15, 20, 25, 30, 35, 40, 45, 50, 55, 60, 65, 70, 75, 80, 85, 90, 100, 105, 110, 115, 120, 125, 130, 135, 140, 145, 150, 155, 160, 165, 170, 175, 180, 185, 190, 195, and 200, 300, 400, 500, 1000 μg/kg, mg/kg, μg/day, or mg/day or any range derivable therein. Furthermore, such doses can be administered at multiple times during a day, and/or on multiple days, weeks, or months.

In certain embodiments, the effective dose of the pharmaceutical composition is one which can provide a blood level of about 1 μM to 150 μM. In another embodiment, the effective dose provides a blood level of about 4 μM to 100 μM.; or about 1 μM to 100 μM; or about 1 μM to 50 μM; or about 1 μM to 40 μM; or about 1 μM to 30 μM; or about 1 μM to 20 μM; or about 1 μM to 10 μM; or about 10 μM to 150 μM; or about 10 μM to 100 μM; or about 10 μM to 50 μM; or about 25 μM to 150 μM; or about 25 μM to 100 μM; or about 25 μM to 50 μM; or about 50 μM to 150 μM; or about 50 μM to 100 μM (or any range derivable therein). In other embodiments, the dose can provide the following blood level of the agent that results from a therapeutic agent being administered to a subject: about, at least about, or at most about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 μM or any range derivable therein. In certain embodiments, the therapeutic agent that is administered to a subject is metabolized in the body to a metabolized therapeutic agent, in which case the blood levels may refer to the amount of that agent. Alternatively, to the extent the therapeutic agent is not metabolized by a subject, the blood levels discussed herein may refer to the unmetabolized therapeutic agent.

Precise amounts of the therapeutic composition also depend on the judgment of the practitioner and are peculiar to each individual. Factors affecting dose include physical and clinical state of the patient, the route of administration, the intended goal of treatment (alleviation of symptoms versus cure) and the potency, stability and toxicity of the particular therapeutic substance or other therapies a subject may be undergoing.

It will be understood by those skilled in the art and made aware that dosage units of μg/kg or mg/kg of body weight can be converted and expressed in comparable concentration units of μg/ml or mM (blood levels), such as 4 μM to 100 μM. It is also understood that uptake is species and organ/tissue dependent. The applicable conversion factors and physiological assumptions to be made concerning uptake and concentration measurement are well-known and would permit those of skill in the art to convert one concentration measurement to another and make reasonable comparisons and conclusions regarding the doses, efficacies and results described herein.

### V. KITS

Certain aspects of the present disclosure also concern kits comprising compositions of the invention or compositions to implement methods of the invention. In particular embodiments, the kit comprises NK cells, fresh or frozen, and that may or may not have been pre-activated or expanded. The NK cells may or may not already express one or more components of the TCR/CD3 complex. In cases wherein the NK cells do not already express one or more components of the TCR/CD3 complex, the kit may comprise reagents for corresponding transfection or transduction of the NK cells, including reagents such as vectors that express the component(s), primers for amplification of the component(s), and so forth. In some cases, the NK cells may or may not also express one or more heterologous proteins as defined herein, and when they do not, the kit may comprise vectors that express the heterologous protein(s), primers for amplification of the heterologous protein(s), and so forth.

Kits may comprise components which may be individually packaged or placed in a container, such as a tube, bottle, vial, syringe, or other suitable container means. Individual components may also be provided in a kit in concentrated amounts; in some embodiments, a component is provided individually in the same concentration as it would be in a solution with other components. Concentrations of components may be provided as 1×, 2×, 5×, 10×, or 20× or more.

### VI. EXAMPLES

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

### Preparation and Effective Use of CD3-Expressing NK Cells

The present example concerns cancer immunotherapeutics as a strategy to redirect the specificity of NK cells against one or more target antigens by ‘arming’ or pre-complexing them with bispecific or multi-specific antibodies, such as either prior to infusion or by co-infusing the two products separately. The NK cells are transduced with one or multiple CD3 chains, including CD3ζ, CD3γ, CD3δ and CD3ε chains and can be from any source. The cells can be expanded or non-expanded, they can be pre-activated with one or more inflammatory cytokines, such as IL12/15/18, and/or they can be genetically modified to express one or more heterologous proteins, including, for example, engineered antigen receptors, such as chimeric antigen receptor or a TCR, and/or a cytokine gene and/or a homing/chemokine receptor.

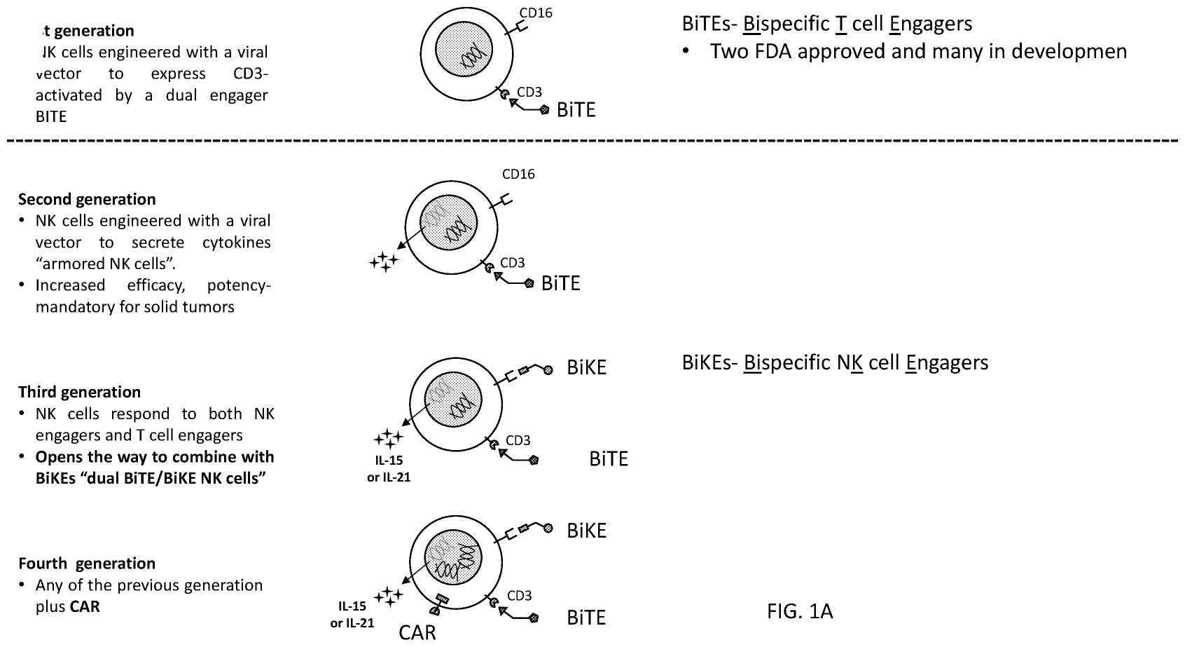

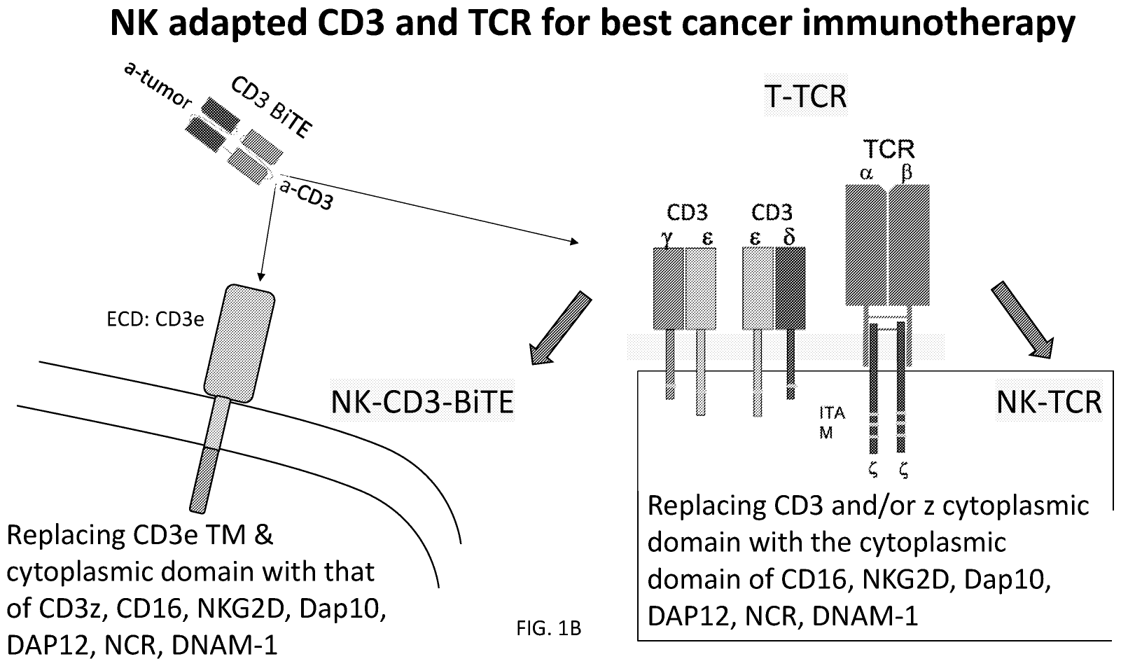

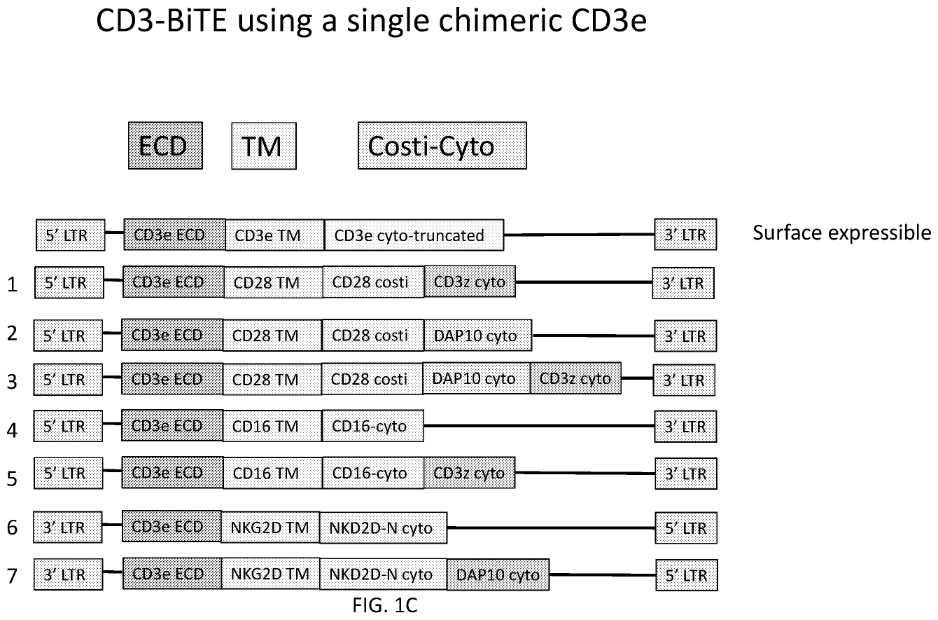

FIGS. 1A and 1B illustrate different embodiments of NK cells engineered to be utilized with bispecific or multi-specific antibodies. As shown in FIG. 1A, in a first generation of NK cells, the cells are engineered to express CD3 that may be activated with a bispecific or multi-specific antibody, including a bispecific T cell engager (BiTE) that comprises an anti-CD3 antibody that binds heterologous CD3 expressed on the surface of the NK cells. In another embodiment, CD3-expressing NK cells are able to be bound by a BiTE that comprises an anti-CD3 antibody, and the NK cells are also expressing one or more particular cytokines (e.g., IL-15 and/or IL-21), resulting in increased efficacy and potency that are particularly useful for treating solid tumors. In another embodiment, the NK cells are engineered to express not only CD3 to be able to be activated by a BiTE that comprises an anti-CD3 antibody but also are utilized with a bispecific or multi-specific antibody (e.g., bispecific NK cell engager, or BiKE) that comprises an antibody that binds a surface antigen naturally present on NK cells, such as CD16, CS1, CD56, NKG2D, NKG2C, DNAM, 2B4, CD2, an NCR, or KIR, for example. In this manner, the NK cells respond to both NK engagers and T cell engagers. In another embodiment, the NK cells in addition to expressing CD3 to engage with T cell engagers also express an engineered antigen receptor, such as a CAR or engineered TCR.

FIG. 1B illustrates different embodiments wherein the NK cells are modified to express both CD3 and a TCR. On the right, T cell TCR is illustrated having α and β chains with an antigen binding site wherein the TCR is complexed with CD3ζ to effect signal transduction. The T cell TCR is co-complexed with two CD3ε chains, a CD3δ chain, and a CD3γ chain. In some embodiments, the NK cells express a TCR in which one or more of the cytoplasmic domains of any of the CD3 molecules is a heterologous intracellular domain, such as one from CD16, NKG2D, DAP10, DAP12, NCR, and DNAM-1. As shown on the left of FIG. 1B, the NK cells are configured to express a CD3 co-receptor component, and in one example the CD3 component is CD3ε. In such a case, a standard BiTE (top left that comprises an antibody against a tumor antigen and an antibody against CD3) normally utilized with T cells that naturally express CD3 may be utilized in conjunction with the CD3-expressing NK cells. In this particular example, the NK cells express a polypeptide that comprises the extracellular domain of CD3ε (although the extracellular domains of other CD3 components may be utilized) and the extracellular domain of CD3ε is linked to a transmembrane domain and/or cytoplasmic domain of another molecule, such as the transmembrane domain and/or cytoplasmic domain of CD3ζ, CD16, NKG2D, DAP10, DAP12, NCR, or DNAM-1, for example.

As described above, FIG. 1C. schematically depicts the generation of surface-expressible single chimeric CD3 constructs that can be used in conjunction with anti-CD3 BiTEs. As one example, the CD3 epsilon extracellular domain (ECD) is fused with CD28, CD16, or NKG2D transmembrane (TM), and CD28, CD16, or NKG2D intracellular domain (ICD), with or without CD3 zeta and/or DAP10 intracellular domains. In one example, the constructs are encompassed within the Moloney murine virus-derived SFG retroviral vector backbone, which may be used with packing plasmids for viral production. In instances wherein the CD3-BiTE is used with such constructs in FIG. 1C, the antibody will bind the extracellular domain of CD3ε accordingly.

Embodiments of the disclosure utilize part or all of the CD3 receptor complex. As illustrated in FIGS. 2A and 2B, the NK cells may be transfected or transduced with full length CD3zeta, CD3 gamma, CD3 delta, and CD3 epsilon. In such cases, the full length of each of CD3zeta, CD3 gamma, CD3 delta, and CD3 epsilon include the extracellular domain, the transmembrane domain, and the intracellular domain. When the different components of the receptor are expressed from the same vector, they may be configured to be produced as separate polypeptides, such as utilizing IRES or 2A elements. In any case, any expression construct may be configured to express one or more cytokines, including at least IL-15.

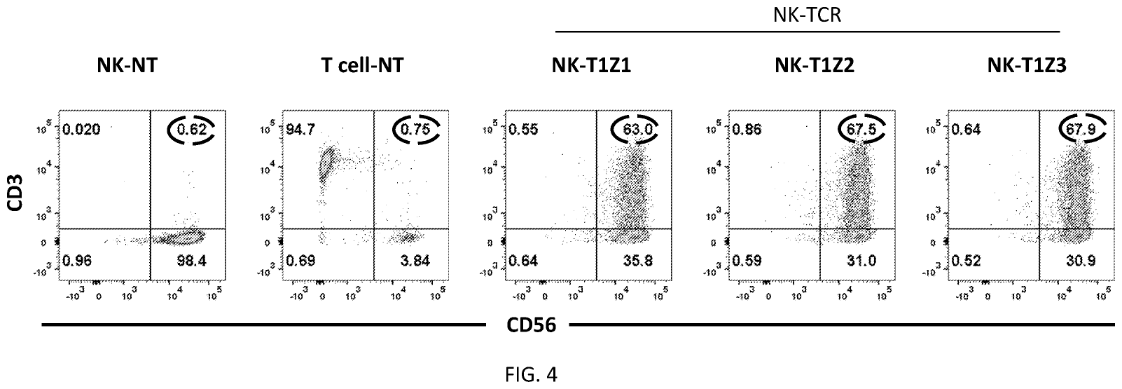

FIG. 4 demonstrates CD3 expression on NK cells after CMV TCR complex transduction, at day 4. The figure provides FACS plots showing CD3 expression on NK cells 4 days after CMV TCR complex transduction. Non transduced (NT) NK cells (CD56+ CD3−) serve as a negative control and T cells (CD3+ CD56−) serve as a positive control.

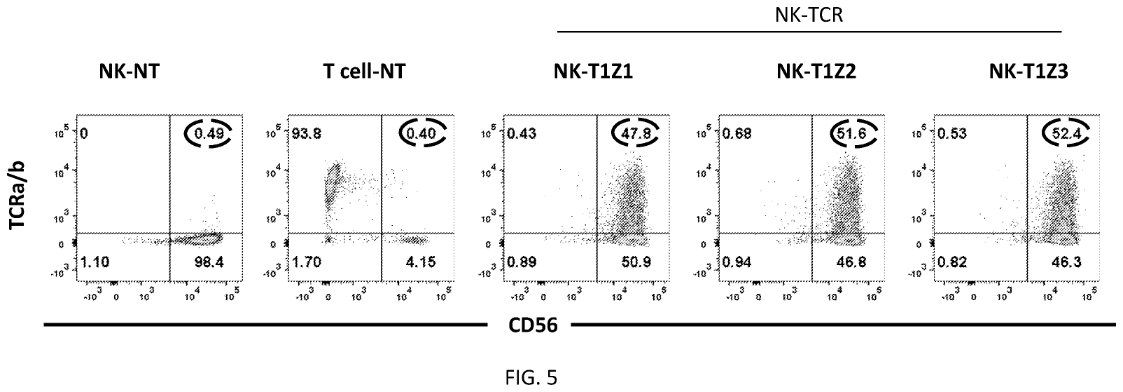

FIG. 5 demonstrates TCR expression on NK cells after CMV TCR complex transduction of NK, day 4. In particular, provided are FACS plots showing TCRa/b expression on NK cells 4 days after CMV TCR complex transduction. Non transduced (NT) NK cells (CD56+CD3−TCRa/b−) serve as a negative control and T cells (CD3+TCRa/b+CD56−) serve as a positive control.

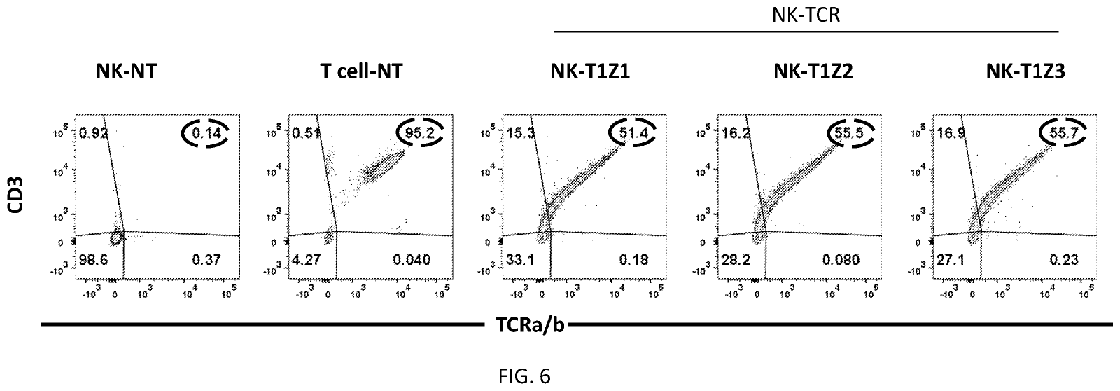

FIG. 6 shows TCR/CD3 expression on NK after CMV TCR complex transduction, day 6. Specifically, FACS plots show dual CD3 and TCRa/b expression on NK cells 6 days after CMV TCR complex transduction. Non transduced (NT) NK cells (CD56+CD3−TCRa/b−) serve as a negative control and T cells (CD3+TCRa/b+CD56−) serve as a positive control.

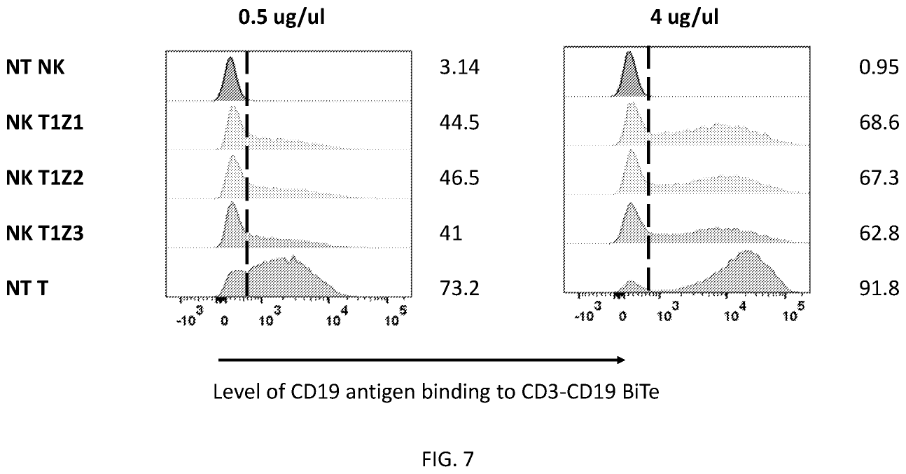

In FIG. 7, shown are the binding of CD3-CD19 BiTE on NK cells through the CD3/TCR at different concentrations. Specifically, the various cells (non-transduced (NT) NK cells, T cells, or the three different NK-TCR cells) were incubated with blinatumomab a CD3-CD19 bispecific engager (BiTe) for one hour at 37° C. using two different concentrations (0.5 μg/μl or 4 μg/μl). Then, a biotin-labeled CD19 antigen (CD19 CAR Detection Reagent from Miltenyi) was added for 20 min followed by an anti-biotin antibody for 15 min at room temperature. This strategy was used to detect any BiTe engaged with a CD3+ cell. The Histograms in FIG. 7 show the level of CD19 binding to CD3-CD19 bispecific engager (BiTe) that correlates with CD3 expression on NK-TCR and T cells.

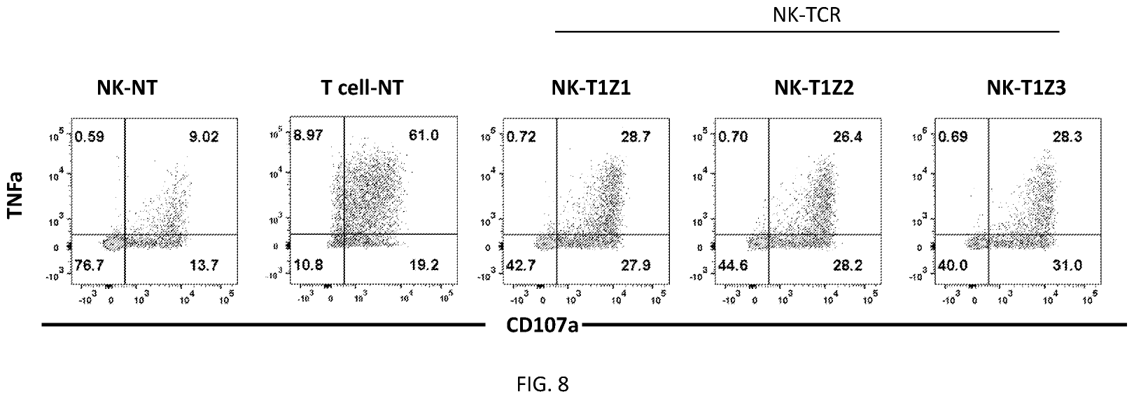

FIG. 8 shows NK-TCR cytokine production after stimulation with a plate-bound CD3 antibody. In particular, CD3-OKT3 clone 20 μg/ml was incubated overnight in flat bottom 96-well plates at 4° C. to form a plate-bound antigen. The next day, T cells or NK cells (NT or TCR-transduced) were added to the wells for 4 hrs and with Brefeldin A (that prevents the cytokine from being released, trapping it in the cytoplasm such that it can be detected by intracellular cytokine staining). They were then harvested for surface and intracellular staining to assess cytokine production and degranulation (TNFα and CD107a). FACS plots in FIG. 8 show TNFα and CD107a double-positive populations in NK cells transduced with TCR. Non-transduced (NT) NK cells (CD56+CD3−) serve as a negative control and T cells (CD3+CD56−) serve as a positive control.

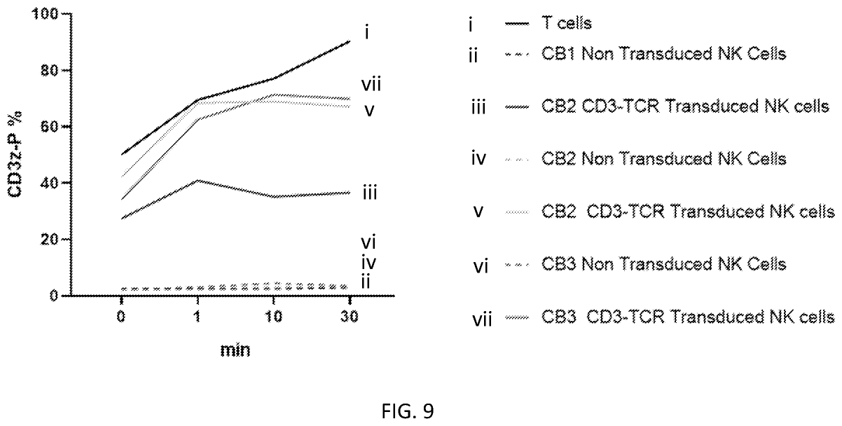

FIG. 9 demonstrates phosphorylation of CD3ζ in NK TCR/CD3 cells after crosslinking CD3. The various cells tested included non-transduced (NT) NK cells; non-transduced (NT) T cells, or three different CD3-TCR transduced NK cells (where CD1, CD2, or CD3 represent different donors). Each of the NK cell groups were transduced with CD3ZFLGDEFL15 (see FIGS. 2A and 2B). The NK cells were incubated with CD3 OKT3 clone (Miltenyi, 130-093-387) at 20 μg/ml concentration for 20 min on ice. Cells were then cross-linked with Fab2 IgG1 antibody for various time points and stained to check for CD3z phosphorylation. This analysis of CD3ζ is useful because, as an internalization signal from the surface, it would only be able to be crosslinked with a CD3 monoclonal antibody if the NK cells expressed it. NK cells that are not transduced with CD3 will not show any phosphorylation or activation after the stimulation.

NK cells transduced with CD3-TCR also show basal level of tonic signaling, which increases upon stimulation with CD3 OKT3 and is similar to T cells, while non-transduced NK cells did not show any CD3ζ phosphorylation neither at basal nor upon CD3 OKT3 stimulation.

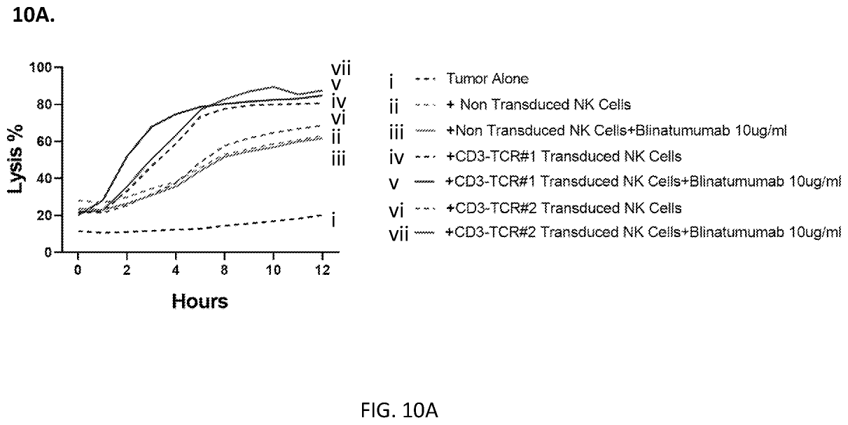

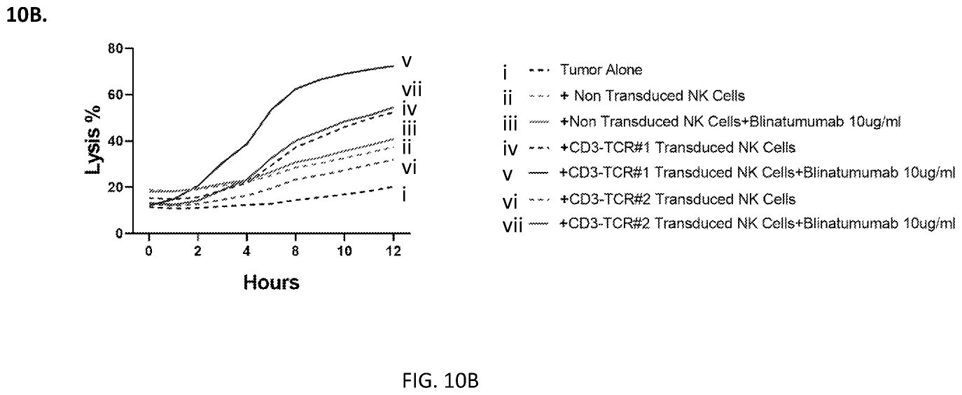

FIG. 10 shows that pre-culturing CD3-CD19 BiTEs with TCR/CD3-expressing NK cells increased its killing activity against Raji cells. NK cells were either transduced with CD3-TCR #1 (CD3ZFLGDEFL15 (see FIGS. 2A and 2B)) or CD3-TCR #2 (Z2, also called CD3ZGDEFL8SP21CD8, that includes full length CD3ζ, full length CD3γ, full length CD3δ, and full length CD3 ε linked to membrane bound IL21 (with CD8 transmembrane domain for the membrane bound IL21). NK cells transduced with the CD3/TCR constructs or non-transduced NK cells were loaded with Blinatumumab and incubated for 1 hour and washed with PBS. They were then co-cultured with CD19+ B cell lymphoma cells at different Effector cell:Target cell ratios (FIG. 10A is a 1:1 ratio, and FIG. 10B is a 1:5 ratio) for various time points. As utilized herein, Effector cells are the CD-3-TCR NK Cells, and Target cells are the Raji cells. Blinatumumab-loaded CD3-TCR transduced NK cells showed enhanced anti-tumor activity compared to Blinatumumab-loaded non-transduced NK cells or CD3/TCR transduced NK cells, but not loaded with Blinatumomab at both E:T ratios.

### Example 2

### NY-ESO TCRs in NK Cells

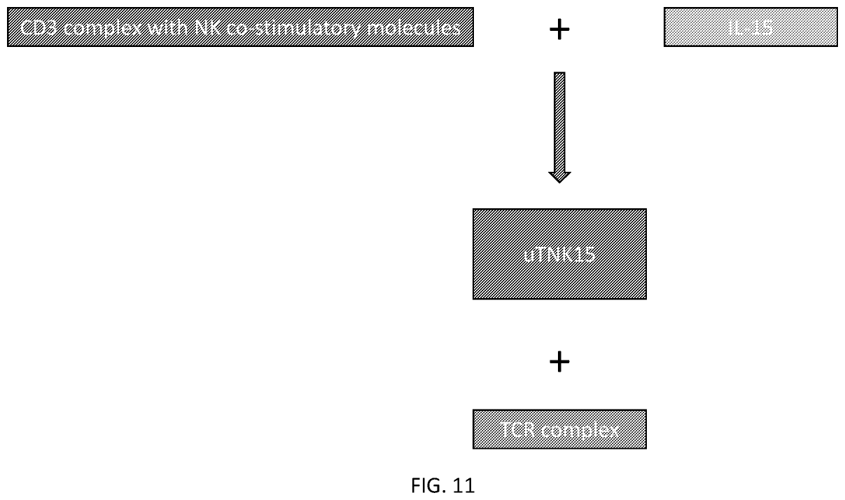

The present examples concern generation and use of NY-ESO TCRs in NK cells. In FIG. 11, there is one example for production of the cells. The schematic overview shows one case wherein the NK cells are first transduced with the uTNK15 construct that incorporates signaling domains from the CD3 complex, NK costimulatory molecules and IL-15, followed by a second transduction step that introduces the TCR molecule, thus generating NK cells that co-express CD3 and NK signaling molecules, IL-15, and a TCR complex. In one embodiment, NK cells were derived from cord blood and were expanded with irradiated (100 Gy) universal antigen presenting cells (uAPC) feeder cells (2:1 feeder cell:NK ratio) and recombinant human IL-2 (200 U/ml) in complete media. To generate a universal T cell-like NK cell (uTNK15 cells) that can secrete IL-15, NK cells were purified and transduced with a retroviral construct containing a CD3 complex with NK co-stimulatory molecules and an IL-15 gene 4 days after isolation. Forty-eight hours after the initial transduction, NK cells expressing uTNK15 were then transduced with a TCR targeting an antigen of choice.

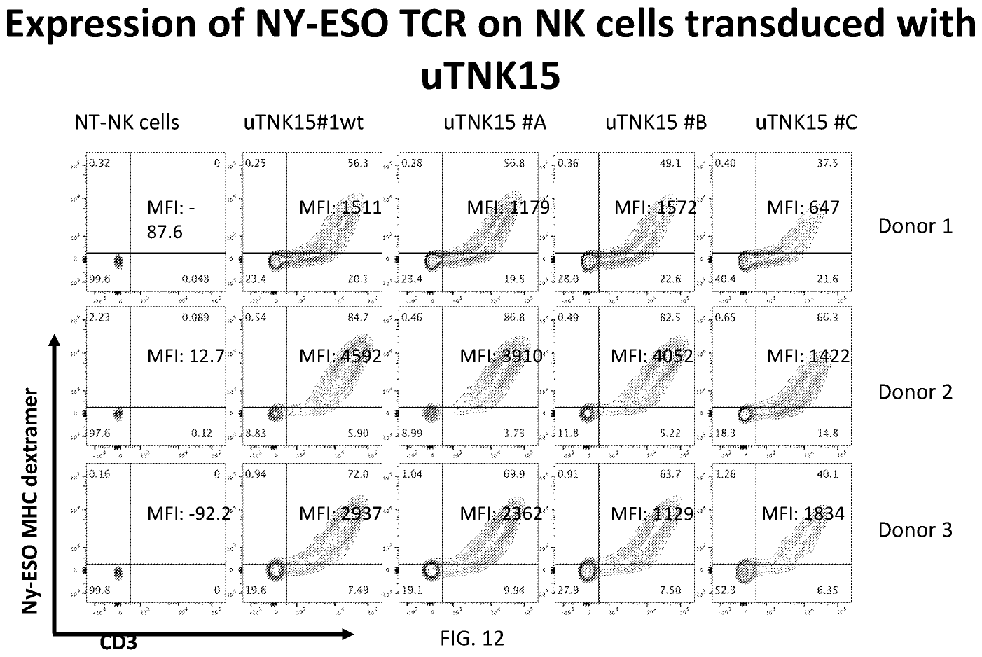

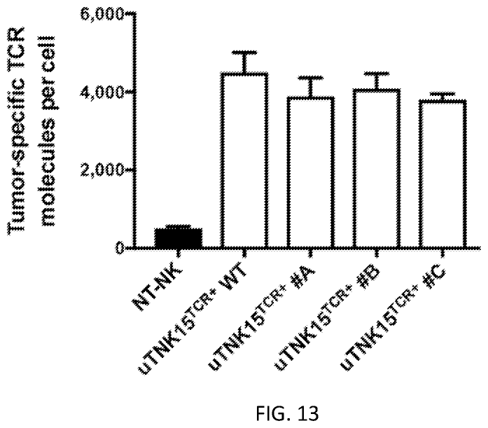

Expression of NY-ESO TCR on NK cells transduced with uTNK15 is shown in FIG. 12. NK cells were derived from cord blood and were expanded with irradiated (100 Gy) universal antigen presenting cells (uAPC) feeder cells (2:1 feeder cell:NK ratio) and recombinant human IL-2 (200 U/ml) in complete media. To generate a universal T cell-like NK cell that can secrete IL-15, NK cells were purified and transduced with a retroviral construct containing a CD3 complex with NK co-stimulatory molecules and an IL-15 gene 4 days after isolation. Forty-eight hours after the initial transduction, uTNK15 cells were then transduced with a TCR complex targeting an antigen of choice. Forty-eight hours later, flow cytometry was used to assess the expression of CD3 and NY-ESO TCR on the various uTNK15 constructs. Non transduced (NT) NK cells served as negative control. CD3 and NY-ESO TCR were highly expressed on all uTNK15 cells compared to NT NK cells. The number of tumor specific TCR molecules expressed on TCR engineered NK cells using the various TCR constructs are provided in FIG. 13, and NT NK cells were used as negative control.

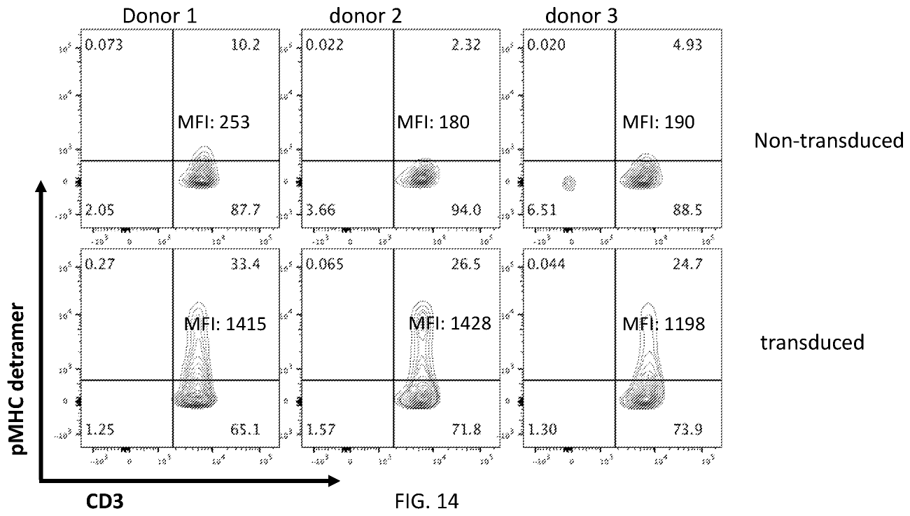

FIG. 14 demonstrates NY-ESO TCR expression on non-transduced and transduced T cells. T cells were isolated from cord blood (the same donor as NK cells to serve as a paired positive control) and were activated with CD3/CD28 microbeads at a concentration of 25 μl/1 million for 48 hours in RPMI complete media. T cells were then transduced with a retroviral construct containing NY-ESO TCR. Forty-eight hours after transduction, flow cytometry revealed that NY-ESO TCR was highly expressed on transduced T cells compared to non-transduced T cells.

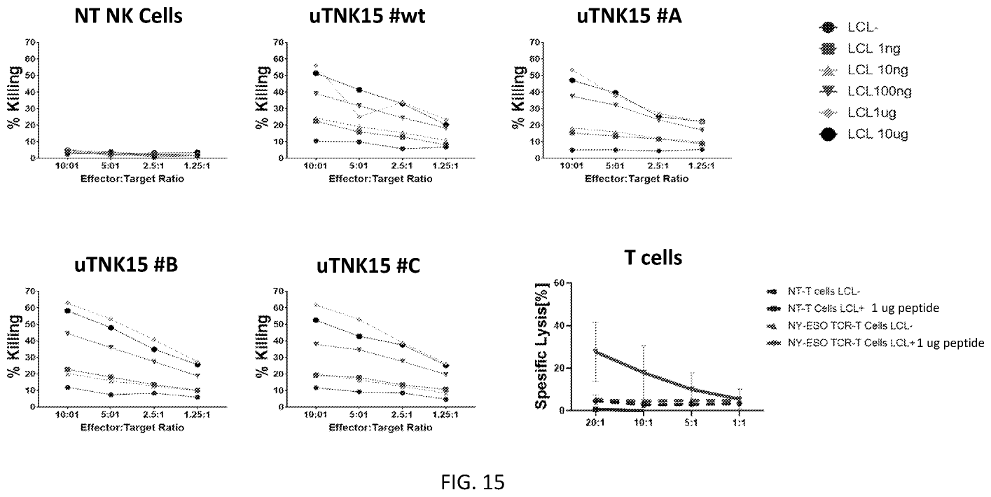

NK cells transduced with NY-ESO TCR kill NY-ESO peptide-pulsed target cells in a dose-dependent manner (FIG. 15). Chromium ⁵¹CR killing assay was performed 7 days following TCR transduction to determine the killing capacity of TCR-engineered NK and T cells against LCL cells loaded with different concentrations of NY-ESO peptide for 2 hours. NY-ESO TCR transduced uTNK15 cells show enhanced killing of peptide-pulsed LCL cells compared to non-transduced NK cells. NY-ESO TCR transduced T cells show enhanced killing of peptide-pulsed LCL cells compared to non-transduced T cells.

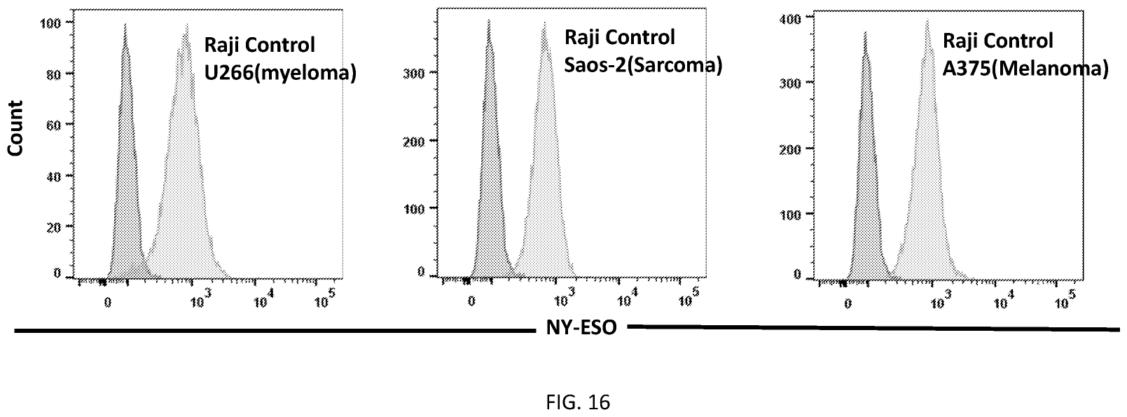

FIG. 16 shows that NY-ESO is expressed endogenously on myeloma, sarcoma, and melanoma cell lines. Flow cytometry was used to determine the expression of NY-ESO on U266 (myeloma), Saos-2 (Sarcoma), and A375 (melanoma) cell lines. U266, Saos-2, and A375 cell lines showed higher levels of NY-ESO expression compared to the Raji cell line which served as negative control.

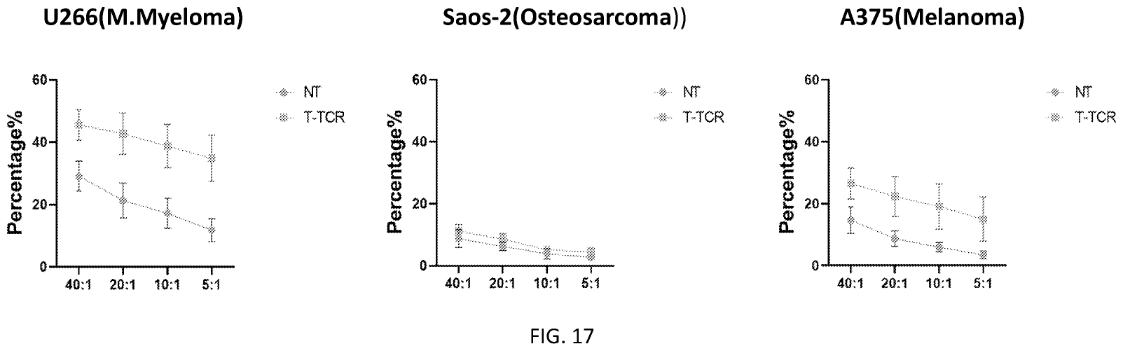

NY-ESO TCR-transduced T cells kill NY-ESO expressing tumor targets at higher E:T ratios (FIG. 17). Chromium ⁵¹CR killing assay was performed 7 days following TCR transduction to determine the killing capacity of NY-ESO TCR-engineered T cells against NY-ESO expressing myeloma, osteosarcoma and melanoma cell lines. NY-ESO TCR transduced T cells show enhanced killing of NY-ESO positive cell lines compared to non-transduced T cells.

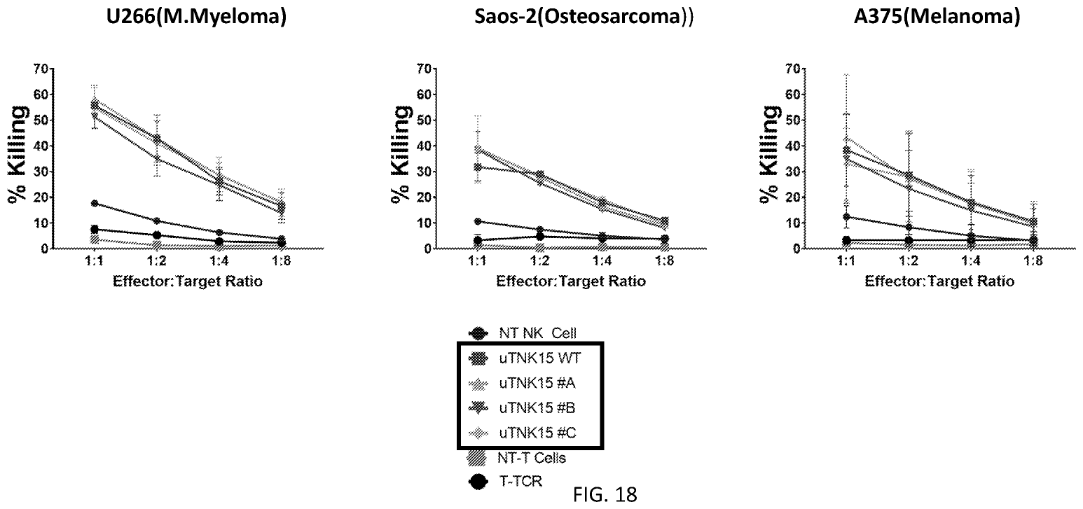

FIG. 18 demonstrates that NY-ESO TCR transduced NK cells kill NY-ESO expressing tumor targets even at low E:T ratios. Chromium ⁵¹CR killing assay was performed 7 days following TCR transduction to determine the killing capacity of NY-ESO TCR-engineered NK cells against NY-ESO-expressing myeloma, osteosarcoma and melanoma cell lines. NY-ESO TCR-transduced NK cells show enhanced killing of NY-ESO positive cell lines compared to non-transduced NK cells even at very low effector:target ratios.

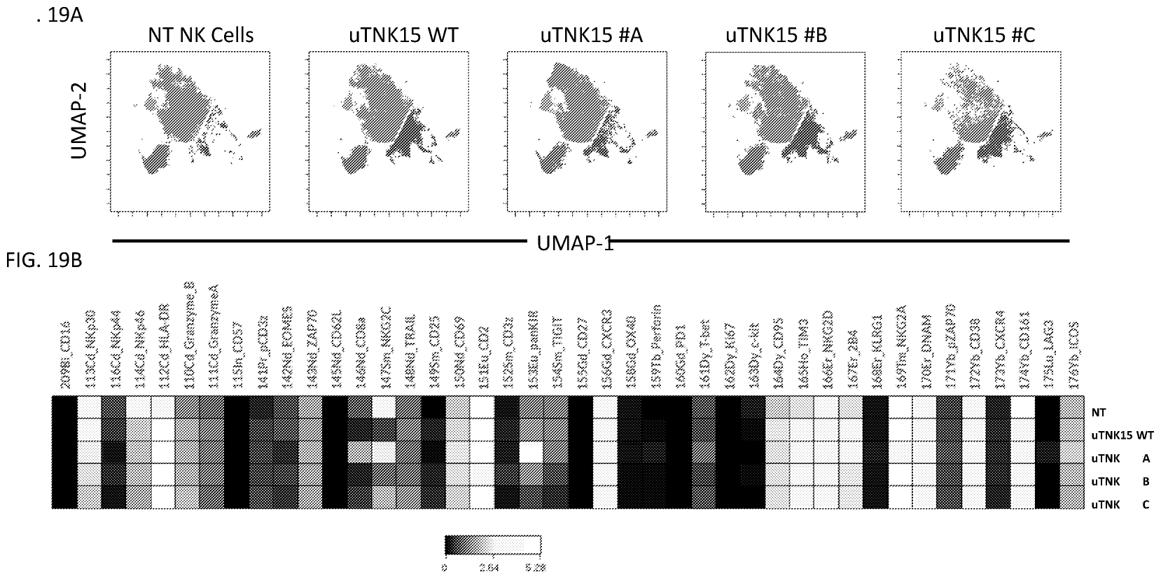

FIG. 19 shows that NY-ESO transduced NK cells have a similar phenotype to NT NK cells. CytoF imaging revealed that non-transduced NK cells and NY-ESO TCR transduced uTNK15 cells share a similar phenotype. FIG. 19A shows a u-map plot with similar clusters, and FIG. 19B shows a heat map with similar expression of various markers on NT and NY-ESO TCR transduced uTNK15 cells.

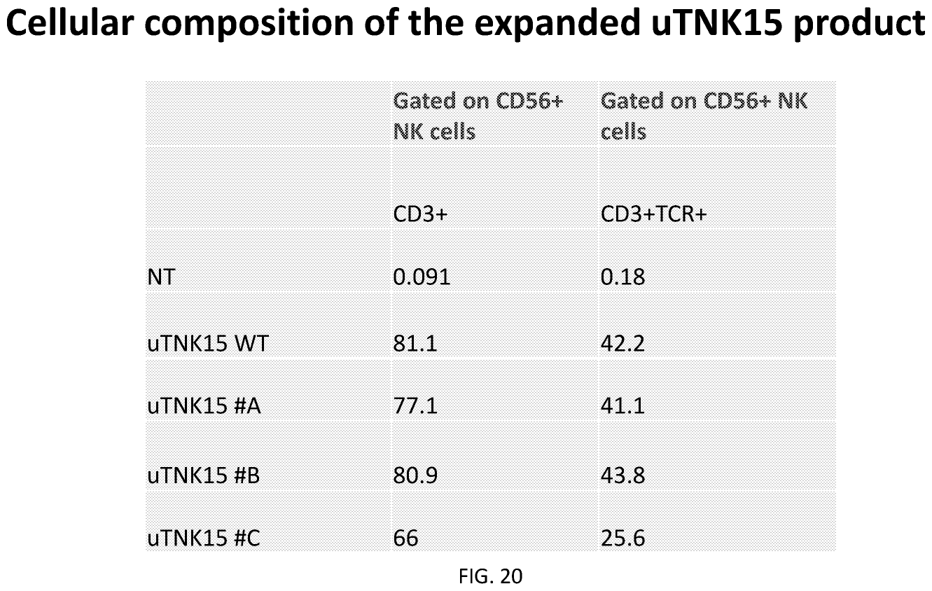

FIG. 20 provides a table representing the percentage of CD3+ and CD3+TCR+NK cells in each uTNK15 product. Flow cytometry was used to assess the composition of single positive CD3 NK cells (CD3+) and double positive CD3/TCR NK cells (CD3+TCR+). Non transduced NK cells are comprised of less than 1% CD3+ and CD3+TCR+NK cells, while the TCR transduced uTNK15 cell products are comprised of over 60% CD3+ and over 25% CD3+TCR+NK cells.

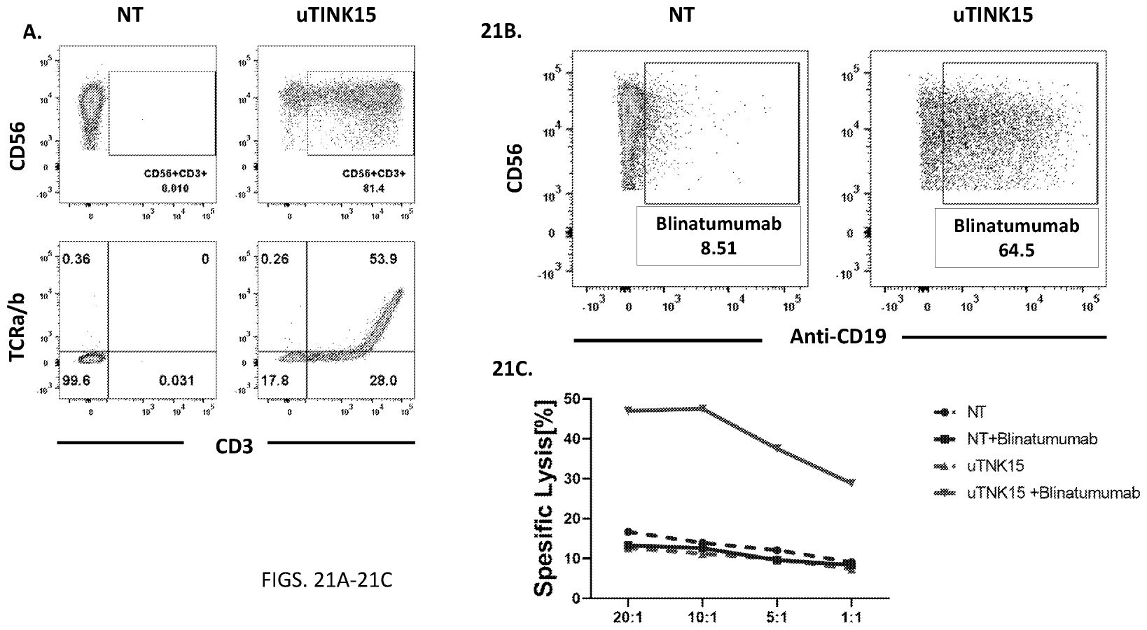

FIG. 21A provides FACS plots that show successful CD3 expression on NK cells 4 days after transduction with TCR constant alpha-beta (TCRCab; TCR6 construct). Non transduced (NT) NK cells (CD56+CD3−) serve as negative control. In FIG. 21B, NT NK and uTNK15 NK cells were incubated with Blinatumumab, a CD3-CD19 bispecific engager (BiTe), for one hour at 37° C. using 10 μg/μl. Then, a biotin-labeled CD19 antigen (CD19 CAR Detection Reagent from Miltenyi) was added for 20 min, followed by an anti-biotin antibody for 15 min at room temperature. This strategy was used to detect any BiTe engaged with a CD3+ cell. The histograms in this figure are showing the level of CD19 binding to CD3-CD19 bispecific engager (BiTe) that correlates with CD3 expression on uTINK15 NK cells. In FIG. 21C, CD3/TCR transduced or non-transduced NK cells were loaded with Blinatumumab and incubated for 1 hour and washed with PBS. They were then co-cultured with LCL cells at different E:T ratios (A.1:1,B.1:5) for various time points. Blinatumumab-loaded CD3-TCR transduced NK cells showed enhanced anti-tumor activity compared to Blinatumumab-loaded non-transduced NK cells or CD3/TCR transduced NK cells but not loaded with Blinatumumab at both E:T ratios.

### Example 3

### NY-ESO TCRs in CD3 Expressing NK Cells In Vivo

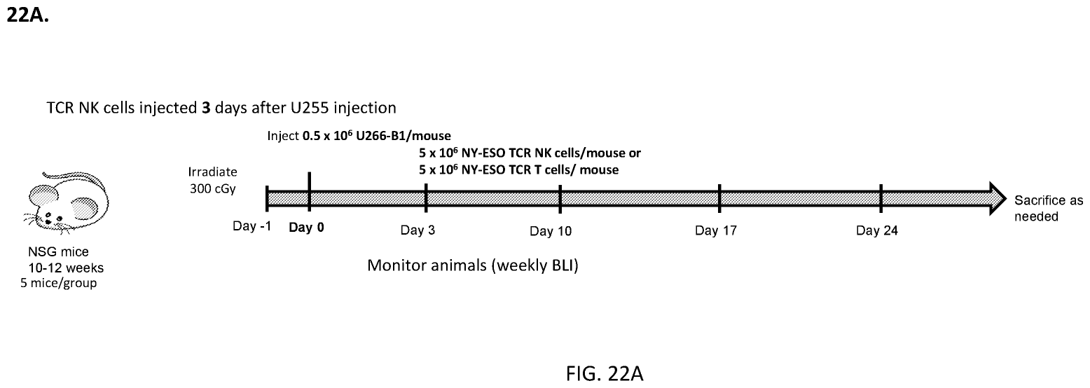

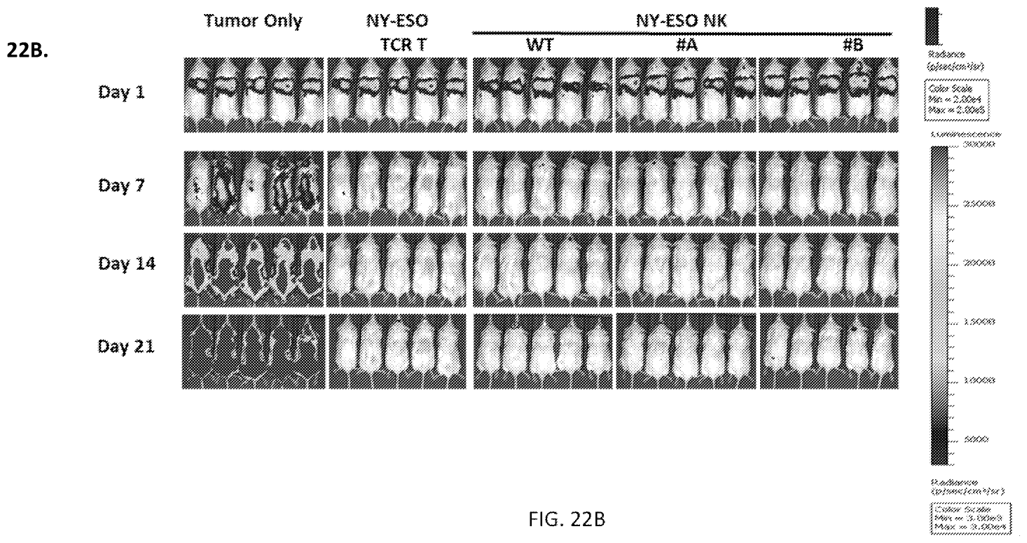

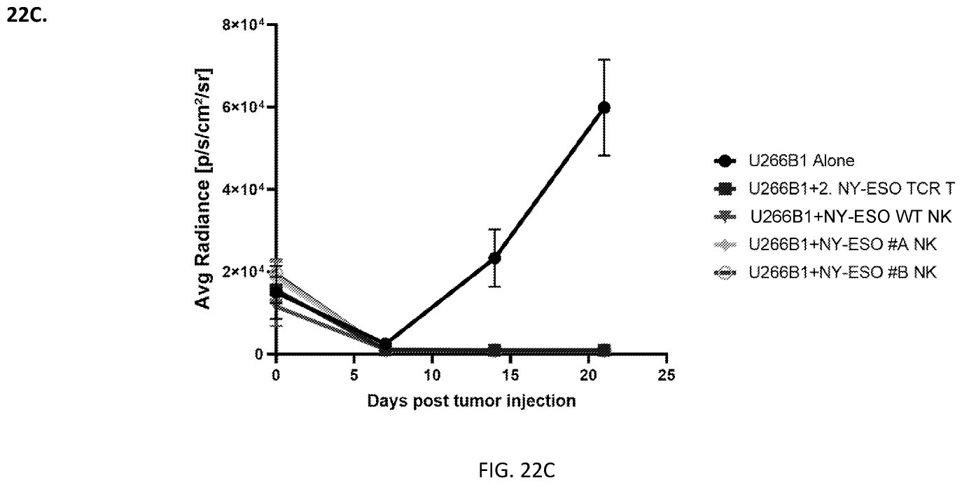

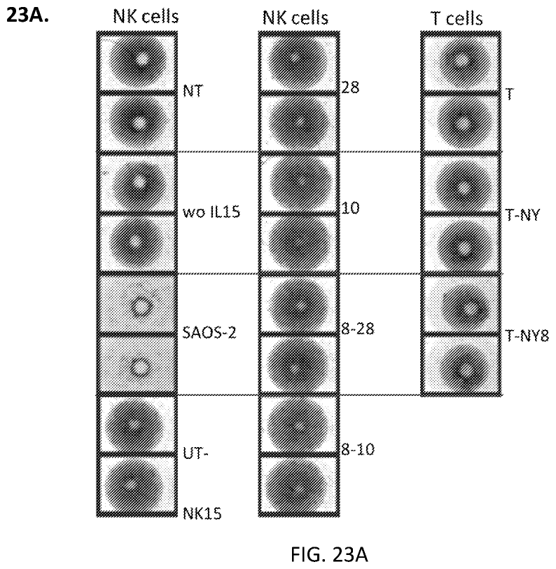

As shown in FIGS. 22A-22C, NK cells comprising constructs described herein were tested in-vivo and found to robustly inhibit tumor growth. Shown in FIG. 22A is a schematic outlining the experimental procedure performed. In brief, NSG mice were irradiated with 300 cGy on day −1, then on day 0 individual mice received tail vein injections of 0.5×10⁶ U266-B1 cells (a myeloma cell line that expresses both HLA-A2 and NY-ESO antigens) that were transduced with FireFlyluciferase (FFluc), on day 3 mice were infused with 5×10⁶ effector cells (NY-ESO TCR NK cells with WT, #A, or #B UT-NK15-NY ESO TCR constructs respectively; WT refers to wild type CD3 molecules with IL-15; #A refers to CD3-CD28 with IL-15 (e.g., UT-NK15-28); and #B refers to CD3-DAP10 with IL-15 (e.g., UT-NK15-DAP10); or NY-ESO TCR T cells), animals were then monitored over time and sacrificed as appropriate (N=5 mice per group). FIG. 22B displays the results of the monitoring of the experiment described in FIG. 22A as a function of bioluminescent imaging over time (displayed are representative images from day 1, day 7, day 14, and day 21 respectively). FIG. 22C is a graphical quantification of the bioluminescence average radiance displayed in FIG. 22B, the Y axis denotes average radiance in p/s/cm²/sr, while the X axis denotes time.

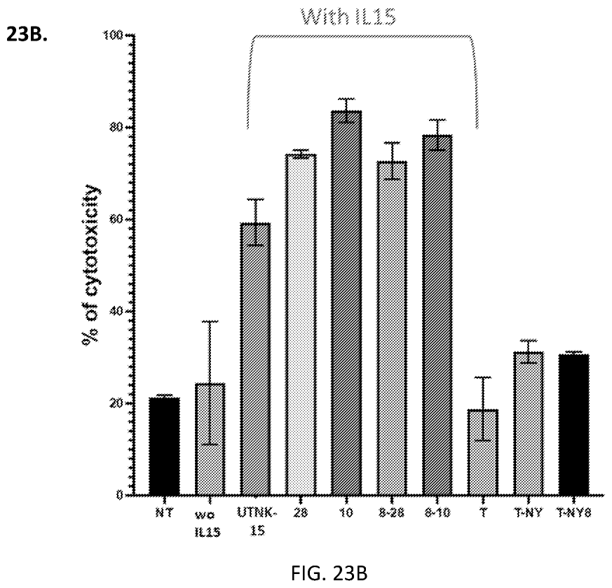

As shown in FIGS. 23A-B the in vitro activity of effector cells (e.g., NK cells or T cells) comprising NY-ESO targeted TCRs and UT-NK15 constructs was tested. FIG. 22A are images of spheroids formed by osteosarcoma tumor cell line Saos-2 that were used to test the activity of NY-ESO1-specific TCR expressing NK and T cells cytotoxicity. Saos-2 cells were stably transduced to express GFP; 10,000 of these cells were seeded per well in a 96 well plate overnight and 40,000 of NK or T cells were then added. Images of the coculture were scanned over time and analyzed by the IncuCyte cell analysis system. Shown in FIG. 22B is a graph displaying the percentage of cytotoxicity (Y axis) for effector cells captured from representative images after 3 days of co-culture. NK cells were co-transduced with NY-ESO-TCR, and the UT-NK15 signaling complex co-expressing different co-stimulatory molecules fused to the CD3ζ signaling chain (e.g., UTNK-15-28, or UTNK-15-DAP10). T cells were only transduced with NY-ESO TCR. Abbreviations in the graph are as follows: 28=CD3ζ fused to a CD28 co-stimulatory domain; 10=CD3ζ fused to a Dap10 co-stimulatory domain; 8=CD8 alpha/beta co-receptor as part of the NY ESO TCR construct; wo IL-15=the construct only contains CD3 zeta, epsilon, gamma and delta without co-stimulation or IL-15. The best in vitro cytotoxicity was observed with TCR NK cells expression UTNK15 with CD28, or DAP10 costimulatory domains fused to CD3ζ (e.g., UTNK-15-28, or UTNK-15-DAP10; SEQ ID NO: 121 and SEQ ID NO: 119 respectively) when compared to NK cells transduced with CD3 complex only or the UT-NK15 without a co-stimulatory domain. The addition of the CD8 alpha/beta coreceptor to the TCR did not significantly improve on the cytotoxicity of NK or T cells.

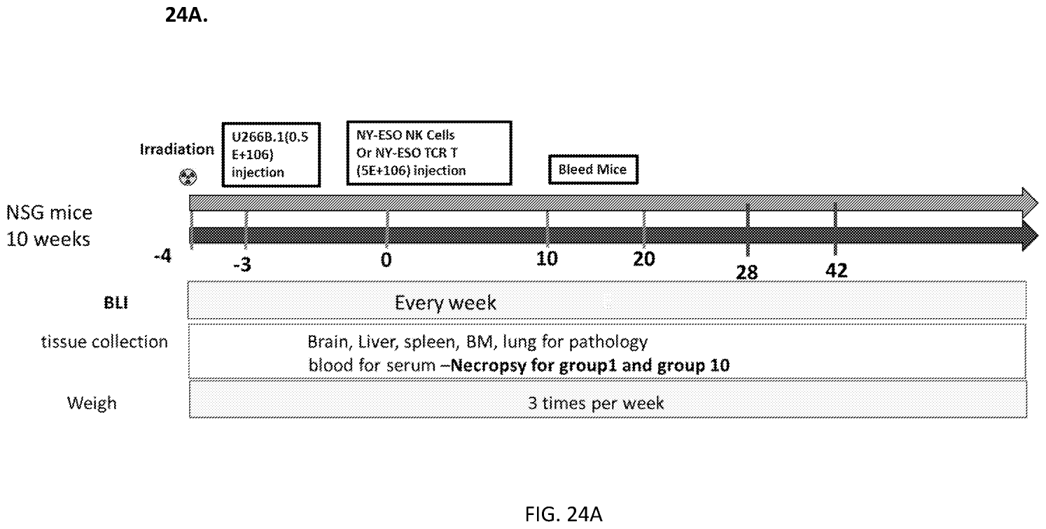

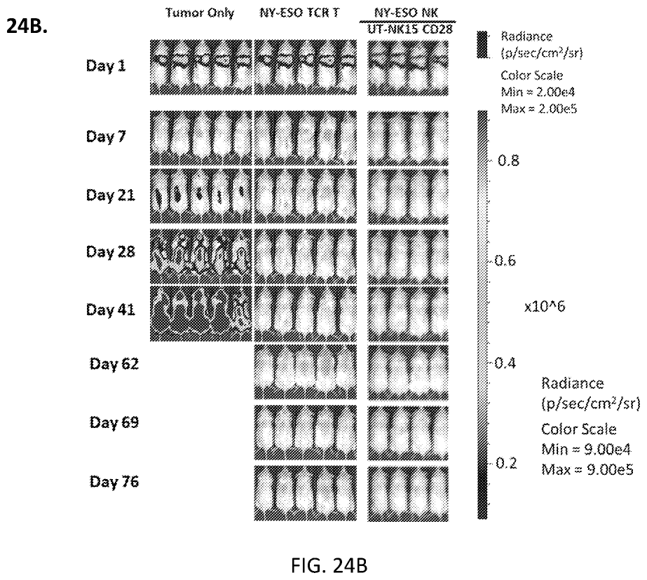

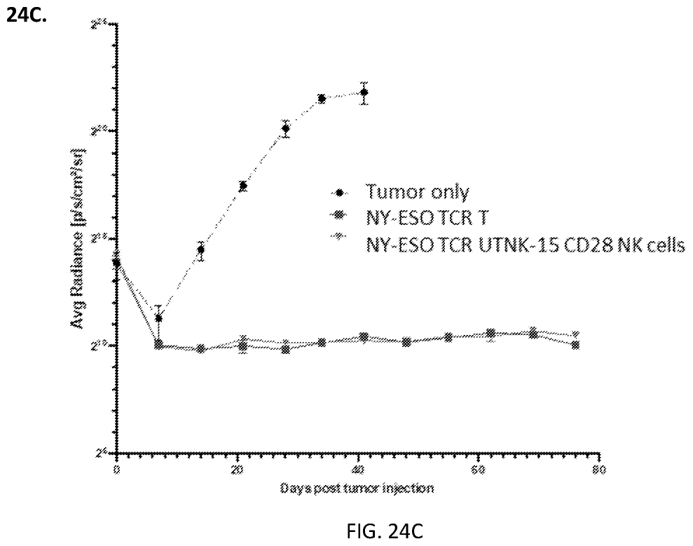

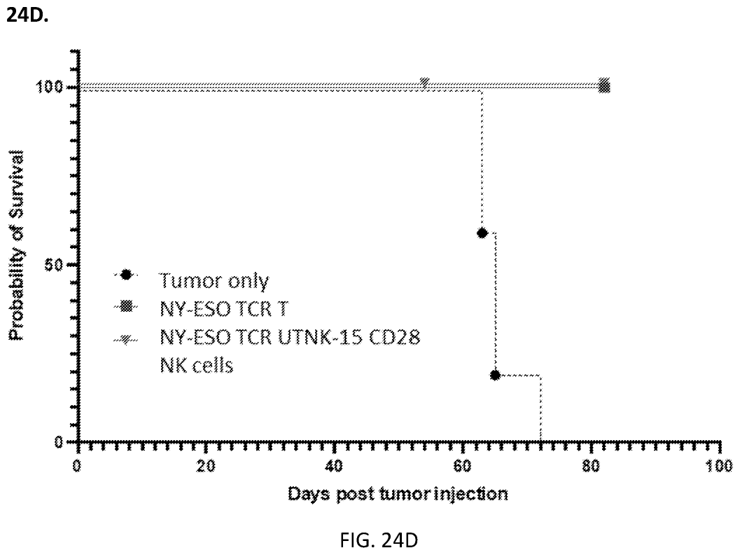

As shown in FIGS. 24A-D the in vivo activity of effector cells (e.g., NK cells or T cells) comprising NY-ESO targeted TCRs and UT-NK15 constructs was tested. FIG. 24A depicts a plan for an in vivo study to test the activity of different NY ESO TCR transduced NK and T cells. The plan was performed, wherein ten week old NSG mice were irradiated (300 cGy) and the next day they were injected with 500,000 U266 cells (HLA-A2 positive, NY-ESO-expressing myeloma cell line) via the tail vein. Three days later, the mice received 5 million TCR transduced T or TCR-transduced NK cells. Mice were then monitored for tumor control by BLI imaging. Shown in FIG. 24B are said BLI imaging results of the test outlined and performed according to FIG. 24A. Mice were injected with U266 tumor cells only, or also with T cells transduced with NY-ESO-specific TCR, or also with NK cells co-transduced with NY-ESO TCR and UT-NK15 with CD3ζ fused to CD28 (labelled as NY-ESO NK UT-NK15 CD28 or NY-ESO TCR UTNK-15 CD28 NK cells). Shown in FIG. 24C are quantifications of region of interest average radiance intensity for the animals tested according to FIG. 24A and imaged in FIG. 24B. Shown in FIG. 24D is a graph depicting the cohort survival curves for the aforementioned animals. The results showed that NY ESO TCR T and NY-ESO TCR UTNK-15-CD28 NK cells mediated strong antitumor activity in vivo.

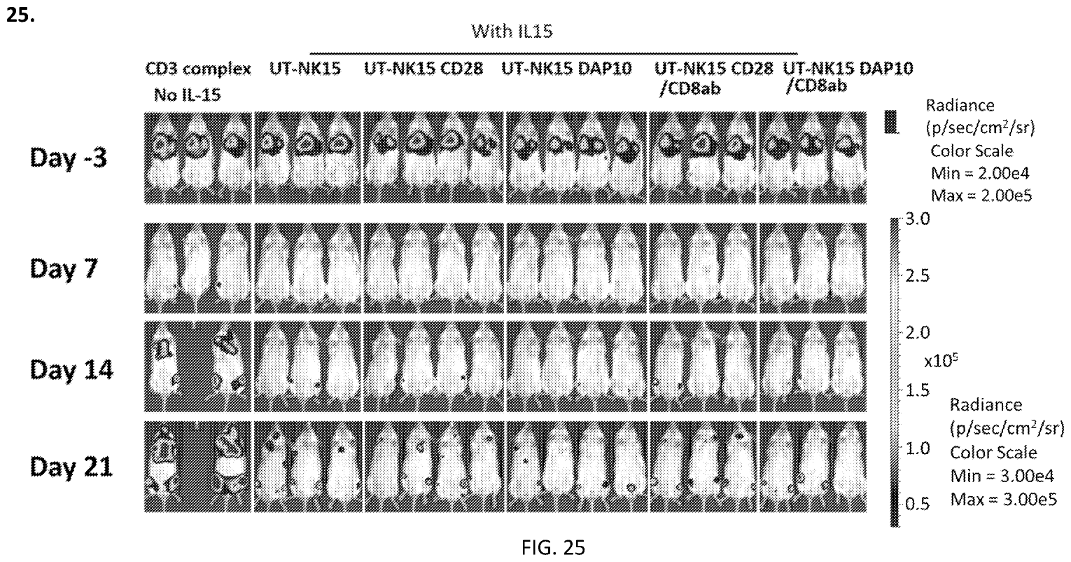

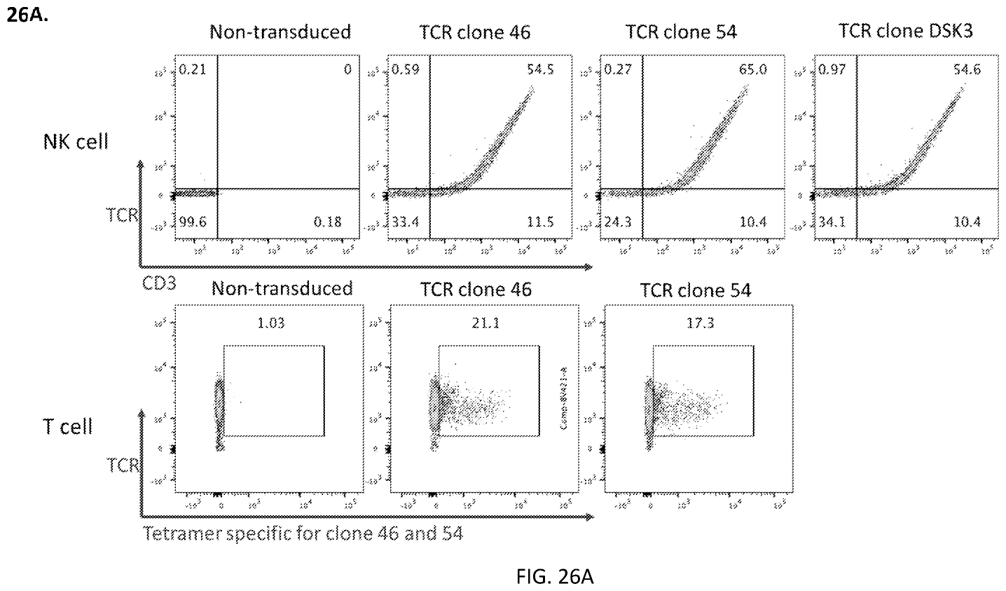

As shown in FIG. 25 the in vivo activity of effector cells (e.g., NK cells) comprising NY ESO TCR and CD3 complex with or without IL-15 was tested. NSG mice were irradiated (300 cGy) and the next day were injected with 500,000 U266 cells (HLA-A2 positive, NY-ESO-expressing myeloma cell line) via the tail vein. Three days later, mice received 5 million TCR transduced T or NK cells. Mice were monitored for tumor control by BLI imaging. NK cells were transduced with NY-ESO-specific TCR, and co-transduced with CD3 complex without IL-15 or with UT-NK15 expressing CD3ζ fused to CD28 (UT-NK15-28) or CD3ζ fused to DAP10 (UT-NK15-DAP10) co-stimulatory molecules, with or without expression of CD8 alpha/beta co-receptors. The results showed that absence of IL-15 resulted in a reduced anti-tumor activity in vivo.

Together these results showed that effector cells (e.g., NK cells) comprising constructs described herein (e.g., NY-ESO TCR constructs and/or CD3 constructs such as UT-NK15 or modified versions thereof, e.g., UT-NK-15-28 or UT-NK15-DAP10) were sufficient to robustly inhibit tumor growth in vivo.

### Example 4

### PRAME TCRs in CD3 Expressing NK Cells In Vitro

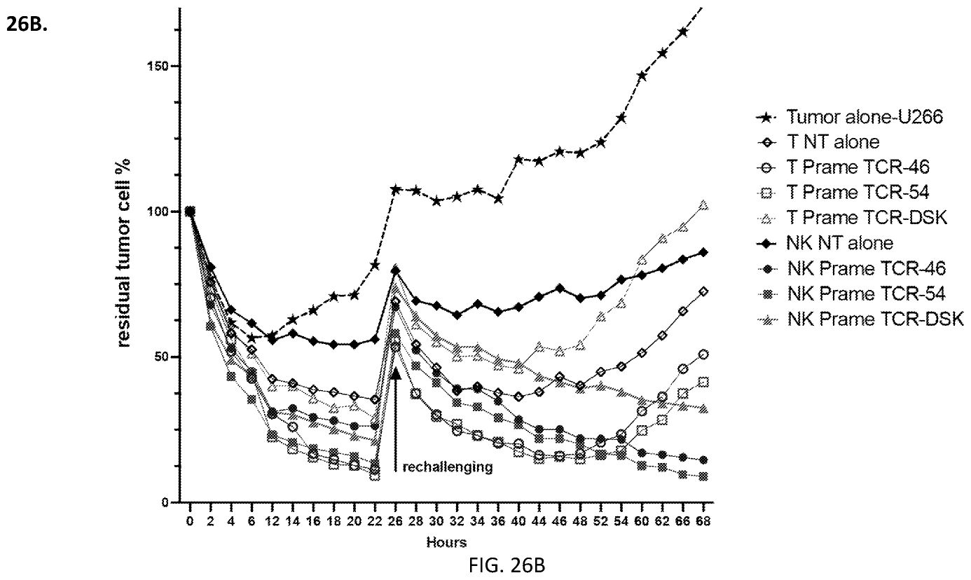

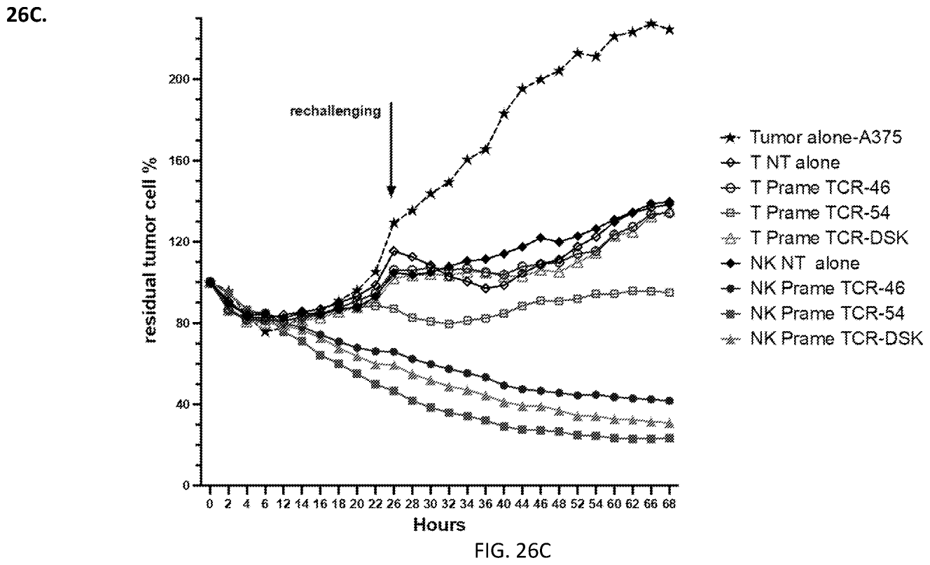

As shown in FIGS. 26A-C, NK cells comprising constructs targeting Preferentially Expressed Antigen In Melanoma (PRAME) antigen described herein were tested in-vitro and found to robustly inhibit tumor cell growth. FIG. 26A shows the expression of both UT-NK15 (x-axis, CD3) and PRAME-specific TCRs (y-axis, TCR) in NK cells (TCR clones 46, 54, or DSK3 respectively), or the expression of PRAME-specific TCRs in T cells transduced with the same (TCR clones 46 or 54). PRAME-specific TCR expression on NK cells was confirmed using antibodies against the TCR and against CD3. Expression of PRAME-specific TCR in T cells was confirmed by tetramer staining using the 46/54 peptide/MHC-specific tetramer. FIG. 26B shows the in vitro cytotoxicity of NK cells expressing a PRAME-specific TCR against the U266 myeloma cell line. Incucyte live cell imaging was used to measure the cytotoxicity of T cells transduced with PRAME-specific TCR and NK cells transduced with UT-NK15 and PRAME-specific TCR against U266 myeloma cells. GFP-expressing U266 cells were co-cultured with PRAME-specific TCR expressing T cell or NK cells at 1:1 effector:target ratio (50,000 effector and 50,000 target cells were seeded in each well of a 96 well plate). A reduction in GFP expression indicated cell death. After 26 hours, a second round of 50,000 tumor cells was added (noted as “rechallenging”) to each well for the tumor rechallenge assay. NK cells expressing UT-NK15 and PRAME-specific TCR clone 46 or PRAME-specific TCR clone 54 exerted the best anti-tumor activity upon rechallenge with U266 cells and displayed superior cytotoxicity when compared to control T cells transduced with PRAME-specific TCR clones 46 or 54 respectively. FIG. 26C shows the in vitro cytotoxicity of NK cells expressing a PRAME-specific TCR against the UA375 melanoma cell line. Incucyte live cell imaging was used to measure the cytotoxicity of T cells transduced with PRAME-specific TCR and NK cells transduced with UT-NK15 and PRAME-specific TCR against UA375 melanoma cells. GFP-expressing UA375 cells were co-cultured with PRAME-expressing T cell or NK cells at 1:1 effector:target ratio (50,000 effector and 50,000 target cells were seeded in each well of a 96 well plate). A reduction in GFP expression indicated cell death. After 26 hours, a second round of 50,000 tumor cells was added to each well for the tumor rechallenge assay. Open symbols represent T cells, while closed symbols represent NK cells. NT=non-transduced. NK cells expressing UT-NK15 and PRAME-specific TCR clone 46 (TCR-46), PRAME-specific TCR clone 54 (TCR-54), or PRAME-specific TCR clone DSK3 (DSK) exerted strong anti-tumor activity upon rechallenge with UA375 cells, and displayed superior cytotoxicity when compared to control T cells transduced with PRAME-specific TCR clones 46, 54, or DSK3 respectively.

Together these results show that effector cells (e.g., NK cells) comprising constructs described herein (e.g., PRAME-specific TCR constructs) were sufficient to robustly inhibit tumor growth in vivo. Furthermore, NK cells comprising CD3 constructs described herein coupled with PRAME-specific TCR constructs displayed increased cytotoxicity when compared to T cell control cells comprising the same TCR constructs, particularly in cases of continuous and/or rechallenge by tumor cells.

### Example 5

### TCRs in CD3 Expressing NK Cells In Vivo

NK cells comprising constructs described herein are tested in-vivo and robustly inhibit tumor growth. Experiments are performed according to schematics and experimental procedures described herein. In brief, NSG mice are irradiated (e.g., with about 300 cGy) on day −1, then on day 0 individual mice receive tail vein injections of cancer cells (e.g., 0.5×10⁶ cells e.g., cells expressing (naturally and/or transduced with) an antigen described herein) that are transduced with an appropriate marker (e.g., FireFlyluciferase (FFluc)), on day 3 mice are infused with effector cells transduced with a transgenic TCR (e.g., TCR constructs comprising gamma/delta TCR chains and/or alpha/beta TCR chains, e.g., targeting antigens described herein, e.g., NY-ESO, Tyrosinase, MAGEA3, MAGEA4, HPV E7, WT1, PRAME, gp100, MART-1, etc.) and with or without other constructs described herein (e.g., with about 5×10⁶ TCR NK cells with a UT-NK15 construct with or without IL15, with or without CD3 fusion to a costimulatory molecule, and/or with or without additional control constructs). Animals are then monitored over time and sacrificed as appropriate. Results of the monitoring of the experiment described above are recorded, e.g., as a function of bioluminescent imaging over time (e.g., on day 1, day 7, day 14, day 21, etc).

The in vitro activity of effector cells (e.g., NK cells or T cells) comprising TCR(s) (e.g., TCR constructs comprising gamma/delta TCR chains and/or alpha/beta TCR chains, e.g., targeting antigens described herein, e.g., NY-ESO, Tyrosinase, MAGEA3, MAGEA4, HPV E7, WT1, PRAME, gp100, MART-1, etc.) and UT-NK15 constructs are tested. Spheroids formed by an appropriate tumor cell line(s) comprising an antigen of interest (e.g., 0.5×10⁶ cells e.g., cells expressing (naturally and/or transduced with) an antigen described herein) are used to test the activity of specific TCR expressing NK and/or T cells cytotoxicity. Cancer cells are stably transduced to express an appropriate marker (e.g., GFP, FFluc, etc.); a number of these cells (e.g., about 10,000) are seeded per well in a 96 well plate overnight and a number of effector cells (e.g., about 40,000) are then added. Images of the coculture are scanned over time and analyzed by an appropriate system (e.g., an IncuCyte cell analysis system). The percentage of cytotoxicity for effector cells are captured from representative images after a number of days (e.g., 1 day, 3 days, 7 days, etc.) of co-culture. NK cells are co-transduced with antigen targeting TCRs, and UT-NK15 signaling complex co-expressing different co-stimulatory molecules fused to the CD3ζ signaling chain (e.g., UTNK-15-28, or UTNK-15-DAP10). Appropriate control cells are transduced with appropriate constructs described herein. Superior in vitro cytotoxicity is observed with TCR NK cells expression UTNK15 with CD28, or DAP10 costimulatory domains fused to CD3ζ (e.g., UTNK-15-28, or UTNK-15-DAP10; e.g., SEQ ID NO: 121 and SEQ ID NO: 119 respectively) when compared to NK cells transduced with CD3 complex only or UT-NK15 without a co-stimulatory domain.

The in vivo activity of effector cells (e.g., NK cells or T cells) comprising antigen specific TCRs (e.g., TCR constructs comprising gamma/delta TCR chains and/or alpha/beta TCR chains, e.g., targeting antigens described herein, e.g., NY-ESO, Tyrosinase, MAGEA3, MAGEA4, HPV E7, WT1, PRAME, gp100, MART-1, etc.) and UT-NK15 constructs are tested. Assays for in vivo analysis of effector cells (e.g., NK cells or T cells) comprising engineered constructs are performed similar to experimental plans described in FIG. 24. In brief, appropriately aged NSG mice (e.g., ten week old NSG mice) are irradiated (e.g., with about 300 cGy) and the next day they are injected with tumor cells comprising the target antigen of interest (e.g., about 500,000 cells; e.g., naturally expressing and/or transduced with an antigen described herein) via the tail vein. Three days later, the mice receive an effector cell bolus (e.g., about 5 million TCR transduced T and/or TCR-transduced NK cells). Mice are then monitored for tumor control (e.g., by BLI imaging). Average radiance for regions of interest are measured and quantified, animals comprising test constructs comprising TCRs targeting an antigen of interest and UT-NK15 constructs with or without CD3 fusions and/or IL-15 expression display improved survival relative to control animals and/or a reduction in average radiance. The results show that TCR UTNK-15 NK cells mediate strong antitumor activity in vivo.

The in vivo activity of effector cells (e.g., NK cells) comprising TCR (e.g., TCR constructs comprising gamma/delta TCR chains and/or alpha/beta TCR chains, e.g., targeting antigens described herein, e.g., NY-ESO, Tyrosinase, MAGEA3, MAGEA4, HPV E7, WT1, PRAME, gp100, MART-1, etc.) and CD3 complex with or without IL-15 are tested. NSG mice are irradiated (e.g., with about 300 cGy) and the next day are injected with tumor cells expressing an antigen of (e.g., about 500,000 cells; e.g., naturally expressing and/or transduced with an antigen described herein) via the tail vein. Three days later, mice receive an effector cell bolus (e.g., about 5 million TCR transduced T and/or TCR transduced NK cells). Mice are monitored for tumor control (e.g., by BLI imaging). NK cells are transduced with antigen-specific TCR, and co-transduced with CD3 complex without IL-15 or with UT-NK15 expressing CD3ζ fused to CD28 (UT-NK15-28) or CD3ζ fused to DAP10 (UT-NK15-DAP10) co-stimulatory molecules, with or without expression of CD8 alpha/beta co-receptors. The results show that absence of IL-15 results in a reduced anti-tumor activity in vivo.

Together these results show that effector cells (e.g., NK cells) comprising constructs described herein (e.g., TCR constructs and/or CD3 constructs such as UT-NK15 or modified versions thereof, e.g., UT-NK-15-28 or UT-NK15-DAP10) are sufficient to robustly inhibit tumor growth in vivo.

All of the methods disclosed and claimed herein can be made and executed without undue experimentation in light of the present disclosure. While the compositions and methods of this invention have been described in terms of preferred embodiments, it will be apparent to those of skill in the art that variations may be applied to the methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

1-95. (canceled)

96. A composition, comprising NK cells modified to express part or all of a single chain or any combination of CD3δ, CD3ε, CD3γ, or CD3′.

97. The composition of claim 96, wherein the NK cells are modified to express one of more of the TCRα chain, the TCRβ chain, the TCRγ chain, and the TCR6 chain.

98. The composition of claim 96, wherein any one or more of the CD3ζ, CD3ε, CD3d, and CD3γ are heterologously linked to one or more intracellular signaling domains.

99. The composition of claim 98, wherein the intracellular signaling domain is selected from the group consisting of CD16, NKG2D, DAP10, DAP 12, 2B4, 4-1BB, CD2, 1D28, and a combination thereof.

100. The composition of claim 99, wherein the intracellular signaling domain comprises an amino acid sequence at least about 85% identical to SEQ ID NO: 115; at least about 85% identical to SEQ ID NO: 116, or at least about 85% identical to SEQ ID NO: 117.

101. The composition of claim 96, wherein the composition further comprises one or more bispecific or multi-specific antibodies, wherein the bispecific or multi specific antibody comprises an anti-CD3 antibody.

102. The composition of claim 101, wherein the NK cells express the antibody and/or the antibody is complexed to the NK cells.

103. The composition of claim 96, wherein the NK cells are modified to express one or more heterologous proteins selected from an engineered antigen receptor, a cytokine, a homing receptor, or a chemokine receptor.

104. The composition of claim 103, wherein the engineered antigen receptor is a chimeric antigen receptor (CAR) and/or engineered T cell receptor (TCR).

105. The composition of claim 104, wherein the engineered antigen receptor is an engineered TCR, and wherein the engineered TCR targets a NY-ESO antigen or a PRAME antigen epitope.

106. The composition of claim 105, wherein the T cell receptor comprises a sequence at least 85% identical to SEQ ID NO: 25 and a sequence at least 85% identical to SEQ ID NO: 26.

107. The composition of claim 105, wherein the target PRAME antigen epitope is SLLQHLIGL (SEQ ID NO: 131) and/or QLLALLPSL (SEQ ID NO: 132).

108. The composition of claim 105, wherein the T cell receptor comprises
(i) a sequence at least 85% identical to SEQ ID NO: 135 and a sequence at least 85% identical to SEQ ID NO: 136,
(ii) a sequence at least 85% identical to SEQ ID NO: 139 and a sequence at least 85% identical to SEQ ID NO: 140; or
(iii) a sequence at least 85% identical to SEQ ID NO: 142 and a sequence at least 85% identical to SEQ ID NO: 144.

109. The composition of claim 103, where in the heterologous protein is a cytokine and wherein the cytokine is selected from the group consisting of:
(i) IL-15, IL-12, IL-2, IL-18, IL-21, IL-23, IL-7, GMCSF, or a combination thereof, or
(ii) IL-15, IL-12, IL-2, IL-18, IL-21, IL-23, IL-7, GMCSF, or a combination thereof, and the cytokine is membrane-bound and comprises a transmembrane domain from CD8, CD28, CD27, B7H3, IgG1, IgG4, CD4, DAP10, or DAP12.

110. The composition of claim 101, wherein the bispecific antibody comprises an antibody that targets a cancer antigen.

111. A composition comprising a complex, comprising:
(i) NK cells modified to express part or all of the CD3 receptor complex and optionally modified to express the T-cell receptor (TCR) ab chains or the TCR gd chains; and
(i) a bispecific or multi-specific antibody, wherein the bispecific or multi-specific antibody comprises an anti-CD3 antibody that is bound to CD3 on the NK cells.

112. The composition of claim 111, wherein the NK cells are modified to express TCR ab chains that are at least 85% identical to SEQ ID NO: 25 and SEQ ID NO: 26, the TCR ab chains target a NY-ESO antigen, and the bispecific antibody is Blinatumomab.

113. The composition of claim 111, wherein any one or more of CD3ζ, CD3ε, CD3δ, and CD3y are heterologously linked to one or more intracellular signaling domains.

114. The composition of claim 113, wherein the intracellular signaling domain is selected from the group consisting of CD16, NKG2D, DAP10, DAP 12, 2B4, 4-1BB, CD2, CD28, DNAM, and a combination thereof.

115. The composition of claim 113, wherein the intracellular signaling domain comprises an amino acid sequence at least about 85% identical to SEQ ID NO: 115; at least about 85% identical to SEQ ID NO: 116; or at least about 85% identical to SEQ ID NO: 117.

116. A method of treating cancer in an individual, comprising the step of administering to the individual a therapeutically effective amount of the composition of claim 96.

117. A method of redirecting the specificity of NK cells against a cancer antigen for treatment of an individual with a bispecific or multi-specific anti-CD3 antibody, comprising the steps of administering to the individual the antibody and NK cells that optionally express part or all of a CD3 receptor complex and that optionally express part or all of TCR ab chains or the TCR gd chains.

118. The method of claim 117, wherein the NK cells are modified to express part of or all of CD3ζ, CD3ε, CD3δ, and CD3γ, and wherein any one or more of CD3ζ, CD3ε, CD3δ, and CD3y are heterologously linked to one or more intracellular signaling domains.

119. The method of claim 118, wherein the intracellular signaling domain is selected from the group consisting of CD16, NKG2D, DAP10, DAP 12, 2B4, 4-1BB, CD2, CD28, DNAM, and a combination thereof.

120. The method of claim 119, wherein the intracellular signaling domain comprises an amino acid sequence at least about 85% identical to SEQ ID NO: 115; at least about 85% identical to SEQ ID NO.: 116; at least about 85% identical to SEQ ID NO: 117.

121. The method of claim 117, further comprising the step of modifying NK cells to express part or all of the TCR ab chains or the TCR gd chains and wherein the TCR ab chains or the TCR gd chains are targeted to an NY-ESO antigen or a PRAME antigen epitope.

122. The method of claim 121, wherein the TCR chains are TCR ab chains, and are at least 85% identical to SEQ ID NO: 25 and SEQ ID NO: 26.

123. The method of 121, wherein the target PRAME antigen epitope is SLLQHLIGL (SEQ ID NO: 131) and/or QLLALLPSL (SEQ ID NO: 132).

124. The method of claim 121, wherein the TCR chains comprise,
(i) a sequence at least 85% identical to SEQ ID NO: 135 and a sequence at least 85% identical to SEQ ID NO: 136;
(ii) a sequence at least 85% identical to SEQ ID NO: 139 and a sequence at least 85% identical to SEQ ID NO: 140; or
(iii) a sequence at least 85% identical to SEQ ID NO: 142 and a sequence at least 85% identical to SEQ ID NO: 144.

125. The method of claim 117, further comprising the step of modifying the NK cells to express one or more additional heterologous proteins.

126. A polynucleotide or polypeptide comprising a sequence at least 85% identical to any one or more of SEQ ID NOs: 118-123.


SEQUENCE LISTING (291 sequences)

SEQ ID NO: 1 - protein, 20 aa, Homo sapiens
MQSGTHWRVLGLCLLSVGVW

SEQ ID NO: 2 - protein, 104 aa, Homo sapiens
DGNEEMGGITQTPYKVSISGTTVILTCPQYPGSEILWQHNDKNIGGDEDDKNIGSDEDHLSLKEFSELEQSGYYVCYPRGSKPEDANFYLYLRARVCENCMEMD

SEQ ID NO: 3 - protein, 26 aa, Homo sapiens
VMSVATIVIVDICITGGLLLLVYYWS

SEQ ID NO: 4 - protein, 55 aa, Homo sapiens
KNRKAKAKPVTRGAGAGGRQRGQNKERPPPVPNPDYEPIRKGQRDLYSGLNQRRI

SEQ ID NO: 5 - DNA, 624 bp, Homo sapiens
  source 1..624: mol_type = genomic DNA
atgcagtcgggcactcactggagagttctgggcctctgcctcttatcagttggcgtttgggggcaagatggtaatgaagaaatgggtggtattacacagacaccatataaagtctccatctctggaaccacagtaatattgacatgccctcagtatcctggatctgaaatactatggcaacacaatgataaaaacataggcggtgatgaggatgataaaaacataggcagtgatgaggatcacctgtcactgaaggaattttcagaattggagcaaagtggttattatgtctgctaccccagaggaagcaaaccagaagatgcgaacttttatctctacctgagggcaagagtgtgtgagaactgcatggagatggatgtgatgtcggtggccacaattgtcatagtggacatctgcatcactgggggcttgctgctgctggtttactactggagcaagaatagaaaggccaaggccaagcctgtgacacgaggagcgggtgctggcggcaggcaaaggggacaaaacaaggagaggccaccacctgttcccaacccagactatgagcccatccggaaaggccagcgggacctgtattctggcctgaatcagagacgcatctga

SEQ ID NO: 6 - protein, 20 aa, Homo sapiens
MEHSTFLSGLVLATLLSQVS

SEQ ID NO: 7 - protein, 84 aa, Homo sapiens [sequence omitted]

SEQ ID NO: 8 - protein, 21 aa, Homo sapiens
GIIVTDVIATLLLALGVFCFA

SEQ ID NO: 9 - protein, 45 aa, Homo sapiens
GHETGRLSGAADTQALLRNDQVYQPLRDRDDAQYSHLGGNWARNK

SEQ ID NO: 10 - DNA, 516 bp, Homo sapiens
  source 1..516: mol_type = genomic DNA
atggaacatagcacgtttctctctggcctggtactggctacccttctctcgcaagtgagccccttcaagatacctatagaggaacttgaggacagagtgtttgtgaattgcaataccagcatcacatgggtagagggaacggtgggaacactgctctcagacattacaagactggacctgggaaaacgcatcctggacccacgaggaatatataggtgtaatgggacagatatatacaaggacaaagaatctaccgtgcaagttcattatcgaatgtgccagagctgtgtggagctggatccagccaccgtggctggcatcattgtcactgatgtcattgccactctgctccttgctttgggagtcttctgctttgctggacatgagactggaaggctgtctggggctgccgacacacaagctctgttgaggaatgaccaggtctatcagcccctccgagatcgagatgatgctcagtacagccaccttggaggaaactgggctcggaacaagtga

SEQ ID NO: 11 - protein, 22 aa, Homo sapiens
MEQGKGLAVLILAIILLQGTLA

SEQ ID NO: 12 - protein, 94 aa, Homo sapiens
QSIKGNHLVKVYDYQEDGSVLLTCDAEAKNITWFKDGKMIGFLTEDKKKWNLGSNAKDPRGMYQCKGSQNKSKPLQVYYRMCQNCIELNAATIS

SEQ ID NO: 13 - protein, 21 aa, Homo sapiens
GFLFAEIVSIFVLAVGVYFIA

SEQ ID NO: 14 - protein, 45 aa, Homo sapiens
GQDGVRQSRASDKQTLLPNDQLYQPLKDREDDQYSHLQGNQLRRN

SEQ ID NO: 15 - DNA, 549 bp, Homo sapiens
  source 1..549: mol_type = genomic DNA
atggaacaggggaagggcctggctgtcctcatcctggctatcattcttcttcaaggtactttggcccagtcaatcaaaggaaaccacttggttaaggtgtatgactatcaagaagatggttcggtacttctgacttgtgatgcagaagccaaaaatatcacatggtttaaagatgggaagatgatcggcttcctaactgaagataaaaaaaaatggaatctgggaagtaatgccaaggaccctcgagggatgtatcagtgtaaaggatcacagaacaagtcaaaaccactccaagtgtattacagaatgtgtcagaactgcattgaactaaatgcagccaccatatctggctttctctttgctgaaatcgtcagcattttcgtccttgctgttggggtctacttcattgctggacaggatggagttcgccagtcgagagcttcagacaagcagactctgttgcccaatgaccagctctaccagcccctcaaggatcgagaagatgaccagtacagccaccttcaaggaaaccagttgaggaggaattga

SEQ ID NO: 16 - protein, 21 aa, Homo sapiens
MKWKALFTAAILQAQLPITEA

SEQ ID NO: 17 - protein, 9 aa, Homo sapiens
QSFGLLDPK

SEQ ID NO: 18 - protein, 21 aa, Homo sapiens
LCYLLDGILFIYGVILTALFL

SEQ ID NO: 19 - protein, 113 aa, Homo sapiens
RVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 20 - DNA, 495 bp, Homo sapiens
  source 1..495: mol_type = genomic DNA
atgaagtggaaggcgcttttcaccgcggccatcctgcaggcacagttgccgattacagaggcacagagctttggcctgctggatcccaaactctgctacctgctggatggaatcctcttcatctatggtgtcattctcactgccttgttcctgagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgcagagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctaa

SEQ ID NO: 21 - protein, 21 aa, synthetic construct
GSGEGRGSLLTCGDVEENPGP

SEQ ID NO: 22 - protein, 22 aa, synthetic construct
GSGATNFSLLKQAGDVEENPGP

SEQ ID NO: 23 - protein, 23 aa, synthetic construct
GSGQCTNYALLKLAGDVESNPGP

SEQ ID NO: 24 - protein, 25 aa, synthetic construct
GSGVKQTLNFDLLKLAGDVESNPGP

SEQ ID NO: 25 - protein, 255 aa, synthetic construct
XQEVTQIPAALSVPEGENLVLNCSFTDSAIYNLQWFRQDPGKGLTSLLLIQSSQREQTSGRLNASLDKSSGRSTLYIAASQPGDSATYLCAVRPLYGGSYIPTFGRGTSLIVHPYIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 26 - protein, 290 aa, synthetic construct
GVTQTPKFQVLKTGQSMTLQCAQDMNHEYMSWYRQDPGMGLRLIHYSVGAGITDQGEVPNGYNVSRSTTEDFPLRLLSAAPSQTSVYFCASSYVGNTGELFFGEGSRLTVLEDLKNVFPPKVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 27 - DNA, 822 bp, synthetic construct
  source 1..822: mol_type = other DNA
atggactcctggaccttctgctgtgtgtccctttgcatcctggtagcaaagcacacagatgctggacaacagctgaatcagagtcctcaatctatgtttatccaggaaggagaagatgtctccatgaactgcacttcttcaagcatatttaacacctggctatggtacaagcaggaccctggggaaggtcctgtcctcttgatagccttatataaggctggtgaattgacctcaaatggaagactgactgctcagtttggtataaccagaaaggacagcttcctgaatatctcagcatccatacccagtgatgtaggcatctacttctgtgctggacccatgaaaacctcctacgacaaggtgatatttgggccagggacaagcttatcagtcattccaaatatccagaaccctgaccctgccgtgtaccagctgagagactctaaatccagtgacaagtctgtctgcctattcaccgattttgattctcaaacaaatgtgtcacaaagtaaggattctgatgtgtatatcacagacaaaactgtgctagacatgaggtctatggacttcaagagcaacagtgctgtggcctggagcaacaaatctgactttgcatgtgcaaacgccttcaacaacagcattattccagaagacaccttcttccccagcccagaaagttcctgtgatgtcaagctggtcgagaaaagctttgaaacagatacgaacctaaactttcaaaacctgtcagtgattgggttccgaatcctcctcctgaaagtggccgggtttaatctgctcatgacgctgcggctgtggtccagc

SEQ ID NO: 28 - protein, 274 aa, synthetic construct
MDSWTFCCVSLCILVAKHTDAGQQLNQSPQSMFIQEGEDVSMNCTSSSIFNTWLWYKQDPGEGPVLLIALYKAGELTSNGRLTAQFGITRKDSFLNISASIPSDVGIYFCAGPMKTSYDKVIFGPGTSLSVIPNIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 29 - DNA, 924 bp, synthetic construct
  source 1..924: mol_type = other DNA
atggactcctggaccttctgctgtgtgtccctttgcatcctggtagcaaagcacacagatgctggagttatccagtcaccccggcacgaggtgacagagatgggacaagaagtgactctgagatgtaaaccaatttcaggacacgactaccttttctggtacagacagaccatgatgcggggactggagttgctcatttactttaacaacaacgttccgatagatgattcagggatgcccgaggatcgattctcagctaagatgcctaatgcatcattctccactctgaagatccagccctcagaacccagggactcagctgtgtacttctgtgccagcagttcggcaaactatggctacaccttcggttcggggaccaggttaaccgttgtagaggacctgaacaaggtgttcccacccgaggtcgctgtgtttgagccatcagaagcagagatctcccacacccaaaaggccacactggtgtgcctggccacaggcttcttccctgaccacgtggagctgagctggtgggtgaatgggaaggaggtgcacagtggggtcagcacggacccgcagcccctcaaggagcagcccgccctcaatgactccagatactgcctgagcagccgcctgagggtctcggccaccttctggcagaacccccgcaaccacttccgctgtcaagtccagttctacgggctctcggagaatgacgagtggacccaggatagggccaaacccgtcacccagatcgtcagcgccgaggcctggggtagagcagactgtggctttacctcggtgtcctaccagcaaggggtcctgtctgccaccatcctctatgagatcctgctagggaaggccaccctgtatgctgtgctggtcagcgcccttgtgttgatggccatggtcaagagaaaggatttc

SEQ ID NO: 30 - protein, 308 aa, synthetic construct [sequence omitted]

SEQ ID NO: 31 - DNA, 492 bp, Homo sapiens
  source 1..492: mol_type = genomic DNA
atgaagtggaaggcgcttttcaccgcggccatcctgcaggcacagttgccgattacagaggcacagagctttggcctgctggatcccaaactctgctacctgctggatggaatcctcttcatctatggtgtcattctcactgccttgttcctgagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgcagagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgc

SEQ ID NO: 32 - protein, 164 aa, Homo sapiens
MKWKALFTAAILQAQLPITEAQSFGLLDPKLCYLLDGILFIYGVILTALFLRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 33 - DNA, 546 bp, Homo sapiens
  source 1..546: mol_type = genomic DNA
atggaacaggggaagggcctggctgtcctcatcctggctatcattcttcttcaaggtactttggcccagtcaatcaaaggaaaccacttggttaaggtgtatgactatcaagaagatggttcggtacttctgacttgtgatgcagaagccaaaaatatcacatggtttaaagatgggaagatgatcggcttcctaactgaagataaaaaaaaatggaatctgggaagtaatgccaaggaccctcgtgggatgtatcagtgtaaaggatcacagaacaagtcaaaaccactccaagtgtattacagaatgtgtcagaactgcattgaactaaatgcagccaccatatctggctttctctttgctgaaatcgtcagcattttcgtccttgctgttggggtctacttcattgctggacaggatggagttcgccagtcgagagcttcagacaagcagactctgttgcccaatgaccagctctaccagcccctcaaggatcgagaagatgaccagtacagccaccttcaaggaaaccagttgaggaggaat

SEQ ID NO: 34 - protein, 182 aa, Homo sapiens
MEQGKGLAVLILAIILLQGTLAQSIKGNHLVKVYDYQEDGSVLLTCDAEAKNITWFKDGKMIGFLTEDKKKWNLGSNAKDPRGMYQCKGSQNKSKPLQVYYRMCQNCIELNAATISGFLFAEIVSIFVLAVGVYFIAGQDGVRQSRASDKQTLLPNDQLYQPLKDREDDQYSHLQGNQLRRN

SEQ ID NO: 35 - DNA, 513 bp, Homo sapiens
  source 1..513: mol_type = genomic DNA
atggagcacagcaccttcctgagcggcctggtgctggccaccctgctgagccaggtgagccccttcaagatccccatcgaggagctggaggacagagtgttcgtgaactgcaacaccagcatcacctgggtggagggcaccgtgggcaccctgctgagcgacatcaccagactggacctgggcaagagaatcctggaccccagaggcatctacagatgcaacggcaccgacatctacaaggacaaggagagcaccgtgcaggtgcactacagaatgtgccagagctgcgtggagctggaccccgccaccgtggccggcatcatcgtgaccgacgtgatcgccaccctgctgctggccctgggcgtgttctgcttcgccggccacgagaccggcagactgagcggcgccgccgacacccaggccctgctgagaaacgaccaggtgtaccagcccctgagagacagagacgacgcccagtacagccacctgggcggcaactgggccagaaacaag

SEQ ID NO: 36 - protein, 171 aa, Homo sapiens
MEHSTFLSGLVLATLLSQVSPFKIPIEELEDRVFVNCNTSITWVEGTVGTLLSDITRLDLGKRILDPRGIYRCNGTDIYKDKESTVQVHYRMCQSCVELDPATVAGIIVTDVIATLLLALGVFCFAGHETGRLSGAADTQALLRNDQVYQPLRDRDDAQYSHLGGNWARNK

SEQ ID NO: 37 - DNA, 621 bp, synthetic construct
  source 1..621: mol_type = other DNA
atgcagagcggcacccactggagagtgctgggcctgtgcctgctgagcgtgggcgtgtggggccaggacggcaacgaggagatgggcggcatcacccagaccccctacaaggtgagcatcagcggcaccaccgtgatcctgacctgcccccagtaccccggcagcgagatcctgtggcagcacaacgacaagaacatcggcggcgacgaggacgacaagaacatcggcagcgacgaggaccacctgagcctgaaggagttcagcgagctggagcagagcggctactacgtgtgctaccccagaggcagcaagcccgaggacgccaacttctacctgtacctgagagccagagtgtgcgagaactgcatggagatggacgtgatgagcgtggccaccatcgtgatcgtggacatctgcatcaccggcggcctgctgctgctggtgtactactggagcaagaacagaaaggccaaggccaagcccgtgaccagaggcgccggcgccggcggcagacagagaggccagaacaaggagagacccccccccgtgcccaaccccgactacgagcccatcagaaagggccagagagacctgtacagcggcctgaaccagagaagaatc

SEQ ID NO: 38 - protein, 207 aa, Homo sapiens
MQSGTHWRVLGLCLLSVGVWGQDGNEEMGGITQTPYKVSISGTTVILTCPQYPGSEILWQHNDKNIGGDEDDKNIGSDEDHLSLKEFSELEQSGYYVCYPRGSKPEDANFYLYLRARVCENCMEMDVMSVATIVIVDICITGGLLLLVYYWSKNRKAKAKPVTRGAGAGGRQRGQNKERPPPVPNPDYEPIRKGQRDLYSGLNQRRI

SEQ ID NO: 39 - protein, 1499 aa, Homo sapiens
MLEGVTQTPKFQVLKTGQSMTLQCAQDMNHEYMSWYRQDPGMGLRLIHYSVGAGITDQGEVPNGYNVSRSTTEDFPLRLLSAAPSQTSVYFCASSPVTGGIYGYTFGSGTRLTVVEDLNKVFPPEVAVFEPSEAEISHTQKATLVCLATGFFPDHVELSWWVNGKEVHSGVSTDPQPLKEQPALNDSRYCLSSRLRVSATFWQNPRNHFRCQVQFYGLSENDEWTQDRAKPVTQIVSAEAWGRADRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPRATNFSLLKQAGDVEENPGPMILNVEQSPQSLHVQEGDSTNFTCSFPSSNFYALHWYRWETAKSPEALFVMTLNGDEKKKGRISATLNTKEGYSYLYIKGSQPEDSATYLCARNTGNQFYFGTGTSLTVIPNIQNPDPAVYQLRDSKSSDKSVCLFTDFDSQTNVSQSKDSDAYITDKTVLDMRSMDFKSNSAVAWSNKSDFACANAFNNSIIPEDTFFPSPESSRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPRQCTNYALLKLAGDVESNPGPMEQGKGLAVLILAIILLQGTLAQSIKGNHLVKVYDYQEDGSVLLTCDAEAKNITWFKDGKMIGFLTEDKKKWNLGSNAKDPRGMYQCKGSQNKSKPLQVYYRMCQNCIELNAATISGFLFAEIVSIFVLAVGVYFIAGQDGVRQSRASDKQTLLPNDQLYQPLKDREDDQYSHLQGNQLRRNVKQTLNFDLLKLAGDVESNPGPMEHSTFLSGLVLATLLSQVSPFKIPIEELEDRVFVNCNTSITWVEGTVGTLLSDITRLDLGKRILDPRGIYRCNGTDIYKDKESTVQVHYRMCQSCVELDPATVAGIIVTDVIATLLLALGVFCFAGHETGRLSGAADTQALLRNDQVYQPLRDRDDAQYSHLGGNWARNKEGRGSLLTCGDVEENPGPMQSGTHWRVLGLCLLSVGVWGQDGNEEMGGITQTPYKVSISGTTVILTCPQYPGSEILWQHNDKNIGGDEDDKNIGSDEDHLSLKEFSELEQSGYYVCYPRGSKPEDANFYLYLRARVCENCMEMDVMSVATIVIVDICITGGLLLLVYYWSKNRKAKAKPVTRGAGAGGRQRGQNKERPPPVPNPDYEPIRKGQRDLYSGLNQRRIGPQCTNYALLKLAGDVESNPGPMRISKPHLRSISIQCYLCLLLNSHFLTEAGIHVFILGCFSAGLPKTEANWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 40 - protein, 245 aa, synthetic construct
MLEGVTQTPKFQVLKTGQSMTLQCAQDMNHEYMSWYRQDPGMGLRLIHYSVGAGITDQGEVPNGYNVSRSTTEDFPLRLLSAAPSQTSVYFCASSPVTGGIYGYTFGSGTRLTVVEDLNKVFPPEVAVFEPSEAEISHTQKATLVCLATGFFPDHVELSWWVNGKEVHSGVSTDPQPLKEQPALNDSRYCLSSRLRVSATFWQNPRNHFRCQVQFYGLSENDEWTQDRAKPVTQIVSAEAWGRAD

SEQ ID NO: 41 - DNA, 735 bp, synthetic construct
  source 1..735: mol_type = other DNA
atgctcgagggagtgacccagacccccaagttccaggtgctgaagaccggacagagcatgaccctgcagtgcgcccaggacatgaaccacgagtacatgagctggtaccggcaggaccccggaatgggactgcggctgatccactacagcgtgggagccggaatcaccgaccagggagaggtgcccaacggatacaacgtgagccggagcaccaccgaggacttccccctgcggctgctgagcgccgcccccagccagaccagcgtgtacttctgcgccagcagccccgtgaccggaggaatctacggatacaccttcggaagcggaacccggctgaccgtggtggaggacctgaacaaggtgttcccccccgaggtggccgtgttcgagcccagcgaggccgagatcagccacacccagaaggccaccctggtgtgcctggccaccggattcttccccgaccacgtggagctgagctggtgggtgaacggaaaggaggtgcacagcggagtgagcaccgacccccagcccctgaaggagcagcccgccctgaacgacagccggtactgcctgagcagccggctgcgggtgagcgccaccttctggcagaacccccggaaccacttccggtgccaggtgcagttctacggactgagcgagaacgacgagtggacccaggaccgggccaagcccgtgacccagatcgtgagcgccgaggcctggggacgggccgac

SEQ ID NO: 42 - protein, 132 aa, synthetic construct
RVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPRATNFSLLKQAGDVEENPGP

SEQ ID NO: 43 - DNA, 396 bp, synthetic construct
  source 1..396: mol_type = other DNA
agagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgcagagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgcgccaccaacttctccctgctgaagcaggccggcgacgtggaggagaaccccggcccc

SEQ ID NO: 44 - protein, 205 aa, synthetic construct
MILNVEQSPQSLHVQEGDSTNFTCSFPSSNFYALHWYRWETAKSPEALFVMTLNGDEKKKGRISATLNTKEGYSYLYIKGSQPEDSATYLCARNTGNQFYFGTGTSLTVIPNIQNPDPAVYQLRDSKSSDKSVCLFTDFDSQTNVSQSKDSDAYITDKTVLDMRSMDFKSNSAVAWSNKSDFACANAFNNSIIPEDTFFPSPESS

SEQ ID NO: 45 - DNA, 615 bp, synthetic construct
  source 1..615: mol_type = other DNA
atgatcctgaacgtggagcagagcccccagagcctgcacgtgcaggagggagacagcaccaacttcacctgcagcttccccagcagcaacttctacgccctgcactggtaccggtgggagaccgccaagagccccgaggccctgttcgtgatgaccctgaacggagacgagaagaagaagggacggatcagcgccaccctgaacaccaaggagggatacagctacctgtacatcaagggaagccagcccgaggacagcgccacctacctgtgcgcccggaacaccggaaaccagttctacttcggaaccggaaccagcctgaccgtgatccccaacatccagaaccccgaccccgccgtgtaccagctgcgggacagcaagagcagcgacaagagcgtgtgcctgttcaccgacttcgacagccagaccaacgtgagccagagcaaggacagcgacgcctacatcaccgacaagaccgtgctggacatgcggagcatggacttcaagagcaacagcgccgtggcctggagcaacaagagcgacttcgcctgcgccaacgccttcaacaacagcatcatccccgaggacaccttcttccccagccccgagagcagc

SEQ ID NO: 46 - protein, 622 aa, synthetic construct
MEQGKGLAVLILAIILLQGTLAQSIKGNHLVKVYDYQEDGSVLLTCDAEAKNITWFKDGKMIGFLTEDKKKWNLGSNAKDPRGMYQCKGSQNKSKPLQVYYRMCQNCIELNAATISGFLFAEIVSIFVLAVGVYFIAGQDGVRQSRASDKQTLLPNDQLYQPLKDREDDQYSHLQGNQLRRNVKQTLNFDLLKLAGDVESNPGPMEHSTFLSGLVLATLLSQVSPFKIPIEELEDRVFVNCNTSITWVEGTVGTLLSDITRLDLGKRILDPRGIYRCNGTDIYKDKESTVQVHYRMCQSCVELDPATVAGIIVTDVIATLLLALGVFCFAGHETGRLSGAADTQALLRNDQVYQPLRDRDDAQYSHLGGNWARNKEGRGSLLTCGDVEENPGPMQSGTHWRVLGLCLLSVGVWGQDGNEEMGGITQTPYKVSISGTTVILTCPQYPGSEILWQHNDKNIGGDEDDKNIGSDEDHLSLKEFSELEQSGYYVCYPRGSKPEDANFYLYLRARVCENCMEMDVMSVATIVIVDICITGGLLLLVYYWSKNRKAKAKPVTRGAGAGGRQRGQNKERPPPVPNPDYEPIRKGQRDLYSGLNQRRIGPQCTNYALLKLAGDVESNPGP

SEQ ID NO: 47 - DNA, 1866 bp, synthetic construct
  source 1..1866: mol_type = other DNA
atggaacaggggaagggcctggctgtcctcatcctggctatcattcttcttcaaggtactttggcccagtcaatcaaaggaaaccacttggttaaggtgtatgactatcaagaagatggttcggtacttctgacttgtgatgcagaagccaaaaatatcacatggtttaaagatgggaagatgatcggcttcctaactgaagataaaaaaaaatggaatctgggaagtaatgccaaggaccctcgtgggatgtatcagtgtaaaggatcacagaacaagtcaaaaccactccaagtgtattacagaatgtgtcagaactgcattgaactaaatgcagccaccatatctggctttctctttgctgaaatcgtcagcattttcgtccttgctgttggggtctacttcattgctggacaggatggagttcgccagtcgagagcttcagacaagcagactctgttgcccaatgaccagctctaccagcccctcaaggatcgagaagatgaccagtacagccaccttcaaggaaaccagttgaggaggaatgtgaagcagaccctgaacttcgacctgctgaagctggccggcgacgtggagagcaaccccggccccatggagcacagcaccttcctgagcggcctggtgctggccaccctgctgagccaggtgagccccttcaagatccccatcgaggagctggaggacagagtgttcgtgaactgcaacaccagcatcacctgggtggagggcaccgtgggcaccctgctgagcgacatcaccagactggacctgggcaagagaatcctggaccccagaggcatctacagatgcaacggcaccgacatctacaaggacaaggagagcaccgtgcaggtgcactacagaatgtgccagagctgcgtggagctggaccccgccaccgtggccggcatcatcgtgaccgacgtgatcgccaccctgctgctggccctgggcgtgttctgcttcgccggccacgagaccggcagactgagcggcgccgccgacacccaggccctgctgagaaacgaccaggtgtaccagcccctgagagacagagacgacgcccagtacagccacctgggcggcaactgggccagaaacaaggagggcagaggcagcctgctgacctgcggcgacgtggaggagaaccccggccccatgcagagcggcacccactggagagtgctgggcctgtgcctgctgagcgtgggcgtgtggggccaggacggcaacgaggagatgggcggcatcacccagaccccctacaaggtgagcatcagcggcaccaccgtgatcctgacctgcccccagtaccccggcagcgagatcctgtggcagcacaacgacaagaacatcggcggcgacgaggacgacaagaacatcggcagcgacgaggaccacctgagcctgaaggagttcagcgagctggagcagagcggctactacgtgtgctaccccagaggcagcaagcccgaggacgccaacttctacctgtacctgagagccagagtgtgcgagaactgcatggagatggacgtgatgagcgtggccaccatcgtgatcgtggacatctgcatcaccggcggcctgctgctgctggtgtactactggagcaagaacagaaaggccaaggccaagcccgtgaccagaggcgccggcgccggcggcagacagagaggccagaacaaggagagacccccccccgtgcccaaccccgactacgagcccatcagaaagggccagagagacctgtacagcggcctgaaccagagaagaatcggaccgcagtgtactaattatgctctcttgaaattggctggagatgttgagagcaatcccgggccc

SEQ ID NO: 48 - protein, 162 aa, synthetic construct
MRISKPHLRSISIQCYLCLLLNSHFLTEAGIHVFILGCFSAGLPKTEANWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 49 - DNA, 486 bp, synthetic construct
  source 1..486: mol_type = other DNA
atgcgcattagcaagccccacctgcggagcatcagcatccagtgctacctgtgcctgctgctgaacagccacttcctgaccgaggccggcatccacgtgttcatcctgggctgcttcagcgccggactgcccaagaccgaggccaactgggtgaacgtgatcagcgacctgaagaagatcgaggacctgatccagagcatgcacatcgacgccaccctgtacaccgagagcgacgtgcaccccagctgcaaggtgaccgccatgaagtgctttctgctggaactgcaggtgatcagcctggaaagcggcgacgccagcatccacgacaccgtggagaacctgatcatcctggccaacaacagcctgagcagcaacggcaacgtgaccgagagcggctgcaaagagtgcgaggaactggaagagaagaacatcaaagagtttctgcagagcttcgtgcacatcgtgcagatgttcatcaacaccagc

SEQ ID NO: 50 - DNA, 3816 bp, synthetic construct
  source 1..3816: mol_type = other DNA
ctcgagggagtgacccagacccccaagttccaggtgctgaagaccggacagagcatgaccctgcagtgcgcccaggacatgaaccacgagtacatgagctggtaccggcaggaccccggaatgggactgcggctgatccactacagcgtgggagccggaatcaccgaccagggagaggtgcccaacggatacaacgtgagccggagcaccaccgaggacttccccctgcggctgctgagcgccgcccccagccagaccagcgtgtacttctgcgccagcagccccgtgaccggaggaatctacggatacaccttcggaagcggaacccggctgaccgtggtggaggacctgaacaaggtgttcccccccgaggtggccgtgttcgagcccagcgaggccgagatcagccacacccagaaggccaccctggtgtgcctggccaccggattcttccccgaccacgtggagctgagctggtgggtgaacggaaaggaggtgcacagcggagtgagcaccgacccccagcccctgaaggagcagcccgccctgaacgacagccggtactgcctgagcagccggctgcgggtgagcgccaccttctggcagaacccccggaaccacttccggtgccaggtgcagttctacggactgagcgagaacgacgagtggacccaggaccgggccaagcccgtgacccagatcgtgagcgccgaggcctggggacgggccgacgccaccaacttcagcctgctgaagcaggccggcgacgtggaggagaaccccggccccatgatcctgaacgtggagcagagcccccagagcctgcacgtgcaggagggagacagcaccaacttcacctgcagcttccccagcagcaacttctacgccctgcactggtaccggtgggagaccgccaagagccccgaggccctgttcgtgatgaccctgaacggagacgagaagaagaagggacggatcagcgccaccctgaacaccaaggagggatacagctacctgtacatcaagggaagccagcccgaggacagcgccacctacctgtgcgcccggaacaccggaaaccagttctacttcggaaccggaaccagcctgaccgtgatccccaacatccagaaccccgaccccgccgtgtaccagctgcgggacagcaagagcagcgacaagagcgtgtgcctgttcaccgacttcgacagccagaccaacgtgagccagagcaaggacagcgacgcctacatcaccgacaagaccgtgctggacatgcggagcatggacttcaagagcaacagcgccgtggcctggagcaacaagagcgacttcgcctgcgccaacgccttcaacaacagcatcatccccgaggacaccttcttccccagccccgagagcagcgagggcagaggcagcctgctgacctgcggcgacgtggaggagaaccccggccccatgaagtggaaggcgcttttcaccgcggccatcctgcaggcacagttgccgattacagaggcacagagctttggcctgctggatcccaaactctgctacctgctggatggaatcctcttcatctatggtgtcattctcactgccttgttcctgagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgcagagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgccagtgcaccaactacgccctgctgaagctggccggcgacgtggagagcaaccccggccccatggaacaggggaagggcctggctgtcctcatcctggctatcattcttcttcaaggtactttggcccagtcaatcaaaggaaaccacttggttaaggtgtatgactatcaagaagatggttcggtacttctgacttgtgatgcagaagccaaaaatatcacatggtttaaagatgggaagatgatcggcttcctaactgaagataaaaaaaaatggaatctgggaagtaatgccaaggaccctcgtgggatgtatcagtgtaaaggatcacagaacaagtcaaaaccactccaagtgtattacagaatgtgtcagaactgcattgaactaaatgcagccaccatatctggctttctctttgctgaaatcgtcagcattttcgtccttgctgttggggtctacttcattgctggacaggatggagttcgccagtcgagagcttcagacaagcagactctgttgcccaatgaccagctctaccagcccctcaaggatcgagaagatgaccagtacagccaccttcaaggaaaccagttgaggaggaatgtgaagcagaccctgaacttcgacctgctgaagctggccggcgacgtggagagcaaccccggccccatggagcacagcaccttcctgagcggcctggtgctggccaccctgctgagccaggtgagccccttcaagatccccatcgaggagctggaggacagagtgttcgtgaactgcaacaccagcatcacctgggtggagggcaccgtgggcaccctgctgagcgacatcaccagactggacctgggcaagagaatcctggaccccagaggcatctacagatgcaacggcaccgacatctacaaggacaaggagagcaccgtgcaggtgcactacagaatgtgccagagctgcgtggagctggaccccgccaccgtggccggcatcatcgtgaccgacgtgatcgccaccctgctgctggccctgggcgtgttctgcttcgccggccacgagaccggcagactgagcggcgccgccgacacccaggccctgctgagaaacgaccaggtgtaccagcccctgagagacagagacgacgcccagtacagccacctgggcggcaactgggccagaaacaaggagggcagaggcagcctgctgacctgcggcgacgtggaggagaaccccggccccatgcagagcggcacccactggagagtgctgggcctgtgcctgctgagcgtgggcgtgtggggccaggacggcaacgaggagatgggcggcatcacccagaccccctacaaggtgagcatcagcggcaccaccgtgatcctgacctgcccccagtaccccggcagcgagatcctgtggcagcacaacgacaagaacatcggcggcgacgaggacgacaagaacatcggcagcgacgaggaccacctgagcctgaaggagttcagcgagctggagcagagcggctactacgtgtgctaccccagaggcagcaagcccgaggacgccaacttctacctgtacctgagagccagagtgtgcgagaactgcatggagatggacgtgatgagcgtggccaccatcgtgatcgtggacatctgcatcaccggcggcctgctgctgctggtgtactactggagcaagaacagaaaggccaaggccaagcccgtgaccagaggcgccggcgccggcggcagacagagaggccagaacaaggagagacccccccccgtgcccaaccccgactacgagcccatcagaaagggccagagagacctgtacagcggcctgaaccagagaagaatcggaccg

SEQ ID NO: 51 - protein, 1272 aa, synthetic construct
LEGVTQTPKFQVLKTGQSMTLQCAQDMNHEYMSWYRQDPGMGLRLIHYSVGAGITDQGEVPNGYNVSRSTTEDFPLRLLSAAPSQTSVYFCASSPVTGGIYGYTFGSGTRLTVVEDLNKVFPPEVAVFEPSEAEISHTQKATLVCLATGFFPDHVELSWWVNGKEVHSGVSTDPQPLKEQPALNDSRYCLSSRLRVSATFWQNPRNHFRCQVQFYGLSENDEWTQDRAKPVTQIVSAEAWGRADATNFSLLKQAGDVEENPGPMILNVEQSPQSLHVQEGDSTNFTCSFPSSNFYALHWYRWETAKSPEALFVMTLNGDEKKKGRISATLNTKEGYSYLYIKGSQPEDSATYLCARNTGNQFYFGTGTSLTVIPNIQNPDPAVYQLRDSKSSDKSVCLFTDFDSQTNVSQSKDSDAYITDKTVLDMRSMDFKSNSAVAWSNKSDFACANAFNNSIIPEDTFFPSPESSEGRGSLLTCGDVEENPGPMKWKALFTAAILQAQLPITEAQSFGLLDPKLCYLLDGILFIYGVILTALFLRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPRQCTNYALLKLAGDVESNPGPMEQGKGLAVLILAIILLQGTLAQSIKGNHLVKVYDYQEDGSVLLTCDAEAKNITWFKDGKMIGFLTEDKKKWNLGSNAKDPRGMYQCKGSQNKSKPLQVYYRMCQNCIELNAATISGFLFAEIVSIFVLAVGVYFIAGQDGVRQSRASDKQTLLPNDQLYQPLKDREDDQYSHLQGNQLRRNVKQTLNFDLLKLAGDVESNPGPMEHSTFLSGLVLATLLSQVSPFKIPIEELEDRVFVNCNTSITWVEGTVGTLLSDITRLDLGKRILDPRGIYRCNGTDIYKDKESTVQVHYRMCQSCVELDPATVAGIIVTDVIATLLLALGVFCFAGHETGRLSGAADTQALLRNDQVYQPLRDRDDAQYSHLGGNWARNKEGRGSLLTCGDVEENPGPMQSGTHWRVLGLCLLSVGVWGQDGNEEMGGITQTPYKVSISGTTVILTCPQYPGSEILWQHNDKNIGGDEDDKNIGSDEDHLSLKEFSELEQSGYYVCYPRGSKPEDANFYLYLRARVCENCMEMDVMSVATIVIVDICITGGLLLLVYYWSKNRKAKAKPVTRGAGAGGRQRGQNKERPPPVPNPDYEPIRKGQRDLYSGLNQRRIGP

SEQ ID NO: 52 - protein, 880 aa, synthetic construct
MLEGVTQTPKFQVLKTGQSMTLQCAQDMNHEYMSWYRQDPGMGLRLIHYSVGAGITDQGEVPNGYNVSRSTTEDFPLRLLSAAPSQTSVYFCASSPVTGGIYGYTFGSGTRLTVVEDLNKVFPPEVAVFEPSEAEISHTQKATLVCLATGFFPDHVELSWWVNGKEVHSGVSTDPQPLKEQPALNDSRYCLSSRLRVSATFWQNPRNHFRCQVQFYGLSENDEWTQDRAKPVTQIVSAEAWGRADRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPRATNFSLLKQAGDVEENPGPMILNVEQSPQSLHVQEGDSTNFTCSFPSSNFYALHWYRWETAKSPEALFVMTLNGDEKKKGRISATLNTKEGYSYLYIKGSQPEDSATYLCARNTGNQFYFGTGTSLTVIPNIQNPDPAVYQLRDSKSSDKSVCLFTDFDSQTNVSQSKDSDAYITDKTVLDMRSMDFKSNSAVAWSNKSDFACANAFNNSIIPEDTFFPSPESSRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPRPGPQCTNYALLKLAGDVESNPGPMRISKPHLRSISIQCYLCLLLNSHFLTEAGIHVFILGCFSAGLPKTEANWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 53 - protein, 136 aa, synthetic construct
RVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPRPGPQCTNYALLKLAGDVESNPGP

SEQ ID NO: 54 - DNA, 399 bp, synthetic construct
  source 1..399: mol_type = other DNA
agagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgcagagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgccagtgcaccaactacgccctgctgaagctggccggcgacgtggagagcaaccccggcccc

SEQ ID NO: 55 - protein, 880 aa, synthetic construct; identical to SEQ ID NO: 52

SEQ ID NO: 56 - DNA, 1794 bp, synthetic construct
  source 1..1794: mol_type = other DNA
atggactcctggaccttctgctgtgtgtccctttgcatcctggtagcaaagcacacagatgctggagttatccagtcaccccggcacgaggtgacagagatgggacaagaagtgactctgagatgtaaaccaatttcaggacacgactaccttttctggtacagacagaccatgatgcggggactggagttgctcatttactttaacaacaacgttccgatagatgattcagggatgcccgaggatcgattctcagctaagatgcctaatgcatcattctccactctgaagatccagccctcagaacccagggactcagctgtgtacttctgtgccagcagttcggcaaactatggctacaccttcggttcggggaccaggttaaccgttgtagaggacctgaacaaggtgttcccacccgaggtcgctgtgtttgagccatcagaagcagagatctcccacacccaaaaggccacactggtgtgcctggccacaggcttcttccctgaccacgtggagctgagctggtgggtgaatgggaaggaggtgcacagtggggtcagcacggacccgcagcccctcaaggagcagcccgccctcaatgactccagatactgcctgagcagccgcctgagggtctcggccaccttctggcagaacccccgcaaccacttccgctgtcaagtccagttctacgggctctcggagaatgacgagtggacccaggatagggccaaacccgtcacccagatcgtcagcgccgaggcctggggtagagcagactgtggctttacctcggtgtcctaccagcaaggggtcctgtctgccaccatcctctatgagatcctgctagggaaggccaccctgtatgctgtgctggtcagcgcccttgtgttgatggccatggtcaagagaaaggatttcgagggcaggggaagtcttctaacatgcggggacgtggaggaaaatcccgggcccatgctccttgaacatttattaataatcttgtggatgcagctgacatgggtcagtggtcaacagctgaatcagagtcctcaatctatgtttatccaggaaggagaagatgtctccatgaactgcacttcttcaagcatatttaacacctggctatggtacaagcaggaccctggggaaggtcctgtcctcttgatagccttatataaggctggtgaattgacctcaaatggaagactgactgctcagtttggtataaccagaaaggacagcttcctgaatatctcagcatccatacccagtgatgtaggcatctacttctgtgctggacccatgaaaacctcctacgacaaggtgatatttgggccagggacaagcttatcagtcattccaaatatccagaaccctgaccctgccgtgtaccagctgagagactctaaatccagtgacaagtctgtctgcctattcaccgattttgattctcaaacaaatgtgtcacaaagtaaggattctgatgtgtatatcacagacaaaactgtgctagacatgaggtctatggacttcaagagcaacagtgctgtggcctggagcaacaaatctgactttgcatgtgcaaacgccttcaacaacagcattattccagaagacaccttcttccccagcccagaaagttcctgtgatgtcaagctggtcgagaaaagctttgaaacagatacgaacctaaactttcaaaacctgtcagtgattgggttccgaatcctcctcctgaaagtggccgggtttaatctgctcatgacgctgcggctgtggtccagctga

SEQ ID NO: 57 - protein, 597 aa, synthetic construct
MDSWTFCCVSLCILVAKHTDAGVIQSPRHEVTEMGQEVTLRCKPISGHDYLFWYRQTMMRGLELLIYFNNNVPIDDSGMPEDRFSAKMPNASFSTLKIQPSEPRDSAVYFCASSSANYGYTFGSGTRLTVVEDLNKVFPPEVAVFEPSEAEISHTQKATLVCLATGFFPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDFEGRGSLLTCGDVEENPGPMLLEHLLIILWMQLTWVSGQQLNQSPQSMFIQEGEDVSMNCTSSSIFNTWLWYKQDPGEGPVLLIALYKAGELTSNGRLTAQFGITRKDSFLNISASIPSDVGIYFCAGPMKTSYDKVIFGPGTSLSVIPNIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 58 - protein, 971 aa, synthetic construct
MLEMKWKALFTAAILQAQLPITEAQSFGLLDPKLCYLLDGILFIYGVILTALFLRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPRQCTNYALLKLAGDVESNPGPMEQGKGLAVLILAIILLQGTLAQSIKGNHLVKVYDYQEDGSVLLTCDAEAKNITWFKDGKMIGFLTEDKKKWNLGSNAKDPRGMYQCKGSQNKSKPLQVYYRMCQNCIELNAATISGFLFAEIVSIFVLAVGVYFIAGQDGVRQSRASDKQTLLPNDQLYQPLKDREDDQYSHLQGNQLRRNVKQTLNFDLLKLAGDVESNPGPMEHSTFLSGLVLATLLSQVSPFKIPIEELEDRVFVNCNTSITWVEGTVGTLLSDITRLDLGKRILDPRGIYRCNGTDIYKDKESTVQVHYRMCQSCVELDPATVAGIIVTDVIATLLLALGVFCFAGHETGRLSGAADTQALLRNDQVYQPLRDRDDAQYSHLGGNWARNKEGRGSLLTCGDVEENPGPMQSGTHWRVLGLCLLSVGVWGQDGNEEMGGITQTPYKVSISGTTVILTCPQYPGSEILWQHNDKNIGGDEDDKNIGSDEDHLSLKEFSELEQSGYYVCYPRGSKPEDANFYLYLRARVCENCMEMDVMSVATIVIVDICITGGLLLLVYYWSKNRKAKAKPVTRGAGAGGRQRGQNKERPPPVPNPDYEPIRKGQRDLYSGLNQRRIGPQCTNYALLKLAGDVESNPGPMRISKPHLRSISIQCYLCLLLNSHFLTEAGIHVFILGCFSAGLPKTEANWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 59 - DNA, 2913 bp, synthetic construct
  source 1..2913: mol_type = other DNA
atgctcgagatgaagtggaaggcgcttttcaccgcggccatcctgcaggcacagttgccgattacagaggcacagagctttggcctgctggatcccaaactctgctacctgctggatggaatcctcttcatctatggtgtcattctcactgccttgttcctgagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgcagagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgccagtgcaccaactacgccctgctgaagctggccggcgacgtggagagcaaccccggccccatggaacaggggaagggcctggctgtcctcatcctggctatcattcttcttcaaggtactttggcccagtcaatcaaaggaaaccacttggttaaggtgtatgactatcaagaagatggttcggtacttctgacttgtgatgcagaagccaaaaatatcacatggtttaaagatgggaagatgatcggcttcctaactgaagataaaaaaaaatggaatctgggaagtaatgccaaggaccctcgtgggatgtatcagtgtaaaggatcacagaacaagtcaaaaccactccaagtgtattacagaatgtgtcagaactgcattgaactaaatgcagccaccatatctggctttctctttgctgaaatcgtcagcattttcgtccttgctgttggggtctacttcattgctggacaggatggagttcgccagtcgagagcttcagacaagcagactctgttgcccaatgaccagctctaccagcccctcaaggatcgagaagatgaccagtacagccaccttcaaggaaaccagttgaggaggaatgtgaagcagaccctgaacttcgacctgctgaagctggccggcgacgtggagagcaaccccggccccatggagcacagcaccttcctgagcggcctggtgctggccaccctgctgagccaggtgagccccttcaagatccccatcgaggagctggaggacagagtgttcgtgaactgcaacaccagcatcacctgggtggagggcaccgtgggcaccctgctgagcgacatcaccagactggacctgggcaagagaatcctggaccccagaggcatctacagatgcaacggcaccgacatctacaaggacaaggagagcaccgtgcaggtgcactacagaatgtgccagagctgcgtggagctggaccccgccaccgtggccggcatcatcgtgaccgacgtgatcgccaccctgctgctggccctgggcgtgttctgcttcgccggccacgagaccggcagactgagcggcgccgccgacacccaggccctgctgagaaacgaccaggtgtaccagcccctgagagacagagacgacgcccagtacagccacctgggcggcaactgggccagaaacaaggagggcagaggcagcctgctgacctgcggcgacgtggaggagaaccccggccccatgcagagcggcacccactggagagtgctgggcctgtgcctgctgagcgtgggcgtgtggggccaggacggcaacgaggagatgggcggcatcacccagaccccctacaaggtgagcatcagcggcaccaccgtgatcctgacctgcccccagtaccccggcagcgagatcctgtggcagcacaacgacaagaacatcggcggcgacgaggacgacaagaacatcggcagcgacgaggaccacctgagcctgaaggagttcagcgagctggagcagagcggctactacgtgtgctaccccagaggcagcaagcccgaggacgccaacttctacctgtacctgagagccagagtgtgcgagaactgcatggagatggacgtgatgagcgtggccaccatcgtgatcgtggacatctgcatcaccggcggcctgctgctgctggtgtactactggagcaagaacagaaaggccaaggccaagcccgtgaccagaggcgccggcgccggcggcagacagagaggccagaacaaggagagacccccccccgtgcccaaccccgactacgagcccatcagaaagggccagagagacctgtacagcggcctgaaccagagaagaatcggaccgcagtgtactaattatgctctcttgaaattggctggagatgttgagagcaatcccgggcccatgcgcattagcaagccccacctgcggagcatcagcatccagtgctacctgtgcctgctgctgaacagccacttcctgaccgaggccggcatccacgtgttcatcctgggctgcttcagcgccggactgcccaagaccgaggccaactgggtgaacgtgatcagcgacctgaagaagatcgaggacctgatccagagcatgcacatcgacgccaccctgtacaccgagagcgacgtgcaccccagctgcaaggtgaccgccatgaagtgctttctgctggaactgcaggtgatcagcctggaaagcggcgacgccagcatccacgacaccgtggagaacctgatcatcctggccaacaacagcctgagcagcaacggcaacgtgaccgagagcggctgcaaagagtgcgaggaactggaagagaagaacatcaaagagtttctgcagagcttcgtgcacatcgtgcagatgttcatcaacaccagc

SEQ ID NO: 60 - protein, 1043 aa, synthetic construct
MLEMKWKALFTAAILQAQLPITEAQSFGLLDPKLCYLLDGILFIYGVILTALFLRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPRQCTNYALLKLAGDVESNPGPMEQGKGLAVLILAIILLQGTLAQSIKGNHLVKVYDYQEDGSVLLTCDAEAKNITWFKDGKMIGFLTEDKKKWNLGSNAKDPRGMYQCKGSQNKSKPLQVYYRMCQNCIELNAATISGFLFAEIVSIFVLAVGVYFIAGQDGVRQSRASDKQTLLPNDQLYQPLKDREDDQYSHLQGNQLRRNVKQTLNFDLLKLAGDVESNPGPMEHSTFLSGLVLATLLSQVSPFKIPIEELEDRVFVNCNTSITWVEGTVGTLLSDITRLDLGKRILDPRGIYRCNGTDIYKDKESTVQVHYRMCQSCVELDPATVAGIIVTDVIATLLLALGVFCFAGHETGRLSGAADTQALLRNDQVYQPLRDRDDAQYSHLGGNWARNKEGRGSLLTCGDVEENPGPMQSGTHWRVLGLCLLSVGVWGQDGNEEMGGITQTPYKVSISGTTVILTCPQYPGSEILWQHNDKNIGGDEDDKNIGSDEDHLSLKEFSELEQSGYYVCYPRGSKPEDANFYLYLRARVCENCMEMDVMSVATIVIVDICITGGLLLLVYYWSKNRKAKAKPVTRGAGAGGRQRGQNKERPPPVPNPDYEPIRKGQRDLYSGLNQRRIGPQCTNYALLKLAGDVESNPGPMRICLTSDRLAPAAGLAAPRRQAVHKSSSQGQDRHMIRMRQLIDIVDQLKNYVNDLVPEFLPAPEDVETNCEWSAFSCFQKAQLKSANTGNNERIINVSIKKLKRKPPSTNAGRRQKHRLTCPSCDSYEKKPPKEFLERFKSLLQKMIHQHLSSRTHGSEDSTTTPAPRPPTPAPTIASQPLSLRPEACRPAAGGAVHTRGLDFACDFWVLVVVGGVLACYSLLVTVAFIIFWV

SEQ ID NO: 61 - protein, 809 aa, synthetic construct
MLEMKWKALFTAAILQAQLPITEAQSFGLLDPKLCYLLDGILFIYGVILTALFLRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPRQCTNYALLKLAGDVESNPGPMEQGKGLAVLILAIILLQGTLAQSIKGNHLVKVYDYQEDGSVLLTCDAEAKNITWFKDGKMIGFLTEDKKKWNLGSNAKDPRGMYQCKGSQNKSKPLQVYYRMCQNCIELNAATISGFLFAEIVSIFVLAVGVYFIAGQDGVRQSRASDKQTLLPNDQLYQPLKDREDDQYSHLQGNQLRRNVKQTLNFDLLKLAGDVESNPGPMEHSTFLSGLVLATLLSQVSPFKIPIEELEDRVFVNCNTSITWVEGTVGTLLSDITRLDLGKRILDPRGIYRCNGTDIYKDKESTVQVHYRMCQSCVELDPATVAGIIVTDVIATLLLALGVFCFAGHETGRLSGAADTQALLRNDQVYQPLRDRDDAQYSHLGGNWARNKEGRGSLLTCGDVEENPGPMQSGTHWRVLGLCLLSVGVWGQDGNEEMGGITQTPYKVSISGTTVILTCPQYPGSEILWQHNDKNIGGDEDDKNIGSDEDHLSLKEFSELEQSGYYVCYPRGSKPEDANFYLYLRARVCENCMEMDVMSVATIVIVDICITGGLLLLVYYWSKNRKAKAKPVTRGAGAGGRQRGQNKERPPPVPNPDYEPIRKGQRDLYSGLNQRRIGPQCTNYALLKLAGDVESNPGP

SEQ ID NO: 62 - DNA, 2427 bp, synthetic construct
  source 1..2427: mol_type = other DNA
atgctcgagatgaagtggaaggcgcttttcaccgcggccatcctgcaggcacagttgccgattacagaggcacagagctttggcctgctggatcccaaactctgctacctgctggatggaatcctcttcatctatggtgtcattctcactgccttgttcctgagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgcagagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgccagtgcaccaactacgccctgctgaagctggccggcgacgtggagagcaaccccggccccatggaacaggggaagggcctggctgtcctcatcctggctatcattcttcttcaaggtactttggcccagtcaatcaaaggaaaccacttggttaaggtgtatgactatcaagaagatggttcggtacttctgacttgtgatgcagaagccaaaaatatcacatggtttaaagatgggaagatgatcggcttcctaactgaagataaaaaaaaatggaatctgggaagtaatgccaaggaccctcgtgggatgtatcagtgtaaaggatcacagaacaagtcaaaaccactccaagtgtattacagaatgtgtcagaactgcattgaactaaatgcagccaccatatctggctttctctttgctgaaatcgtcagcattttcgtccttgctgttggggtctacttcattgctggacaggatggagttcgccagtcgagagcttcagacaagcagactctgttgcccaatgaccagctctaccagcccctcaaggatcgagaagatgaccagtacagccaccttcaaggaaaccagttgaggaggaatgtgaagcagaccctgaacttcgacctgctgaagctggccggcgacgtggagagcaaccccggccccatggagcacagcaccttcctgagcggcctggtgctggccaccctgctgagccaggtgagccccttcaagatccccatcgaggagctggaggacagagtgttcgtgaactgcaacaccagcatcacctgggtggagggcaccgtgggcaccctgctgagcgacatcaccagactggacctgggcaagagaatcctggaccccagaggcatctacagatgcaacggcaccgacatctacaaggacaaggagagcaccgtgcaggtgcactacagaatgtgccagagctgcgtggagctggaccccgccaccgtggccggcatcatcgtgaccgacgtgatcgccaccctgctgctggccctgggcgtgttctgcttcgccggccacgagaccggcagactgagcggcgccgccgacacccaggccctgctgagaaacgaccaggtgtaccagcccctgagagacagagacgacgcccagtacagccacctgggcggcaactgggccagaaacaaggagggcagaggcagcctgctgacctgcggcgacgtggaggagaaccccggccccatgcagagcggcacccactggagagtgctgggcctgtgcctgctgagcgtgggcgtgtggggccaggacggcaacgaggagatgggcggcatcacccagaccccctacaaggtgagcatcagcggcaccaccgtgatcctgacctgcccccagtaccccggcagcgagatcctgtggcagcacaacgacaagaacatcggcggcgacgaggacgacaagaacatcggcagcgacgaggaccacctgagcctgaaggagttcagcgagctggagcagagcggctactacgtgtgctaccccagaggcagcaagcccgaggacgccaacttctacctgtacctgagagccagagtgtgcgagaactgcatggagatggacgtgatgagcgtggccaccatcgtgatcgtggacatctgcatcaccggcggcctgctgctgctggtgtactactggagcaagaacagaaaggccaaggccaagcccgtgaccagaggcgccggcgccggcggcagacagagaggccagaacaaggagagacccccccccgtgcccaaccccgactacgagcccatcagaaagggccagagagacctgtacagcggcctgaaccagagaagaatcggaccgcagtgtactaattatgctctcttgaaattggctggagatgttgagagcaatcccgggccc

SEQ ID NO: 63 - protein, 24 aa, synthetic construct
MRICLTSDRLAPAAGLAAPRRQAV

SEQ ID NO: 64 - DNA, 72 bp, synthetic construct
  source 1..72: mol_type = other DNA
atgcgcatttgcctgaccagcgatcgcctggcgccggcggcgggcctggcggcgccgcgccgccaggcggtg

SEQ ID NO: 65 - protein, 138 aa, synthetic construct
HKSSSQGQDRHMIRMRQLIDIVDQLKNYVNDLVPEFLPAPEDVETNCEWSAFSCFQKAQLKSANTGNNERIINVSIKKLKRKPPSTNAGRRQKHRLTCPSCDSYEKKPPKEFLERFKSLLQKMIHQHLSSRTHGSEDS

SEQ ID NO: 66 - DNA, 414 bp, synthetic construct
  source 1..414: mol_type = other DNA
cataaatcttcctctcaaggtcaggaccgccatatgattcgaatgcggcagctgattgacatagtcgatcaactgaagaactatgtgaatgatcttgtgcccgagtttttgccagcccctgaagacgtagaaactaattgtgagtggagtgccttttcctgctttcaaaaggcacagctgaaatccgccaacacgggcaataacgaacggataattaacgtatccattaagaagctgaagcggaagccgccctcaaccaatgcgggacggcggcaaaagcatcgcttgacctgtccgtcatgcgacagctacgagaaaaagcccccgaaggagttcttggaacgcttcaagagtctccttcagaaaatgattcaccagcacctgtcctcacggacgcacggaagcgaggacagt

SEQ ID NO: 67 - protein, 45 aa, synthetic construct
TTTPAPRPPTPAPTIASQPLSLRPEACRPAAGGAVHTRGLDFACD

SEQ ID NO: 68 - DNA, 135 bp, synthetic construct
  source 1..135: mol_type = other DNA
accacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgat

SEQ ID NO: 69 - protein, 27 aa, synthetic construct
FWVLVVVGGVLACYSLLVTVAFIIFWV

SEQ ID NO: 70 - DNA, 81 bp, synthetic construct
  source 1..81: mol_type = other DNA
ttttgggtgctggtggtggttggtggagtcctggcttgctatagcttgctagtaacagtggcctttattattttctgggtg

SEQ ID NO: 71 - protein, 1037 aa, synthetic construct
MLEMKWKALFTAAILQAQLPITEAQSFGLLDPKLCYLLDGILFIYGVILTALFLRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPRQCTNYALLKLAGDVESNPGPMEQGKGLAVLILAIILLQGTLAQSIKGNHLVKVYDYQEDGSVLLTCDAEAKNITWFKDGKMIGFLTEDKKKWNLGSNAKDPRGMYQCKGSQNKSKPLQVYYRMCQNCIELNAATISGFLFAEIVSIFVLAVGVYFIAGQDGVRQSRASDKQTLLPNDQLYQPLKDREDDQYSHLQGNQLRRNVKQTLNFDLLKLAGDVESNPGPMEHSTFLSGLVLATLLSQVSPFKIPIEELEDRVFVNCNTSITWVEGTVGTLLSDITRLDLGKRILDPRGIYRCNGTDIYKDKESTVQVHYRMCQSCVELDPATVAGIIVTDVIATLLLALGVFCFAGHETGRLSGAADTQALLRNDQVYQPLRDRDDAQYSHLGGNWARNKEGRGSLLTCGDVEENPGPMQSGTHWRVLGLCLLSVGVWGQDGNEEMGGITQTPYKVSISGTTVILTCPQYPGSEILWQHNDKNIGGDEDDKNIGSDEDHLSLKEFSELEQSGYYVCYPRGSKPEDANFYLYLRARVCENCMEMDVMSVATIVIVDICITGGLLLLVYYWSKNRKAKAKPVTRGAGAGGRQRGQNKERPPPVPNPDYEPIRKGQRDLYSGLNQRRIGPQCTNYALLKLAGDVESNPGPMRICLTSDRLAPAAGLAAPRRQAVHKSSSQGQDRHMIRMRQLIDIVDQLKNYVNDLVPEFLPAPEDVETNCEWSAFSCFQKAQLKSANTGNNERIINVSIKKLKRKPPSTNAGRRQKHRLTCPSCDSYEKKPPKEFLERFKSLLQKMIHQHLSSRTHGSEDSTTTPAPRPPTPAPTIASQPLSLRPEACRPAAGGAVHTRGLDFACDIYIWAPLAGTCGVLLLSLVIT

SEQ ID NO: 72 - protein, 21 aa, synthetic construct
IYIWAPLAGTCGVLLLSLVIT

SEQ ID NO: 73 - DNA, 63 bp, synthetic construct
  source 1..63: mol_type = other DNA
atctacatctgggcgcccttggccgggacttgtggggtccttctcctgtcactggttatcacc

SEQ ID NO: 74 - protein, 1455 aa, synthetic construct
MLEGVTQTPKFQVLKTGQSMTLQCAQDMNHEYMSWYRQDPGMGLRLIHYSVGAGITDQGEVPNGYNVSRSTTEDFPLRLLSAAPSQTSVYFCASSPVTGGIYGYTFGSGTRLTVVEDLNKVFPPEVAVFEPSEAEISHTQKATLVCLATGFFPDHVELSWWVNGKEVHSGVSTDPQPLKEQPALNDSRYCLSSRLRVSATFWQNPRNHFRCQVQFYGLSENDEWTQDRAKPVTQIVSAEAWGRADATNFSLLKQAGDVEENPGPMILNVEQSPQSLHVQEGDSTNFTCSFPSSNFYALHWYRWETAKSPEALFVMTLNGDEKKKGRISATLNTKEGYSYLYIKGSQPEDSATYLCARNTGNQFYFGTGTSLTVIPNIQNPDPAVYQLRDSKSSDKSVCLFTDFDSQTNVSQSKDSDAYITDKTVLDMRSMDFKSNSAVAWSNKSDFACANAFNNSIIPEDTFFPSPESSEGRGSLLTCGDVEENPGPMKWKALFTAAILQAQLPITEAQSFGLLDPKLCYLLDGILFIYGVILTALFLRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPRQCTNYALLKLAGDVESNPGPMEQGKGLAVLILAIILLQGTLAQSIKGNHLVKVYDYQEDGSVLLTCDAEAKNITWFKDGKMIGFLTEDKKKWNLGSNAKDPRGMYQCKGSQNKSKPLQVYYRMCQNCIELNAATISGFLFAEIVSIFVLAVGVYFIAGQDGVRQSRASDKQTLLPNDQLYQPLKDREDDQYSHLQGNQLRRNVKQTLNFDLLKLAGDVESNPGPMEHSTFLSGLVLATLLSQVSPFKIPIEELEDRVFVNCNTSITWVEGTVGTLLSDITRLDLGKRILDPRGIYRCNGTDIYKDKESTVQVHYRMCQSCVELDPATVAGIIVTDVIATLLLALGVFCFAGHETGRLSGAADTQALLRNDQVYQPLRDRDDAQYSHLGGNWARNKEGRGSLLTCGDVEENPGPMQSGTHWRVLGLCLLSVGVWGQDGNEEMGGITQTPYKVSISGTTVILTCPQYPGSEILWQHNDKNIGGDEDDKNIGSDEDHLSLKEFSELEQSGYYVCYPRGSKPEDANFYLYLRARVCENCMEMDVMSVATIVIVDICITGGLLLLVYYWSKNRKAKAKPVTRGAGAGGRQRGQNKERPPPVPNPDYEPIRKGQRDLYSGLNQRRIGPQCTNYALLKLAGDVESNPGPMRISKPHLRSISIQCYLCLLLNSHFLTEAGIHVFILGCFSAGLPKTEANWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 75 - protein, 264 aa, synthetic construct
MLEGVTQTPKFQVLKTGQSMTLQCAQDMNHEYMSWYRQDPGMGLRLIHYSVGAGITDQGEVPNGYNVSRSTTEDFPLRLLSAAPSQTSVYFCASSPVTGGIYGYTFGSGTRLTVVEDLNKVFPPEVAVFEPSEAEISHTQKATLVCLATGFFPDHVELSWWVNGKEVHSGVSTDPQPLKEQPALNDSRYCLSSRLRVSATFWQNPRNHFRCQVQFYGLSENDEWTQDRAKPVTQIVSAEAWGRADATNFSLLKQAGDVEENPGP

SEQ ID NO: 76 - DNA, 735 bp, synthetic construct; identical to SEQ ID NO: 41
  source 1..735: mol_type = other DNA

SEQ ID NO: 77 - protein, 223 aa, synthetic construct
MILNVEQSPQSLHVQEGDSTNFTCSFPSSNFYALHWYRWETAKSPEALFVMTLNGDEKKKGRISATLNTKEGYSYLYIKGSQPEDSATYLCARNTGNQFYFGTGTSLTVIPNIQNPDPAVYQLRDSKSSDKSVCLFTDFDSQTNVSQSKDSDAYITDKTVLDMRSMDFKSNSAVAWSNKSDFACANAFNNSIIPEDTFFPSPESSEGRGSLLTCGDVEENPGP

SEQ ID NO: 78 - DNA, 672 bp, synthetic construct
  source 1..672: mol_type = other DNA
atgatcctgaacgtggagcagagcccccagagcctgcacgtgcaggagggagacagcaccaacttcacctgcagcttccccagcagcaacttctacgccctgcactggtaccggtgggagaccgccaagagccccgaggccctgttcgtgatgaccctgaacggagacgagaagaagaagggacggatcagcgccaccctgaacaccaaggagggatacagctacctgtacatcaagggaagccagcccgaggacagcgccacctacctgtgcgcccggaacaccggaaaccagttctacttcggaaccggaaccagcctgaccgtgatccccaacatccagaaccccgaccccgccgtgtaccagctgcgggacagcaagagcagcgacaagagcgtgtgcctgttcaccgacttcgacagccagaccaacgtgagccagagcaaggacagcgacgcctacatcaccgacaagaccgtgctggacatgcggagcatggacttcaagagcaacagcgccgtggcctggagcaacaagagcgacttcgcctgcgccaacgccttcaacaacagcatcatccccgaggacaccttcttccccagccccgagagcagcgccaccaacttctccctgctgaagcaggccggcgacgtggaggagaaccccggcccc

SEQ ID NO: 79 - protein, 806 aa, synthetic construct
MKWKALFTAAILQAQLPITEAQSFGLLDPKLCYLLDGILFIYGVILTALFLRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPRQCTNYALLKLAGDVESNPGPMEQGKGLAVLILAIILLQGTLAQSIKGNHLVKVYDYQEDGSVLLTCDAEAKNITWFKDGKMIGFLTEDKKKWNLGSNAKDPRGMYQCKGSQNKSKPLQVYYRMCQNCIELNAATISGFLFAEIVSIFVLAVGVYFIAGQDGVRQSRASDKQTLLPNDQLYQPLKDREDDQYSHLQGNQLRRNVKQTLNFDLLKLAGDVESNPGPMEHSTFLSGLVLATLLSQVSPFKIPIEELEDRVFVNCNTSITWVEGTVGTLLSDITRLDLGKRILDPRGIYRCNGTDIYKDKESTVQVHYRMCQSCVELDPATVAGIIVTDVIATLLLALGVFCFAGHETGRLSGAADTQALLRNDQVYQPLRDRDDAQYSHLGGNWARNKEGRGSLLTCGDVEENPGPMQSGTHWRVLGLCLLSVGVWGQDGNEEMGGITQTPYKVSISGTTVILTCPQYPGSEILWQHNDKNIGGDEDDKNIGSDEDHLSLKEFSELEQSGYYVCYPRGSKPEDANFYLYLRARVCENCMEMDVMSVATIVIVDICITGGLLLLVYYWSKNRKAKAKPVTRGAGAGGRQRGQNKERPPPVPNPDYEPIRKGQRDLYSGLNQRRIGPQCTNYALLKLAGDVESNPGP

SEQ ID NO: 80 - DNA, 2418 bp, synthetic construct
  source 1..2418: mol_type = other DNA
atgaagtggaaggcgcttttcaccgcggccatcctgcaggcacagttgccgattacagaggcacagagctttggcctgctggatcccaaactctgctacctgctggatggaatcctcttcatctatggtgtcattctcactgccttgttcctgagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgcagagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgccagtgcaccaactacgccctgctgaagctggccggcgacgtggagagcaaccccggccccatggaacaggggaagggcctggctgtcctcatcctggctatcattcttcttcaaggtactttggcccagtcaatcaaaggaaaccacttggttaaggtgtatgactatcaagaagatggttcggtacttctgacttgtgatgcagaagccaaaaatatcacatggtttaaagatgggaagatgatcggcttcctaactgaagataaaaaaaaatggaatctgggaagtaatgccaaggaccctcgtgggatgtatcagtgtaaaggatcacagaacaagtcaaaaccactccaagtgtattacagaatgtgtcagaactgcattgaactaaatgcagccaccatatctggctttctctttgctgaaatcgtcagcattttcgtccttgctgttggggtctacttcattgctggacaggatggagttcgccagtcgagagcttcagacaagcagactctgttgcccaatgaccagctctaccagcccctcaaggatcgagaagatgaccagtacagccaccttcaaggaaaccagttgaggaggaatgtgaagcagaccctgaacttcgacctgctgaagctggccggcgacgtggagagcaaccccggccccatggagcacagcaccttcctgagcggcctggtgctggccaccctgctgagccaggtgagccccttcaagatccccatcgaggagctggaggacagagtgttcgtgaactgcaacaccagcatcacctgggtggagggcaccgtgggcaccctgctgagcgacatcaccagactggacctgggcaagagaatcctggaccccagaggcatctacagatgcaacggcaccgacatctacaaggacaaggagagcaccgtgcaggtgcactacagaatgtgccagagctgcgtggagctggaccccgccaccgtggccggcatcatcgtgaccgacgtgatcgccaccctgctgctggccctgggcgtgttctgcttcgccggccacgagaccggcagactgagcggcgccgccgacacccaggccctgctgagaaacgaccaggtgtaccagcccctgagagacagagacgacgcccagtacagccacctgggcggcaactgggccagaaacaaggagggcagaggcagcctgctgacctgcggcgacgtggaggagaaccccggccccatgcagagcggcacccactggagagtgctgggcctgtgcctgctgagcgtgggcgtgtggggccaggacggcaacgaggagatgggcggcatcacccagaccccctacaaggtgagcatcagcggcaccaccgtgatcctgacctgcccccagtaccccggcagcgagatcctgtggcagcacaacgacaagaacatcggcggcgacgaggacgacaagaacatcggcagcgacgaggaccacctgagcctgaaggagttcagcgagctggagcagagcggctactacgtgtgctaccccagaggcagcaagcccgaggacgccaacttctacctgtacctgagagccagagtgtgcgagaactgcatggagatggacgtgatgagcgtggccaccatcgtgatcgtggacatctgcatcaccggcggcctgctgctgctggtgtactactggagcaagaacagaaaggccaaggccaagcccgtgaccagaggcgccggcgccggcggcagacagagaggccagaacaaggagagacccccccccgtgcccaaccccgactacgagcccatcagaaagggccagagagacctgtacagcggcctgaaccagagaagaatcggaccgcagtgtactaattatgctctcttgaaattggctggagatgttgagagcaatcccgggccc

SEQ ID NO: 81 - DNA, 1200 bp, synthetic construct
  source 1..1200: mol_type = other DNA
atgcggtgggccctactggtgcttctagctttcctgtctcctgccagtcaggataaacaacttgatgcagatgtttcccccaagcccactatttttcttccttcgattgctgaaacaaaactccagaaggctggaacatacctttgtcttcttgagaaatttttcccagatattattaagatacattggcaagaaaagaagagcaacacgattctgggatcccaggaggggaacaccatgaagactaacgacacatacatgaaatttagctggttaacggtgccagaagagtcactggacaaagaacacagatgtatcgtcagacatgagaataataaaaacggaattgatcaagaaattatctttcctccaataaagacagatgtcaccacagtggatcccaaatacaattattcaaaggatgcaaatgatgtcatcacaatggatcccaaagacaattggtcaaaagatgcaaatgatacactactgctgcagctcacaaacacctctgcatattacacgtacctcctcctgctcctcaagagtgtggtctattttgccatcatcacctgctgtctgcttagaagaacggctttctgctgcaatggagagaaatcaggaagcggagctactaactttagcctgctgaagcaggctggagatgtggaggagaaccctggacctatgattcttactgtgggctttagctttttgtttttctacaggggcacgctgtgtagtcagcctcataccaaaccatccgtttttgtcatgaaaaatggaacaaatgtcgcttgtctggtgaaggaattctaccccaaggatataagaataaatctcgtgtcatccaagaagataacagagtttgatcctgctattgtcatctctcccagtgggaagtacaatgctgtcaagcttggtaaatatgaagattcaaattcagtgacatgttcagttcaacacgacaataaaactgtgcactccactgactttgaagtgaagacagattctacagatcacgtaaaaccaaaggaaactgaaaacacaaagcaaccttcaaagagctgccataaacccaaagccatagttcataccgagaaggtgaacatgatgtccctcacagtgcttgggctacgaatgctgtttgcaaagactgttgccgtcaattttctcttgactgccaagttatttttcttgtaa

SEQ ID NO: 82 - protein, 399 aa, synthetic construct
MRWALLVLLAFLSPASQDKQLDADVSPKPTIFLPSIAETKLQKAGTYLCLLEKFFPDIIKIHWQEKKSNTILGSQEGNTMKTNDTYMKFSWLTVPEESLDKEHRCIVRHENNKNGIDQEIIFPPIKTDVTTVDPKYNYSKDANDVITMDPKDNWSKDANDTLLLQLTNTSAYYTYLLLLLKSVVYFAIITCCLLRRTAFCCNGEKSGSGATNFSLLKQAGDVEENPGPMILTVGFSFLFFYRGTLCSQPHTKPSVFVMKNGTNVACLVKEFYPKDIRINLVSSKKITEFDPAIVISPSGKYNAVKLGKYEDSNSVTCSVQHDNKTVHSTDFEVKTDSTDHVKPKETENTKQPSKSCHKPKAIVHTEKVNMMSLTVLGLRMLFAKTVAVNFLLTAKLFFL

SEQ ID NO: 83 - protein, 380 aa, synthetic construct
METLLGLLILWLQLQWVSSIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSSGSGATNFSLLKQAGDVEENPGPMSIGLLCCAALSLLWAGPVNADLKNVFPPKVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 84 - DNA, 1143 bp, synthetic construct
  source 1..1143: mol_type = other DNA
atggagaccctcttgggcctgcttatcctttggctgcagctgcaatgggtgagcagcatccagaaccctgaccctgccgtgtaccagctgagagactctaaatccagtgacaagtctgtctgcctattcaccgattttgattctcaaacaaatgtgtcacaaagtaaggattctgatgtgtatatcacagacaaaactgtgctagacatgaggtctatggacttcaagagcaacagtgctgtggcctggagcaacaaatctgactttgcatgtgcaaacgccttcaacaacagcattattccagaagacaccttcttccccagcccagaaagttcctgtgatgtcaagctggtcgagaaaagctttgaaacagatacgaacctaaactttcaaaacctgtcagtgattgggttccgaatcctcctcctgaaagtggccgggtttaatctgctcatgacgctgcggctgtggtccagcggaagcggagctactaactttagcctgctgaagcaggctggagatgtggaggagaaccctggacctatgagcatcggcctcctgtgctgtgcagccttgtctctcctgtgggcaggtccagtgaatgctgacctgaaaaacgtgttcccacccaaggtcgctgtgtttgagccatcagaagcagagatctcccacacccaaaaggccacactggtatgcctggccacaggcttctaccccgaccacgtggagctgagctggtgggtgaatgggaaggaggtgcacagtggggtcagcacagacccgcagcccctcaaggagcagcccgccctcaatgactccagatactgcctgagcagccgcctgagggtctcggccaccttctggcagaacccccgcaaccacttccgctgtcaagtccagttctacgggctctcggagaatgacgagtggacccaggatagggccaaacccgtcacccagatcgtcagcgccgaggcctggggtagagcagactgtggcttcacctccgagtcttaccagcaaggggtcctgtctgccaccatcctctatgagatcttgctagggaaggccaccttgtatgccgtgctggtcagtgccctcgtgctgatggccatggtcaagagaaaggattccagaggctaa

SEQ ID NO: 85 - DNA, 345 bp, synthetic construct
  source 1..345: mol_type = other DNA
aaacaggaggtgacacagattcctgcagctctgagtgtcccagaaggagaaaacttggttctcaactgcagtttcactgatagcgctatttacaacctccagtggtttaggcaggaccctgggaaaggtctcacatctctgttgcttattcagtcaagtcagagagagcaaacaagtggaagacttaatgcctcgctggataaatcatcaggacgtagtactttatacattgcagcttctcagcctggtgactcagccacctacctctgtgctgtgaggcccctttatggaggaagctacatacctacatttggaagaggaaccagccttattgttcatccgtat

SEQ ID NO: 86 - DNA, 420 bp, synthetic construct
  source 1..420: mol_type = other DNA
atccagaaccctgaccctgccgtgtaccagctgagagactctaaatccagtgacaagtctgtctgcctattcaccgattttgattctcaaacaaatgtgtcacaaagtaaggattctgatgtgtatatcacagacaaaactgtgctagacatgaggtctatggacttcaagagcaacagtgctgtggcctggagcaacaaatctgactttgcatgtgcaaacgccttcaacaacagcattattccagaagacaccttcttccccagcccagaaagttcctgtgatgtcaagctggtcgagaaaagctttgaaacagatacgaacctaaactttcaaaacctgtcagtgattgggttccgaatcctcctcctgaaagtggccgggtttaatctgctcatgacgctgcggctgtggtccagc

SEQ ID NO: 87 - DNA, 822 bp, synthetic construct
  source 1..822: mol_type = other DNA
atggagaccctcttgggcctgcttatcctttggctgcagctgcaatgggtgagcagcaaacaggaggtgacacagattcctgcagctctgagtgtcccagaaggagaaaacttggttctcaactgcagtttcactgatagcgctatttacaacctccagtggtttaggcaggaccctgggaaaggtctcacatctctgttgcttattcagtcaagtcagagagagcaaacaagtggaagacttaatgcctcgctggataaatcatcaggacgtagtactttatacattgcagcttctcagcctggtgactcagccacctacctctgtgctgtgaggcccctttatggaggaagctacatacctacatttggaagaggaaccagccttattgttcatccgtatatccagaaccctgaccctgccgtgtaccagctgagagactctaaatccagtgacaagtctgtctgcctattcaccgattttgattctcaaacaaatgtgtcacaaagtaaggattctgatgtgtatatcacagacaaaactgtgctagacatgaggtctatggacttcaagagcaacagtgctgtggcctggagcaacaaatctgactttgcatgtgcaaacgccttcaacaacagcattattccagaagacaccttcttccccagcccagaaagttcctgtgatgtcaagctggtcgagaaaagctttgaaacagatacgaacctaaactttcaaaacctgtcagtgattgggttccgaatcctcctcctgaaagtggccgggtttaatctgctcatgacgctgcggctgtggtccagc

SEQ ID NO: 88 - protein, 115 aa, synthetic construct
XQEVTQIPAALSVPEGENLVLNCSFTDSAIYNLQWFRQDPGKGLTSLLLIQSSQREQTSGRLNASLDKSSGRSTLYIAASQPGDSATYLCAVRPLYGGSYIPTFGRGTSLIVHPY

SEQ ID NO: 89 - protein, 140 aa, synthetic construct
IQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 90 - protein, 6 aa, synthetic construct
DSAIYN

SEQ ID NO: 91 - protein, 7 aa, synthetic construct
IQSSQRE

SEQ ID NO: 92 - protein, 15 aa, synthetic construct
CAVRPLYGGSYIPTF

SEQ ID NO: 93 - DNA, 336 bp, synthetic construct
  source 1..336: mol_type = other DNA
ggtgtcactcagaccccaaaattccaggtcctgaagacaggacagagcatgacactgcagtgtgcccaggatatgaaccatgaatacatgtcctggtatcgacaagacccaggcatggggctgaggctgattcattactcagttggtgctggtatcactgaccaaggagaagtccccaatggctacaatgtctccagatcaaccacagaggatttcccgctcaggctgctgtcggctgctccctcccagacatctgtgtacttctgtgccagcagttacgtcgggaacaccggggagctgttttttggagaaggctctaggctgaccgtactggag

SEQ ID NO: 94 - DNA, 534 bp, synthetic construct
  source 1..534: mol_type = other DNA
gacctgaaaaacgtgttcccacccaaggtcgctgtgtttgagccatcagaagcagagatctcccacacccaaaaggccacactggtatgcctggccacaggcttctaccccgaccacgtggagctgagctggtgggtgaatgggaaggaggtgcacagtggggtcagcacagacccgcagcccctcaaggagcagcccgccctcaatgactccagatactgcctgagcagccgcctgagggtctcggccaccttctggcagaacccccgcaaccacttccgctgtcaagtccagttctacgggctctcggagaatgacgagtggacccaggatagggccaaacccgtcacccagatcgtcagcgccgaggcctggggtagagcagactgtggcttcacctccgagtcttaccagcaaggggtcctgtctgccaccatcctctatgagatcttgctagggaaggccaccttgtatgccgtgctggtcagtgccctcgtgctgatggccatggtcaagagaaaggattccagaggc

SEQ ID NO: 95 - DNA, 933 bp, synthetic construct
  source 1..933: mol_type = other DNA
atgagcatcggcctcctgtgctgtgcagccttgtctctcctgtgggcaggtccagtgaatgctggtgtcactcagaccccaaaattccaggtcctgaagacaggacagagcatgacactgcagtgtgcccaggatatgaaccatgaatacatgtcctggtatcgacaagacccaggcatggggctgaggctgattcattactcagttggtgctggtatcactgaccaaggagaagtccccaatggctacaatgtctccagatcaaccacagaggatttcccgctcaggctgctgtcggctgctccctcccagacatctgtgtacttctgtgccagcagttacgtcgggaacaccggggagctgttttttggagaaggctctaggctgaccgtactggaggacctgaaaaacgtgttcccacccaaggtcgctgtgtttgagccatcagaagcagagatctcccacacccaaaaggccacactggtatgcctggccacaggcttctaccccgaccacgtggagctgagctggtgggtgaatgggaaggaggtgcacagtggggtcagcacagacccgcagcccctcaaggagcagcccgccctcaatgactccagatactgcctgagcagccgcctgagggtctcggccaccttctggcagaacccccgcaaccacttccgctgtcaagtccagttctacgggctctcggagaatgacgagtggacccaggatagggccaaacccgtcacccagatcgtcagcgccgaggcctggggtagagcagactgtggcttcacctccgagtcttaccagcaaggggtcctgtctgccaccatcctctatgagatcttgctagggaaggccaccttgtatgccgtgctggtcagtgccctcgtgctgatggccatggtcaagagaaaggattccagaggc

SEQ ID NO: 96 - protein, 112 aa, synthetic construct
GVTQTPKFQVLKTGQSMTLQCAQDMNHEYMSWYRQDPGMGLRLIHYSVGAGITDQGEVPNGYNVSRSTTEDFPLRLLSAAPSQTSVYFCASSYVGNTGELFFGEGSRLTVLE

SEQ ID NO: 97 - protein, 178 aa, synthetic construct
DLKNVFPPKVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 98 - protein, 5 aa, synthetic construct
MNHEY

SEQ ID NO: 99 - protein, 6 aa, synthetic construct
SVGAGI

SEQ ID NO: 100 - protein, 14 aa, synthetic construct
CASSYVGNTGELFF

SEQ ID NO: 101 - DNA, 57 bp, synthetic construct
  source 1..57: mol_type = other DNA
atggagaccctcttgggcctgcttatcctttggctgcagctgcaatgggtgagcagc

SEQ ID NO: 102 - DNA, 63 bp, synthetic construct
  source 1..63: mol_type = other DNA
atgagcatcggcctcctgtgctgtgcagccttgtctctcctgtgggcaggtccagtgaatgct

SEQ ID NO: 103 - protein, 19 aa, synthetic construct
METLLGLLILWLQLQWVSS

SEQ ID NO: 104 - protein, 21 aa, synthetic construct
MSIGLLCCAALSLLWAGPVNA

SEQ ID NO: 105 - protein, 9 aa, synthetic construct
SLLMWITQC

SEQ ID NO: 106 - DNA, 624 bp, synthetic construct
  source 1..624: mol_type = other DNA
atgaagtggaaggcgcttttcaccgcggccatcctgcaggcacagttgccgattacagaggcacagagctttggcctgctggatcccaaactctgctacctgctggatggaatcctcttcatctatggtgtcattctcactgccttgttcctgctttgcgcacgcccacgccgcagccccgcccaagaagatggcaaagtctacatcaacatgccaggcaggggcagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgcagagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgccagtgcaccaactacgccctgctgaagctggccggcgacgtggagagcaaccccggcccc

SEQ ID NO: 107 - DNA, 675 bp, synthetic construct
  source 1..675: mol_type = other DNA
atgaagtggaaggcgcttttcaccgcggccatcctgcaggcacagttgccgattacagaggcacagagctttggcctgctggatcccaaactctgctacctgctggatggaatcctcttcatctatggtgtcattctcactgccttgttcctgaggagtaagaggagcaggctcctgcacagtgactacatgaacatgactccccgccgccccgggcccacccgcaagcattaccagccctatgccccaccacgcgacttcgcagcctatcgctcaagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgcagagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgccagtgcaccaactacgccctgctgaagctggccggcgacgtggagagcaaccccggcccc

SEQ ID NO: 108 - DNA, 747 bp, synthetic construct
  source 1..747: mol_type = other DNA
atgaagtggaaggcgcttttcaccgcggccatcctgcaggcacagttgccgattacagaggcacagagctttggcctgctggatcccaaactctgctacctgctggatggaatcctcttcatctatggtgtcattctcactgccttgttcctgaggagtaagaggagcaggctcctgcacagtgactacatgaacatgactccccgccgccccgggcccacccgcaagcattaccagccctatgccccaccacgcgacttcgcagcctatcgctcactttgcgcacgcccacgccgcagccccgcccaagaagatggcaaagtctacatcaacatgccaggcaggggcagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgcagagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgccagtgcaccaactacgccctgctgaagctggccggcgacgtggagagcaaccccggcccc

SEQ ID NO: 109 - protein, 208 aa, synthetic construct
MKWKALFTAAILQAQLPITEAQSFGLLDPKLCYLLDGILFIYGVILTALFLLCARPRRSPAQEDGKVYINMPGRGRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPRQCTNYALLKLAGDVESNPGP

SEQ ID NO: 110 - protein, 225 aa, synthetic construct
MKWKALFTAAILQAQLPITEAQSFGLLDPKLCYLLDGILFIYGVILTALFLRSKRSRLLHSDYMNMTPRRPGPTRKHYQPYAPPRDFAAYRSRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPRQCTNYALLKLAGDVESNPGP

SEQ ID NO: 111 - protein, 249 aa, synthetic construct
MKWKALFTAAILQAQLPITEAQSFGLLDPKLCYLLDGILFIYGVILTALFLRSKRSRLLHSDYMNMTPRRPGPTRKHYQPYAPPRDFAAYRSLCARPRRSPAQEDGKVYINMPGRGRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPRQCTNYALLKLAGDVESNPGP

SEQ ID NO: 112 - protein, 72 aa, synthetic construct
CTTTGCGCACGCCCACGCCGCAGCCCCGCCCAAGAAGATGGCAAAGTCTACATCAACATGCCAGGCAGGGGC

SEQ ID NO: 113 - DNA, 123 bp, synthetic construct
  source 1..123: mol_type = other DNA
aggagtaagaggagcaggctcctgcacagtgactacatgaacatgactccccgccgccccgggcccacccgcaagcattaccagccctatgccccaccacgcgacttcgcagcctatcgctca

SEQ ID NO: 114 - DNA, 195 bp, synthetic construct
  source 1..195: mol_type = other DNA
aggagtaagaggagcaggctcctgcacagtgactacatgaacatgactccccgccgccccgggcccacccgcaagcattaccagccctatgccccaccacgcgacttcgcagcctatcgctcactttgcgcacgcccacgccgcagccccgcccaagaagatggcaaagtctacatcaacatgccaggcaggggc

SEQ ID NO: 115 - protein, 24 aa, synthetic construct
LCARPRRSPAQEDGKVYINMPGRG

SEQ ID NO: 116 - protein, 41 aa, synthetic construct
RSKRSRLLHSDYMNMTPRRPGPTRKHYQPYAPPRDFAAYRS

SEQ ID NO: 117 - protein, 65 aa, synthetic construct
RSKRSRLLHSDYMNMTPRRPGPTRKHYQPYAPPRDFAAYRSLCARPRRSPAQEDGKVYINMPGRG

SEQ ID NO: 118 - DNA, 2976 bp, synthetic construct
  source 1..2976: mol_type = other DNA
atgaagtggaaggcgcttttcaccgcggccatcctgcaggcacagttgccgattacagaggcacagagctttggcctgctggatcccaaactctgctacctgctggatggaatcctcttcatctatggtgtcattctcactgccttgttcctgctttgcgcacgcccacgccgcagccccgcccaagaagatggcaaagtctacatcaacatgccaggcaggggcagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgcagagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgccagtgcaccaactacgccctgctgaagctggccggcgacgtggagagcaaccccggccccatggaacaggggaagggcctggctgtcctcatcctggctatcattcttcttcaaggtactttggcccagtcaatcaaaggaaaccacttggttaaggtgtatgactatcaagaagatggttcggtacttctgacttgtgatgcagaagccaaaaatatcacatggtttaaagatgggaagatgatcggcttcctaactgaagataaaaaaaaatggaatctgggaagtaatgccaaggaccctcgtgggatgtatcagtgtaaaggatcacagaacaagtcaaaaccactccaagtgtattacagaatgtgtcagaactgcattgaactaaatgcagccaccatatctggctttctctttgctgaaatcgtcagcattttcgtccttgctgttggggtctacttcattgctggacaggatggagttcgccagtcgagagcttcagacaagcagactctgttgcccaatgaccagctctaccagcccctcaaggatcgagaagatgaccagtacagccaccttcaaggaaaccagttgaggaggaatgtgaagcagaccctgaacttcgacctgctgaagctggccggcgacgtggagagcaaccccggccccatggagcacagcaccttcctgagcggcctggtgctggccaccctgctgagccaggtgagccccttcaagatccccatcgaggagctggaggacagagtgttcgtgaactgcaacaccagcatcacctgggtggagggcaccgtgggcaccctgctgagcgacatcaccagactggacctgggcaagagaatcctggaccccagaggcatctacagatgcaacggcaccgacatctacaaggacaaggagagcaccgtgcaggtgcactacagaatgtgccagagctgcgtggagctggaccccgccaccgtggccggcatcatcgtgaccgacgtgatcgccaccctgctgctggccctgggcgtgttctgcttcgccggccacgagaccggcagactgagcggcgccgccgacacccaggccctgctgagaaacgaccaggtgtaccagcccctgagagacagagacgacgcccagtacagccacctgggcggcaactgggccagaaacaaggagggcagaggcagcctgctgacctgcggcgacgtggaggagaaccccggccccatgcagagcggcacccactggagagtgctgggcctgtgcctgctgagcgtgggcgtgtggggccaggacggcaacgaggagatgggcggcatcacccagaccccctacaaggtgagcatcagcggcaccaccgtgatcctgacctgcccccagtaccccggcagcgagatcctgtggcagcacaacgacaagaacatcggcggcgacgaggacgacaagaacatcggcagcgacgaggaccacctgagcctgaaggagttcagcgagctggagcagagcggctactacgtgtgctaccccagaggcagcaagcccgaggacgccaacttctacctgtacctgagagccagagtgtgcgagaactgcatggagatggacgtgatgagcgtggccaccatcgtgatcgtggacatctgcatcaccggcggcctgctgctgctggtgtactactggagcaagaacagaaaggccaaggccaagcccgtgaccagaggcgccggcgccggcggcagacagagaggccagaacaaggagagacccccccccgtgcccaaccccgactacgagcccatcagaaagggccagagagacctgtacagcggcctgaaccagagaagaatcggaccgcagtgtactaattatgctctcttgaaattggctggagatgttgagagcaatcccgggcccatgcgcattagcaagccccacctgcggagcatcagcatccagtgctacctgtgcctgctgctgaacagccacttcctgaccgaggccggcatccacgtgttcatcctgggctgcttcagcgccggactgcccaagaccgaggccaactgggtgaacgtgatcagcgacctgaagaagatcgaggacctgatccagagcatgcacatcgacgccaccctgtacaccgagagcgacgtgcaccccagctgcaaggtgaccgccatgaagtgctttctgctggaactgcaggtgatcagcctggaaagcggcgacgccagcatccacgacaccgtggagaacctgatcatcctggccaacaacagcctgagcagcaacggcaacgtgaccgagagcggctgcaaagagtgcgaggaactggaagagaagaacatcaaagagtttctgcagagcttcgtgcacatcgtgcagatgttcatcaacaccagc

SEQ ID NO: 119 - protein, 992 aa, synthetic construct
MKWKALFTAAILQAQLPITEAQSFGLLDPKLCYLLDGILFIYGVILTALFLLCARPRRSPAQEDGKVYINMPGRGRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPRQCTNYALLKLAGDVESNPGPMEQGKGLAVLILAIILLQGTLAQSIKGNHLVKVYDYQEDGSVLLTCDAEAKNITWFKDGKMIGFLTEDKKKWNLGSNAKDPRGMYQCKGSQNKSKPLQVYYRMCQNCIELNAATISGFLFAEIVSIFVLAVGVYFIAGQDGVRQSRASDKQTLLPNDQLYQPLKDREDDQYSHLQGNQLRRNVKQTLNFDLLKLAGDVESNPGPMEHSTFLSGLVLATLLSQVSPFKIPIEELEDRVFVNCNTSITWVEGTVGTLLSDITRLDLGKRILDPRGIYRCNGTDIYKDKESTVQVHYRMCQSCVELDPATVAGIIVTDVIATLLLALGVFCFAGHETGRLSGAADTQALLRNDQVYQPLRDRDDAQYSHLGGNWARNKEGRGSLLTCGDVEENPGPMQSGTHWRVLGLCLLSVGVWGQDGNEEMGGITQTPYKVSISGTTVILTCPQYPGSEILWQHNDKNIGGDEDDKNIGSDEDHLSLKEFSELEQSGYYVCYPRGSKPEDANFYLYLRARVCENCMEMDVMSVATIVIVDICITGGLLLLVYYWSKNRKAKAKPVTRGAGAGGRQRGQNKERPPPVPNPDYEPIRKGQRDLYSGLNQRRIGPQCTNYALLKLAGDVESNPGPMRISKPHLRSISIQCYLCLLLNSHFLTEAGIHVFILGCFSAGLPKTEANWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 120 - DNA, 3027 bp, synthetic construct
  source 1..3027: mol_type = other DNA
atgaagtggaaggcgcttttcaccgcggccatcctgcaggcacagttgccgattacagaggcacagagctttggcctgctggatcccaaactctgctacctgctggatggaatcctcttcatctatggtgtcattctcactgccttgttcctgaggagtaagaggagcaggctcctgcacagtgactacatgaacatgactccccgccgccccgggcccacccgcaagcattaccagccctatgccccaccacgcgacttcgcagcctatcgctcaagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgcagagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgccagtgcaccaactacgccctgctgaagctggccggcgacgtggagagcaaccccggccccatggaacaggggaagggcctggctgtcctcatcctggctatcattcttcttcaaggtactttggcccagtcaatcaaaggaaaccacttggttaaggtgtatgactatcaagaagatggttcggtacttctgacttgtgatgcagaagccaaaaatatcacatggtttaaagatgggaagatgatcggcttcctaactgaagataaaaaaaaatggaatctgggaagtaatgccaaggaccctcgtgggatgtatcagtgtaaaggatcacagaacaagtcaaaaccactccaagtgtattacagaatgtgtcagaactgcattgaactaaatgcagccaccatatctggctttctctttgctgaaatcgtcagcattttcgtccttgctgttggggtctacttcattgctggacaggatggagttcgccagtcgagagcttcagacaagcagactctgttgcccaatgaccagctctaccagcccctcaaggatcgagaagatgaccagtacagccaccttcaaggaaaccagttgaggaggaatgtgaagcagaccctgaacttcgacctgctgaagctggccggcgacgtggagagcaaccccggccccatggagcacagcaccttcctgagcggcctggtgctggccaccctgctgagccaggtgagccccttcaagatccccatcgaggagctggaggacagagtgttcgtgaactgcaacaccagcatcacctgggtggagggcaccgtgggcaccctgctgagcgacatcaccagactggacctgggcaagagaatcctggaccccagaggcatctacagatgcaacggcaccgacatctacaaggacaaggagagcaccgtgcaggtgcactacagaatgtgccagagctgcgtggagctggaccccgccaccgtggccggcatcatcgtgaccgacgtgatcgccaccctgctgctggccctgggcgtgttctgcttcgccggccacgagaccggcagactgagcggcgccgccgacacccaggccctgctgagaaacgaccaggtgtaccagcccctgagagacagagacgacgcccagtacagccacctgggcggcaactgggccagaaacaaggagggcagaggcagcctgctgacctgcggcgacgtggaggagaaccccggccccatgcagagcggcacccactggagagtgctgggcctgtgcctgctgagcgtgggcgtgtggggccaggacggcaacgaggagatgggcggcatcacccagaccccctacaaggtgagcatcagcggcaccaccgtgatcctgacctgcccccagtaccccggcagcgagatcctgtggcagcacaacgacaagaacatcggcggcgacgaggacgacaagaacatcggcagcgacgaggaccacctgagcctgaaggagttcagcgagctggagcagagcggctactacgtgtgctaccccagaggcagcaagcccgaggacgccaacttctacctgtacctgagagccagagtgtgcgagaactgcatggagatggacgtgatgagcgtggccaccatcgtgatcgtggacatctgcatcaccggcggcctgctgctgctggtgtactactggagcaagaacagaaaggccaaggccaagcccgtgaccagaggcgccggcgccggcggcagacagagaggccagaacaaggagagacccccccccgtgcccaaccccgactacgagcccatcagaaagggccagagagacctgtacagcggcctgaaccagagaagaatcggaccgcagtgtactaattatgctctcttgaaattggctggagatgttgagagcaatcccgggcccatgcgcattagcaagccccacctgcggagcatcagcatccagtgctacctgtgcctgctgctgaacagccacttcctgaccgaggccggcatccacgtgttcatcctgggctgcttcagcgccggactgcccaagaccgaggccaactgggtgaacgtgatcagcgacctgaagaagatcgaggacctgatccagagcatgcacatcgacgccaccctgtacaccgagagcgacgtgcaccccagctgcaaggtgaccgccatgaagtgctttctgctggaactgcaggtgatcagcctggaaagcggcgacgccagcatccacgacaccgtggagaacctgatcatcctggccaacaacagcctgagcagcaacggcaacgtgaccgagagcggctgcaaagagtgcgaggaactggaagagaagaacatcaaagagtttctgcagagcttcgtgcacatcgtgcagatgttcatcaacaccagc

SEQ ID NO: 121 - protein, 1009 aa, synthetic construct
MKWKALFTAAILQAQLPITEAQSFGLLDPKLCYLLDGILFIYGVILTALFLRSKRSRLLHSDYMNMTPRRPGPTRKHYQPYAPPRDFAAYRSRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPRQCTNYALLKLAGDVESNPGPMEQGKGLAVLILAIILLQGTLAQSIKGNHLVKVYDYQEDGSVLLTCDAEAKNITWFKDGKMIGFLTEDKKKWNLGSNAKDPRGMYQCKGSQNKSKPLQVYYRMCQNCIELNAATISGFLFAEIVSIFVLAVGVYFIAGQDGVRQSRASDKQTLLPNDQLYQPLKDREDDQYSHLQGNQLRRNVKQTLNFDLLKLAGDVESNPGPMEHSTFLSGLVLATLLSQVSPFKIPIEELEDRVFVNCNTSITWVEGTVGTLLSDITRLDLGKRILDPRGIYRCNGTDIYKDKESTVQVHYRMCQSCVELDPATVAGIIVTDVIATLLLALGVFCFAGHETGRLSGAADTQALLRNDQVYQPLRDRDDAQYSHLGGNWARNKEGRGSLLTCGDVEENPGPMQSGTHWRVLGLCLLSVGVWGQDGNEEMGGITQTPYKVSISGTTVILTCPQYPGSEILWQHNDKNIGGDEDDKNIGSDEDHLSLKEFSELEQSGYYVCYPRGSKPEDANFYLYLRARVCENCMEMDVMSVATIVIVDICITGGLLLLVYYWSKNRKAKAKPVTRGAGAGGRQRGQNKERPPPVPNPDYEPIRKGQRDLYSGLNQRRIGPQCTNYALLKLAGDVESNPGPMRISKPHLRSISIQCYLCLLLNSHFLTEAGIHVFILGCFSAGLPKTEANWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 122 - DNA, 3099 bp, synthetic construct
  source 1..3099: mol_type = other DNA
atgaagtggaaggcgcttttcaccgcggccatcctgcaggcacagttgccgattacagaggcacagagctttggcctgctggatcccaaactctgctacctgctggatggaatcctcttcatctatggtgtcattctcactgccttgttcctgaggagtaagaggagcaggctcctgcacagtgactacatgaacatgactccccgccgccccgggcccacccgcaagcattaccagccctatgccccaccacgcgacttcgcagcctatcgctcactttgcgcacgcccacgccgcagccccgcccaagaagatggcaaagtctacatcaacatgccaggcaggggcagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgcagagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgccagtgcaccaactacgccctgctgaagctggccggcgacgtggagagcaaccccggccccatggaacaggggaagggcctggctgtcctcatcctggctatcattcttcttcaaggtactttggcccagtcaatcaaaggaaaccacttggttaaggtgtatgactatcaagaagatggttcggtacttctgacttgtgatgcagaagccaaaaatatcacatggtttaaagatgggaagatgatcggcttcctaactgaagataaaaaaaaatggaatctgggaagtaatgccaaggaccctcgtgggatgtatcagtgtaaaggatcacagaacaagtcaaaaccactccaagtgtattacagaatgtgtcagaactgcattgaactaaatgcagccaccatatctggctttctctttgctgaaatcgtcagcattttcgtccttgctgttggggtctacttcattgctggacaggatggagttcgccagtcgagagcttcagacaagcagactctgttgcccaatgaccagctctaccagcccctcaaggatcgagaagatgaccagtacagccaccttcaaggaaaccagttgaggaggaatgtgaagcagaccctgaacttcgacctgctgaagctggccggcgacgtggagagcaaccccggccccatggagcacagcaccttcctgagcggcctggtgctggccaccctgctgagccaggtgagccccttcaagatccccatcgaggagctggaggacagagtgttcgtgaactgcaacaccagcatcacctgggtggagggcaccgtgggcaccctgctgagcgacatcaccagactggacctgggcaagagaatcctggaccccagaggcatctacagatgcaacggcaccgacatctacaaggacaaggagagcaccgtgcaggtgcactacagaatgtgccagagctgcgtggagctggaccccgccaccgtggccggcatcatcgtgaccgacgtgatcgccaccctgctgctggccctgggcgtgttctgcttcgccggccacgagaccggcagactgagcggcgccgccgacacccaggccctgctgagaaacgaccaggtgtaccagcccctgagagacagagacgacgcccagtacagccacctgggcggcaactgggccagaaacaaggagggcagaggcagcctgctgacctgcggcgacgtggaggagaaccccggccccatgcagagcggcacccactggagagtgctgggcctgtgcctgctgagcgtgggcgtgtggggccaggacggcaacgaggagatgggcggcatcacccagaccccctacaaggtgagcatcagcggcaccaccgtgatcctgacctgcccccagtaccccggcagcgagatcctgtggcagcacaacgacaagaacatcggcggcgacgaggacgacaagaacatcggcagcgacgaggaccacctgagcctgaaggagttcagcgagctggagcagagcggctactacgtgtgctaccccagaggcagcaagcccgaggacgccaacttctacctgtacctgagagccagagtgtgcgagaactgcatggagatggacgtgatgagcgtggccaccatcgtgatcgtggacatctgcatcaccggcggcctgctgctgctggtgtactactggagcaagaacagaaaggccaaggccaagcccgtgaccagaggcgccggcgccggcggcagacagagaggccagaacaaggagagacccccccccgtgcccaaccccgactacgagcccatcagaaagggccagagagacctgtacagcggcctgaaccagagaagaatcggaccgcagtgtactaattatgctctcttgaaattggctggagatgttgagagcaatcccgggcccatgcgcattagcaagccccacctgcggagcatcagcatccagtgctacctgtgcctgctgctgaacagccacttcctgaccgaggccggcatccacgtgttcatcctgggctgcttcagcgccggactgcccaagaccgaggccaactgggtgaacgtgatcagcgacctgaagaagatcgaggacctgatccagagcatgcacatcgacgccaccctgtacaccgagagcgacgtgcaccccagctgcaaggtgaccgccatgaagtgctttctgctggaactgcaggtgatcagcctggaaagcggcgacgccagcatccacgacaccgtggagaacctgatcatcctggccaacaacagcctgagcagcaacggcaacgtgaccgagagcggctgcaaagagtgcgaggaactggaagagaagaacatcaaagagtttctgcagagcttcgtgcacatcgtgcagatgttcatcaacaccagc

SEQ ID NO: 123 - protein, 1033 aa, synthetic construct
MKWKALFTAAILQAQLPITEAQSFGLLDPKLCYLLDGILFIYGVILTALFLRSKRSRLLHSDYMNMTPRRPGPTRKHYQPYAPPRDFAAYRSLCARPRRSPAQEDGKVYINMPGRGRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPRQCTNYALLKLAGDVESNPGPMEQGKGLAVLILAIILLQGTLAQSIKGNHLVKVYDYQEDGSVLLTCDAEAKNITWFKDGKMIGFLTEDKKKWNLGSNAKDPRGMYQCKGSQNKSKPLQVYYRMCQNCIELNAATISGFLFAEIVSIFVLAVGVYFIAGQDGVRQSRASDKQTLLPNDQLYQPLKDREDDQYSHLQGNQLRRNVKQTLNFDLLKLAGDVESNPGPMEHSTFLSGLVLATLLSQVSPFKIPIEELEDRVFVNCNTSITWVEGTVGTLLSDITRLDLGKRILDPRGIYRCNGTDIYKDKESTVQVHYRMCQSCVELDPATVAGIIVTDVIATLLLALGVFCFAGHETGRLSGAADTQALLRNDQVYQPLRDRDDAQYSHLGGNWARNKEGRGSLLTCGDVEENPGPMQSGTHWRVLGLCLLSVGVWGQDGNEEMGGITQTPYKVSISGTTVILTCPQYPGSEILWQHNDKNIGGDEDDKNIGSDEDHLSLKEFSELEQSGYYVCYPRGSKPEDANFYLYLRARVCENCMEMDVMSVATIVIVDICITGGLLLLVYYWSKNRKAKAKPVTRGAGAGGRQRGQNKERPPPVPNPDYEPIRKGQRDLYSGLNQRRIGPQCTNYALLKLAGDVESNPGPMRISKPHLRSISIQCYLCLLLNSHFLTEAGIHVFILGCFSAGLPKTEANWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 124 - DNA, 3288 bp, synthetic construct
  source 1..3288: mol_type = other DNA
atggagaccctcttgggcctgcttatcctttggctgcagctgcaatgggtgagcagcaaacaggaggtgacacagattcctgcagctctgagtgtcccagaaggagaaaacttggttctcaactgcagtttcactgatagcgctatttacaacctccagtggtttaggcaggaccctgggaaaggtctcacatctctgttgcttattcagtcaagtcagagagagcaaacaagtggaagacttaatgcctcgctggataaatcatcaggacgtagtactttatacattgcagcttctcagcctggtgactcagccacctacctctgtgctgtgaggcccctttatggaggaagctacatacctacatttggaagaggaaccagccttattgttcatccgtatatccagaaccctgaccctgccgtgtaccagctgagagactctaaatccagtgacaagtctgtctgcctattcaccgattttgattctcaaacaaatgtgtcacaaagtaaggattctgatgtgtatatcacagacaaaactgtgctagacatgaggtctatggacttcaagagcaacagtgctgtggcctggagcaacaaatctgactttgcatgtgcaaacgccttcaacaacagcattattccagaagacaccttcttccccagcccagaaagttcctgtgatgtcaagctggtcgagaaaagctttgaaacagatacgaacctaaactttcaaaacctgtcagtgattgggttccgaatcctcctcctgaaagtggccgggtttaatctgctcatgacgctgcggctgtggtccagcggaagcggagctactaactttagcctgctgaagcaggctggagatgtggaggagaaccctggacctatgagcatcggcctcctgtgctgtgcagccttgtctctcctgtgggcaggtccagtgaatgctggtgtcactcagaccccaaaattccaggtcctgaagacaggacagagcatgacactgcagtgtgcccaggatatgaaccatgaatacatgtcctggtatcgacaagacccaggcatggggctgaggctgattcattactcagttggtgctggtatcactgaccaaggagaagtccccaatggctacaatgtctccagatcaaccacagaggatttcccgctcaggctgctgtcggctgctccctcccagacatctgtgtacttctgtgccagcagttacgtcgggaacaccggggagctgttttttggagaaggctctaggctgaccgtactggaggacctgaaaaacgtgttcccacccaaggtcgctgtgtttgagccatcagaagcagagatctcccacacccaaaaggccacactggtatgcctggccacaggcttctaccccgaccacgtggagctgagctggtgggtgaatgggaaggaggtgcacagtggggtcagcacagacccgcagcccctcaaggagcagcccgccctcaatgactccagatactgcctgagcagccgcctgagggtctcggccaccttctggcagaacccccgcaaccacttccgctgtcaagtccagttctacgggctctcggagaatgacgagtggacccaggatagggccaaacccgtcacccagatcgtcagcgccgaggcctggggtagagcagactgtggcttcacctccgagtcttaccagcaaggggtcctgtctgccaccatcctctatgagatcttgctagggaaggccaccttgtatgccgtgctggtcagtgccctcgtgctgatggccatggtcaagagaaaggattccagaggcagtggacagtgcaccaactacgccctgctgaagctggccggcgacgtggagagcaaccccggccccatggccttgcccgtcactgcgcttttgctcccgctcgctcttctcctgcatgcagcccgaccatctcaatttagagtttctccactcgacaggacgtggaacctcggcgaaaccgtcgaacttaaatgtcaagtacttctctcaaatccgacttctggttgctcatggctctttcagccgagaggagcagctgccagccccaccttcctgctgtatctctcccagaacaagccgaaggccgccgaagggctcgatactcaacgatttagcgggaagcgactcggggacacgttcgttcttactctcagcgattttagaagagagaacgagggatattatttttgttccgcactctctaacagcatcatgtacttcagtcattttgtaccagtctttctccctgcaaaaccaacgactactccagcaccaagaccgcccactcccgcacctactattgcaagccaacctttgagtctccgaccagaggcatgcagacctgctgctggaggtgcagtacatacgcgagggttggattttgcctgcgatatctatatctgggcccccttggccggcacgtgcggggtgctcctgctgagtctcgtaattactctttattgtaatcatagaaaccgcagaagggtgtgtaagtgtccccggcctgtcgtgaaaagcggggataagcccagtttgtctgctcggtacgtcggaagcggtgagggcaggggaagtcttctaacatgcggggacgtggaggaaaatcccggacccatgaggccacgactttggctgctgctcgctgcacagttgactgtactgcatggcaatagtgtgttgcagcagacacctgcatacatcaaggttcagacaaataagatggttatgctgagttgcgaggcaaaaattagtttgagcaatatgcggatctactggttgcgacagagacaggctcccagtagtgatagtcaccacgaattcctggctctttgggattccgcaaaaggaacgattcatggggaagaagtagagcaggagaagattgcggttttccgcgatgcatctcgctttatccttaatcttacatccgttaagcctgaggacagtgggatctatttttgtatgattgtagggtcccccgaattgacatttgggaagggtacgcagctctccgtagttgactttctgcccacaacggcacaacccactaagaagtccaccctgaagaagcgcgtctgtcgcttgcccagacctgaaacccaaaagggtccactctgttcccctataaccctggggttgttggtggcgggcgtcttggtcctgcttgttagcttgggcgtagccattcatctgtgttgccgaagacgcagagcccgacttagatttatgaagcaattctataagtga

SEQ ID NO: 125 - protein, 1095 aa, synthetic construct
METLLGLLILWLQLQWVSSKQEVTQIPAALSVPEGENLVLNCSFTDSAIYNLQWFRQDPGKGLTSLLLIQSSQREQTSGRLNASLDKSSGRSTLYIAASQPGDSATYLCAVRPLYGGSYIPTFGRGTSLIVHPYIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSSGSGATNFSLLKQAGDVEENPGPMSIGLLCCAALSLLWAGPVNAGVTQTPKFQVLKTGQSMTLQCAQDMNHEYMSWYRQDPGMGLRLIHYSVGAGITDQGEVPNGYNVSRSTTEDFPLRLLSAAPSQTSVYFCASSYVGNTGELFFGEGSRLTVLEDLKNVFPPKVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRGSGQCTNYALLKLAGDVESNPGPMALPVTALLLPLALLLHAARPSQFRVSPLDRTWNLGETVELKCQVLLSNPTSGCSWLFQPRGAAASPTFLLYLSQNKPKAAEGLDTQRFSGKRLGDTFVLTLSDFRRENEGYYFCSALSNSIMYFSHFVPVFLPAKPTTTPAPRPPTPAPTIASQPLSLRPEACRPAAGGAVHTRGLDFACDIYIWAPLAGTCGVLLLSLVITLYCNHRNRRRVCKCPRPVVKSGDKPSLSARYVGSGEGRGSLLTCGDVEENPGPMRPRLWLLLAAQLTVLHGNSVLQQTPAYIKVQTNKMVMLSCEAKISLSNMRIYWLRQRQAPSSDSHHEFLALWDSAKGTIHGEEVEQEKIAVFRDASRFILNLTSVKPEDSGIYFCMIVGSPELTFGKGTQLSVVDFLPTTAQPTKKSTLKKRVCRLPRPETQKGPLCSPITLGLLVAGVLVLLVSLGVAIHLCCRRRRARLRFMKQFYK

SEQ ID NO: 126 - DNA, 633 bp, synthetic construct
  source 1..633: mol_type = other DNA
atgaggccacgactttggctgctgctcgctgcacagttgactgtactgcatggcaatagtgtgttgcagcagacacctgcatacatcaaggttcagacaaataagatggttatgctgagttgcgaggcaaaaattagtttgagcaatatgcggatctactggttgcgacagagacaggctcccagtagtgatagtcaccacgaattcctggctctttgggattccgcaaaaggaacgattcatggggaagaagtagagcaggagaagattgcggttttccgcgatgcatctcgctttatccttaatcttacatccgttaagcctgaggacagtgggatctatttttgtatgattgtagggtcccccgaattgacatttgggaagggtacgcagctctccgtagttgactttctgcccacaacggcacaacccactaagaagtccaccctgaagaagcgcgtctgtcgcttgcccagacctgaaacccaaaagggtccactctgttcccctataaccctggggttgttggtggcgggcgtcttggtcctgcttgttagcttgggcgtagccattcatctgtgttgccgaagacgcagagcccgacttagatttatgaagcaattctataagtga

SEQ ID NO: 127 - DNA, 705 bp, synthetic construct
  source 1..705: mol_type = other DNA
atggccttgcccgtcactgcgcttttgctcccgctcgctcttctcctgcatgcagcccgaccatctcaatttagagtttctccactcgacaggacgtggaacctcggcgaaaccgtcgaacttaaatgtcaagtacttctctcaaatccgacttctggttgctcatggctctttcagccgagaggagcagctgccagccccaccttcctgctgtatctctcccagaacaagccgaaggccgccgaagggctcgatactcaacgatttagcgggaagcgactcggggacacgttcgttcttactctcagcgattttagaagagagaacgagggatattatttttgttccgcactctctaacagcatcatgtacttcagtcattttgtaccagtctttctccctgcaaaaccaacgactactccagcaccaagaccgcccactcccgcacctactattgcaagccaacctttgagtctccgaccagaggcatgcagacctgctgctggaggtgcagtacatacgcgagggttggattttgcctgcgatatctatatctgggcccccttggccggcacgtgcggggtgctcctgctgagtctcgtaattactctttattgtaatcatagaaaccgcagaagggtgtgtaagtgtccccggcctgtcgtgaaaagcggggataagcccagtttgtctgctcggtacgtc

SEQ ID NO: 128 - protein, 210 aa, synthetic construct
MRPRLWLLLAAQLTVLHGNSVLQQTPAYIKVQTNKMVMLSCEAKISLSNMRIYWLRQRQAPSSDSHHEFLALWDSAKGTIHGEEVEQEKIAVFRDASRFILNLTSVKPEDSGIYFCMIVGSPELTFGKGTQLSVVDFLPTTAQPTKKSTLKKRVCRLPRPETQKGPLCSPITLGLLVAGVLVLLVSLGVAIHLCCRRRRARLRFMKQFYK

SEQ ID NO: 129 - protein, 235 aa, synthetic construct
MALPVTALLLPLALLLHAARPSQFRVSPLDRTWNLGETVELKCQVLLSNPTSGCSWLFQPRGAAASPTFLLYLSQNKPKAAEGLDTQRFSGKRLGDTFVLTLSDFRRENEGYYFCSALSNSIMYFSHFVPVFLPAKPTTTPAPRPPTPAPTIASQPLSLRPEACRPAAGGAVHTRGLDFACDIYIWAPLAGTCGVLLLSLVITLYCNHRNRRRVCKCPRPVVKSGDKPSLSARYV

SEQ ID NO: 130 - skipped

SEQ ID NO: 131 - protein, 9 aa, synthetic construct
SLLQHLIGL

SEQ ID NO: 132 - protein, 9 aa, synthetic construct
QLLALLPSL

SEQ ID NO: 133 - DNA, 816 bp, synthetic construct
  source 1..816: mol_type = other DNA
atgcttctggaacacctgctgattatcctgtggatgcaactcacgtgggtctccgggcaacaactgaatcaaagcccccaatccatgtttatacaggagggagaggacgtaagtatgaattgcacatcttcatctatctttaacacctggctgtggtacaaacaagaccccggagaaggtcctgtacttctcatcgcactttacaaagcaggtgagcttaccagtaacgggagactcaccgcacagttcggtattacaagaaaggattcctttctcaacatctccgcttctatcccttcagacgtcggaatttatttttgtgctggtatccctcgagacaattacggtcaaaactttgtatttgggcctgggactcggctgtcagttttgccgtatatccagaaccccgaccccgccgtgtaccagctgcgggacagcaagagcagcgacaagagcgtgtgcctgttcaccgacttcgacagccagaccaacgtgtcccagagcaaggacagcgacgtgtacatcaccgataagtgcgtgctggacatgcggagcatggacttcaagagcaacagcgccgtggcctggtccaacaagagcgacttcgcctgcgccaacgccttcaacaacagcatcatccccgaggacacattcttcccaagccccgagagcagctgcgacgtgaagctggtggagaagtccttcgagacagacaccaacctgaacttccagaacctgtccgtgatcggcttcagaatcctgctgctgaaagtggccggcttcaacctgctgatgaccctgcggctgtggtccagc

SEQ ID NO: 134 - DNA, 936 bp, synthetic construct
  source 1..936: mol_type = other DNA
atgggcattaggctgctgtgcagagtagcattttgctttctggcagtaggattggtcgatgtaaaggttacacagtcctcacggtacttggtaaagcgcactggtgaaaaggtctttctggaatgtgtacaagatatggatcacgaaaatatgttttggtacaggcaagatcccggccttggacttagactgatatatttctcctacgatgttaaaatgaaggagaagggcgatattccagaaggatattccgtgagccgcgaaaagaaggagcgattcagtttgatactcgaaagtgcctccacaaaccaaacctctatgtacctttgcgcgtcaacgccgtggctggccggtggcaatgaacaattcttcgggccgggtacgcgcctcactgtcctggaggacctcaagaatgtgtttccgcccgaagtcgcggtttttgaaccatcagaagccgagatctctcatacacaaaaggcgacgctcgtatgccttgcgacgggattttatccggaccacgtcgagctttcctggtgggttaatggaaaggaggtgcattccggagtttgcacggaccctcagccattgaaggaacagcccgcactgaacgacagtaggtattgcctttcatctcgcctgcgcgtgtctgcgacattctggcaaaacccaagaaatcacttcagatgtcaagttcagttctacggtctcagcgagaatgatgagtggacacaagatagggctaaacccgtgactcaaatagtctctgccgaggcctgggggagggcggattgcggcttcacatcagaatcataccaacaaggagtattgagcgcgacaattctttacgaaattctgcttgggaaagcgactctgtacgcggtgctcgtgtccgctttggttcttatggcaatggttaaacgaaaggatagtaggggc

SEQ ID NO: 135 - protein, 272 aa, synthetic construct
MLLEHLLIILWMQLTWVSGQQLNQSPQSMFIQEGEDVSMNCTSSSIFNTWLWYKQDPGEGPVLLIALYKAGELTSNGRLTAQFGITRKDSFLNISASIPSDVGIYFCAGIPRDNYGQNFVFGPGTRLSVLPYIQNPDPAVYQLRDSKSSDKSVCLFTDFDSQTNVSQSKDSDVYITDKCVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 136 - protein, 312 aa, synthetic construct
MGIRLLCRVAFCFLAVGLVDVKVTQSSRYLVKRTGEKVFLECVQDMDHENMFWYRQDPGLGLRLIYFSYDVKMKEKGDIPEGYSVSREKKERFSLILESASTNQTSMYLCASTPWLAGGNEQFFGPGTRLTVLEDLKNVFPPEVAVFEPSEAEISHTQKATLVCLATGFYPDHVELSWWVNGKEVHSGVCTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 137 - DNA, 822 bp, synthetic construct
  source 1..822: mol_type = other DNA
atgctgctgctgctggtgcccgtgctggaagtgatcttcaccctgggcggcaccagagcccagagcgtgacacagctgggcagccacgtgtccgtgtctgagagggccctggtgctgctgagatgcaactactcttctagcgtgcccccctacctgttttggtacgtgcagtaccccaaccaggggctgcagctgctcctgaagtacaccagcgccgccacactggtgaagggcatcaacggcttcgaggccgagttcaagaagtccgagacaagcttccacctgaccaagcccagcgcccacatgtctgacgccgccgagtacttctgtgccgtgagcggccagaccggcgccaacaacctgttcttcggcaccggcacccggctgacagtgatcccttacatccagaaccccgaccccgccgtgtaccagctgcgggacagcaagagcagcgacaagagcgtgtgcctgttcaccgacttcgacagccagaccaacgtgtcccagagcaaggacagcgacgtgtacatcaccgataagtgcgtgctggacatgcggagcatggacttcaagagcaacagcgccgtggcctggtccaacaagagcgacttcgcctgcgccaacgccttcaacaacagcatcatccccgaggacacattcttcccaagccccgagagcagctgcgacgtgaagctggtggagaagtccttcgagacagacaccaacctgaacttccagaacctgtccgtgatcggcttcagaatcctgctgctgaaagtggccggcttcaacctgctgatgaccctgcggctgtggtccagc

SEQ ID NO: 138 - DNA, 933 bp, synthetic construct
  source 1..933: mol_type = other DNA
atgggcttccggctgctgtgctgcgtggccttttgtctgctgggagccggacctgtggatagcggcgtgacccagacccccaagcacctgatcaccgccaccggccagagagtgaccctgcgctgcagccctagaagcggcgacctgagcgtgtactggtatcagcagagcctcgaccagggcctgcagttcctgatccagtactacaacggcgaggaacgggccaagggcaacatcctggaacggttcagcgcccagcagttccccgatctgcacagcgagctgaacctgagcagcctggaactgggcgacagcgccctgtacttctgcgccagcgccagatgggatagaggcggcgagcagtacttcggccctggcaccagactgaccgtgaccgaggacctcaagaatgtgtttccgcccgaagtcgcggtttttgaaccatcagaagccgagatctctcatacacaaaaggcgacgctcgtatgccttgcgacgggattttatccggaccacgtcgagctttcctggtgggttaatggaaaggaggtgcattccggagtttgcacggaccctcagccattgaaggaacagcccgcactgaacgacagtaggtattgcctttcatctcgcctgcgcgtgtctgcgacattctggcaaaacccaagaaatcacttcagatgtcaagttcagttctacggtctcagcgagaatgatgagtggacacaagatagggctaaacccgtgactcaaatagtctctgccgaggcctgggggagggcggattgcggcttcacatcagaatcataccaacaaggagtattgagcgcgacaattctttacgaaattctgcttgggaaagcgactctgtacgcggtgctcgtgtccgctttggttcttatggcaatggttaaacgaaaggatagtaggggc

SEQ ID NO: 139 - protein, 274 aa, synthetic construct
MLLLLVPVLEVIFTLGGTRAQSVTQLGSHVSVSERALVLLRCNYSSSVPPYLFWYVQYPNQGLQLLLKYTSAATLVKGINGFEAEFKKSETSFHLTKPSAHMSDAAEYFCAVSGQTGANNLFFGTGTRLTVIPYIQNPDPAVYQLRDSKSSDKSVCLFTDFDSQTNVSQSKDSDVYITDKCVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 140 - protein, 311 aa, synthetic construct
MGFRLLCCVAFCLLGAGPVDSGVTQTPKHLITATGQRVTLRCSPRSGDLSVYWYQQSLDQGLQFLIQYYNGEERAKGNILERFSAQQFPDLHSELNLSSLELGDSALYFCASARWDRGGEQYFGPGTRLTVTEDLKNVFPPEVAVFEPSEAEISHTQKATLVCLATGFYPDHVELSWWVNGKEVHSGVCTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 141 - DNA, 819 bp, synthetic construct
  source 1..819: mol_type = other DNA
atgaagagcctgagggtactgctggtgatattgtggcttcagcttagttgggtctggtcacaacaaaaggaagttgagcaaaactcaggaccactgagtgtacccgagggcgctatagcatcactgaactgtacctactcagatcggggaagccaatcctttttctggtacagacagtattccgggaagagtcctgagttgatcatgtttatatactccaatggcgataaggaggatggacgcttcaccgctcagcttaataaagcgtcacagtatgtatccctcctgattcgggactcacaaccatctgactctgcaacatacctttgtgccgtaaaggacaacgccgggaacatgctcacttttggaggaggtacccggcttatggtaaaaccacatatccagaaccccgaccccgccgtgtaccagctgcgggacagcaagagcagcgacaagagcgtgtgcctgttcaccgacttcgacagccagaccaacgtgtcccagagcaaggacagcgacgtgtacatcaccgataagtgcgtgctggacatgcggagcatggacttcaagagcaacagcgccgtggcctggtccaacaagagcgacttcgcctgcgccaacgccttcaacaacagcatcatccccgaggacacattcttcccaagccccgagagcagctgcgacgtgaagctggtggagaagtccttcgagacagacaccaacctgaacttccagaacctgtccgtgatcggcttcagaatcctgctgctgaaagtggccggcttcaacctgctgatgaccctgcggctgtggtccagc

SEQ ID NO: 142 - protein, 273 aa, synthetic construct
MKSLRVLLVILWLQLSWVWSQQKEVEQNSGPLSVPEGAIASLNCTYSDRGSQSFFWYRQYSGKSPELIMFIYSNGDKEDGRFTAQLNKASQYVSLLIRDSQPSDSATYLCAVKDNAGNMLTFGGGTRLMVKPHIQNPDPAVYQLRDSKSSDKSVCLFTDFDSQTNVSQSKDSDVYITDKCVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 143 - DNA, 933 bp, synthetic construct
  source 1..933: mol_type = other DNA
atgggattccggcttctttgttgtgtggcattttgtctgttgggtgcgggtccagtcgatagtggtgtaactcagacaccaaaacaccttatcacggcaactgggcaacgagtgacgctccgctgtagcccgaggtccggtgatttgagtgtgtactggtaccagcaatctttggaccagggcttgcagttcctcatacagtattacaatggtgaagaaagagcgaagggtaatatcctggaaagattctccgcacaacagtttcctgatctccacagcgaactgaacctgagttctctcgagctcggggatagtgctttgtacttctgcgcgtcatccgacggtggcggagtctatgaacaatatttcggcccagggactaggcttacggtgacggaggacctcaagaatgtgtttccgcccgaagtcgcggtttttgaaccatcagaagccgagatctctcatacacaaaaggcgacgctcgtatgccttgcgacgggattttatccggaccacgtcgagctttcctggtgggttaatggaaaggaggtgcattccggagtttgcacggaccctcagccattgaaggaacagcccgcactgaacgacagtaggtattgcctttcatctcgcctgcgcgtgtctgcgacattctggcaaaacccaagaaatcacttcagatgtcaagttcagttctacggtctcagcgagaatgatgagtggacacaagatagggctaaacccgtgactcaaatagtctctgccgaggcctgggggagggcggattgcggcttcacatcagaatcataccaacaaggagtattgagcgcgacaattctttacgaaattctgcttgggaaagcgactctgtacgcggtgctcgtgtccgctttggttcttatggcaatggttaaacgaaaggatagtaggggc

SEQ ID NO: 144 - protein, 311 aa, synthetic construct
MGFRLLCCVAFCLLGAGPVDSGVTQTPKHLITATGQRVTLRCSPRSGDLSVYWYQQSLDQGLQFLIQYYNGEERAKGNILERFSAQQFPDLHSELNLSSLELGDSALYFCASSDGGGVYEQYFGPGTRLTVTEDLKNVFPPEVAVFEPSEAEISHTQKATLVCLATGFYPDHVELSWWVNGKEVHSGVCTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 145 - DNA, 402 bp, synthetic construct
  source 1..402: mol_type = other DNA
atggagacactgctgaaggtgctgtctggcacactgctgtggcagctgacctgggtccgatctcagcagcctgttcagtctcctcaggccgtgatcctgagagaaggcgaggacgccgtgatcaactgcagcagctctaaggccctgtacagcgtgcactggtacagacagaagcacggcgaggcccctgtgttcctgatgatcctgctgaaaggcggcgagcagaagggccacgagaagatcagcgccagcttcaacgagaagaagcagcagtccagcctgtacctgacagccagccagctgagctacagcggcacctacttttgcggcacagccaatagcggcggcagcaactacaagctgaccttcggcaagggcaccctgctgaccgtgaatcccaat

SEQ ID NO: 146 - DNA, 396 bp, synthetic construct
  source 1..396: mol_type = other DNA
atgctgctgatcacctccatgctggtgctgtggatgcagctgagccaagtgaacggccagcaagtgatgcagatccctcagtaccagcacgtgcaagaaggcgaggacttcaccacctactgcaacagcagcaccacactgagcaacatccagtggtacaagcagcggcctggcggacaccctgtgtttctgatccagctggtcaagtccggcgaagtgaagaagcagaagcggctgaccttccagttcggcgaggccaagaagaacagcagcctgcacatcaccgccacacagaccaccgatgtgggcacctacttttgtgctggcgccctgcctagagccggcagctatcaactgacattcggcaagggcaccaagctgagcgtgatccccaac

SEQ ID NO: 147 - DNA, 810 bp, synthetic construct
  source 1..810: mol_type = other DNA
atggagacactgctgaaggtgctgtctggcacactgctgtggcagctgacctgggtccgatctcagcagcctgttcagtctcctcaggccgtgatcctgagagaaggcgaggacgccgtgatcaactgcagcagctctaaggccctgtacagcgtgcactggtacagacagaagcacggcgaggcccctgtgttcctgatgatcctgctgaaaggcggcgagcagaagggccacgagaagatcagcgccagcttcaacgagaagaagcagcagtccagcctgtacctgacagccagccagctgagctacagcggcacctacttttgcggcacagccaatagcggcggcagcaactacaagctgaccttcggcaagggcaccctgctgaccgtgaatcccaatatccagaatccggagcccgccgtataccagctgaaggaccctagaagccaggacagcaccctgtgcctgttcaccgacttcgacagccagatcaacgtgcccaagaccatggaaagcggcaccttcatcaccgacaagacagtgctggacatgaaggccatggacagcaagtccaacggcgcaatcgcctggtccaaccagaccagcttcacatgccaggacatcttcaaagagacaaacgccacataccccagcagcgacgtgccctgtgatgccaccctgacagagaagtccttcgagacagacatgaacctgaacttccagaatctgtccgtgatgggcctgagaatcctgctgctgaaggtggccggcttcaatctgctgatgaccctgcggctgtggtccagc

SEQ ID NO: 148 - DNA, 804 bp, synthetic construct
  source 1..804: mol_type = other DNA
atgctgctgatcacctccatgctggtgctgtggatgcagctgagccaagtgaacggccagcaagtgatgcagatccctcagtaccagcacgtgcaagaaggcgaggacttcaccacctactgcaacagcagcaccacactgagcaacatccagtggtacaagcagcggcctggcggacaccctgtgtttctgatccagctggtcaagtccggcgaagtgaagaagcagaagcggctgaccttccagttcggcgaggccaagaagaacagcagcctgcacatcaccgccacacagaccaccgatgtgggcacctacttttgtgctggcgccctgcctagagccggcagctatcaactgacattcggcaagggcaccaagctgagcgtgatccccaacatccagaatccggagcccgccgtataccagctgaaggaccctagaagccaggacagcaccctgtgcctgttcaccgacttcgacagccagatcaacgtgcccaagaccatggaaagcggcaccttcatcaccgacaagacagtgctggacatgaaggccatggacagcaagtccaacggcgcaatcgcctggtccaaccagaccagcttcacatgccaggacatcttcaaagagacaaacgccacataccccagcagcgacgtgccctgtgatgccaccctgacagagaagtccttcgagacagacatgaacctgaacttccagaatctgtccgtgatgggcctgagaatcctgctgctgaaggtggccggcttcaatctgctgatgaccctgcggctgtggtccagc

SEQ ID NO: 149 - DNA, 393 bp, synthetic construct
  source 1..393: mol_type = other DNA
atgggcaccagactgttcttctacgtggccctgtgtctgctgtggacaggccatgtggatgccggaatcacacagagccccagacacaaagtgaccgagacaggcacccctgtgacactgagatgtcaccagaccgagaaccatcggtacatgtattggtacagacaggaccccggccacggcctgagactgatccactatagctacggcgtgaaggacaccgacaagggcgaagtgtctgacggctacagcgtgtccagaagcaagaccgaggacttcctgctgaccctggaaagcgccacaagcagccagaccagcgtgtacttctgcgccatcagcgactacgagggcaccgaggccttttttggccaaggcacaagactgaccgtggtg

SEQ ID NO: 150 - DNA, 393 bp, synthetic construct
  source 1..393: mol_type = other DNA
atgctgtgttctctgctggctctgctgctgggcaccttttttggcgtcagaagccagaccatccaccagtggcctgctacactggtgcagcctgttggaagccctctgagcctggaatgtaccgtggaaggcaccagcaatcccaacctgtactggtacagacaggccgctggaagaggactgcagctgctgttttacagcgtcggcatcggccagatcagcagcgaggttccacagaatctgagcgccagcagaccccaggacagacagtttatcctgagcagcaagaagctgctgctgagcgacagcggcttctacctgtgtgcttggagcctcggagccggctacaccgacacacagtattttggccctggcaccagactgaccgtgctg

SEQ ID NO: 151 - DNA, 912 bp, synthetic construct
  source 1..912: mol_type = other DNA
atgggcaccagactgttcttctacgtggccctgtgtctgctgtggacaggccatgtggatgccggaatcacacagagccccagacacaaagtgaccgagacaggcacccctgtgacactgagatgtcaccagaccgagaaccatcggtacatgtattggtacagacaggaccccggccacggcctgagactgatccactatagctacggcgtgaaggacaccgacaagggcgaagtgtctgacggctacagcgtgtccagaagcaagaccgaggacttcctgctgaccctggaaagcgccacaagcagccagaccagcgtgtacttctgcgccatcagcgactacgagggcaccgaggccttttttggccaaggcacaagactgaccgtggtggaagatctccggaacgtgaccccccctaaagtgaccctgttcgaacccagcaaggccgagatcgccaacaagcagaaagccaccctcgtgtgcctggccagaggcttcttccccgaccatgtggaactgtcttggtgggtcaacggcaaagaggtgcacagcggagtgtccaccgaccctcaggcctacaaagagagcaactacagctactgcctgagcagcagactgcgggtgtccgccaccttctggcacaacccccggaaccacttcagatgccaggtgcagtttcacggcctgagcgaagaggacaagtggcccgaaggctcccccaagcccgtgacccagaatatctctgccgaggcctggggcagagccgactgtggaattaccagcgccagctaccaccagggcgtgctgtctgccaccatcctgtacgagatcctgctgggcaaggccaccctgtacgccgtgctggtgtctggcctggtgctgatggccatggtcaagaagaagaacagc

SEQ ID NO: 152 - DNA, 912 bp, synthetic construct
  source 1..912: mol_type = other DNA
atgctgtgttctctgctggctctgctgctgggcaccttttttggcgtcagaagccagaccatccaccagtggcctgctacactggtgcagcctgttggaagccctctgagcctggaatgtaccgtggaaggcaccagcaatcccaacctgtactggtacagacaggccgctggaagaggactgcagctgctgttttacagcgtcggcatcggccagatcagcagcgaggttccacagaatctgagcgccagcagaccccaggacagacagtttatcctgagcagcaagaagctgctgctgagcgacagcggcttctacctgtgtgcttggagcctcggagccggctacaccgacacacagtattttggccctggcaccagactgaccgtgctggaagatctccggaacgtgaccccccctaaagtgaccctgttcgaacccagcaaggccgagatcgccaacaagcagaaagccaccctcgtgtgcctggccagaggcttcttccccgaccatgtggaactgtcttggtgggtcaacggcaaagaggtgcacagcggagtgtccaccgaccctcaggcctacaaagagagcaactacagctactgcctgagcagcagactgcgggtgtccgccaccttctggcacaacccccggaaccacttcagatgccaggtgcagtttcacggcctgagcgaagaggacaagtggcccgaaggctcccccaagcccgtgacccagaatatctctgccgaggcctggggcagagccgactgtggaattaccagcgccagctaccaccagggcgtgctgtctgccaccatcctgtacgagatcctgctgggcaaggccaccctgtacgccgtgctggtgtctggcctggtgctgatggccatggtcaagaagaagaacagc

SEQ ID NO: 153 - protein, 134 aa, synthetic construct
METLLKVLSGTLLWQLTWVRSQQPVQSPQAVILREGEDAVINCSSSKALYSVHWYRQKHGEAPVFLMILLKGGEQKGHEKISASFNEKKQQSSLYLTASQLSYSGTYFCGTANSGGSNYKLTFGKGTLLTVNPN

SEQ ID NO: 154 - protein, 132 aa, synthetic construct
MLLITSMLVLWMQLSQVNGQQVMQIPQYQHVQEGEDFTTYCNSSTTLSNIQWYKQRPGGHPVFLIQLVKSGEVKKQKRLTFQFGEAKKNSSLHITATQTTDVGTYFCAGALPRAGSYQLTFGKGTKLSVIPN

SEQ ID NO: 155 - protein, 136 aa, synthetic construct
IQNPEPAVYQLKDPRSQDSTLCLFTDFDSQINVPKTMESGTFITDKTVLDMKAMDSKSNGAIAWSNQTSFTCQDIFKETNATYPSSDVPCDATLTEKSFETDMNLNFQNLSVMGLRILLLKVAGFNLLMTLRLWSS

SEQ ID NO: 156 - protein, 140 aa, synthetic construct; identical to SEQ ID NO: 89

SEQ ID NO: 157 - protein, 140 aa, synthetic construct
IQNPDPAVYQLRDSKSSDKSVCLFTDFDSQTNVSQSKDSDVYITDKTVLDMRSMDFKSNSAVAWSNKSDFACANAFNNSIIPEDTFFPSSDVPCDVKLVEKSFETDTNLNFQNLSVIGFRILLLKVAGFNLLMTLRLWSS

SEQ ID NO: 158 - protein, 270 aa, synthetic construct
METLLKVLSGTLLWQLTWVRSQQPVQSPQAVILREGEDAVINCSSSKALYSVHWYRQKHGEAPVFLMILLKGGEQKGHEKISASFNEKKQQSSLYLTASQLSYSGTYFCGTANSGGSNYKLTFGKGTLLTVNPNIQNPEPAVYQLKDPRSQDSTLCLFTDFDSQINVPKTMESGTFITDKTVLDMKAMDSKSNGAIAWSNQTSFTCQDIFKETNATYPSSDVPCDATLTEKSFETDMNLNFQNLSVMGLRILLLKVAGFNLLMTLRLWSS

SEQ ID NO: 159 - protein, 268 aa, synthetic construct
MLLITSMLVLWMQLSQVNGQQVMQIPQYQHVQEGEDFTTYCNSSTTLSNIQWYKQRPGGHPVFLIQLVKSGEVKKQKRLTFQFGEAKKNSSLHITATQTTDVGTYFCAGALPRAGSYQLTFGKGTKLSVIPNIQNPEPAVYQLKDPRSQDSTLCLFTDFDSQINVPKTMESGTFITDKTVLDMKAMDSKSNGAIAWSNQTSFTCQDIFKETNATYPSSDVPCDATLTEKSFETDMNLNFQNLSVMGLRILLLKVAGFNLLMTLRLWSS

SEQ ID NO: 160 - protein, 131 aa, synthetic construct
MGTRLFFYVALCLLWTGHVDAGITQSPRHKVTETGTPVTLRCHQTENHRYMYWYRQDPGHGLRLIHYSYGVKDTDKGEVSDGYSVSRSKTEDFLLTLESATSSQTSVYFCAISDYEGTEAFFGQGTRLTVV

SEQ ID NO: 161 - protein, 131 aa, synthetic construct
MLCSLLALLLGTFFGVRSQTIHQWPATLVQPVGSPLSLECTVEGTSNPNLYWYRQAAGRGLQLLFYSVGIGQISSEVPQNLSASRPQDRQFILSSKKLLLSDSGFYLCAWSLGAGYTDTQYFGPGTRLTVL

SEQ ID NO: 162 - protein, 173 aa, synthetic construct
EDLRNVTPPKVTLFEPSKAEIANKQKATLVCLARGFFPDHVELSWWVNGKEVHSGVSTDPQAYKESNYSYCLSSRLRVSATFWHNPRNHFRCQVQFHGLSEEDKWPEGSPKPVTQNISAEAWGRADCGITSASYHQGVLSATILYEILLGKATLYAVLVSGLVLMAMVKKKNS

SEQ ID NO: 163 - protein, 176 aa, synthetic construct
DLNKVFPPEVAVFEPSEAEISHTQKATLVCLATGFFPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDF

SEQ ID NO: 164 - protein, 177 aa, synthetic construct
EDLNKVFPPEVAVFEPSKAEIAHTQKATLVCLATGFFPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGITSASYHQGVLSATILYEILLGKATLYAVLVSALVLMAMVKRKDF

SEQ ID NO: 165 - protein, 304 aa, synthetic construct
MGTRLFFYVALCLLWTGHVDAGITQSPRHKVTETGTPVTLRCHQTENHRYMYWYRQDPGHGLRLIHYSYGVKDTDKGEVSDGYSVSRSKTEDFLLTLESATSSQTSVYFCAISDYEGTEAFFGQGTRLTVVEDLRNVTPPKVTLFEPSKAEIANKQKATLVCLARGFFPDHVELSWWVNGKEVHSGVSTDPQAYKESNYSYCLSSRLRVSATFWHNPRNHFRCQVQFHGLSEEDKWPEGSPKPVTQNISAEAWGRADCGITSASYHQGVLSATILYEILLGKATLYAVLVSGLVLMAMVKKKNS

SEQ ID NO: 166 - protein, 304 aa, synthetic construct
MLCSLLALLLGTFFGVRSQTIHQWPATLVQPVGSPLSLECTVEGTSNPNLYWYRQAAGRGLQLLFYSVGIGQISSEVPQNLSASRPQDRQFILSSKKLLLSDSGFYLCAWSLGAGYTDTQYFGPGTRLTVLEDLRNVTPPKVTLFEPSKAEIANKQKATLVCLARGFFPDHVELSWWVNGKEVHSGVSTDPQAYKESNYSYCLSSRLRVSATFWHNPRNHFRCQVQFHGLSEEDKWPEGSPKPVTQNISAEAWGRADCGITSASYHQGVLSATILYEILLGKATLYAVLVSGLVLMAMVKKKNS

SEQ ID NO: 167 - protein, 9 aa, synthetic construct
LYVDSLFFL

SEQ ID NO: 168 - protein, 9 aa, synthetic construct
KTWGQYWQV

SEQ ID NO: 169 - DNA, 804 bp, synthetic construct
  source 1..804: mol_type = other DNA
atgaaatccttgagtgtttccctagtggtcctgtggctccagttaaactgggtgaacagccagcagaaggtgcagcagagcccagaatccctcattgtcccagagggagccatgacctctctcaactgcactttcagcgacagtgcttctcagtattttgcatggtacagacagcattctgggaaagcccccaaggcactgatgtccatcttctccaatggtgaaaaagaagaaggcagattcacaattcacctcaataaagccagtctgcatttctcgctacacatcagagactcccagcccagtgactctgctctctacctctgtgcagccaataactatgcccagggattaaccttcggtcttggcaccagagtatctgtgtttccctacatccagaacccagaacctgctgtgtaccagttaaaagatcctcggtctcaggacagcaccctctgcctgttcaccgactttgactcccaaatcaatgtgccgaaaaccatggaatctggaacgttcatcactgacaaaactgtgctggacatgaaagctatggattccaagagcaatggggccattgcctggagcaaccagacaagcttcacctgccaagatatcttcaaagagaccaacgccacctaccccagttcagacgttccctgtgatgccacgttgactgagaaaagctttgaaacagatatgaacctaaactttcaaaacctgtcagttatgggactccgaatcctcctgctgaaagtagccggatttaacctgctcatgacgctgaggctgtggtccagttga

SEQ ID NO: 170 - DNA, 912 bp, synthetic construct
  source 1..912: mol_type = other DNA
atgggctccagactcttctttgtggttttgattctcctgtgtgcaaaacacatggaggctgcagtcacccaaagtccaagaagcaaggtggcagtaacaggaggaaaggtgacattgagctgtcaccagactaataaccatgactatatgtactggtatcggcaggacacggggcatgggctgaggctgatccattactcatatgtcgctgacagcacggagaaaggagatatccctgatgggtacaaggcctccagaccaagccaagagaatttctctctcattctggagttggcttccctttctcagacagctgtatatttctgtgccagcagccctgggggggggggggaacagtacttcggtcccggcaccaggctcacggttttagaggatctgagaaatgtgactccacccaaggtctccttgtttgagccatcaaaagcagagattgcaaacaaacgaaaggctaccctcgtgtgcttggccaggggcttcttccctgaccacgtggagctgagctggtgggtgaatggcaaggaggtccacagtggggtcagcacggaccctcaggcctacaaggagagcaattatagctactgcctgagcagccgcctgagggtctctgctaccttctggcacaatcctcgaaaccacttccgctgccaagtgcagttccatgggctttcagaggaggacaagtggccagagggctcacccaaacctgtcacacagaacatcagtgcagaggcctggggccgagcagactgtgggattacctcagcatcctatcaacaaggggtcttgtctgccaccatcctctatgagatcctgctagggaaagccaccctgtatgctgtgcttgtcagtacactggtggtgatggctatggtcaaaagaaagaattcatga

SEQ ID NO: 171 - protein, 267 aa, synthetic construct
MKSLSVSLVVLWLQLNWVNSQQKVQQSPESLIVPEGAMTSLNCTFSDSASQYFAWYRQHSGKAPKALMSIFSNGEKEEGRFTIHLNKASLHFSLHIRDSQPSDSALYLCAANNYAQGLTFGLGTRVSVFPYIQNPEPAVYQLKDPRSQDSTLCLFTDFDSQINVPKTMESGTFITDKTVLDMKAMDSKSNGAIAWSNQTSFTCQDIFKETNATYPSSDVPCDATLTEKSFETDMNLNFQNLSVMGLRILLLKVAGFNLLMTLRLWSS

SEQ ID NO: 172 - protein, 303 aa, synthetic construct
MGSRLFFVVLILLCAKHMEAAVTQSPRSKVAVTGGKVTLSCHQTNNHDYMYWYRQDTGHGLRLIHYSYVADSTEKGDIPDGYKASRPSQENFSLILELASLSQTAVYFCASSPGGGGEQYFGPGTRLTVLEDLRNVTPPKVSLFEPSKAEIANKRKATLVCLARGFFPDHVELSWWVNGKEVHSGVSTDPQAYKESNYSYCLSSRLRVSATFWHNPRNHFRCQVQFHGLSEEDKWPEGSPKPVTQNISAEAWGRADCGITSASYQQGVLSATILYEILLGKATLYAVLVSTLVVMAMVKRKNS

SEQ ID NO: 173 - protein, 111 aa, synthetic construct
QQKVQQSPESLIVPEGAMTSLNCTFSDSASQYFAWYRQHSGKAPKALMSIFSNGEKEEGRFTIHLNKASLHFSLHIRDSQPSDSALYLCAANNYAQGLTFGLGTRVSVFPY

SEQ ID NO: 174 - protein, 112 aa, synthetic construct
EAAVTQSPRSKVAVTGGKVTLSCHQTNNHDYMYWYRQDTGHGLRLIHYSYVADSTEKGDIPDGYKASRPSQENFSLILELASLSQTAVYFCASSPGGGGEQYFGPGTRLTVL

SEQ ID NO: 175 - protein, 9 aa, synthetic construct
AAGIGILTV

SEQ ID NO: 176 - DNA, 804 bp, synthetic construct
  source 1..804: mol_type = other DNA
atgttgcttgaacatttattaataatcttgtggatgcagctgacatgggtcagtggtcaacagctgaatcagagtcctcaatctatgtttatccaggaaggagaagatgtctccatgaactgcacttcttcaagcatatttaacacctggctatggtacaagcaggaccctggggaaggtcctgtcctcttgatagccttatataaggctggtgaattgacctcaaatggaagactgactgctcagtttggtataaccagaaaggacagcttcctgaatatctcagcatccatacctagtgatgtaggcatctacttctgtgctggtgggaccggtaaccagttctattttgggacagggacaagtttgacggtcattccaaatatccagaaccctgaccctgccgtgtaccagctgagagactctaaatccagtgacaagtctgtctgcctattcaccgattttgattctcaaacaaatgtgtcacaaagtaaggattctgatgtgtatatcacagacaaaactgtgctagacatgaggtctatggacttcaagagcaacagtgctgtggcctggagcaacaaatctgactttgcatgtgcaaacgccttcaacaacagcattattccagaagacaccttcttccccagcccagaaagttcctgtgatgtcaagctggtcgagaaaagctttgaaacagatacgaacctaaactttcaaaacctgtcagtgattgggttccgaatcctcctcctgaaggtggccgggtttaatctgctcatgacgctgcggctgtggtccagc

SEQ ID NO: 177 - DNA, 921 bp, synthetic construct
  source 1..921: mol_type = other DNA
atgggcacaaggttgttcttctatgtggccctttgtctcctgtggacaggacacatggatgctggaatcacccagagcccaagacacaaggtcacagagacaggaacaccagtgactctgagatgtcaccagactgagaaccaccgctatatgtactggtatcgacaagacccggggcatgggctgaggctgatccattactcatatggtgttaaagatactgacaaaggagaagtctcagatggctatagtgtctctagatcaaagacagaggatttcctcctcactctggagtccgctaccagctcccagacatctgtgtacttctgtgccatcagtgaggtaggggttgggcagccccagcattttggtgatgggactcgactctccatcctagaggacctgaacaaggtgttcccacccgaggtcgctgtgtttgagccatcagaagcagagatctcccacacccaaaaggccacactggtgtgcctggccacaggcttcttccccgaccacgtggagctgagctggtgggtgaatgggaaggaggtgcacagtggggtcagcacggacccgcagcccctcaaggagcagcccgccctcaatgactccagatactgcctgagcagccgcctgagggtctcggccaccttctggcagaacccccgcaaccacttccgctgtcaagtccagttctacgggctctcggagaatgacgagtggacccaggatagggccaaacccgtcacccagatcgtcagcgccgaggcctggggtagagcatgtggctttacctcgtcctaccagcaaggggtcctgtctgccaccatcctctatgagatcctgctagggaaggccaccctgtatgctgtgctggtcagcgcccttgtgttgatggccatggtcaagagaaaggatttc

SEQ ID NO: 178 - DNA, 822 bp, synthetic construct
  source 1..822: mol_type = other DNA
atgatgaaatccttgagagttttactagtgatcctgtggcttcagttgagctgggtttggagccaacagaaggaggtggagcagaattctggacccctcagtgttccagagggagccattgcctctctcaactgcacttacagtgaccgaggttcccagtccttcttctggtacagacaatattctgggaaaagccctgagttgataatgttcatatactccaatggtgacaaagaagatggaaggtttacagcacagctcaataaagccagccagtatgtttctctgctcatcagagactcccagcccagtgattcagccacctacctctgtgccgtgaacttcggaggaggaaagcttatcttcggacagggaacggagttatctgtgaaacccaatatccagaaccctgaccctgccgtgtaccagctgagagactctaaatccagtgacaagtctgtctgcctattcaccgattttgattctcaaacaaatgtgtcacaaagtaaggattctgatgtgtatatcacagacaaaactgtgctagacatgaggtctatggacttcaagagcaacagtgctgtggcctggagcaacaaatctgactttgcatgtgcaaacgccttcaacaacagcattattccagaagacaccttcttccccagcccagaaagttcctgtgatgtcaagctggtcgagaaaagctttgaaacagatacgaacctaaactttcaaaacctgtcagtgattgggttccgaatcctcctcctgaaagtggccgggtttaatctgctcatgacgctgcggctgtggtccagctga

SEQ ID NO: 179 - DNA, 918 bp, synthetic construct
  source 1..918: mol_type = other DNA
atgagaatcaggctcctgtgctgtgtggccttttctctcctgtgggcaggtccagtgattgctgggatcacccaggcaccaacatctcagatcctggcagcaggacggcgcatgacactgagatgtacccaggatatgagacataatgccatgtactggtatagacaagatctaggactggggctaaggctcatccattattcaaatactgcaggtaccactggcaaaggagaagtccctgatggttatagtgtctccagagcaaacacagatgatttccccctcacgttggcgtctgctgtaccctctcagacatctgtgtacttctgtgccagcagcctaagtttcggcactgaagctttctttggacaaggcaccagactcacagttgtagaggacctgaacaaggtgttcccacccgaggtcgctgtgtttgagccatcagaagcagagatctcccacacccaaaaggccacactggtgtgcctggccacaggcttcttccccgaccacgtggagctgagctggtgggtgaatgggaaggaggtgcacagtggggtcagcacggacccgcagcccctcaaggagcagcccgccctcaatgactccagatactgcctgagcagccgcctgagggtctcggccaccttctggcagaacccccgcaaccacttccgctgtcaagtccagttctacgggctctcggagaatgacgagtggacccaggatagggccaaacccgtcacccagatcgtcagcgccgaggcctggggtagagcatgtggctttacctcgtcctaccagcaaggggtcctgtctgccaccatcctctatgagatcctgctagggaaggccaccctgtatgctgtgctggtcagcgcccttgtgttgatggccatggtcaagagaaaggatttc

SEQ ID NO: 180 - protein, 250 aa, synthetic construct
GQQLNQSPQSMFIQEGEDVSMNCTSSSIFNTWLWYKQDPGEGPVLLIALYKAGELTSNGRLTAQFGITRKDSFLNISASIPSDVGIYFCAGGTGNQFYFGTGTSLTVIPNIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 181 - protein, 288 aa, synthetic construct
DAGITQSPRHKVTETGTPVTLRCHQTENHRYMYWYRQDPGHGLRLIHYSYGVKDTDKGEVSDGYSVSRSKTEDFLLTLESATSSQTSVYFCAISEVGVGQPQHFGDGTRLSILEDLNKVFPPEVAVFEPSEAEISHTQKATLVCLATGFFPDHVELSWWVNGKEVHSGVSTDPQPLKEQPALNDSRYCLSSRLRVSATFWQNPRNHFRCQVQFYGLSENDEWTQDRAKPVTQIVSAEAWGRACGFTSSYQQGVLSATILYEILLGKATLYAVLVSALVLMAMVKRKDF

SEQ ID NO: 182 - protein, 251 aa, synthetic construct
QKEVEQNSGPLSVPEGAIASLNCTYSDRGSQSFFWYRQYSGKSPELIMFIYSNGDKEDGRFTAQLNKASQYVSLLIRDSQPSDSATYLCAVNFGGGKLIFGQGTELSVKPNIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 183 - protein, 287 aa, synthetic construct
IAGITQAPTSQILAAGRRMTLRCTQDMRHNAMYWYRQDLGLGLRLIHYSNTAGTTGKGEVPDGYSVSRANTDDFPLTLASAVPSQTSVYFCASSLSFGTEAFFGQGTRLTVVEDLNKVFPPEVAVFEPSEAEISHTQKATLVCLATGFFPDHVELSWWVNGKEVHSGVSTDPQPLKEQPALNDSRYCLSSRLRVSATFWQNPRNHFRCQVQFYGLSENDEWTQDRAKPVTQIVSAEAWGRACGFTSSYQQGVLSATILYEILLGKATLYAVLVSALVLMAMVKRKDF

SEQ ID NO: 184 - protein, 9 aa, synthetic construct
FLWGPRALV

SEQ ID NO: 185 - protein, 9 aa, synthetic construct
KVAELVHFL

SEQ ID NO: 186 - DNA, 369 bp, synthetic construct
  source 1..369: mol_type = other DNA
atgggtcctgtcacctgctcagttcttgtgctcctcctaatgctcaggaggagcaatggcgatggagactccgtgacccagacagaaggcctggtcactctcacagaagggttgcctgtgatgctgaactgcacctatcagactatttactcaaatcctttccttttctggtatgtgcaacatctcaatgaatcccctcggctactcctgaagagcttcacagacaacaagaggaccgagcaccaagggttccacgccactctccataagagcagcagctccttccatctgcagaagtcctcagcgcagctgtcagactctgccctgtactactgtgctttcgacacaaatgcttacaaagtcatcttt

SEQ ID NO: 187 - DNA, 363 bp, synthetic construct
  source 1..363: mol_type = other DNA
atgagagttaggctcatctctgctgtggtgctgtgttccctaggaacaggccttgtggacatgaaagtaacccagatgccaagatacctgatcaaaagaatgggagagaatgttttgctggaatgtggacaggacatgagccatgaaacaatgtactggtatcgacaagaccctggtctggggctacagctgatttatatctcatacgatgttgatagtaacagcgaaggagacatccctaaaggatacagggtctcacggaagaagcgggagcatttctccctgattctggattctgctaaaacaaaccagacatctgtgtacttctgtgctagcagttcaacaaacacagaagtcttcttt

SEQ ID NO: 188 - DNA, 813 bp, synthetic construct
  source 1..813: mol_type = other DNA
atgggtcctgtcacctgctcagttcttgtgctcctcctaatgctcaggaggagcaatggcgatggagactccgtgacccagacagaaggcctggtcactctcacagaagggttgcctgtgatgctgaactgcacctatcagactatttactcaaatcctttccttttctggtatgtgcaacatctcaatgaatcccctcggctactcctgaagagcttcacagacaacaagaggaccgagcaccaagggttccacgccactctccataagagcagcagctccttccatctgcagaagtcctcagcgcagctgtcagactctgccctgtactactgtgctttcgacacaaatgcttacaaagtcatctttggaaaagggacacatcttcatgttctccctaacatccagaacccagaacctgctgtgtaccagttaaaagatcctcggtctcaggacagcaccctctgcctgttcaccgactttgactcccaaatcaatgtgccgaaaaccatggaatctggaacgttcatcactgacaaaactgtgctggacatgaaagctatggattccaagagcaatggggccattgcctggagcaaccagacaagcttcacctgccaagatatcttcaaagagaccaacaccacctaccccagttcagacgttccctgtgatgccacgttgactgagaaaagctttgaaacagatatgaacctaaactttcaaaacctgtcagttatgggactccgaatcctcctgctgaaagtagccggatttaacctgctcatgacgctgaggctgtggtccagttga

SEQ ID NO: 189 - DNA, 912 bp, synthetic construct
  source 1..912: mol_type = other DNA
atgagagttaggctcatctctgctgtggtgctgtgttccctaggaacaggccttgtggacatgaaagtaacccagatgccaagatacctgatcaaaagaatgggagagaatgttttgctggaatgtggacaggacatgagccatgaaacaatgtactggtatcgacaagaccctggtctggggctacagctgatttatatctcatacgatgttgatagtaacagcgaaggagacatccctaaaggatacagggtctcacggaagaagcgggagcatttctccctgattctggattctgctaaaacaaaccagacatctgtgtacttctgtgctagcagttcaacaaacacagaagtcttctttggtaaaggaaccagactcacagttgtagaggatctgagaaatgtgactccacccaaggtctccttgtttgagccatcaaaagcagagattgcaaacaaacaaaaggctaccctcgtgtgcttggccaggggcttcttccctgaccacgtggagctgagctggtgggtgaatggcaaggaggtccacagtggggtcagcacggaccctcaggcctacaaggagagcaattatagctactgcctgagcagccgcctgagggtctctgctaccttctggcacaatcctcgcaaccacttccgctgccaagtgcagttccatgggctttcagaggaggacaagtggccagagggctcacccaaacctgtcacacagaacatcagtgcagaggcctggggccgagcagactgtgggattacctcagcatcctatcaacaaggggtcttgtctgccaccatcctctatgagatcctgctagggaaagccaccctgtatgctgtgcttgtcagtacactggtggtgatggctatggtcaaaagaaagaactcgtga

SEQ ID NO: 190 - DNA, 371 bp, synthetic construct
  source 1..371: mol_type = other DNA
atggtcctagtgaccattctgctgctcagcgcgttcttctcactgagaggaaacagtgcccagtccgtggaccagcctgatgctcatgtcacgctctctgaaggagcctccctggagctcagatgcagttattcatacagtgcagcaccttacctcttctggtacgtgcagtatcctggccagagcctccagtttctcctcaaatacatcacaggagacaccgttgttaaaggcaccaagggctttgaggccgagtttaggaagagtaactcctctttcaacctgaagaaatccccagcccattggagcgactcagccaagtacttctgtgcactggagggcccggatacaggaaactacaaatacgtctt

SEQ ID NO: 191 - DNA, 375 bp, synthetic construct
  source 1..375: mol_type = other DNA
atgggcatccagaccctctgttgtgtgatcttttatgttctgatagcaaatcacacagatgctggagttacccagacacccagacatgaggtggcagagaaaggacaaacaataatcctgaagtgtgagccagtttcaggccacaatgaccttttctggtacagacagaccaagatacagggactagagttgctgagctacttccgcagcaagtctcttatggaagatggtggggctttcaaggatcgattcaaagctgagatgctaaattcatccttctccactctgaagattcaacctacagaacccagggactcagctgtgtatctgtgtgccagcagttttgggacagctagtgcagaaacgctgtatttt

SEQ ID NO: 192 - DNA, 816 bp, synthetic construct
  source 1..816: mol_type = other DNA
atggtcctagtgaccattctgctgctcagcgcgttcttctcactgagaggaaacagtgcccagtccgtggaccagcctgatgctcatgtcacgctctctgaaggagcctccctggagctcagatgcagttattcatacagtgcagcaccttacctcttctggtacgtgcagtatcctggccagagcctccagtttctcctcaaatacatcacaggagacaccgttgttaaaggcaccaagggctttgaggccgagtttaggaagagtaactcctctttcaacctgaagaaatccccagcccattggagcgactcagccaagtacttctgtgcactggagggcccggatacaggaaactacaaatacgtctttggagcaggtaccagactgaaggttatagcacacatccagaacccagaacctgctgtgtaccagttaaaagatcctcggtctcaggacagcaccctctgcctgttcaccgactttgactcccaaatcaatgtgccgaaaaccatggaatctggaacgttcatcactgacaaaactgtgctggacatgaaagctatggattccaagagcaatggggccattgcctggagcaaccagacaagcttcacctgccaagatatcttcaaagagaccaacgccacctaccccagttcagacgttccctgtgatgccacgttgactgagaaaagctttgaaacagatatgaacctaaacttccaaaacctgtcagttatgggactccgaatcctcctgctgaaagtagccggatttaacctgctcatgacgctgaggctgtggtccagttga

SEQ ID NO: 193 - DNA, 924 bp, synthetic construct
  source 1..924: mol_type = other DNA
atgggcatccagaccctctgttgtgtgatcttttatgttctgatagcaaatcacacagatgctggagttacccagacacccagacatgaggtggcagagaaaggacaaacaataatcctgaagtgtgagccagtttcaggccacaatgaccttttctggtacagacagaccaagatacagggactagagttgctgagctacttccgcagcaagtctcttatggaagatggtggggctttcaaggatcgattcaaagctgagatgctaaattcatccttctccactctgaagattcaacctacagaacccagggactcagctgtgtatctgtgtgccagcagttttgggacagctagtgcagaaacgctgtattttggctcaggaaccagactgactgttctcgaggatctgagaaatgtgactccacccaaggtctccttgtttgagccatcaaaagcagagattgcaaacaaacaaaaggctaccctcgtgtgcttggccaggggcttcttcccctgacacgtggagctgagctggtgggtgaatggcaaggaggtccacagtggggtcagcacggaccctcaggcctacaaggagagcaattatagctactgcctgagcagccgcctgagggtctctgctaccttctggcacaatcctcgaaaccacttccgctgtcaagtgcagttccatgggctttcagaggaggacaagtggccagagggctcacccaaacctgtcacacagaacatcagtgcagaggcctggggccgagcagactgtggaatcacttcagcatcctatcatcagggggttctgtctgcaaccatcctctatgagatcctactggggaaggccaccctatatgctgtgctggtcagtggcctggtgctgatggccatggtcaagaaaaaaaattcctga

SEQ ID NO: 194 - protein, 123 aa, synthetic construct
MGPVTCSVLVLLLMLRRSNGDGDSVTQTEGLVTLTEGLPVMLNCTYQTIYSNPFLFWYVQHLNESPRLLLKSFTDNKRTEHQGFHATLHKSSSSFHLQKSSAQLSDSALYYCAFDTNAYKVIF

SEQ ID NO: 195 - protein, 120 aa, synthetic construct
MRVRLISAVVLCSLGTGLVDMKVTQMPRYLIKRMGENVLLECGQDMSHETMYWYRQDPGLGLQLIYISYDVDSNSEGDIPKGYRVSRKKREHFSLILDSAKTNQTSVYFCASSSTNTEVF

SEQ ID NO: 196 - protein, 271 aa, synthetic construct
MGPVTCSVLVLLLMLRRSNGDGDSVTQTEGLVTLTEGLPVMLNCTYQTIYSNPFLFWYVQHLNESPRLLLKSFTDNKRTEHQGFHATLHKSSSSFHLQKSSAQLSDSALYYCAFDTNAYKVIFGKGTHLHVLPNIQNPEPAVYQLKDPRSQDSTLCLFTDFDSQINVPKTMESGTFITDKTVLDMKAMDSKSNGAIAWSNQTSFTCQDIFKETNTTYPSSDVPCDATLTEKSFETDMNLNFQNLSVMGLRILLLKVAGFNLLMTLRLWSSL

SEQ ID NO: 197 - protein, 295 aa, synthetic construct
MRVRLISAVVLCSLGTGLVDMKVTQMPRYLIKRMGENVLLECGQDMSHETMYWYRQDPGLGLQLIYISYDVDSNSEGDIPKGYRVSRKKREHFSLILDSAKTNQTSVYFCASSSTNTEVFFGKGTRLTVVEDLRNVTPPKVSLFEPSKAEIANKQKATLVCLARGFFPDHVELSWWVNGKEVHSGVSTDPQAYKESNYSYCLSSRLRVSATFWHNPRNHFRCQVQFHGLSEEDKWPEGSPKPVTQNISAEAWGRADCGITSASYQQGVLSATILYEILLGKATLYAVLVSTLVVM

SEQ ID NO: 198 - protein, 123 aa, synthetic construct
MVLVTILLLSAFFSLRGNSAQSVDQPDAHVTLSEGASLELRCSYSYSAAPYLFWYVQYPGQSLQFLLKYITGDTVVKGTKGFEAEFRKSNSSFNLKKSPAHWSDSAKYFCALEGPDTGNYKYV

SEQ ID NO: 199 - protein, 124 aa, synthetic construct
MGIQTLCCVIFYVLIANHTDAGVTQTPRHEVAEKGQTIILKCEPVSGHNDLFWYRQTKIQGLELLSYFRSKSLMEDGGAFKDRFKAEMLNSSFSTLKIQPTEPRDSAVYLCASSFGTASAETLY

SEQ ID NO: 200 - protein, 271 aa, synthetic construct
MVLVTILLLSAFFSLRGNSAQSVDQPDAHVTLSEGASLELRCSYSYSAAPYLFWYVQYPGQSLQFLLKYITGDTVVKGTKGFEAEFRKSNSSFNLKKSPAHWSDSAKYFCALEGPDTGNYKYVFGAGTRLKVIAHIQNPEPAVYQLKDPRSQDSTLCLFTDFDSQINVPKTMESGTFITDKTVLDMKAMDSKSNGAIAWSNQTSFTCQDIFKETNATYPSSDVPCDATLTEKSFETDMNLNFQNLSVMGLRILLLKVAGFNLLMTLRLWSS

SEQ ID NO: 201 - protein, 306 aa, synthetic construct
MGIQTLCCVIFYVLIANHTDAGVTQTPRHEVAEKGQTIILKCEPVSGHNDLFWYRQTKIQGLELLSYFRSKSLMEDGGAFKDRFKAEMLNSSFSTLKIQPTEPRDSAVYLCASSFGTASAETLYFGSGTRLTVLEDLRNVTPPKVSLFEPSKAEIANKQKATLVCLARGFFPHVELSWWVNGKEVHSGVSTDPQAYKESNYSYCLSSRLRVSATFWHNPRNHFRCQVQFHGLSEEDKWPEGSPKPVTQNISAEAWGRADCGITSASYHQGVLSATILYEILLGKATLYAVLVSGLVLMAMVKKKNS

SEQ ID NO: 202 - protein, 10 aa, synthetic construct
GVYDGREHTV

SEQ ID NO: 203 - protein, 9 aa, synthetic construct
FMNKFIYEI

SEQ ID NO: 204 - DNA, 846 bp, synthetic construct
  source 1..846: mol_type = other DNA
atgaagaagcacctgaccacctttctcgtgatcctgtggctgtacttctaccggggcaacggcaagaaccaggtggaacagagcccccagagcctgatcatcctggaaggcaagaactgcaccctgcagtgcaactacaccgtgtcccccttcagcaacctgcggtggtacaagcaggacaccggcagaggccctgtgtccctgaccatcctgaccttcagcgagaacaccaagagcaacggccggtacaccgccaccctggacgccgatacaaagcagagcagcctgcacatcaccgccagccagctgagcgatagcgccagctacatctgcgtggtgtccggcggcacagacagctggggcaagctgcagtttggcgccggaacacaggtggtcgtgacccccgacatccagaaccctgaccctgccgtgtaccagctgcgggacagcaagagcagcgacaagagcgtgtgcctgttcaccgacttcgacagccagaccaacgtgtcccagagcaaggacagcgacgtgtacatcaccgacaagaccgtgctggacatgcggagcatggacttcaagagcaatagcgccgtggcctggtccaacaagagcgacttcgcctgcgccaacgccttcaacaacagcattatccccgaggacacattcttcccaagccccgagagcagctgcgacgtcaagctggtggaaaagagcttcgagacagacaccaacctgaacttccagaacctgagcgtgatcggcttcagaatcctgctgctgaaggtggccggcttcaacctgctgatgaccctgagactgtggtccagcggcagccgggccaagaga

SEQ ID NO: 205 - DNA, 933 bp, synthetic construct
  source 1..933: mol_type = other DNA
atggccagcctgctgttcttctgcggcgccttctacctgctgggcaccggctctatggatgccgacgtgacccagaccccccggaacagaatcaccaagaccggcaagcggatcatgctggaatgctcccagaccaagggccacgaccggatgtactggtacagacaggaccctggcctgggcctgcggctgatctactacagcttcgacgtgaaggacatcaacaagggcgagatcagcgacggctacagcgtgtccagacaggctcaggccaagttcagcctgtccctggaaagcgccatccccaaccagaccgccctgtacttttgtgccacaagcggccagggcgcctacgaggagcagttctttggccctggcacccggctgacagtgctggaagatctgaagaacgtgttccccccagaggtggccgtgttcgagccttctgaggccgaaatcagccacacccagaaagccacactcgtgtgtctggccaccggcttctaccccgaccacgtggaactgtcttggtgggtcaacggcaaagaggtgcacagcggcgtgtccaccgatccccagcctctgaaagaacagcccgccctgaacgacagccggtactgcctgagcagcagactgagagtgtccgccaccttctggcagaaccccagaaaccacttcagatgccaggtgcagttttacggcctgagcgagaacgacgagtggacccaggacagagccaagcccgtgacacagatcgtgtctgccgaagcttgggggcgcgccgattgtggctttaccagcgagagctaccagcagggcgtgctgagcgccaccatcctgtacgagatcctgctgggaaaggccacactgtacgccgtgctggtgtctgccctggtgctgatggccatggtcaagcggaaggacagccggggc

SEQ ID NO: 206 - protein, 282 aa, synthetic construct
MKKHLTTFLVILWLYFYRGNGKNQVEQSPQSLIILEGKNCTLQCNYTVSPFSNLRWYKQDTGRGPVSLTILTFSENTKSNGRYTATLDADTKQSSLHITASQLSDSASYICVVSGGTDSWGKLQFGAGTQVVVTPDIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSSGSRAKR

SEQ ID NO: 207 - protein, 136 aa, synthetic construct
MKKHLTTFLVILWLYFYRGNGKNQVEQSPQSLIILEGKNCTLQCNYTVSPFSNLRWYKQDTGRGPVSLTILTFSENTKSNGRYTATLDADTKQSSLHITASQLSDSASYICVVSGGTDSWGKLQFGAGTQVVVTPD

SEQ ID NO: 208 - protein, 311 aa, synthetic construct
MASLLFFCGAFYLLGTGSMDADVTQTPRNRITKTGKRIMLECSQTKGHDRMYWYRQDPGLGLRLIYYSFDVKDINKGEISDGYSVSRQAQAKFSLSLESAIPNQTALYFCATSGQGAYEEQFFGPGTRLTVLEDLKNVFPPEVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 209 - protein, 133 aa, synthetic construct
MASLLFFCGAFYLLGTGSMDADVTQTPRNRITKTGKRIMLECSQTKGHDRMYWYRQDPGLGLRLIYYSFDVKDINKGEISDGYSVSRQAQAKFSLSLESAIPNQTALYFCATSGQGAYEEQFFGPGTRLTVLE

SEQ ID NO: 210 - protein, 105 aa, synthetic construct
MKNQVEQSPQSLIILEGKNCTLQCNYTVSPFSNLRWYKQDTGRGPVSLTIMTFSENTKSNGRYTATLDADTKQSSLHITASQLSDSASYICVVSGGTDSWGKLQF

SEQ ID NO: 211 - protein, 105 aa, synthetic construct
MKNQVEQSPQSLIILEGKNCTLQCNYTVSPFSNLRWYKQDTGRGPVSLTIVTFSENTKSNGRYTATLDADTKQSSLHITASQLSDSASYICVVSGGTDSWGKLQF

SEQ ID NO: 212 - protein, 105 aa, synthetic construct
MKNQVEQSPQSLIILEGKNCTLQCNYTVSPFSNLRWYKQDTGRGPVSLTILTFSENTKSNGRYTATLDADTKQSSLHITASQLSDSASYICVVSGGTDSWGKLQF

SEQ ID NO: 213 - protein, 123 aa, synthetic construct
MASLLFFCGAFYLLGTGSMDADVTQTPRNRITKTGKRIMLECSQTKGHDRMYWYRQDPGLGLRLIYYSFDVKDINKGEISDGYSVSRQAQAKFSLSLESAIPNQTALYFCATSGQGAYNEQFF

SEQ ID NO: 214 - protein, 123 aa, synthetic construct
MASLLFFCGAFYLLGTGSMDADVTQTPRNRITKTGKRIMLECSQTKGHDRMYWYRQDPGLGLRLIYYSFDVKDINKGEISDGYSVSRQAQAKFSLSLESAIPNQTALYFCATSGQGAYEEQFF

SEQ ID NO: 215 - protein, 9 aa, synthetic construct
VLDFAPPGA

SEQ ID NO: 216 - protein, 9 aa, synthetic construct
RMFPNAPYL

SEQ ID NO: 217 - DNA, 396 bp, synthetic construct
  source 1..396: mol_type = other DNA
atggagacactgctgggactactgattctgtggctgcaactgcaatgggtgagcagcaaacaggaggttacccagattcctgctgctctgtctgttcctgaaggcgagaatctggtgctgaactgcagcttcacagatagcgccatctacaacctgcagtggttcagacaggatcctggaaaaggcctgacaagcctgctgctgattcagagctctcagagagagcagacatctggaagactgaatgctagcctggacaagtctagcggcagaagcaccctgtatattgccgcctctcaacctggagattctgccacatacctgtgtgctgtgaaggagacatctggctctagactgacctttggcgagggaacacaactgaccgtgaatcctgac

SEQ ID NO: 218 - DNA, 417 bp, synthetic construct
  source 1..417: mol_type = other DNA
atgaccagagttagcctgttatgggctgtggtggtgagcacatgtctggaatctggaatggcccagacagtgacacagtctcagcctgaaatgtctgtgcaggaagccgaaaccgttacactgagctgcacctacgatacaagcgagaacaactactacctgttctggtacaagcagcccccctctaggcagatgatcctggtgatcagacaggaggcctataaacagcagaatgccacagagaaccggttcagcgtgaacttccagaaagccgccaagagcttcagcctgaagatctctgattctcagctgggcgatacagccatgtacttttgcgccttcatctaccccagctacacaagcggcacatacaagtacatcttcggcaccggcacaagactgaaggttctggccaac

SEQ ID NO: 219 - DNA, 420 bp, synthetic construct
  source 1..420: mol_type = other DNA
atggccatgttactaggagcgagcgtgctgattctgtggttacagcctgattgggtgaactctcagcagaagaacgatgatcagcaggtgaagcagaacagcccctctctgtctgtgcaggaaggcagaatcagcatcctgaattgcgattacaccaacagcatgttcgactacttcctgtggtacaagaagtaccccgccgagggccctacctttctgatcagcatctctagcatcaaggacaagaacgaagatggcagattcaccgtgttcctgaacaagagcgccaagcacctgagcctgcacattgtgccttctcaacctggagattctgccgtgtacttttgtgctgcctctggaacaggcggaagctatatccccacatttggaagaggaacaagcctgatcgtgcacccttac

SEQ ID NO: 220 - DNA, 417 bp, synthetic construct
  source 1..417: mol_type = other DNA
atggccatgttactaggagcgagcgtgctgattctgtggttacagcctgattgggtgaactctcagcagaagaacgatgatcagcaggtgaagcagaacagcccctctctgtctgtgcaggaaggcagaatcagcatcctgaattgcgattacaccaacagcatgttcgactacttcctgtggtacaagaagtaccccgccgagggccctacctttctgatcagcatctctagcatcaaggacaagaacgaagatggcagattcaccgtgttcctgaacaagagcgccaagcacctgagcctgcacattgtgccttctcaacctggagattctgccgtgtacttttgtgctgcctctggcattggcgactacaaactgagctttggagccggcacaacagtgaccgttagagccaat

SEQ ID NO: 221 - DNA, 393 bp, synthetic construct
  source 1..393: mol_type = other DNA
atggtgaagatccggcagttcctcctggctattctgtggctgcaactgtcttgtgtgtctgctgccaagaatgaagtggagcagtctccccagaaccttacagcccaggaaggcgagtttatcaccatcaactgcagctattctgtgggcattagcgccctgcattggctgcagcaacaccctggaggaggaattgtgtctctgtttatgctgtcttctggcaagaagaagcacggccggctgattgccaccatcaacatccaggagaagcactcttctctgcacattacagcctctcatcccagggattctgccgtgtacatctgtgccgtgagaaccagctacgataaggtgattttcggaccaggcacctctctgagcgtgatccccaat

SEQ ID NO: 222 - DNA, 408 bp, synthetic construct
  source 1..408: mol_type = other DNA
atgaagagcctgagagtcctgctggtgattttgtggctgcagctgtcttgggtttggtctcagcagaaagaagtggagcagaatagcggccctctgtctgttcctgaaggcgctattgctagcctgaattgcacatacagcgatagaggatctcagagcttcttctggtaccggcagtacagcggcaagagcccagaactgatcatgttcatctacagcaatggcgacaaggaggatggcaggtttacagcccagctgaacaaggccagccagtatgtttctctgctgatcagagatagccagcctagcgattctgccacctacctgtgtgccgtgaacttacttggagctacaggatactctacactgaccttcggcaaaggcaccatgctgctggtgagccctgat

SEQ ID NO: 223 - DNA, 384 bp, synthetic construct
  source 1..384: mol_type = other DNA
atgtggggcgttttccttctgtatgtgagcatgaagatgggcggcacaacaggccagaacatcgatcagcctaccgagatgacagccacagaaggagctattgttcagatcaactgcacctaccagacaagcggcttcaacggcctgttctggtaccagcagcatgctggagaagctcctacatttctgagctacaatgtgctggatggcctggaggagaaaggcaggtttagcagcttcctgagcaggtctaagggctattcttatctgctgctgaaggagctgcagatgaaggattccgccagctacctgtgtgccgttaggggcatcaatgattacaagctgagctttggagccggaacaacagtgaccgtgagagccaac

SEQ ID NO: 224 - DNA, 393 bp, synthetic construct
  source 1..393: mol_type = other DNA
atggagaagatgctggagtgtgcgttcatcgttctgtggctgcaacttggatggctgtctggagaggatcaggttacacagtctcctgaagccctgagactgcaagaaggagaaagctctagcctgaactgcagctacacagtgtctggactgagaggcctgttctggtacagacaggatcctggaaaaggcccagagttcctgtttaccctgtattctgccggcgaggagaaggagaaagagagactgaaagctaccctgaccaagaaggagagcttcctgcacattaccgcccccaaacctgaggattctgccacatatctgtgtgccgtgattaccggctttcagaagctggtgtttggcacaggcaccagactgctggtttctcccaat

SEQ ID NO: 225 - DNA, 384 bp, synthetic construct
  source 1..384: mol_type = other DNA
atgagactggtggcacgcgtaactgtgtttctgacctttggcaccatcatcgatgccaagacaacccagcctacaagcatggactgtgccgagggaagagctgctaatctgccatgtaatcacagcacaatcagcggcaacgagtacgtgtactggtaccggcagatccactctcaaggacctcagtacatcattcatggcctgaagaacaacgagaccaacgagatggccagcctgatcatcaccgaggacaggaagtcttctaccctgattctgcctcatgctacactgagagataccgccgtgtactactgcattgccggagtgggaagaggccagaatttcgtgtttggacctggaacaagactgagcgttctgccctat

SEQ ID NO: 226 - DNA, 408 bp, synthetic construct
  source 1..408: mol_type = other DNA
atggagaagaaccccttggcagcacctctgcttattctgtggttccacctggattgtgtgagcagcatcctgaatgtggagcagtctcctcagagcctgcatgtgcaagaaggcgatagcaccaatttcacctgcagctttccaagcagcaacttctacgccctgcactggtacagatgggaaaccgccaaatctcctgaagccctgtttgtgatgaccctgaatggcgacgagaagaagaagggcagaattagcgccaccctgaataccaaggagggctacagctacctgtacatcaagggctctcaacctgaggattctgccacctacctttgcgcctttcaccccaatttcggcaacgagaaactgacctttggaaccggaacaaggctgaccatcatccccaac

SEQ ID NO: 227 - DNA, 411 bp, synthetic construct
  source 1..411: mol_type = other DNA
atggagaagatgctggagtgtgcgttcatcgttctgtggctgcaacttggatggctgtctggagaggatcaggttacacagtctcctgaagccctgagactgcaagaaggagaaagctctagcctgaactgcagctacacagtgtctggactgagaggcctgttctggtacagacaggatcctggaaaaggcccagagttcctgtttaccctgtattctgccggcgaggagaaggagaaagagagactgaaagctaccctgaccaagaaggagagcttcctgcacattaccgcccccaaacctgaggattctgccacatatctgtgtgctgttcagcctagaggagatggctctagcaataccggcaagctgatctttggccagggaacaacactgcaggtgaagcctgat

SEQ ID NO: 228 - DNA, 423 bp, synthetic construct
  source 1..423: mol_type = other DNA
atccagaatcccgatcctgctgtgtaccagctgcgggacagcaagagcagcgacaagagcgtgtgcctgttcaccgacttcgacagccagaccaacgtgtcccagagcaaggacagcgacgtgtacatcaccgataagtgcgtgctggacatgcggagcatggacttcaagagcaacagcgccgtggcctggtccaacaagagcgacttcgcctgcgccaacgccttcaacaacagcattatccccgaggacacattcttcccaagccccgagagcagctgcgacgtgaagctggtggaaaagagcttcgagacagacaccaacctgaacttccagaacctcagcgtgatcggcttccggatcctgctgctgaaggtggccggcttcaacctgctgatgaccctgcggctgtggtccagctga

SEQ ID NO: 229 - DNA, 440 bp, synthetic construct
  source 1..440: mol_type = other DNA
ctcaataaaagagcccacaacccctcactcggcgcgccaccatgggcacatctcttctctgttgggtggttctgggctttctgggcacagatcatacaggagctggagttagccagtctcctaggtataaggtgaccaagaggggacaggatgtggctctgagatgtgaccctattagcggacatgtgagcctgtactggtacagacaagctctgggacaaggacccgagtttctgacctacttcaactatgaggcccagcaggacaaatctggactgcccaacgacagattcagcgccgaaagaccagaaggctctattagcacactgaccatccagagaacagagcagagggattctgccatgtacagatgcgccagcagcttaacaggctcttacgagcagtactttggacctggcacaagactgacagtgacagag

SEQ ID NO: 230 - DNA, 440 bp, synthetic construct
  source 1..440: mol_type = other DNA
ctcaataaaagagcccacaacccctcactcggcgcgccaccatgctgcttcttctcctccttctcggacctgctggatctggattaggagctgttgtgtctcagcacccttcttgggtgatctgtaaaagcggcacaagcgtgaagatcgagtgcagaagcctggactttcaggccacaaccatgttctggtataggcagttccccaagcagtctctgatgctgatggccacctctaatgagggctctaaggccacatatgaacagggagtggagaaggacaagttcctgatcaaccacgcctctctgaccctgtctaccctgacagttacatctgcccaccctgaggatagcagcttttacatctgtagcgccacacctgaagcctctagcccatatgagcagtactttggccctggcaccagattaacagtgacagag

SEQ ID NO: 231 - DNA, 440 bp, synthetic construct
  source 1..440: mol_type = other DNA
ctcaataaaagagcccacaacccctcactcggcgcgccaccatgggacctggactgcttcattggatggctctgtgtttgctgggaacaggacatggagatgctatggtgatccagaaccccaggtatcaggtgacccagtttggcaaaccagtgacactgagctgttctcagaccctgaaccacaacgtgatgtactggtaccagcagaagtcttctcaggcccctaagctgctgttccactactacgacaaggacttcaacaacgaggccgatacccctgacaatttccagagcaggaggcccaataccagcttctgtttcctggacattagaagccctggactgggagatgctgccatgtacctgtgtgccaccagcaatttacagggaagacaacctcagcactttggcgatggcacaaggctgtctatcctggag

SEQ ID NO: 232 - DNA, 470 bp, synthetic construct
  source 1..470: mol_type = other DNA
ctcaataaaagagcccacaacccctcactcggcgcgccaccatgctgagccctgatctccctgattctgcctggaataccagactgctgtgtcatgtgatgctgtgtctgcttggagccgtttctgtggctgctggcgtgattcaatctcctagacacctgatcaaggagaagagagaaacagccaccctgaagtgctaccccatccccagacacgatacagtgtactggtatcagcaaggacctggacaagatccccagttcctgatcagcttctacgagaagatgcagagcgacaaaggcagcatcccagacagatttagcgcccagcagtttagcgactatcactctgagctgaacatgagcagcctggaactgggcgattctgctctgtacttctgtgcctcttctctgagactgggaagagaaacccagtactttggacccggcacaagactgctggttcttgag

SEQ ID NO: 233 - DNA, 440 bp, synthetic construct
  source 1..440: mol_type = other DNA
ctcaataaaagagcccacaacccctcactcggcgcgccaccatgggcacaagacttctctgctgggtggtgcttggatttctgggcacagatcatacaggagctggagttagccagtctcctaggtacaaagtggccaagagaggacaggatgtggctctgagatgtgaccctattagcggacatgtgagcctgttttggtaccagcaagctctgggacaaggacccgagtttctgacctacttccagaatgaagcccagctggataaatctggactgcctagcgaccggttcttcgccgaaagacctgaaggatctgttagcaccctgaagattcagagaacacagcaggaggactctgccgtgtacctgtgtgcctcttctttaggacaggcctatgagcagtattttggacctggcaccagactgaccgtgacagag

SEQ ID NO: 234 - DNA, 440 bp, synthetic construct
  source 1..440: mol_type = other DNA
ctcaataaaagagcccacaacccctcactcggcgcgccaccatgggcacaagacttctctgctgggtggccttttgtctgctggtggaagagctgattgaagctggagttgtgcagtctcctaggtacaagatcatcgagaagaagcagcccgtggccttctggtgtaatcccatttctggccacaacaccctgtactggtatctgcagaatctgggacagggccctgaactgctgatcagatacgagaacgaagaagccgtggacgattctcaactgcctaaggaccgcttttctgccgagaggctgaaaggagtggattctaccctgaagatccaacctgctgaactgggcgattctgctgtgtacctgtgcgcttctagcctgacaagaggagctgaagccttttttggacagggcacaagactgacagtggtggag

SEQ ID NO: 235 - DNA, 440 bp, synthetic construct
  source 1..440: mol_type = other DNA
ctcaataaaagagcccacaacccctcactcggcgcgccaccatgggacctcagcttcttggatacgttgtgctgtgtctgcttggagctggacctcttgaagctcaggttacccagaaccccagatacctgattaccgtgacaggcaaaaagctgaccgtgacatgtagccagaacatgaaccacgagtacatgagctggtaccggcaggatcctggattaggcctgagacagatctactacagcatgaacgtggaggtgaccgataaaggcgacgtgcctgagggatacaaggtgagcagaaaggagaagaggaatttccccctgatcctggaaagcccaagccccaatcagacaagcctgtacttttgtgccagcagcttttctggcggcacatatgagcagtacttcggccctggcacaagactgacagttacagag

SEQ ID NO: 236 - DNA, 473 bp, synthetic construct
  source 1..473: mol_type = other DNA
ctcaataaaagagcccacaacccctcactcggcgcgccaccatgctgagccctgatctccctgattctgcctggaataccagactgctgtgtcatgtgatgctgtgtctgcttggagccgtttctgtggctgctggcgtgattcaatctcctagacacctgatcaaggagaagagagaaacagccaccctgaagtgctaccccatccccagacacgatacagtgtactggtatcagcaaggacctggacaagatccccagttcctgatcagcttctacgagaagatgcagagcgacaaaggcagcatcccagacagatttagcgcccagcagtttagcgactatcactctgagctgaacatgagcagcctggaactgggcgattctgctctgtacttctgtgccagcagctatagaggaggcagcacatatgagcagtactttggccctggcacaagactgacagtgacagag

SEQ ID NO: 237 - DNA, 446 bp, synthetic construct
  source 1..446: mol_type = other DNA
ctcaataaaagagcccacaacccctcactcggcgcgccaccatgagcaccagactcctttgctggatggctttgtgtctgcttggagctgagctgtctgaagctgaagttgcccagtctcccagatacaagatcaccgagaaatctcaggctgtggccttctggtgtgaccctatttctggacacgccaccctgtactggtataggcaaattctgggacaaggccctgaactgctggtgcaatttcaggatgagagcgtggtggacgattctcaactgcctaaggacaggttttctgccgagcggctgaaaggagttgatagcaccctgaagatccaacctgctgaactgggcgattctgctatgtacctgtgcgcctcttctcagagagatagccctaacgagaagctgttctttggctctggaacccagctgtctgtgctggag

SEQ ID NO: 238 - DNA, 449 bp, synthetic construct
  source 1..449: mol_type = other DNA
ctcaataaaagagcccacaacccctcactcggcgcgccaccatgggctgtagactgttgtgttgtgctgtgctgtgtctgttgggagctgtgcctatggaaacaggcgttacccagacacctagacatctggttatgggcatgaccaacaagaagagcctgaagtgcgagcagcatctgggccataacgccatgtactggtataagcagagcgccaagaaaccactggaactgatgttcgtgtacagcctggaggagagggtggagaataatagcgtgcccagcagatttagccctgagtgcccaaattcttctcacctgttcctgcacctgcacacattacagcccgaggattctgccctgtacctgtgtgcttcttctcaagacccttacaagctgagcggcaataccatctacttcggcgaaggctcttggctgacagtggttgaa

SEQ ID NO: 239 - DNA, 528 bp, synthetic construct
  source 1..528: mol_type = other DNA
gatctgaacaaggtgttccccccagaggtggccgtgttcgagccttctgaggccgagatctcccacacccagaaagccaccctcgtgtgcctggccaccggctttttccccgaccacgtggaactgtcttggtgggtcaacggcaaagaggtgcactccggcgtgtgcaccgatccccagcctctgaaagaacagcccgccctgaacgacagccggtactgcctgagcagcagactgagagtgtccgccaccttctggcagaacccccggaaccacttcagatgccaggtgcagttctacggcctgagcgagaacgacgagtggacccaggacagagccaagcccgtgacacagatcgtgtctgccgaagcctggggcagagccgattgcggctttacctccgtgtcctatcagcagggcgtgctgagcgccacaatcctgtacgagatcctgctgggcaaggccaccctgtacgccgtgctggtgtctgccctggtgctgatggccatggtcaagcggaaggacttc

SEQ ID NO: 240 - DNA, 534 bp, synthetic construct
  source 1..534: mol_type = other DNA
gacctgaagaacgtgttccccccagaggtggccgtgttcgagcctagcgaggccgagatcagccacacccagaaagccaccctcgtgtgcctggccaccggcttttaccccgaccacgtggaactgtcttggtgggtcaacggcaaagaggtgcacagcggcgtctgcaccgacccccagcccctgaaagagcagcccgccctgaacgacagccggtactgtctgagcagcagactgagagtgtccgccaccttctggcagaacccccggaaccacttcagatgccaggtgcagttctacggcctgagcgagaacgacgagtggacccaggaccgggccaagcccgtgacccagatcgtgtctgctgaggcctggggcagagccgattgcggcttcaccagcgagagctaccagcagggcgtgctgagcgccaccatcctgtacgagatcctgctgggcaaggccaccctgtacgccgtgctggtgtccgccctggtgctgatggccatggtcaagcggaaggacagccggggc

SEQ ID NO: 241 - DNA, 819 bp, synthetic construct
  source 1..819: mol_type = other DNA
atgaaatccttgagagttttactagtgatcctgtggcttcagttgagctgggtttggagccaacagaaggaggtggagcagaattctggacccctcagtgttccagagggagccattgcctctctcaactgcacttacagtgaccgaggttcccagtccttcttctggtacagacaatattctgggaaaagccctgagttgataatgttcatatactccaatggtgacaaagaagatggaaggtttacagcacagctcaataaagccagccagtatgtttctctgctcatcagagactcccagcccagtgattcagccacctacctctgtgccgtgaacataggaaaccatgacatgcgctttggagcagggaccagactgacagtaaaaccaaatatccagaaccctgaccctgccgtgtaccagctgagagactctaaatccagtgacaagtctgtctgcctattcaccgattttgattctcaaacaaatgtgtcacaaagtaaggattctgatgtgtatatcacagacaaaactgtgctagacatgaggtctatggacttcaagagcaacagtgctgtggcctggagcaacaaatctgactttgcatgtgcaaacgccttcaacaacagcattattccagaagacaccttcttccccagcccagaaagttcctgtgatgtcaagctggtcgagaaaagctttgaaacagatacgaacctaaactttcaaaacctgtcagtgattgggttccgaatcctcctcctgaaagtggccgggtttaatctgctcatgacgctgcggctgtggtccagctga

SEQ ID NO: 242 - DNA, 819 bp, synthetic construct
  source 1..819: mol_type = other DNA
atggagaaaatgttggagtgtgcattcatagtcttgtggcttcagcttggctggttgagtggagaagaccaggtgacgcagagtcccgaggccctgagactccaggagggagagagtagcagtctcaactgcagttacacagtcagcggtttaagagggctgttctggtataggcaagatcctgggaaaggccctgaattcctcttcaccctgtattcagctggggaagaaaaggagaaagaaaggctaaaagccacattaacaaagaaggaaagctttctgcacatcacagcccctaaacctgaagactcagccacttatctctgtgctgtgcagaccatggacggtaaccagttctattttgggacagggacaagtttgacggtcattccaaatatccagaaccctgaccctgccgtgtaccagctgagagactctaaatccagtgacaagtctgtctgcctattcaccgattttgattctcaaacaaatgtgtcacaaagtaaggattctgatgtgtatatcacagacaaaactgtgctagacatgaggtctatggacttcaagagcaacagtgctgtggcctggagcaacaaatctgactttgcatgtgcaaacgccttcaacaacagcattattccagaagacaccttcttccccagcccagaaagttcctgtgatgtcaagctggtcgagaaaagctttgaaacagatacgaacctaaactttcaaaacctgtcagtgattgggttccgaatcctcctcctgaaagtggccgggtttaatctgctcatgacgctgcggctgtggtccagctga

SEQ ID NO: 243 - DNA, 825 bp, synthetic construct
  source 1..825: mol_type = other DNA
atggcatgccctggcttcctgtgggcacttgtgatctccacctgtcttgaatttagcatggctcagacagtcactcagtctcaaccagagatgtctgtgcaggaggcagagaccgtgaccctgagctgcacatatgacaccagtgagagtgattattatttattctggtacaagcagcctcccagcaggcagatgattctcgttattcgccaagaagcttataagcaacagaatgcaacagagaatcgtttctctgtgaacttccagaaagcagccaaatccttcagtctcaagatctcagactcacagctgggggatgccgcgatgtatttctgtgcttccagtccaggaacctacaaatacatctttggaacaggcaccaggctgaaggttttagcaaatatccagaaccctgaccctgccgtgtaccagctgagagactctaaatccagtgacaagtctgtctgcctattcaccgattttgattctcaaacaaatgtgtcacaaagtaaggattctgatgtgtatatcacagacaaaactgtgctagacatgaggtctatggacttcaagagcaacagtgctgtggcctggagcaacaaatctgactttgcatgtgcaaacgccttcaacaacagcattattccagaagacaccttcttccccagcccagaaagttcctgtgatgtcaagctggtcgagaaaagctttgaaacagatacgaacctaaactttcaaaacctgtcagtgattgggttccgaatcctcctcctgaaagtggccgggtttaatctgctcatgacgctgcggctgtggtccagctga

SEQ ID NO: 244 - DNA, 834 bp, synthetic construct
  source 1..834: mol_type = other DNA
atgacacgagttagcttgctgtgggcagtcgtggtctccacctgtcttgaatccggcatggcccagacagtcactcagtctcaaccagagatgtctgtgcaggaggcagagactgtgaccctgagttgcacatatgacaccagtgagagtaattattatttgttctggtacaaacagcctcccagcaggcagatgattctcgttattcgccaagaagcttataagcaacagaatgcaacggagaatcgtttctctgtgaacttccagaaagcagccaaatccttcagtctcaagatctcagactcacagctgggggacactgcgatgtatttctgtgctttcaacccttgggagaactatggtcagaattttgtctttggtcccggaaccagattgtccgtgctgccctatatccagaaccctgaccctgccgtgtaccagctgagagactctaaatccagtgacaagtctgtctgcctattcaccgattttgattctcaaacaaatgtgtcacaaagtaaggattctgatgtgtatatcacagacaaaactgtgctagacatgaggtctatggacttcaagagcaacagtgctgtggcctggagcaacaaatctgactttgcatgtgcaaacgccttcaacaacagcattattccagaagacaccttcttccccagcccagaaagttcctgtgatgtcaagctggtcgagaaaagctttgaaacagatacgaacctaaactttcaaaacctgtcagtgattgggttccgaatcctcctcctgaaagtggccgggtttaatctgctcatgacgctgcggctgtggtccagctga

SEQ ID NO: 245 - DNA, 819 bp, synthetic construct
  source 1..819: mol_type = other DNA
atgaagagcctgagagtcctgctggtgattttgtggctgcagctgtcttgggtttggtctcagcagaaagaagtggagcagaatagcggccctctgtctgttcctgaaggcgctattgctagcctgaattgcacatacagcgatagaggatctcagagcttcttctggtaccggcagtacagcggcaagagcccagaactgatcatgttcatctacagcaatggcgacaaggaggatggcaggtttacagcccagctgaacaaggccagccagtatgtttctctgctgatcagagatagccagcctagcgattctgccacctacctgtgtgccgtgaacatcggaaatcacgacatgagatttggagccggcacaagactgaccgtgaagcccaatatccagaaccctgatcctgctgtgtaccagctgcgggacagcaagagcagcgacaagagcgtgtgcctgttcaccgacttcgacagccagaccaacgtgtcccagagcaaggacagcgacgtgtacatcaccgataagtgcgtgctggacatgcggagcatggacttcaagagcaacagcgccgtggcctggtccaacaagagcgacttcgcctgcgccaacgccttcaacaacagcattatccccgaggacacattcttcccaagccccgagagcagctgcgacgtgaagctggtggaaaagagcttcgagacagacaccaacctgaacttccagaacctcagcgtgatcggcttccggatcctgctgctgaaggtggccggcttcaacctgctgatgaccctgcggctgtggtccagctga

SEQ ID NO: 246 - DNA, 819 bp, synthetic construct
  source 1..819: mol_type = other DNA
atggagaagatgctggagtgtgcgttcatcgttctgtggctgcaacttggatggctgtctggagaggatcaggttacacagtctcctgaagccctgagactgcaagaaggagaaagctctagcctgaactgcagctacacagtgtctggactgagaggcctgttctggtacagacaggatcctggaaaaggcccagagttcctgtttaccctgtattctgccggcgaggagaaggagaaagagagactgaaagctaccctgaccaagaaggagagcttcctgcacattaccgcccccaaacctgaggattctgccacatatctgtgtgctgtgcagaccatggatggcaaccagttctacttcggcacaggcacatctctgaccgttatccccaatatccagaaccctgatcctgccgtgtaccagctgcgggacagcaagagcagcgacaagagcgtgtgcctgttcaccgacttcgacagccagaccaacgtgtcccagagcaaggacagcgacgtgtacatcaccgataagtgcgtgctggacatgcggagcatggacttcaagagcaacagcgccgtggcctggtccaacaagagcgacttcgcctgcgccaacgccttcaacaacagcattatccccgaggacacattcttcccaagccccgagagcagctgcgacgtgaagctggtggaaaagagcttcgagacagacaccaacctgaacttccagaacctcagcgtgatcggcttccggatcctgctgctgaaggtggccggcttcaacctgctgatgaccctgcggctgtggtccagctga

SEQ ID NO: 247 - DNA, 825 bp, synthetic construct
  source 1..825: mol_type = other DNA
atggcttgtcctggattcttatgggctctggtgatcagcacctgtctggagttctctatggcccagacagtgacacagtctcagcctgaaatgtctgtgcaggaagccgaaaccgtgacactgtcttgcacctacgatacaagcgagagcgactactacctgttctggtacaagcagcctccctctaggcagatgatcctggtgattagacaggaggcctacaaacagcagaatgccaccgagaaccggtttagcgtgaacttccagaaagccgccaagagcttcagcctgaaaatctctgacagccagctgggagatgctgccatgtacttttgtgccagctctccaggcacctacaagtacatttttggcaccggcaccagactgaaggtgctggccaatatccagaatcccgatcctgccgtgtaccagctgcgggacagcaagagcagcgacaagagcgtgtgcctgttcaccgacttcgacagccagaccaacgtgtcccagagcaaggacagcgacgtgtacatcaccgataagtgcgtgctggacatgcggagcatggacttcaagagcaacagcgccgtggcctggtccaacaagagcgacttcgcctgcgccaacgccttcaacaacagcattatccccgaggacacattcttcccaagccccgagagcagctgcgacgtgaagctggtggaaaagagcttcgagacagacaccaacctgaacttccagaacctcagcgtgatcggcttccggatcctgctgctgaaggtggccggcttcaacctgctgatgaccctgcggctgtggtccagctga

SEQ ID NO: 248 - DNA, 834 bp, synthetic construct
  source 1..834: mol_type = other DNA
atgaccagagttagcctgttatgggctgtggtggtgagcacatgtctggaatctggaatggcccagacagtgacacagtctcagcctgaaatgtctgtgcaggaagccgaaaccgttacactgagctgcacctacgatacaagcgagagcaactactacctgttctggtacaagcagcccccttctaggcagatgatcctggtgatcagacaggaggcctataaacagcagaatgccaccgagaaccggtttagcgtgaacttccagaaagccgccaagagcttcagcctgaaaatctctgacagccagctgggcgatacagccatgtacttttgtgccttcaacccctgggagaactatggccagaatttcgtgttcggccctggcaccagactgtctgttctgccttatatccagaaccccgatcctgctgtgtaccagctgcgggacagcaagagcagcgacaagagcgtgtgcctgttcaccgacttcgacagccagaccaacgtgtcccagagcaaggacagcgacgtgtacatcaccgataagtgcgtgctggacatgcggagcatggacttcaagagcaacagcgccgtggcctggtccaacaagagcgacttcgcctgcgccaacgccttcaacaacagcattatccccgaggacacattcttcccaagccccgagagcagctgcgacgtgaagctggtggaaaagagcttcgagacagacaccaacctgaacttccagaacctcagcgtgatcggcttccggatcctgctgctgaaggtggccggcttcaacctgctgatgaccctgcggctgtggtccagctga

SEQ ID NO: 249 - DNA, 939 bp, synthetic construct
  source 1..939: mol_type = other DNA
atgggctgcaggctgctctgctgtgcggttctctgtctcctgggagcagttcccatagacactgaagttacccagacaccaaaacacctggtcatgggaatgacaaataagaagtctttgaaatgtgaacaacatatggggcacagggctatgtattggtacaagcagaaagctaagaagccaccggagctcatgtttgtctacagctatgagaaactctctataaatgaaagtgtgccaagtcgcttctcacctgaatgccccaacagctctctcttaaaccttcacctacacgccctgcagccagaagactcagccctgtatctctgcgccagcagccaagggactagcggggcagatacgcagtattttggcccaggcacccggctgacagtgctcgaggacctgaaaaacgtgttcccacccgaggtcgctgtgtttgagccatcagaagcagagatctcccacacccaaaaggccacactggtgtgcctggccacaggcttctaccccgaccacgtggagctgagctggtgggtgaatgggaaggaggtgcacagtggggtcagcacagacccgcagcccctcaaggagcagcccgccctcaatgactccagatactgcctgagcagccgcctgagggtctcggccaccttctggcagaacccccgcaaccacttccgctgtcaagtccagttctacgggctctcggagaatgacgagtggacccaggatagggccaaacctgtcacccagatcgtcagcgccgaggcctggggtagagcagactgtggcttcacctccgagtcttaccagcaaggggtcctgtctgccaccatcctctatgagatcttgctagggaaggccaccttgtatgccgtgctggtcagtgccctcgtgctgatggccatggtcaagagaaaggattccagaggctag

SEQ ID NO: 250 - DNA, 942 bp, synthetic construct
  source 1..942: mol_type = other DNA
atgagcatcggcctcctgtgctgtgcagccttgtctctcctgtgggcaggtccagtgaatgctggtgtcactcagaccccaaaattccaggtcctgaagacaggacagagcatgacactgcagtgtgcccaggatatgaaccatgaatacatgtcctggtatcgacaagacccaggcatggggctgaggctgattcattactcagttggtgctggtatcactgaccaaggagaagtccccaatggctacaatgtctccagatcaaccacagaggatttcccgctcaggctgctgtcggctgctccctcccagacatctgtgtacttctgtgccagcagttactctctttgggaccttcaagagacccagtacttcgggccaggcacgcggctcctggtgctcgaggacctgaaaaacgtgttcccacccgaggtcgctgtgtttgagccatcagaagcagagatctcccacacccaaaaggccacactggtgtgcctggccacaggcttctaccccgaccacgtggagctgagctggtgggtgaatgggaaggaggtgcacagtggggtcagcacagacccgcagcccctcaaggagcagcccgccctcaatgactccagatactgcctgagcagccgcctgagggtctcggccaccttctggcagaacccccgcaaccacttccgctgtcaagtccagttctacgggctctcggagaatgacgagtggacccaggatagggccaaacctgtcacccagatcgtcagcgccgaggcctggggtagagcagactgtggcttcacctccgagtcttaccagcaaggggtcctgtctgccaccatcctctatgagatcttgctagggaaggccaccttgtatgccgtgctggtcagtgccctcgtgctgatggccatggtcaagagaaaggattccagaggctag

SEQ ID NO: 251 - DNA, 945 bp, synthetic construct
  source 1..945: mol_type = other DNA
atgggcaccagcctcctctgctggatggccctgtgtctcctgggggcagatcacgcagatactggagtctcccaggaccccagacacaagatcacaaagaggggacagaatgtaactttcaggtgtgatccaatttctgaacacaaccgcctttattggtaccgacagaccctggggcagggcccagagtttctgacttacttccagaatgaagctcaactagaaaaatcaaggctgctcagtgatcggttctctgcagagaggcctaagggatctttctccaccttggagatccagcgcacagagcagggggactcggccatgtatctctgtgccagcagcttttcagacgggggggctacagatacgcagtattttggcccaggcacccggctgacagtgctcgaggacctgaaaaacgtgttcccacccgaggtcgctgtgtttgagccatcagaagcagagatctcccacacccaaaaggccacactggtgtgcctggccacaggcttctaccccgaccacgtggagctgagctggtgggtgaatgggaaggaggtgcacagtggggtcagcacagacccgcagcccctcaaggagcagcccgccctcaatgactccagatactgcctgagcagccgcctgagggtctcggccaccttctggcagaacccccgcaaccacttccgctgtcaagtccagttctacgggctctcggagaatgacgagtggacccaggatagggccaaacctgtcacccagatcgtcagcgccgaggcctggggtagagcagactgtggcttcacctccgagtcttaccagcaaggggtcctgtctgccaccatcctctatgagatcttgctagggaaggccaccttgtatgccgtgctggtcagtgccctcgtgctgatggccatggtcaagagaaaggattccagaggctag

SEQ ID NO: 252 - DNA, 933 bp, synthetic construct
  source 1..933: mol_type = other DNA
atgctgctgcttctgctgcttctggggccagcaggctccgggcttggtgctgtcgtctctcaacatccgagctgggttatctgtaagagtggaacctctgtgaagatcgagtgccgttccctggactttcaggccacaactatgttttggtatcgtcagttcccgaaacagagtctcatgctgatggcaacttccaatgagggctccaaggccacatacgagcaaggcgtcgagaaggacaagtttctcatcaaccatgcaagcctgaccttgtccactctgacagtgaccagtgcccatcctgaagacagcagcttctacatctgcagtgctagaccccattctctcacagatacgcagtattttggcccaggcacccggctgacagtgctcgaggacctgaaaaacgtgttcccacccgaggtcgctgtgtttgagccatcagaagcagagatctcccacacccaaaaggccacactggtgtgcctggccacaggcttctaccccgaccacgtggagctgagctggtgggtgaatgggaaggaggtgcacagtggggtcagcacagacccgcagcccctcaaggagcagcccgccctcaatgactccagatactgcctgagcagccgcctgagggtctcggccaccttctggcagaacccccgcaaccacttccgctgtcaagtccagttctacgggctctcggagaatgacgagtggacccaggatagggccaaacctgtcacccagatcgtcagcgccgaggcctggggtagagcagactgtggcttcacctccgagtcttaccagcaaggggtcctgtctgccaccatcctctatgagatcttgctagggaaggccaccttgtatgccgtgctggtcagtgccctcgtgctgatggccatggtcaagagaaaggattccagaggctag

SEQ ID NO: 253 - DNA, 936 bp, synthetic construct
  source 1..936: mol_type = other DNA
atgggctgtagactgttgtgttgtgctgtgctgtgtctgttgggagctgtgcctatcgatacagaggtgacccagacccctaaacatctggttatgggcatgaccaacaagaagagcctgaagtgcgagcagcacatgggccatagggccatgtattggtataagcagaaggccaagaaacctcctgagctgatgttcgtgtacagctacgagaagctgagcatcaacgagagcgtgcccagcagattttctcctgagtgccctaattctagcctgctgaatctgcacctgcatgctctgcagcctgaggattctgctctgtacctgtgtgcttcttctcagggcacatctggagctgatacacagtacttcggacctggcacaagactgacagtgctggaagacctgaagaacgtgttccccccagaggtggccgtgttcgagcctagcgaggccgagatcagccacacccagaaagccaccctcgtgtgcctggccaccggcttttaccccgaccacgtggaactgtcttggtgggtcaacggcaaagaggtgcacagcggcgtctgcaccgacccccagcccctgaaagagcagcccgccctgaacgacagccggtactgtctgagcagcagactgagagtgtccgccaccttctggcagaacccccggaaccacttcagatgccaggtgcagttctacggcctgagcgagaacgacgagtggacccaggaccgggccaagcccgtgacccagatcgtgtctgctgaggcctggggcagagccgattgcggcttcaccagcgagagctaccagcagggcgtgctgagcgccaccatcctgtacgagatcctgctgggcaaggccaccctgtacgccgtgctggtgtccgccctggtgctgatggccatggtcaagcggaaggacagccggggc

SEQ ID NO: 254 - DNA, 939 bp, synthetic construct
  source 1..939: mol_type = other DNA
atgtctatcggtctgctgtgctgtgctgctctttctctgctttgggctggacctgtgaatgctggagttacacaaacccccaagttccaagtgctgaagacaggacagagcatgaccctgcagtgtgctcaggacatgaatcacgagtacatgagctggtacagacaggatcctggaatgggcctgaggctgatccactactctgttggagccggaattacagatcagggagaagtgccaaatggctacaacgtgagcaggagcacaaccgaggacttccccttaagactgttgtctgctgctccatctcagacaagcgtgtacttttgcgccagctcctactctctgtgggatctgcaggaaacccagtactttggaccaggcacaagactgttagtgctggaggacctgaagaacgtgttccccccagaggtggccgtgttcgagcctagcgaggccgagatcagccacacccagaaagccaccctcgtgtgcctggccaccggcttttaccccgaccacgtggaactgtcttggtgggtcaacggcaaagaggtgcacagcggcgtctgcaccgacccccagcccctgaaagagcagcccgccctgaacgacagccggtactgtctgagcagcagactgagagtgtccgccaccttctggcagaacccccggaaccacttcagatgccaggtgcagttctacggcctgagcgagaacgacgagtggacccaggaccgggccaagcccgtgacccagatcgtgtctgctgaggcctggggcagagccgattgcggcttcaccagcgagagctaccagcagggcgtgctgagcgccaccatcctgtacgagatcctgctgggcaaggccaccctgtacgccgtgctggtgtccgccctggtgctgatggccatggtcaagcggaaggacagccggggc

SEQ ID NO: 255 - DNA, 942 bp, synthetic construct
  source 1..942: mol_type = other DNA
atgggcacatctcttctctgctggatggctctttgtctgcttggagccgatcatgccgatacaggagttagccaggatcctagacacaagatcaccaagagaggccagaatgtgaccttccggtgcgatcctatctctgagcacaacaggctgtactggtacagacaaacactgggacaaggacctgagttcctgacctacttccagaacgaagcccagctggagaagtctagacttctgagcgacagatttagcgccgagagacctaaaggcagctttagcaccctggagatccagagaacagaacagggcgattctgccatgtacctgtgtgctagcagcttttctgatggaggcgccaccgatacacagtatttcggacctggcacaagactgacagtgctggaggacctgaagaacgtgttccccccagaggtggccgtgttcgagcctagcgaggccgagatcagccacacccagaaagccaccctcgtgtgcctggccaccggcttttaccccgaccacgtggaactgtcttggtgggtcaacggcaaagaggtgcacagcggcgtctgcaccgacccccagcccctgaaagagcagcccgccctgaacgacagccggtactgtctgagcagcagactgagagtgtccgccaccttctggcagaacccccggaaccacttcagatgccaggtgcagttctacggcctgagcgagaacgacgagtggacccaggaccgggccaagcccgtgacccagatcgtgtctgctgaggcctggggcagagccgattgcggcttcaccagcgagagctaccagcagggcgtgctgagcgccaccatcctgtacgagatcctgctgggcaaggccaccctgtacgccgtgctggtgtccgccctggtgctgatggccatggtcaagcggaaggacagccggggc

SEQ ID NO: 256 - DNA, 930 bp, synthetic construct
  source 1..930: mol_type = other DNA
atgctgcttcttctcctccttctcggacctgctggatctggattaggagctgttgtgtctcagcacccttcttgggtgatctgtaaaagcggcacaagcgtgaagatcgagtgcagaagcctggactttcaggccacaaccatgttctggtataggcagttccccaagcagtctctgatgctgatggccacctctaatgagggctctaaggccacatatgaacagggagtggagaaggacaagttcctgatcaaccacgcctctctgaccctgtctaccctgacagttacatctgcccaccctgaggatagcagcttttacatctgtagcgccagacctcacagcctgaccgatacacagtactttggccctggcacaagactgacagtgttagaagacctgaagaacgtgttccccccagaggtggccgtgttcgagcctagcgaggccgagatcagccacacccagaaagccaccctcgtgtgcctggccaccggcttttaccccgaccacgtggaactgtcttggtgggtcaacggcaaagaggtgcacagcggcgtctgcaccgacccccagcccctgaaagagcagcccgccctgaacgacagccggtactgtctgagcagcagactgagagtgtccgccaccttctggcagaacccccggaaccacttcagatgccaggtgcagttctacggcctgagcgagaacgacgagtggacccaggaccgggccaagcccgtgacccagatcgtgtctgctgaggcctggggcagagccgattgcggcttcaccagcgagagctaccagcagggcgtgctgagcgccaccatcctgtacgagatcctgctgggcaaggccaccctgtacgccgtgctggtgtccgccctggtgctgatggccatggtcaagcggaaggacagccggggc

SEQ ID NO: 257 - protein, 131 aa, synthetic construct
METLLGLLILWLQLQWVSSKQEVTQIPAALSVPEGENLVLNCSFTDSAIYNLQWFRQDPGKGLTSLLLIQSSQREQTSGRLNASLDKSSGRSTLYIAASQPGDSATYLCAVKETSGSRLTFGEGTQLTVNP

SEQ ID NO: 258 - protein, 139 aa, synthetic construct
MTRVSLLWAVVVSTCLESGMAQTVTQSQPEMSVQEAETVTLSCTYDTSENNYYLFWYKQPPSRQMILVIRQEAYKQQNATENRFSVNFQKAAKSFSLKISDSQLGDTAMYFCAFIYPSYTSGTYKYIFGTGTRLKVLAN

SEQ ID NO: 259 - protein, 140 aa, synthetic construct
MAMLLGASVLILWLQPDWVNSQQKNDDQQVKQNSPSLSVQEGRISILNCDYTNSMFDYFLWYKKYPAEGPTFLISISSIKDKNEDGRFTVFLNKSAKHLSLHIVPSQPGDSAVYFCAASGTGGSYIPTFGRGTSLIVHPY

SEQ ID NO: 260 - protein, 139 aa, synthetic construct
MAMLLGASVLILWLQPDWVNSQQKNDDQQVKQNSPSLSVQEGRISILNCDYTNSMFDYFLWYKKYPAEGPTFLISISSIKDKNEDGRFTVFLNKSAKHLSLHIVPSQPGDSAVYFCAASGIGDYKLSFGAGTTVTVRAN

SEQ ID NO: 261 - protein, 131 aa, synthetic construct
MVKIRQFLLAILWLQLSCVSAAKNEVEQSPQNLTAQEGEFITINCSYSVGISALHWLQQHPGGGIVSLFMLSSGKKKHGRLIATINIQEKHSSLHITASHPRDSAVYICAVRTSYDKVIFGPGTSLSVIPN

SEQ ID NO: 262 - protein, 135 aa, synthetic construct
MKSLRVLLVILWLQLSWVWSQQKEVEQNSGPLSVPEGAIASLNCTYSDRGSQSFFWYRQYSGKSPELIMFIYSNGDKEDGRFTAQLNKASQYVSLLIRDSQPSDSATYLCAVNLLGATGYSTLTFGKGTMLLVSP

SEQ ID NO: 263 - protein, 128 aa, synthetic construct
MWGVFLLYVSMKMGGTTGQNIDQPTEMTATEGAIVQINCTYQTSGFNGLFWYQQHAGEAPTFLSYNVLDGLEEKGRFSSFLSRSKGYSYLLLKELQMKDSASYLCAVRGINDYKLSFGAGTTVTVRAN

SEQ ID NO: 264 - protein, 131 aa, synthetic construct
MEKMLECAFIVLWLQLGWLSGEDQVTQSPEALRLQEGESSSLNCSYTVSGLRGLFWYRQDPGKGPEFLFTLYSAGEEKEKERLKATLTKKESFLHITAPKPEDSATYLCAVITGFQKLVFGTGTRLLVSPN

SEQ ID NO: 265 - protein, 128 aa, synthetic construct
MRLVARVTVFLTFGTIIDAKTTQPTSMDCAEGRAANLPCNHSTISGNEYVYWYRQIHSQGPQYIIHGLKNNETNEMASLIITEDRKSSTLILPHATLRDTAVYYCIAGVGRGQNFVFGPGTRLSVLPY

SEQ ID NO: 266 - protein, 136 aa, synthetic construct
MEKNPLAAPLLILWFHLDCVSSILNVEQSPQSLHVQEGDSTNFTCSFPSSNFYALHWYRWETAKSPEALFVMTLNGDEKKKGRISATLNTKEGYSYLYIKGSQPEDSATYLCAFHPNFGNEKLTFGTGTRLTIIPN

SEQ ID NO: 267 - protein, 136 aa, synthetic construct
MEKMLECAFIVLWLQLGWLSGEDQVTQSPEALRLQEGESSSLNCSYTVSGLRGLFWYRQDPGKGPEFLFTLYSAGEEKEKERLKATLTKKESFLHITAPKPEDSATYLCAVQPRGDGSSNTGKLIFGQGTTLQVKP

SEQ ID NO: 268 - protein, 140 aa, synthetic construct
IQNPDPAVYQLRDSKSSDKSVCLFTDFDSQTNVSQSKDSDVYITDKCVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 269 - protein, 133 aa, synthetic construct
MGTSLLCWVVLGFLGTDHTGAGVSQSPRYKVTKRGQDVALRCDPISGHVSLYWYRQALGQGPEFLTYFNYEAQQDKSGLPNDRFSAERPEGSISTLTIQRTEQRDSAMYRCASSLTGSYEQYFGPGTRLTVTE

SEQ ID NO: 270 - protein, 133 aa, synthetic construct
MLLLLLLLGPAGSGLGAVVSQHPSWVICKSGTSVKIECRSLDFQATTMFWYRQFPKQSLMLMATSNEGSKATYEQGVEKDKFLINHASLTLSTLTVTSAHPEDSSFYICSATPEASSPYEQYFGPGTRLTVTE

SEQ ID NO: 271 - protein, 133 aa, synthetic construct
MGPGLLHWMALCLLGTGHGDAMVIQNPRYQVTQFGKPVTLSCSQTLNHNVMYWYQQKSSQAPKLLFHYYDKDFNNEADTPDNFQSRRPNTSFCFLDIRSPGLGDAAMYLCATSNLQGRQPQHFGDGTRLSILE

SEQ ID NO: 272 - protein, 143 aa, synthetic construct
MLSPDLPDSAWNTRLLCHVMLCLLGAVSVAAGVIQSPRHLIKEKRETATLKCYPIPRHDTVYWYQQGPGQDPQFLISFYEKMQSDKGSIPDRFSAQQFSDYHSELNMSSLELGDSALYFCASSLRLGRETQYFGPGTRLLVLE

SEQ ID NO: 273 - protein, 133 aa, synthetic construct
MGTRLLCWVVLGFLGTDHTGAGVSQSPRYKVAKRGQDVALRCDPISGHVSLFWYQQALGQGPEFLTYFQNEAQLDKSGLPSDRFFAERPEGSVSTLKIQRTQQEDSAVYLCASSLGQAYEQYFGPGTRLTVTE

SEQ ID NO: 274 - protein, 133 aa, synthetic construct
MGTRLLCWVAFCLLVEELIEAGVVQSPRYKIIEKKQPVAFWCNPISGHNTLYWYLQNLGQGPELLIRYENEEAVDDSQLPKDRFSAERLKGVDSTLKIQPAELGDSAVYLCASSLTRGAEAFFGQGTRLTVVE

SEQ ID NO: 275 - protein, 134 aa, synthetic construct
MSNQVLCCVVLCFLGANTVDGGITQSPKYLFRKEGQNVTLSCEQNLNHDAMYWYRQDPGQGLRLIYYSQIVNDFQKGDIAEGYSVSREKKESFPLTVTSAQKNPTAFYLCASSRDREQESPLHFGNGTRLTVTE

SEQ ID NO: 276 - protein, 133 aa, synthetic construct
MGPQLLGYVVLCLLGAGPLEAQVTQNPRYLITVTGKKLTVTCSQNMNHEYMSWYRQDPGLGLRQIYYSMNVEVTDKGDVPEGYKVSRKEKRNFPLILESPSPNQTSLYFCASSFSGGTYEQYFGPGTRLTVTE

SEQ ID NO: 277 - protein, 144 aa, synthetic construct
MLSPDLPDSAWNTRLLCHVMLCLLGAVSVAAGVIQSPRHLIKEKRETATLKCYPIPRHDTVYWYQQGPGQDPQFLISFYEKMQSDKGSIPDRFSAQQFSDYHSELNMSSLELGDSALYFCASSYRGGSTYEQYFGPGTRLTVTE

SEQ ID NO: 278 - protein, 135 aa, synthetic construct
MSTRLLCWMALCLLGAELSEAEVAQSPRYKITEKSQAVAFWCDPISGHATLYWYRQILGQGPELLVQFQDESVVDDSQLPKDRFSAERLKGVDSTLKIQPAELGDSAMYLCASSQRDSPNEKLFFGSGTQLSVLE

SEQ ID NO: 279 - protein, 136 aa, synthetic construct
MGCRLLCCAVLCLLGAVPMETGVTQTPRHLVMGMTNKKSLKCEQHLGHNAMYWYKQSAKKPLELMFVYSLEERVENNSVPSRFSPECPNSSHLFLHLHTLQPEDSALYLCASSQDPYKLSGNTIYFGEGSWLTVVE

SEQ ID NO: 280 - protein, 176 aa, synthetic construct
DLNKVFPPEVAVFEPSEAEISHTQKATLVCLATGFFPDHVELSWWVNGKEVHSGVCTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDF

SEQ ID NO: 281 - protein, 178 aa, synthetic construct
DLKNVFPPEVAVFEPSEAEISHTQKATLVCLATGFYPDHVELSWWVNGKEVHSGVCTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 282 - protein, 132 aa, synthetic construct
MKSLRVLLVILWLQLSWVWSQQKEVEQNSGPLSVPEGAIASLNCTYSDRGSQSFFWYRQYSGKSPELIMFIYSNGDKEDGRFTAQLNKASQYVSLLIRDSQPSDSATYLCAVNIGNHDMRFGAGTRLTVKPN

SEQ ID NO: 283 - protein, 132 aa, synthetic construct
MEKMLECAFIVLWLQLGWLSGEDQVTQSPEALRLQEGESSSLNCSYTVSGLRGLFWYRQDPGKGPEFLFTLYSAGEEKEKERLKATLTKKESFLHITAPKPEDSATYLCAVQTMDGNQFYFGTGTSLTVIPN

SEQ ID NO: 284 - protein, 134 aa, synthetic construct
MACPGFLWALVISTCLEFSMAQTVTQSQPEMSVQEAETVTLSCTYDTSESDYYLFWYKQPPSRQMILVIRQEAYKQQNATENRFSVNFQKAAKSFSLKISDSQLGDAAMYFCASSPGTYKYIFGTGTRLKVLAN

SEQ ID NO: 285 - protein, 137 aa, synthetic construct
MTRVSLLWAVVVSTCLESGMAQTVTQSQPEMSVQEAETVTLSCTYDTSESNYYLFWYKQPPSRQMILVIRQEAYKQQNATENRFSVNFQKAAKSFSLKISDSQLGDTAMYFCAFNPWENYGQNFVFGPGTRLSVLPY

SEQ ID NO: 286 - protein, 140 aa, synthetic construct; identical to SEQ ID NO: 268

SEQ ID NO: 287 - protein, 134 aa, synthetic construct
MGCRLLCCAVLCLLGAVPIDTEVTQTPKHLVMGMTNKKSLKCEQHMGHRAMYWYKQKAKKPPELMFVYSYEKLSINESVPSRFSPECPNSSLLNLHLHALQPEDSALYLCASSQGTSGADTQYFGPGTRLTVLE

SEQ ID NO: 288 - protein, 135 aa, synthetic construct
MSIGLLCCAALSLLWAGPVNAGVTQTPKFQVLKTGQSMTLQCAQDMNHEYMSWYRQDPGMGLRLIHYSVGAGITDQGEVPNGYNVSRSTTEDFPLRLLSAAPSQTSVYFCASSYSLWDLQETQYFGPGTRLLVLE

SEQ ID NO: 289 - protein, 136 aa, synthetic construct
MGTSLLCWMALCLLGADHADTGVSQDPRHKITKRGQNVTFRCDPISEHNRLYWYRQTLGQGPEFLTYFQNEAQLEKSRLLSDRFSAERPKGSFSTLEIQRTEQGDSAMYLCASSFSDGGATDTQYFGPGTRLTVLE

SEQ ID NO: 290 - protein, 132 aa, synthetic construct
MLLLLLLLGPAGSGLGAVVSQHPSWVICKSGTSVKIECRSLDFQATTMFWYRQFPKQSLMLMATSNEGSKATYEQGVEKDKFLINHASLTLSTLTVTSAHPEDSSFYICSARPHSLTDTQYFGPGTRLTVLE

SEQ ID NO: 291 - protein, 178 aa, synthetic construct; identical to SEQ ID NO: 281